(19) United States
(12) Patent Application Publication
(10) Pub. No.: US 2019/0262397 A1
(43) Pub. Date: Aug. 29, 2019

(54) CHIMERIC ANTIGEN RECEPTOR

(71) Applicant: Tessa Therapeutics Pte. Ltd., Singapore (SG)

(72) Inventors: John Edward CONNOLLY, SINGAPORE (SG); Dang L. VU, Singapore (SG)

(21) Appl. No.: 16/320,055

(22) PCT Filed: Jul. 24, 2017
(86) PCT No.: PCT/EP2017/068654

§ 371 Date: Jan. 23, 2019

Related U.S. Application Data
(60) Provisional application No. 62/366,729, filed on Jul. 26, 2016.
(60) Provisional application No. 62/366,731, filed on Jul. 26, 2016.

(51) Int. Cl. A61K 35/17; A61P 31/00; C07K 16/30; C07K 14/705

(52) U.S. Cl. CPC A61K 35/17; A61P 31/00; C07K 14/70578; C07K 14/70521; C07K 14/70517; C07K 16/303

(57) ABSTRACT

Chimeric Antigen Receptors (CARs) comprising a costimulatory sequence which is, or which is derived from, the intracellular domain of CD226, or a fragment thereof, are disclosed. Also disclosed are compositions comprising such CARs, and uses and methods using the same.

### FIELD OF THE INVENTION

The present invention relates to chimeric antigen receptors (CARs), nucleic acids encoding and cells expressing the same, and medical uses thereof.

### BACKGROUND TO THE INVENTION

Immunotherapy with genetically modified T cells has shown great promise in the treatment of hematologic malignancies. The addition of chimeric antigen receptors (CARs) has proven to be a particularly useful approach to generate tumor-specific T cells.

The basic CAR is made up of an ectodomain, derived either from a single-chain variable fragment (scFV) or a recombinant affinity ligand, a structural hinge region, a transmembrane domain, and a cytoplasmic endodomain with signaling domains derived from CD3ζ with or without additional co-stimulatory molecules.

Whilst CAR-T cells have been successful in early phase clinical studies treating CD19-positive hematological malignancies, the success of CARs in solid tumors has been greatly hampered by the lack of unique tumor associated antigens, inefficient homing of T cells to tumor sites and an inability to overcome the immunosuppressive microenvironment of solid tumors.

GPC3 (Glypican 3 also known as DGSX, GTR2-2, MXR7, OCI-5, SDYS, SGB, SGBS and SGBS1) is a cell surface protein of the glypican family of heparan sulphate proteoglycans. GPC3 is not expressed in normal adult liver tissue, but is expressed in hepatocellular carcinoma (Shirakawa et al. 2009 Intl J Oncol 34: 649-656; Ho et al. 2011 Eur J Cancer 47(3):333-338). GPC3 expression has also been observed in other cancers such as melanoma, ovarian clear-cell carcinoma, yolk sac tumors, neuroblastoma, hepatoblastoma, and Wilms' tumor cells (Ho et al. 2011 Eur J Cancer 47(3):333-338). GPC3 is therefore a candidate target for cancer therapy.

EP 2995 682 A1, Gao et al., Clin Cancer Res 20(24): 6418-6428 and WO 2016/049459 A1 disclose CARs comprising a GPC3-binding domain, and cells comprising the CARs.

### SUMMARY OF THE INVENTION

The present invention provides chimeric antigen receptors (CARs), and cells expressing CARs, having desirable or improved properties.

The present invention provides a chimeric antigen receptor (CAR), comprising a costimulatory sequence which is, or which is derived from, the intracellular domain of CD226, or a fragment thereof. In some embodiments, the costimulatory sequence which is, or which is derived from, the intracellular domain of CD226, or a fragment thereof comprises or consists of an amino acid sequence having at least 80% sequence identity to the amino acid sequence of SEQ ID NO:16, 58 or 59.

In some embodiments, the CAR additionally comprises a costimulatory sequence which comprises or consists of an amino acid sequence which is, or which is derived from, the intracellular domain of CD28. In some embodiments, the CAR additionally comprises a costimulatory sequence which comprises or consists of an amino acid sequence which is, or which is derived from, the intracellular domain of 4-1 BB. In some embodiments, the CAR comprises a costimulatory sequence which comprises or consists of an amino acid sequence having at least 80% sequence identity to the amino acid sequence of SEQ ID NO:17. In some embodiments, the CAR comprises a costimulatory sequence which comprises or consists of an amino acid sequence having at least 80% sequence identity to the amino acid sequence of SEQ ID NO:18.

In some embodiments, the CAR additionally comprises a dimerization domain. In some embodiments, the dimerization domain is an inducible dimerization domain. In some embodiments, the dimerization domain comprises or consists of an amino acid sequence having at least 80% sequence identity to the amino acid sequence of SEQ ID NO:20.

In some embodiments, the CAR comprises a transmembrane domain which comprises or consists of an amino acid sequence which is, or which is derived from, the transmembrane domain of CD28, CD8α or CD226. In some embodiments, the CAR comprises a transmembrane domain which comprises or consists of an amino acid sequence having at least 80% sequence identity to the amino acid sequence of SEQ ID NO:11, 10 or 57.

In some embodiments, the CAR additionally comprises a hinge region. In some embodiments, the hinge region is, or is derived from, the human IgG1 hinge region. In some embodiments, the hinge region comprises or consists of an amino acid sequence having at least 80% sequence identity to the amino acid sequence of SEQ ID NO:19.

In some embodiments, the CAR comprises an antigen-binding domain which comprises:
a heavy chain variable region sequence having at least 85% sequence identity to the amino acid sequence of SEQ ID NO:1, and
a light chain variable region sequence having at least 85% sequence identity to the amino acid sequence of SEQ ID NO:5.

In some embodiments, the CAR comprises an antigen-binding domain which comprises:
a heavy chain variable region sequence having at least 85% sequence identity to the amino acid sequence of SEQ ID NO:48, and
a light chain variable region sequence having at least 85% sequence identity to the amino acid sequence of SEQ ID NO:52.

In another aspect, the present invention provides a chimeric antigen receptor (CAR) according to any one of A, B, C, D, E, F, G, H, I, J, K, L or M as shown in Table 1, or V, W, X, Z, AA, BB, CC, DD, EE, FF, GG, HH, II, JJ, KK, LL or MM as shown in Table 3.

In another aspect, the present invention provides a chimeric antigen receptor (CAR) comprising, or consisting of, an amino acid sequence having at least 60% sequence identity to the amino acid sequence of SEQ ID NO:22, 23, 24, 25, 26, 27, 28, 29, 38, 39, 40, 41, 42, 81, 83, 84, 85, 86, 88, 89, 90, 92, 93, 94, 95, 96, 97 or 98.

In another aspect, the present invention provides a chimeric antigen receptor (CAR) comprising, or consisting of, an amino acid sequence having at least 60% sequence identity to the amino acid sequence of SEQ ID NO:30, 31, 32, 33, 34, 35, 36, 37, 43, 44, 45, 46, 47, 62, 64, 65, 66, 67, 69, 70, 71, 73, 74, 75, 76, 77, 78 or 79.

In another aspect, the present invention provides a chimeric antigen receptor (CAR) which is capable of binding to GPC3, comprising: a GPC3-binding domain, a hinge region, a transmembrane domain, and a signalling domain; wherein the hinge region comprises or consists of an amino acid sequence which is, or which is derived from, the human IgG1 hinge region, and; wherein the transmembrane domain comprises or consists of an amino acid sequence which is, or which is derived from, the transmembrane domain of CD8α

In some embodiments, the hinge region comprises or consists of an amino acid sequence having at least 80% sequence identity to the amino acid sequence of SEQ ID NO:19, and wherein the transmembrane domain comprises or consists of an amino acid sequence having at least 80% sequence identity to the amino acid sequence of SEQ ID NO:11.

In another aspect, the present invention provides a chimeric antigen receptor (CAR) which is capable of binding to GPC3, comprising: a GPC3-binding domain, a transmembrane domain, a signalling domain, and an inducible dimerization domain.

In some embodiments, the dimerization domain comprises or consists of an amino acid sequence which is, or which is derived from, the amino acid sequence of F36V-FKBP.

In some embodiments, a CAR according to the present invention comprises a dimerization domain which comprises or consists of an amino acid sequence having at least 80% sequence identity to the amino acid sequence of SEQ ID NO:20.

In another aspect, the present invention provides a chimeric antigen receptor (CAR) which is capable of binding to GPC3, comprising: a GPC3-binding domain, a transmembrane domain, and a signalling domain; wherein the signalling domain comprises a costimulatory sequence which comprises or consists of an amino acid sequence which is, or which is derived from, the intracellular domain of CD226.

In some embodiments, a CAR according to the present invention comprises a signalling domain which comprises a costimulatory sequence which comprises or consists of an amino acid sequence having at least 80% sequence identity to the amino acid sequence of SEQ ID NO:16.

In some embodiments, a CAR according to the present invention comprises a signalling domain which comprises a costimulatory sequence which comprises or consists of an amino acid sequence which is, or which is derived from, the intracellular domain of CD28. In some embodiments, a CAR according to the present invention comprises a signalling domain which comprises a costimulatory sequence which comprises or consists of an amino acid sequence which is, or which is derived from, the intracellular domain of 4-1BB.

In some embodiments, a CAR according to the present invention comprises a signalling domain which comprises a costimulatory sequence which comprises or consists of an amino acid sequence having at least 80% sequence identity to the amino acid sequence of SEQ ID NO:17.

In some embodiments, a CAR according to the present invention comprises a signalling domain which comprises a costimulatory sequence which comprises or consists of an amino acid sequence having at least 80% sequence identity to the amino acid sequence of SEQ ID NO:18.

In another aspect, the present invention provides a chimeric antigen receptor (CAR) which is capable of binding to GPC3 according to any one of A, B, C, D, E, F, G, H, I, J, K, L or M as shown in Table 1 herein.

In another aspect, the present invention provides a chimeric antigen receptor (CAR) which is capable of binding to GPC3, comprising or consisting of, an amino acid sequence having at least 60% sequence identity to the amino acid sequence of SEQ ID NO:38, 39, 40, 22, 23, 41, 42, 24, 25, 26, 27, 28 or 29.

In another aspect, the present invention provides a chimeric antigen receptor (CAR) which is capable of binding to GPC3, comprising or consisting of, an amino acid sequence having at least 60% sequence identity to the amino acid sequence of SEQ ID NO:43, 44, 45, 30, 31, 46, 47, 32, 33, 34, 35, 36 or 37.

In another aspect, the present invention provides a nucleic acid encoding the chimeric antigen receptor (CAR) according to the present invention.

In another aspect, the present invention provides a vector comprising the nucleic acid according to the present invention.

In another aspect, the present invention provides a cell comprising the chimeric antigen receptor (CAR), the nucleic acid, or the vector according to the present invention.

In another aspect, the present invention provides a method for producing a cell expressing a chimeric antigen receptor (CAR), comprising introducing into a cell a nucleic acid or a vector according to the present invention, and culturing the cell under conditions suitable for expression of the nucleic acid or vector by the cell.

In another aspect, the present invention provides a cell which is obtained or obtainable by the method according to the present invention.

In another aspect, the present invention provides a pharmaceutical composition comprising a chimeric antigen receptor (CAR), nucleic acid, vector, or cell according to the present invention, and a pharmaceutically acceptable carrier, adjuvant, excipient, or diluent.

In another aspect, the present invention provides a chimeric antigen receptor (CAR), nucleic acid, vector, cell, or pharmaceutical composition according to the present invention for use in a method of treating or preventing a disease or disorder.

In another aspect, the present invention provides the use of a chimeric antigen receptor (CAR), nucleic acid, vector, cell, or pharmaceutical composition according to the present invention in the manufacture of a medicament for treating or preventing a disease or disorder.

In another aspect, the present invention provides a method of treating or preventing a disease or disorder, comprising administering to a subject a therapeutically or prophylactically effective amount of a chimeric antigen receptor (CAR), nucleic acid, vector, cell, or pharmaceutical composition according to the present invention.

In another aspect, the present invention provides a method of treating or preventing a disease or disorder in a subject, comprising:
(a) isolating at least one T cell from a subject;
(b) modifying the at least one T cell to express or comprise a chimeric antigen receptor (CAR), nucleic acid, or a vector according to the present invention, and;
(c) administering the modified at least one T cell to a subject.

In another aspect, the present invention provides a method of treating or preventing a disease or disorder in a subject, comprising:
(a) isolating at least one T cell from a subject;
(b) introducing into the at least one T cell a nucleic acid or vector according to the present invention, thereby modifying the at least one T cell and;
(c) administering the modified at least one T cell to a subject.

In some embodiments of the CAR, nucleic acid, vector, cell, or pharmaceutical composition for use, the use, or the method according to the present invention, the disease or disorder is a cancer. In some embodiments, the cancer is a GPC3-expressing cancer or an EpCAM-expressing cancer. In some embodiments, the GPC3-expressing cancer or EpCAM-expressing cancer is a hepatocellular carcinoma.

In another aspect, the present invention provides a kit of parts comprising a predetermined quantity of a chimeric antigen receptor (CAR), nucleic acid, vector, cell, or pharmaceutical composition according to the present invention.

### DESCRIPTION

### CD226

CD226 (also known as DNAM-1, PTA1, TLiSA1) is a protein which is encoded in humans by the CD226 gene. CD226 is a ˜65 KDa transmembrane glycoprotein which is expressed at the cell surface of a variety of cell types, including natural killer (NK) cells, platelets, monocytes (dendritic cells and macrophages) and T cells.

The ligands for CD226 are CD112 (also known as nectin-2) and CD155 (also known as poliovirus receptor; PVR).

Studies have shown that CD226 triggers NK cell-mediated killing of tumor cells expressing CD155 and CD112 (Bottino et al., 2003 J Exp Med 198:1829-1839). CD226 also promotes co-stimulation of CD4+ and CD8+ T-cells, and may promote activation of CD8+ T cells by non-professional antigen-presenting cells (Gilfillan et al. 2008 J Exp Med 205: 2965-2973).

T-cell immunoreceptor with Ig and ITIM domains (TIGIT) is a coinhibitory immune receptor which competes with CD226 for binding to CD112 and CD155 (Lozano et al., 2012 J Immunol 188(8): 3869-3875). TIGIT has been shown to inhibit anti-tumor and other CD8+ T cell-dependent chronic immune responses, and this may involve impairment of CD226 homodimerization by TIGIT (Johnston et al., 2014 Cancer Cell 26: 923-937)

### Chimeric Antigen Receptors

The present invention provides a chimeric antigen receptor (CAR). Also provided is a chimeric antigen receptor (CAR) which is capable of binding to GPC3.

Chimeric Antigen Receptors (CARs) are recombinant receptor molecules which provide both antigen-binding and T cell activating functions. CAR structure and engineering is reviewed, for example, in Dotti et al., Immunol Rev (2014) 257(1), which is hereby incorporated by reference in its entirety.

CARs comprise an antigen-binding domain linked to a transmembrane domain and a signaling domain. An optional hinge domain may provide separation between the antigen-binding domain and transmembrane domain, and may act as a flexible linker.

The antigen-binding domain of a CAR may be based on the antigen-binding region of an antibody which is specific for the antigen to which the CAR is targeted. For example, the antigen-binding domain of a CAR may comprise amino acid sequences for the complementarity-determining regions (CDRs) of an antibody which binds specifically to the target protein. The antigen-binding domain of a CAR may comprise or consist of the light chain and heavy chain variable region amino acid sequences of an antibody which binds specifically to the target protein. The antigen-binding domain may be provided as a single chain variable fragment (scFv) comprising the sequences of the light chain and heavy chain variable region amino acid sequences of an antibody. Antigen-binding domains of CARs may target antigen based on other protein:protein interaction, such as ligand:receptor binding; for example an IL-13Rα2-targeted CAR has been developed using an antigen-binding domain based on IL-13 (see e.g. Kahlon et al. 2004 Cancer Res 64(24): 9160-9166).

The transmembrane domain is provided between the antigen-binding domain and the signalling domain of the CAR. The transmembrane domain provides for anchoring the CAR to the cell membrane of a cell expressing a CAR, with the antigen-binding domain in the extracellular space, and signalling domain inside the cell. Transmembrane domains of CARs may be derived from transmembrane region sequences for CD3-ζ, CD4, CD8 or CD28.

The signalling domain allows for activation of the T cell. The CAR signalling domains may comprise the amino acid sequence of the intracellular domain of CD3-ζ, which provides immunoreceptor tyrosine-based activation motifs (ITAMs) for phosphorylation and activation of the CAR-expressing T cell. Signalling domains comprising sequences of other ITAM-containing proteins have also been employed in CARs, such as domains comprising the ITAM containing region of FcγRI (Haynes et al., 2001 J Immunol 166(1):182-187). CARs comprising a signalling domain derived from the intracellular domain of CD3-ζ are often referred to as first generation CARs.

Signalling domains of CARs may also comprise co-stimulatory sequences derived from the signalling domains of co-stimulatory molecules, to facilitate activation of CAR-expressing T cells upon binding to the target protein. Suitable co-stimulatory molecules include CD28, OX40, 4-1 BB, ICOS and CD27. CARs having a signalling domain including additional co-stimulatory sequences are often referred to as second generation CARs.

In some cases CARs are engineered to provide for co-stimulation of different intracellular signalling pathways. For example, signalling associated with CD28 costimulation preferentially activates the phosphatidylinositol 3-kinase (PI3K) pathway, whereas the 4-1 BB-mediated signalling is through TNF receptor associated factor (TRAF) adaptor proteins. Signalling domains of CARs therefore sometimes contain co-stimulatory sequences derived from signalling domains of more than one co-stimulatory molecule. CARs comprising a signalling domain with multiple co-stimulatory sequences are often referred to as third generation CARs.

An optional hinge region may provide separation between the antigen-binding domain and the transmembrane domain, and may act as a flexible linker. Hinge regions may be flexible domains allowing the binding moiety to orient in different directions. Hinge regions may be derived from IgG1 or the CH₂CH₃ region of immunoglobulin.

### Antigen-Binding Domain

The chimeric antigen receptor (CAR) of the present invention comprises an antigen-binding domain.

The antigen-binding domain of the CAR of the present invention preferably displays specific binding to a target molecule, e.g. a target protein. “Specific binding” is interaction which is not non-specific. Specific binding is mediated by non-covalent interactions such as Van der Waals forces, electrostatic interactions, hydrogen bonding, and hydrophobic interactions. The antigen-binding domain of the CAR of the present invention may be derived from an antibody directed against the target molecule, or other target molecule-binding agent e.g. a target molecule-binding peptide or nucleic acid aptamer, ligand or other molecule.

The antigen-binding domain may be directed against any target molecule. In some embodiments, the antigen-binding domain is capable of binding to a target protein whose expression, or whose upregulated expression, is positively associated with a disease or disorder. That is, the target protein may be a marker of a disease or disorder.

The target protein is preferably expressed at the cell surface of a cell expressing the target protein.

In some embodiments, the target protein is expressed by a cell, or a cell of a tissue, against which it is desired to direct an immune response, e.g. a cell mediated immune response, such as a cytotoxic immune response.

In some embodiments the target protein is associated with an infectious disease, an autoimmune disease, or a cancer. In some embodiments, the target protein is expressed by a cell infected with an infectious agent, an autoimmune effector cell (i.e. effectors of an autoimmune pathology), or a cancer cell. In some embodiments, the target protein is expressed by, or expression is upregulated in, a cell in response to infection with an infectious agent (e.g. a virus or intracellular pathogen). In some embodiments, the target protein is expressed by, or expression is upregulated in, an autoimmune effector cell (e.g. an autoreactive T cell). In some embodiments, the target protein is expressed by, or expression is upregulated in, a cancer cell, e.g. a cell of a tumor.

In some embodiments, the antigen-binding domain of the CAR according to the present invention may be directed against a target molecule selected from a target molecule disclosed in Table 1 of Sadelain et al., 2013, Cancer Discov 3(4):388-398, which hereby incorporated by reference in its entirety: α-Folate receptor, CAIX, CD19, CD20, CD22, CD23, CD24, CD30, CD33 CD38, CD44v7/8, CEA, EGFRvIII, EGP-2, EGP-40, EphA2, erb-B2, erb-B 2,3,4, FBP, Fetal acethylcholine e receptor, GD2, GD3, Her-2, HMW-MAA, IL-11Rα, IL-13R-α2, KDR, κ-light chain, Lewis Y, L1-cell adhesion molecule, MAGE-A1, Mesothelin, Murine CMV infected cells, MUC1, MUC16, NKG2D, NY-ESO-1, Oncofetal antigen (h5T4), PSCA, PSMA, ROR1, Targeting via mAb IgE, TAG-72, VEGF-R2, and biotinylated molecules.

The antigen-binding domain may comprise the heavy and light chain variable region sequences of an antibody directed against the target molecule. The heavy and light chain variable region sequences may be provided in any suitable format provided that the antigen-binding domain can be linked to the other domains of the CAR. Formats contemplated in connection with the antigen-binding domain of the present invention include those described in Carter, Nat. Rev. Immunol 2006, 6: 343-357, such as scFv, dsFV, (scFv)₂ diabody, triabody, tetrabody, Fab, minibody, and F(ab)₂ formats.

In some embodiments the heavy chain variable region sequence and light chain variable region sequence may be provided in the CAR with a particular relative orientation. In some embodiments, the heavy chain variable region sequence may be N-terminal to the light chain variable region sequence. In some embodiments, the light chain variable region sequence may be N-terminal to the heavy chain variable region sequence.

In some embodiments, the target molecule-binding domain may comprise or consist of a single chain variable fragment (scFv) comprising a heavy chain variable region sequence and a light chain variable region sequence. In some embodiments, the heavy chain variable region and the light chain variable region sequences are linked by a flexible linker sequence. Flexible linker sequences are known to the skilled person, and are described, for example in Chen et al., Adv Drug Deliv Rev (2013) 65(10): 1357-1369, which is hereby incorporated by reference in its entirety. In some embodiments the flexible linker sequence comprises serine and glycine residues. In some embodiments the flexible linker sequence comprises 1-100, 5-50, 10-30, or 12-20 amino acid residues.

In some embodiments, the target protein is GPC3. That is, in some embodiments the antigen-binding domain is a GPC3-binding domain.

GPC3 (Glypican 3 also known as DGSX, GTR2-2, MXR7, OCI-5, SDYS, SGB, SGBS and SGBS1) is a cell surface protein of the glypican family of heparan sulphate proteoglycans. GPC3 is not expressed in normal adult liver tissue, but is expressed in hepatocellular carcinoma (Shirakawa et al. 2009 Intl J Oncol 34: 649-656; Ho et al. 2011 Eur J Cancer 47(3):333-338). GPC3 expression has also been observed in other cancers such as melanoma, ovarian clear-cell carcinoma, yolk sac tumors, neuroblastoma, hepatoblastoma, and Wilms' tumor cells (Ho et al. 2011 Eur J Cancer 47(3):333-338). GPC3 is therefore a candidate target for cancer therapy.

The GPC3-binding domain is capable of binding to a GPC3 polypeptide. A GPC3 polypeptide to which the GPC3-binding domain is capable of binding may comprise or consist of an amino acid sequence encoded by human GPC3 gene, or the homologous gene in a non-human animal. The non-human animal may be a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

The GPC3-binding domain of the CAR of the present invention preferably displays specific binding to a GPC3 polypeptide. The GPC3-binding domain may be derived from an anti-GPC3 antibody or other GPC3-binding agent, e.g. a GPC3-binding peptide or GPC3-binding small molecule, e.g. a GPC3-binding lipocalin mutein as disclosed in WO 2013/174783 A1.

The GPC3-binding domain may be derived from the antigen-binding region of an anti-GPC3 antibody. Anti-GPC3 antibodies are described e.g. in Feng and Ho, 2014 FEBS Lett 588(2): 377-382, which is hereby incorporated by reference in its entirety. Anti-GPC3 antibodies include the human monoclonal anti-GPC3 antibodies MDX-1414 (Medarex), HN3 (disclosed e.g. in WO 2012/145469 A1), the humanized mouse monoclonal anti-GPC3 antibodies GC33 (also known as R05137382, RG7686; described e.g. in WO 2006/046751 A1) and YP7 (described e.g. in WO 2013/181543 A1), and anti-GPC3 antibodies disclosed in WO 2009/012394 A1, WO 2006/046751 A1, WO 2013/181543 A1, WO 2012/145469 A1, WO 2016/036973 A1, WO 2006/006693 A1, WO 2013/070468 WO 2007/047291, each hereby incorporated by reference in their entirety.

A GPC3-binding domain according to the present invention preferably comprises heavy and light chain variable region sequences of an anti-GPC3 antibody, or comprises heavy and light chain variable region sequences derived from the heavy and light chain variable region sequences of an anti-GPC3 antibody.

The heavy and light chain variable region sequences may be provided in any suitable format provided that the GPC3-binding domain can be linked to the other domains of the CAR.

In some embodiments, the GPC3-binding domain comprises the CDRs of an anti-GPC3 antibody as described herein. In some embodiments, the GPC3-binding domain comprises the heavy and light chain variable region sequences of an anti-GPC3 antibody as described herein. In some embodiments, the CAR comprises the CDRs of the anti-GPC3 antibody GC33. The heavy and light chain variable region sequences, and the heavy and light chain CDRs 1-3 defined according to the Kabat numbering system (Kabat et al., (1991) Sequences of Proteins of Immunological Interest), for antibody GC33 are shown below:

GC33 heavy chain variable region sequence:
(SEQ ID NO: 1)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSA
HC-CDR1:
(SEQ ID NO: 2)
DYEMH
HC-CDR2:
(SEQ ID NO: 3)
ALDPKTGDTAYSQKFKG
HC-CDR3:
(SEQ ID NO: 4)
FYSYTY
GC33 light chain variable region sequence:
(SEQ ID NO: 5)
DVVMTQTPLSLPVSLGDQASISCRSSQSLVHSNGNTYLHWYLQKPGQSPK
LLIYKVSNRFSGVPDRFSGSGSGTDFTLKISRVEAEDLGVYFCSQNTHVP
PTFGSGTKLEIK
LC-CDR1:
(SEQ ID NO: 6)
RSSQSLVHSNGNTYLH
LC-CDR2:
(SEQ ID NO: 7)
KVSNRFS
LC-CDR3:
(SEQ ID NO: 8)
SQNTHVPPT

In some embodiments, the GPC3-binding domain comprises the following amino acid sequences i) to vi):

i) HC-CDR1:

(SEQ ID NO: 2)

DYEMH
ii) HC-CDR2:

(SEQ ID NO: 3)

ALDPKTGDTAYSQKFKG
iii) HC-CDR3:

(SEQ ID NO: 4)

FYSYTY
iv) LC-CDR1:

(SEQ ID NO: 6)

RSSQSLVHSNGNTYLH
v) LC-CDR2:

(SEQ ID NO: 7)

KVSNRFS
vi) LC-CDR3:

(SEQ ID NO: 8)

SQNTHVPPT

or a variant thereof in which one or two or three amino acids in one or more of the sequences i) to vi) are replaced with another amino acid.

In some embodiments, the GPC3-binding domain comprises a heavy chain variable region sequence and a light chain variable region sequence, wherein:
the heavy chain sequence has at least 85% 86%, 87%, 88%, 89%, 90%, 91%, 92%, 93%, 94%, 95%, 96%, 97%, 98%, 99%, or 100% sequence identity to the heavy chain sequence of SEQ ID NO:1, and;
the light chain sequence has at least 85% 86%, 87%, 88%, 89%, 90%, 91%, 92%, 93%, 94%, 95%, 96%, 97%, 98%, 99%, or 100% sequence identity to the light chain sequence of SEQ ID NO:5.

Alignment for purposes of determining percent amino acid or nucleotide sequence identity can be achieved in various ways known to a person of skill in the art, for instance, using publicly available computer software such as Clustal Omega, T-coffee or Megalign (DNASTAR) software. When using such software, the default parameters, e.g. for gap penalty and extension penalty, are preferably used.

In some embodiments, the GPC3-binding domain may comprise or consist of a single chain variable fragment (scFv) comprising a heavy chain variable region sequence and a light chain variable region sequence as described herein. The heavy chain variable region sequence and light chain variable region sequence may be linked by a covalent bond. In some embodiments, the heavy chain variable region and the light chain variable region sequences are linked by a flexible linker sequence, preferably covalently bonded to ends of the heavy chain variable region sequence and light chain variable region sequence.

In some embodiments, the GPC3-binding domain comprises, or consists of, an amino acid sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO:9:

(SEQ ID NO: 9)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQ
ASISCRSSQSLVHSNGNTYLHWYLQKPGQSPKLLIYKVSNRFSGVPDRFS
GSGSGTDFTLKISRVEAEDLGVYFCSQNTHVPPTFGSGTKLEIK

The light and heavy chain CDRs described herein may also be particularly useful in conjunction with a number of different framework regions. Accordingly, light and/or heavy chain variable region sequences comprising LC-CDR1-3 and/or HC-CDR1-3 may possess an alternative framework regions to those shown in SEQ ID NOs:1 and 5, respectively. Suitable framework regions are well known in the art and are described for example in M. Lefranc & G. Lefranc (2001) “The Immunoglobulin FactsBook”, Academic Press, incorporated herein by reference.

A CAR or a cell expressing a CAR comprising a GPC3-binding domain is capable of binding to GCP3. In some embodiments the CAR/cell is capable of binding to the C-terminal domain of GPC3. In some embodiments, the CAR/cell is capable of binding to the epitope of GPC3 which is bound by antibody GC33, e.g. within the region of amino acid positions 524-563 of human GCP3 polypeptide numbered according to UniProt: P51654 (GPC3_HUMAN) (Ho 2011 BioDrugs 25(5):275-284, hereby incorporated by reference in its entirety).

Binding to GPC3 can be analyzed by techniques well known to the person skilled in the art, such as by ELISA, immunoprecipitation, SPR, Bio-Layer Interferometry, flow cytometry or radioimmunoassay (RIA).

In some embodiments, the target protein is EpCAM. That is, in some embodiments the antigen-binding domain of the CAR of the present invention is an EpCAM-binding domain.

EpCAM (epithelial cell adhesion molecule, also known as DIARS, EGP-2, EGP314, EGP40, ESA, HNPCC8, KS1/4, KSA, M4S1, MIC18, MK-1, TACSTD1 and TROP1) is a transmembrane glycoprotein expressed exclusively in epithelia and epithelial-derived neoplasms (i.e. carcinomas). EpCAM structure, function and biology is reviewed for example in Schnell et al. Biochim Biophys Acta. 2013; 1828(8):1989-2001, which is hereby incorporated by reference in its entirety. EpCAM is thought to be involved in the tumorigenesis and metastatic progression of carcinomas, and high EpCAM expression correlates with poor survival in e.g. breast cancer, ovarian cancer, pancreatic carcinoma, urothelial carcinoma and gallbladder carcinoma.

The EpCAM-binding domain is capable of binding to an EpCAM polypeptide. An EpCAM polypeptide to which the EpCAM-binding domain is capable of binding may comprise or consist of an amino acid sequence encoded by human EPCAM gene, or the homologous gene in a non-human animal. The non-human animal may be a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

The EpCAM-binding domain of the CAR of the present invention preferably displays specific binding to an EpCAM polypeptide. The GPC3-binding domain may be derived from an anti-EpCAM antibody or other EpCAM-binding agent, e.g. an EpCAM-binding peptide or nucleic aptamer, or an EpCAM-binding small molecule.

The EpCAM-binding domain may be derived from the antigen-binding region of an anti-EpCAM antibody. Anti-EpCAM antibodies are described e.g. in Munz et al., Cancer Cell Int. (2010) 10:44, which is hereby incorporated by reference in its entirety. Anti-EpCAM antibodies include edrecolomab (Panorex; 17-1A), MOC31, 3622W94, ING-1, adecatumumab (MT201; Naundorf et al., Int J Cancer (2002) 100(1):101-10), and anti-EpCAM antibodies described in WO2004106383 A1, WO2005080428 A2, WO2008122551 A2, WO2010142990 A1, WO2011079283 A1, WO2012153186 A2, WO2013131001 A1, WO2015048901 A1 each of which is hereby incorporated by reference in entirety.

An EpCAM-binding domain according to the present invention preferably comprises heavy and light chain variable region sequences of an anti-EpCAM antibody, or comprises heavy and light chain variable region sequences derived from the heavy and light chain variable region sequences of an anti-EpCAM antibody.

The heavy and light chain variable region sequences may be provided in any suitable format provided that the EpCAM-binding domain can be linked to the other domains of the CAR.

In some embodiments, the EpCAM-binding domain comprises the CDRs of an anti-EpCAM antibody as described herein. In some embodiments, the EPCAM-binding domain comprises the heavy and light chain variable region sequences of an anti-EPCAM antibody as described herein. In some embodiments, the CAR comprises the CDRs of the anti-EPCAM antibody clone 3-171. The heavy and light chain variable region sequences, and the heavy and light chain CDRs 1-3 defined according to the Kabat numbering system (Kabat et al., (1991) Sequences of Proteins of Immunological Interest), for anti-EpCAM antibody clone 3-171 are shown below:

3-17I heavy chain variable region sequence:
(SEQ ID NO: 48)
QVQLVQSGAEVKKPGSSVKVSCKASGGTFSSYAISWVRQAPGQGLEWMG
GIIPIFGTANYAQKFQGRVTITADESTSTAYMELSSLRSEDTAVYYCAR
GLLWNYWGQGTLVTV
HC-CDR1:
(SEQ ID NO: 49)
SYAIS
HC-CDR2:
(SEQ ID NO: 50)
GIIPIFGTANYAQKFQG
HC-CDR3:
(SEQ ID NO: 51)
GLLWNY
3-17I light chain variable region sequence:
(SEQ ID NO: 52)
EIVMTQSPATLSVSPGERATLSCRASQSVSSNLAWYQQKPGQAPRLIIY
GASTTASGIPARFSASGSGTDFTLTISSLQSEDFAVYYCQQYNNWPPA
YTFGQGTKLEIK
LC-CDR1:
(SEQ ID NO: 53)
RASQSVSSNLA
LC-CDR2:
(SEQ ID NO: 54)
GASTTAS
LC-CDR3:
(SEQ ID NO: 55)
QQYNNWPPAYT

In some embodiments, the EpCAM-binding domain comprises the following amino acid sequences i) to vi):

i) HC-CDR1:

(SEQ ID NO: 49)

SYAIS
ii) HC-CDR2:

(SEQ ID NO: 50)

GIIPIFGTANYAQKFQG
iii) HC-CDR3:

(SEQ ID NO: 51)

GLLWNY
iv) LC-CDR1:

(SEQ ID NO: 53)

RASQSVSSNLA
v) LC-CDR2:

(SEQ ID NO: 54)

GASTTAS
vi) LC-CDR3:

(SEQ ID NO: 55)

QQYNNWPPAYT

or a variant thereof in which one or two or three amino acids in one or more of the sequences i) to vi) are replaced with another amino acid.

In some embodiments, the EpCAM-binding domain comprises a heavy chain variable region sequence and a light chain variable region sequence, wherein:
the heavy chain sequence has at least 85% 86%, 87%, 88%, 89%, 90%, 91%, 92%, 93%, 94%, 95%, 96%, 97%, 98%, 99%, or 100% sequence identity to the heavy chain sequence of SEQ ID NO:48, and;
the light chain sequence has at least 85% 86%, 87%, 88%, 89%, 90%, 91%, 92%, 93%, 94%, 95%, 96%, 97%, 98%, 99%, or 100% sequence identity to the light chain sequence of SEQ ID NO:52.

In some embodiments, the EpCAM-binding domain may comprise or consist of a single chain variable fragment (scFv) comprising a heavy chain variable region sequence and a light chain variable region sequence as described herein. The heavy chain variable region sequence and light chain variable region sequence may be linked by a covalent bond. In some embodiments, the heavy chain variable region and the light chain variable region sequences are linked by a flexible linker sequence, preferably covalently bonded to ends of the heavy chain variable region sequence and light chain variable region sequence.

In some embodiments, the EpCAM-binding domain comprises, or consists of, an amino acid sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO:56:

(SEQ ID NO: 56)
QVQLVQSGAEVKKPGSSVKVSCKASGGTFSSYAISWVRQAPGQGLEWMGG
IIPIFGTANYAQKFQGRVTITADESTSTAYMELSSLRSEDTAVYYCARGL
LWNYWGQGTLVTVSSKLSGSASAPKLEEGEFSEARVEIVMTQSPATLSVS
PGERATLSCRASQSVSSNLAWYQQKPGQAPRLIIYGASTTASGIPARFSA
SGSGTDFTLTISSLQSEDFAVYYCQQYNNWPPAYTFGQGTKLEIK

The light and heavy chain CDRs described herein may also be particularly useful in conjunction with a number of different framework regions. Accordingly, light and/or heavy chain variable region sequences comprising LC-CDR1-3 and/or HC-CDR1-3 may possess an alternative framework regions to those shown in SEQ ID NOs:48 and 52, respectively. Suitable framework regions are described for example in M. Lefranc & G. Lefranc (2001) “The Immunoglobulin FactsBook”, Academic Press, incorporated by reference hereinabove.

A CAR or a cell expressing a CAR comprising an EpCAM-binding domain is capable of binding to EpCAM. In some embodiments the CAR/cell is capable of binding to the extracellular domain of EpCAM. In some embodiments, the CAR/cell is capable of binding to the epitope of EpCAM which is bound by anti-EpCAM antibody clone 3-17I.

Binding to EpCAM can be analyzed by techniques such as by ELISA, immunoprecipitation, SPR, Bio-Layer Interferometry, flow cytometry or radioimmunoassay (RIA).

### Transmembrane Domain

The chimeric antigen receptor of the present invention comprises a transmembrane domain.

A transmembrane domain refers to any three-dimensional structure formed by a sequence of amino acids which is thermodynamically stable in a biological membrane, e.g. a cell membrane. In connection with the present invention, the transmembrane domain may be an amino acid sequence which spans the cell membrane of a cell expressing the CAR.

The transmembrane domain may comprise or consist of a sequence of amino acids which forms a hydrophobic alpha helix or beta-barrel. The amino acid sequence of the transmembrane domain of the CAR of the present invention may be, or may be derived from, the amino acid sequence of a transmembrane domain of a protein comprising a transmembrane domain. Transmembrane domains are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as TMHMM (Krogh et al., 2001 J Mol Biol 305: 567-580).

In some embodiments, the amino acid sequence of the transmembrane domain of the CAR of the present invention may be, or may be derived from, the amino acid sequence of the transmembrane domain of a protein expressed at the cell surface. In some embodiments the protein expressed at the cell surface is a receptor or ligand, e.g. an immune receptor or ligand. In some embodiments the amino acid sequence of the transmembrane domain may be, or may be derived from, the amino acid sequence of the transmembrane domain of one of ICOS, ICOSL, CD86, CTLA-4, CD28, CD80, MHC class I α, MHC class II α, MHC class II β, CD3ε, CD3δ, CD3γ, CD3-ζ, TCRα TCRβ, CD4, CD8α, CD8β, CD40, CD40L, PD-1, PD-L1, PD-L2, 4-1BB, 4-1BBL, OX40, OX40L, GITR, GITRL, TIM-3, Galectin 9, LAG3, CD27, CD70, LIGHT, HVEM, TIM-4, TIM-1, ICAM1, LFA-1, LFA-3, CD2, BTLA, CD160, LILRB4, LILRB2, VTCN1, CD2, CD48, 2B4, SLAM, CD30, CD30L, DR3, TL1A, CD226, CD155, CD112 and CD276. In some embodiments, the transmembrane is, or is derived from, the amino acid sequence of the transmembrane domain of CD3-ζ, CD4, CD8α, CD8β, CD28 or CD226.

In some embodiments, the transmembrane domain of the CAR according to the present invention comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:10 or 11:

CD28 transmembrane domain:

(SEQ ID NO: 10)

FWVLVVVGGVLACYSLLVTVAFII
CD8α transmembrane domain:

(SEQ ID NO: 11)

IYIWAPLAGTCGVLLLSLVITLYCNHRN

In some embodiments, the transmembrane domain of the CAR according to the present invention comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:57.

### Signaling Domain

The chimeric antigen receptor of the present invention comprises a signaling domain. In the chimeric antigen receptor of the present invention, the costimulatory sequence which is, or which is derived from, the intracellular domain of CD226, or a fragment thereof is typically provided in a signaling domain. The signaling domain provides sequences for initiating intracellular signaling in the cell expressing the CAR.

### ITAM-Containing Sequence

The signaling domain comprises an amino acid sequence comprising one or more immunoreceptor tyrosine-based activation motifs (ITAMs). ITAMs comprise the amino acid sequence YXXL/I (SEQ ID NO:12), wherein “X” denotes any amino acid. In ITAM-containing proteins, sequences according to SEQ ID NO:12 are often separated by 6 to 8 amino acids; YXXL/I(X)₆₋₈YXXL/I (SEQ ID NO:13). When phosphate groups are added to the tyrosine residue of an ITAM by tyrosine kinases, a signaling cascade is initiated within the cell.

In some embodiments, the signaling domain of the CAR according to the present invention comprises one or more copies of an amino acid sequence according to SEQ ID NO:12 or SEQ ID NO:13. In some embodiments, the signaling domain comprises at least 1, 2, 3, 4, 5 or 6 copies of an amino acid sequence according to SEQ ID NO:12. In some embodiments, the signaling domain comprises at least 1, 2, or 3 copies of an amino acid sequence according to SEQ ID NO:13. In some embodiments, the signaling domain comprises 1 to 10, 2 to 8, 3 to 7 or 4 to 6 copies of an amino acid sequence according to SEQ ID NO:12. In some embodiments, the signaling domain comprises at least 1 to 6, 2 to 5, or 3 to 4 copies of an amino acid sequence according to SEQ ID NO:13.

In some embodiments, the signaling domain comprises an amino acid sequence which is, or which is derived from, the amino acid sequence of an ITAM-containing sequence of a protein having an ITAM-containing amino acid sequence. In some embodiments the signaling domain comprises an amino acid sequence which is, or which is derived from, an ITAM-containing sequence (e.g. the intracellular domain) of the amino acid sequence of one of CD3ε, CD3δ, CD3γ, CD3-ζ, CD79α, CD79β, FcγRI, FcγRIIA, FcγRIIC, FcγRIIIA, FcγRIV or DAP12. In some embodiments the signaling domain comprises an amino acid sequence which is, or which is derived from, an ITAM-containing sequence (e.g. the intracellular domain) of CD3-ζ.

Throughout this specification, an amino acid sequence which is “derived from” a given amino acid sequence may retain structural and/or functional properties of the amino acid sequence from which it is derived. The amino acid sequence may have high sequence identity to the amino acid sequence from which it is derived. For example, an amino acid sequence which is derived from a given sequence may have at least 80%, 85% 86%, 87%, 88%, 89%, 90%, 91%, 92%, 93%, 94%, 95%, 96%, 97%, 98%, 99%, or 100% sequence identity to the amino acid sequence from which it is derived.

The amino acid sequence of a given protein or domain thereof can be retrieved from, or determined from a nucleic acid sequence retrieved from, databases known to the person skilled in the art. Such databases include GenBank, EMBL, DDBJ, UniProt, Swiss-Prot, TrEMBL, Protein Information Resource, Protein Data Bank, Ensembl and InterPro.

By way of example, a CAR according to the present invention which comprises a signaling domain comprising an amino acid sequence which is, or which is derived from, the intracellular domain of CD3-ζ may comprise an amino acid sequence comprising at least 80% sequence identity to the intracellular domain of CD3-ζ represented by positions 52-164 of the amino acid sequence of UniProt: P20963-1 (CD3Z_HUMAN).

In some embodiments, the signaling domain of the CAR according to the present invention comprises an ITAM-containing amino acid sequence which comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:14:

CD3-ζ intracellular domain:
(SEQ ID NO: 14)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTAT
KDTYDALHMQALPPR

### Costimulatory Sequence

The signaling domain may additionally comprise one or more costimulatory sequences. In some embodiments the chimeric antigen receptor of the present invention comprises a costimulatory sequence which is, or which is derived from, the intracellular domain of CD226 or a fragment thereof.

A costimulatory sequence is an amino acid sequence which provides for costimulation of the cell expressing the CAR. Costimulation promotes proliferation and survival of a CAR-expressing cell, and may also promote cytokine production, differentiation, cytotoxic function and memory formation. Molecular mechanisms of T cell costimulation are reviewed in Chen and Flies 2013 Nat Rev Immunol 13(4):227-242.

A costimulatory sequence of the signaling domain of the CAR of the present invention may be, or may be derived from, the amino acid sequence of a costimulatory protein. The costimulatory sequence which is, or which is derived from, the intracellular domain of CD226 or a fragment thereof is capable of initiating CD226-mediated signalling. That is, the CAR of the present invention comprises a costimulatory sequence which is capable of delivering a CD226 costimulation signal.

Costimulatory signaling through CD226 is described e.g. in Martinet and Smyth, Nat Rev Immunol (2015) 15:243-254, which is hereby incorporated by reference in its entirety. Signaling is initiated by phosphorylation of Serine 329 and Tyrosine 322 of CD226, and the phosphorylated residues facilitate activation of protein kinase C (PKC) and association with LFA1, which in turn facilitates FYN-mediated phosphorylation of Tyrosine 322 of CD226 and downstream signaling.

Whether a given amino acid sequence is capable of initiating CD226-mediated signaling can be investigated e.g. by analyzing activation or expression of a molecule whose activation or expression is upregulated or downregulated as a consequence of CD226-mediated signaling. For example, the whether a given amino acid sequence is capable of initiating CD226-mediated signaling can be investigated by analyzing one or more of phosphorylation of Serine 329 and/or Tyrosine 322, association with/activation of PKC, association with/activation of LFA1, association with/activation of FYN, or upregulation of the expression of any other molecule whose expression is upregulated by CD226-mediated signaling. The analysis can be formed e.g. in vitro using cells expressing a CAR comprising the amino acid sequence.

CD226 may be human CD226. Human CD226 may have the amino acid sequence of UniProt Q15762 (CD226_HUMAN) according to SEQ ID NO: 15.

Human CD226; UniProt Q15762 (CD226_HUMAN):
(SEQ ID NO: 15)
MDYPTLLLALLHVYRALCEEVLWHTSVPFAENMSLECVYPSMGILTQVEW
FKIGTQQDSIAIFSPTHGMVIRKPYAERVYFLNSTMASNNMTLFFRNASE
DDVGYYSCSLYTYPQGTWQKVIQVVQSDSFEAAVPSNSHIVSEPGKNVTL
TCQPQMTWPVQAVRWEKIQPRQIDLLTYCNLVHGRNFTSKFPRQIVSNCS
HGRWSVIVIPDVTVSDSGLYRCYLQASAGENETFVMRLTVAEGKTDNQYT
LFVAGGTVLLLLFVISITTIIVIFLNRRRRRERRDLFTESWDTQKAPNNY
RSPISTSQPTNQSMDDTREDIYVNYPTFSRRPKTRV

The intracellular domain of human CD226 may correspond to amino acid positions 271 to 336 of SEQ ID NO:15, i.e. the sequence according to SEQ ID NO:16.

CD226 intracellular domain:
(SEQ ID NO: 16)
IVIFLNRRRRRERRDLFTESWDTQKAPNNYRSPISTSQPTNQSMDDTRE
DIYVNYPTFSRRPKTRV

In some embodiments, the signaling domain of the CAR of the present invention comprises a costimulatory sequence which comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:16, or a fragment thereof.

The intracellular domain of human CD226 may correspond to amino acid positions 276 to 336 of SEQ ID NO:15, i.e. the sequence according to SEQ ID NO:58, herein referred to as “CD226 ICD v1”

In some embodiments, the signaling domain of the CAR of the present invention comprises a costimulatory sequence which comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:58, or a fragment thereof.

The intracellular domain of human CD226 may correspond to amino acid positions 274 to 336 of SEQ ID NO:15, i.e. the sequence according to SEQ ID NO:59, herein referred to as “CD226 ICD v2”

In some embodiments, the signaling domain of the CAR of the present invention comprises a costimulatory sequence which comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:59, or a fragment thereof.

In some embodiments the signaling domain of the CAR of the present invention comprises further costimulatory sequences in addition to the costimulatory sequence which is, or which is derived from, the intracellular domain of CD226.

In some embodiments, the signaling domain comprises more than one costimulatory sequence. In some embodiments the signaling domain comprises 2, 3, 4 or 5 costimulatory sequences. In some embodiments, a costimulatory sequence of the signaling domain of the CAR of the present invention may be, or may be derived from, the amino acid sequence of a costimulatory protein. In some embodiments, the sequence may be, or may be derived from, the intracellular domain of a costimulatory protein. In some embodiments, the costimulatory protein may be a member of the B7-CD28 superfamily (e.g. CD28, ICOS), or a member of the TNF receptor superfamily (e.g. 4-1BB, OX40, CD27, DR3, GITR, CD30, HVEM). In some embodiments, the signaling domain of the CAR comprises a costimulatory sequence which is, or which is derived from, the intracellular domain of one of CD28, ICOS, 4-1BB, CD27, OX40, HVEM, CD2, SLAM, TIM-1, CD30, GITR, DR3, LIGHT and CD226. In some embodiments, the signaling domain comprises a costimulatory sequence which is, or which is derived from, the intracellular domain of CD28 or 4-1 BB. In some embodiments, the signaling domain comprises a costimulatory sequence which is, or which is derived from, the intracellular domain of one of CD28 or, 4-1BB, and CD226.

Costimulatory proteins upregulate expression of genes promoting cell growth, effector function and survival through several transduction pathways. For example, CD28 and ICOS signal through phosphatidylinositol 3 kinase (PI3K) and AKT to upregulate expression of genes promoting cell growth, effector function and survival through NF-κB, mTOR, NFAT and AP1/2. CD28 also activates AP1/2 via CDC42/RAC1 and ERK1/2 via RAS, and ICOS activates C-MAF. 4-1BB, OX40, and CD27 recruit TNF receptor associated factor (TRAF) and signal through MAPK pathways, as well as through PI3K.

In some embodiments, the signaling domain of the CAR comprises a costimulatory sequence which comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:17 or 18:

CD28 intracellular domain:

(SEQ ID NO: 17)

FWVRSKRSRLLHSDYMNMTPRRPGPTRKHYQPYAPPRDFAAYRS
4-1BB intracellular domain:

(SEQ ID NO: 18)

KRGRKKLLYIFKQPFMRPVQTTQEEDGCSCRFPEEEEGGCEL

In some embodiments, the signaling domain of the CAR comprises: (i) a costimulatory sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:16; and (ii) a costimulatory sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:17.

In some embodiments, the signaling domain of the CAR comprises: (i) a costimulatory sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:16; and (ii) a costimulatory sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:18.

In some embodiments, the signaling domain of the CAR comprises: (i) a costimulatory sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:16; (ii) a costimulatory sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:17; and (iii) a costimulatory sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:18.

### Hinge Region

The chimeric antigen receptor of the present invention may comprise a hinge region between the antigen-binding domain and the transmembrane domain. A hinge region is an amino acid sequence which provides for flexible linkage of the antigen-binding and transmembrane domains of the CAR.

The presence, absence and length of hinge regions has been shown to influence CAR function (reviewed e.g. in Dotti et al., Immunol Rev (2014) 257(1) supra).

In some embodiments, the CAR comprises a hinge region comprising, or consisting of, an amino acid sequence which is, or which is derived from, the human IgG1 hinge region, the CH₂CH₃ (i.e. Fc) region of IgG1, the CH₂ region of IgG1, the CH₃ region of IgG1, IgG4, amino acids 187-189 of human IgD (Wilkie et al., 2008 J IMmunol 180(7): 4901-4909), a hinge region derived from CD8α, e.g. as described in WO 2012/031744 A1, or a hinge region derived from CD28, e.g. as described in WO 2011/041093 A1.

In some embodiments, the hinge domain of the CAR comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:19:

Human IgG1 hinge region:

(SEQ ID NO: 19)

EPKSCDKTHTCPPCP

### Dimerization Domain

The chimeric antigen receptor of the present invention may comprise a dimerization domain.

As used herein, a “dimerization domain” refers to a sequence of amino acids through which a protein may associate with another protein to form a dimer, oligomer or multimer. In some embodiments the other protein may be a membrane-bound molecule, e.g. a receptor or ligand. In some embodiments, the dimerization domain may provide for self-association of the CAR to form a homodimer, or may provide for association with another, different protein to form a heterodimer.

CAR monomers may also form higher-order oligomers/multimers, e.g. trimers, tetramers, pentamers, hexamers, heptamers, octamers, etc. In some embodiments, CAR monomers may associate to form higher-order oligomers/multimers through association via the dimerization domain. Accordingly, in some embodiments the dimerization domain may be an oligomerization domain or a multimerization domain, e.g. a trimerization domain, a tetramerization domain, a pentamerization domain, a hexamerization domain, a heptamerization domain, an octamerization domain, etc.

Dimerization domains have been employed in CARs for modulating CAR activity. Wu et al., 2015 Science 350(6258) (hereby incorporated by reference in its entirety) describes “ON-switch CAR”, in which antigen-binding and signal transduction domains were provided in separate molecules each including domains through which dimerization to form a functional CAR could be controlled using a small molecule.

The dimerization domain of a CAR according to the present invention may be spontaneous or inducible.

A spontaneous dimerization domain provides for association through said domain to form a dimer in the absence of an external factor/signal. Spontaneous dimerization domains are found e.g. in proteins which spontaneously form homodimers or heterodimers.

An inducible dimerization domain provides for association to form dimers in response to e.g. an agent/signal, with the result that dimerization can be controlled.

In some embodiments, dimerization may be inducible in response to treatment with a chemical. Examples of chemically-inducible dimerization include FKBP/FKBP homodimerization inducible with FK1012 (Spencer et al., 1993 Science 262(5136): 1019-1024); FKBP/CyP-Fas heterodimerization inducible with FKCsA (Belshaw et al 1996 PNAS 93(10): 4604-4607); FKBP/CNA heterodimerization inducible with FK506 (Ho et al., 1996 Nature 382(6594):822-826) FKBP/FRB domain of mTOR heterodimerization inducible with rapamycin (Rivera et al., 1996 Nature Medicine 2(9): 1028-1032); GAI/GID1 heterodimerization inducible with gibberellin (Miyamoto et al., 2012 Nature Chemical Biology 8(5): 465-470); GyrB/GyrB homodimerization inducible with coumermycin (Farrar et al., 1996 Nature 383 (6596):178-181); HalTag/SNAP-tag heterodimerization inducible using HaXS (Erhart et al., 2013 Chem Biol 20(4): 549-557); and F36V-FKBP/F36V-FKBP homodimerization inducible with AP1903 (Clackson et al., 1998 95(18): 10437-10442).

An inducible dimerization domain provides for selective upregulation of signaling through the CAR. For example, a CAR comprising a chemically-inducible dimerization domain can be stimulated to dimerize by treatment with the appropriate agent, resulting in increased CAR-mediated signaling. In this way, a cell comprising a CAR according to the invention can selectively be stimulated to proliferate (i.e. grow and divide).

Proliferation and survival of cells expressing a CAR having a chemically-inducible dimerization domain can be selectively stimulated using the appropriate agent. For example, cells expressing a CAR having a dimerization domain according to SEQ ID NO:19 can be selectively stimulated to grow and divide by treatment with AP1903, as a result of enhanced signalling through the CAR. Importantly, cells not comprising the CAR will not be stimulated to grow and divide by treatment with AP1903, and so cells expressing the CAR can be expanded from within a heterogenous population comprising cells expressing the CAR, and cells not expressing the CAR.

In some embodiments, the amino acid sequence of a dimerization domain of the CAR of the present invention may be, or may be derived from, the amino acid sequence of a protein known or predicted to form homodimers or heterodimers. The amino acid sequence of the dimerization domain of the CAR of the present invention may be, or may be derived from, the amino acid sequence of a dimerization domain for a protein comprising a dimerization domain. Amino acid sequences through which proteins form dimers are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as meta-PPISP (Qin et al., 2007 Bioinformatics 23:3386-3387).

In some embodiments, the amino acid sequence of the dimerization domain of the CAR of the present invention may be, or may be derived from, the amino acid sequence of FKBP or a mutant thereof, e.g. F36V, F36M.

In some embodiments, the dimerization domain of the CAR comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:20:

F36V-FKBP:
(SEQ ID NO: 20)
GVQVETISPGDGRTFPKRGQTCVVHYTGMLEDGKKVDSSRDRNKPFKFM
LGKQEVIRGWEEGVAQMSVGQRAKLTISPDYAYGATGHPGIIPPHATLV
FDVELLKLE

The dimerization domain may be located in the CAR N-terminal to the transmembrane domain, or C-terminal to the transmembrane domain. That is, when the CAR is expressed at the cell surface, the dimerization domain may be in the extracellular portion of the CAR, or the intracellular portion of the CAR.

### Signal Sequence

In some embodiments, the CAR of the present invention may comprise a signal sequence (also known as a signal peptide or leader sequence). Signal sequences normally consist of a sequence of 5-30 hydrophobic amino acids, which form a single alpha helix. Secreted proteins and proteins expressed at the cell surface often comprise signal sequences.

The signal sequence may be present at the N-terminus of the CAR, and may be present in the newly synthesized CAR. The signal sequence provides for efficient trafficking of the CAR to the cell surface. Signal sequences are often removed by cleavage, and thus are not comprised in the mature CAR expressed at the cell surface.

Signal sequences are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176).

In some embodiments, the signal sequence of the CAR of the present invention comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:21:

Human Ig heavy chain signal sequence:

(SEQ ID NO: 21)

MDWIWRILFLVGAATGAHS

### Linker Sequence

In some embodiments, the CAR of the present invention may comprise one or more linker sequences between regions/domains of the CAR. For example, the CAR may comprise the following structure:
N term-[ . . . ]-[hinge region]-{linker sequence}-[transmembrane domain]-[ . . . ]-C term

Linker sequences are known to the skilled person, and are described, for example in Chen et al., Adv Drug Deliv Rev (2013) 65(10): 1357-1369, incorporated by reference hereinabove.

In some embodiments, the linker sequence may be a rigid linker sequence. In some embodiments, the linker sequence may be a flexible linker sequence. In some embodiments, the linker sequence may be a cleavable linker sequence.

In some embodiments, a linker sequence may comprise 1-25, 1-20, 1-15, 1-10 or 1-5 amino acids. In some embodiments, a linker sequence may comprise fewer than 25, 20, 15, 10 or 5 amino acids.

### Additional Sequences

In some embodiments, the chimeric antigen receptor may comprise further functional amino acid sequences.

For example, the CAR may comprise amino acid sequence(s) to facilitate expression, folding, trafficking, processing or purification of the CAR. For example, the CAR may comprise a sequence encoding a protein tag, e.g. a His, (e.g. 6×His), Myc, GST, MBP, FLAG, HA, E, or Biotin tag, optionally at the N- or C-terminus.

Exemplary CARs The chimeric antigen receptor of the present invention may be provided with particular combinations and relative arrangements of domains.

The antigen-binding, transmembrane and signaling domains are arranged so that when the CAR is expressed at the cell surface, the antigen-binding domain is in the extracellular space and the signaling domain is inside the cell.

In some embodiments, the domains/sequences CAR of the present invention may be provided with a relative arrangement according to one of the following:
N term-[signal sequence]-[antigen-binding domain]-[hinge region]-[transmembrane domain]-[signalling domain]-C term
N term-[signal sequence]-[antigen-binding domain]-[hinge region]-[transmembrane domain]-[dimerization domain]-[signalling domain]-C term
N term-[signal sequence]-[GPC3-binding domain]-[hinge region]-[transmembrane domain]-[signalling domain]-C term
N term-[signal sequence]-[GPC3-binding domain]-[hinge region]-[transmembrane domain]-[dimerization domain]-[signalling domain]-C term
N term-[signal sequence]-[EpCAM-binding domain]-[hinge region]-[transmembrane domain]-[signalling domain]-C term
N term-[signal sequence]-[EpCAM-binding domain]-[hinge region]-[transmembrane domain]-[dimerization domain]-[signalling domain]-C term

In some embodiments, within the signalling domain, the ITAM-containing sequence and costimulatory sequence(s) may be provided with a relative arrangement according to one of the following:
N term-[ . . . ]-[costimulatory sequence]-[ITAM-containing sequence]-[ . . . ]-C term
N term-[ . . . ]-[costimulatory sequence 1]-[costimulatory sequence 2]-[ITAM-containing sequence]-[ . . . ]-C term
N term-[ . . . ]-[costimulatory sequence 1]-[costimulatory sequence 2]-[costimulatory sequence 3]-[ITAM-containing sequence]-[ . . . ]-C term

In accordance with aspects of the present invention wherein the CAR comprises a costimulatory sequence which is, or which is derived from, the intracellular domain of CD226, or a fragment thereof, in the costimulatory sequence which is, or which is derived from, the intracellular domain of CD226, or a fragment thereof may be adjacent to the transmembrane domain.

In some embodiments the costimulatory sequence which is, or which is derived from, the intracellular domain of CD226, or a fragment thereof is N-terminal to other costimulatory sequence(s) and/or ITAM-containing sequence(s) within the signalling domain.

In some embodiments, within the signalling domain, the ITAM-containing sequence and costimulatory sequence(s) may be provided with a relative arrangement according to one of the following:
N term-[ . . . ]-[CD226 costimulatory sequence]-[ITAM-containing sequence]-[ . . . ]-C term
N term-[ . . . ]-[CD226 costimulatory sequence]-[costimulatory sequence 2]-[ITAM-containing sequence]-[ . . . ]-C term
N term-[ . . . ]-[CD226 costimulatory sequence]-[costimulatory sequence 2]-[costimulatory sequence 3]-[ITAM-containing sequence]-[ . . . ]-C term

In some embodiments, the CAR may comprise the combination of domains/sequences according to any one of A to M as shown in Table 1:

TABLE 1

| | | Trans- | | |
|---|---|---|---|---|
| | Antigen-binding | membrane | Dimerization | |
| | domain | domain | domain | Signaling domain |
| A | GPC3-binding scFV | CD8α | — | CD226, CD3ζ |
| B | GPC3-binding scFV | CD8α | F36V-FKBP | CD226, CD3ζ |
| C | GPC3-binding scFV | CD8α | — | CD226, CD28, CD3ζ |
| D | GPC3-binding scFV | CD8α | F36V-FKBP | CD226, CD28, CD3ζ |
| E | GPC3-binding scFV | CD8α | — | CD226, 4-1BB, |
| | | | | CD3ζ |
| F | GPC3-binding scFV | CD8α | F36V-FKBP | CD226, 4-1BB, |
| | | | | CD3ζ |
| G | GPC3-binding scFV | CD8α | — | CD226, CD28, |
| | | | | 4-1BB, CD3ζ |
| H | GPC3-binding scFV | CD8α | F36V-FKBP | CD226, CD28, |
| | | | | 4-1BB, CD3ζ |
| I | GPC3-binding scFV | CD28 | F36V-FKBP | 4-1BB, CD3ζ |
| J | GPC3-binding scFV | CD8α | — | 4-1BB, CD3ζ |
| K | GPC3-binding scFV | CD8α | F36V-FKBP | 4-1BB, CD3ζ |
| L | GPC3-binding scFV | CD8α | — | CD28, CD3ζ |
| M | GPC3-binding scFV | CD8α | F36V-FKBP | CD28, CD3ζ |

It will be appreciated that Table 1 provides short-hand representations for the combinations of domains/sequences of the CARs A to M. CARs A to M comprise the following combinations of domains/sequences:

(A) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a signalling domain which comprises:
   a costimulatory sequence which comprises or consists of an amino acid sequence which is, or which is derived from, the intracellular domain of CD226, and
   an ITAM-containing sequence which comprises or consists of an amino acid sequence which is, or which is derived from, the intracellular domain of CD3ζ.
      (B) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a dimerization domain which comprises or consists of an amino acid sequence which is, or which is derived from, the amino acid sequence of F36V-FKBP;
a signalling domain which comprises:
   a costimulatory sequence which comprises or consists of an amino acid sequence which is, or which is derived from, the intracellular domain of CD226, and
   an ITAM-containing sequence which comprises or consists of an amino acid sequence which is, or which is derived from, the intracellular domain of CD3ζ.
      (C) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a signalling domain which comprises:
   a costimulatory sequence which comprises or consists of an amino acid sequence which is, or which is derived from, the intracellular domain of CD226,
   a costimulatory sequence which comprises or consists of an amino acid sequence which is, or which is derived from, the intracellular domain of CD28, and
   an ITAM-containing sequence which comprises or consists of an amino acid sequence which is, or which is derived from, the intracellular domain of CD3ζ.
      (D) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a dimerization domain which comprises or consists of an amino acid sequence which is, or which is derived from, the amino acid sequence of F36V-FKBP;
a signalling domain which comprises:
   a costimulatory sequence which comprises or consists of an amino acid sequence which is, or which is derived from, the intracellular domain of CD226,
   a costimulatory sequence which comprises or consists of an amino acid sequence which is, or which is derived from, the intracellular domain of CD28, and an ITAM-containing sequence which comprises or consists of an amino acid sequence which is, or which is derived from, the intracellular domain of CD3ζ.
      (E) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a signalling domain which comprises:
   a costimulatory sequence which comprises or consists of an amino acid sequence which is, or which is derived from, the intracellular domain of CD226,
   a costimulatory sequence which comprises or consists of an amino acid sequence which is, or which is derived from, the intracellular domain of 4-1BB, and
   an ITAM-containing sequence which comprises or consists of an amino acid sequence which is, or which is derived from, the intracellular domain of CD3ζ.
      (F) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a dimerization domain which comprises or consists of an amino acid sequence which is, or which is derived from, the amino acid sequence of F36V-FKBP;
a signalling domain which comprises:
   a costimulatory sequence which comprises or consists of an amino acid sequence which is, or which is derived from, the intracellular domain of CD226,
   a costimulatory sequence which comprises or consists of an amino acid sequence which is, or which is derived from, the intracellular domain of 4-1BB, and
   an ITAM-containing sequence which comprises or consists of an amino acid sequence which is, or which is derived from, the intracellular domain of CD3ζ.
      (G) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a signalling domain which comprises:
   a costimulatory sequence which comprises or consists of an amino acid sequence which is, or which is derived from, the intracellular domain of CD226,
   a costimulatory sequence which comprises or consists of an amino acid sequence which is, or which is derived from, the intracellular domain of CD28,
   a costimulatory sequence which comprises or consists of an amino acid sequence which is, or which is derived from, the intracellular domain of 4-1BB, and
   an ITAM-containing sequence which comprises or consists of an amino acid sequence which is, or which is derived from, the intracellular domain of CD3ζ.
      (H) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a dimerization domain which comprises or consists of an amino acid sequence which is, or which is derived from, the amino acid sequence of F36V-FKBP;
a signalling domain which comprises:
   a costimulatory sequence which comprises or consists of an amino acid sequence which is, or which is derived from, the intracellular domain of CD226,
   a costimulatory sequence which comprises or consists of an amino acid sequence which is, or which is derived from, the intracellular domain of CD28,
   a costimulatory sequence which comprises or consists of an amino acid sequence which is, or which is derived from, the intracellular domain of 4-1BB, and
   an ITAM-containing sequence which comprises or consists of an amino acid sequence which is, or which is derived from, the intracellular domain of CD3ζ.
      (I) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD28;
a dimerization domain which comprises or consists of an amino acid sequence which is, or which is derived from, the amino acid sequence of F36V-FKBP;
a signalling domain which comprises:
   a costimulatory sequence which comprises or consists of an amino acid sequence which is, or which is derived from, the intracellular domain of 4-1BB, and
   an ITAM-containing sequence which comprises or consists of an amino acid sequence which is, or which is derived from, the intracellular domain of CD3ζ.
      (J) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a signalling domain which comprises:
   a costimulatory sequence which comprises or consists of an amino acid sequence which is, or which is derived from, the intracellular domain of 4-1BB, and
   an ITAM-containing sequence which comprises or consists of an amino acid sequence which is, or which is derived from, the intracellular domain of CD3ζ.
      (K) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a dimerization domain which comprises or consists of an amino acid sequence which is, or which is derived from, the amino acid sequence of F36V-FKBP;
a signalling domain which comprises:
   a costimulatory sequence which comprises or consists of an amino acid sequence which is, or which is derived from, the intracellular domain of 4-1BB, and
   an ITAM-containing sequence which comprises or consists of an amino acid sequence which is, or which is derived from, the intracellular domain of CD3ζ.
      (L) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a signalling domain which comprises:
   a costimulatory sequence which comprises or consists of an amino acid sequence which is, or which is derived from, the intracellular domain of CD28, and
   an ITAM-containing sequence which comprises or consists of an amino acid sequence which is, or which is derived from, the intracellular domain of CD3ζ.
      (M) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD8α;
a dimerization domain which comprises or consists of an amino acid sequence which is, or which is derived from, the amino acid sequence of F36V-FKBP;
a signalling domain which comprises:
   a costimulatory sequence which comprises or consists of an amino acid sequence which is, or which is derived from, the intracellular domain of CD28, and
   an ITAM-containing sequence which comprises or consists of an amino acid sequence which is, or which is derived from, the intracellular domain of CD3ζ.

In some embodiments, the CAR according to any one of A, B, C, D, E, F, G, H, I, J K, L or M additionally comprises a hinge region between the antigen-binding domain and the transmembrane domain as described herein. In some embodiments the CARs comprise a hinge region which comprises or consists of an amino acid sequence which is, or which is derived from, the human IgG1 hinge region.

In some embodiments, the CAR according to any one of A, B, C, D, E, F, G, H, I, J K, L or M additionally comprises a signal sequence as described herein. In some embodiments the CARs comprise a signal sequence which comprises or consists of an amino acid sequence which is, or which is derived from, the human Ig heavy chain signal sequence.

In some embodiments, the CAR may comprise the combination of domains/sequences arranged as set out in one of (1) to (13) below. Optionally, in some embodiments the CAR may exclude the signal sequence. In some preferred embodiments the domains and sequences are present in the CAR from the N terminus to C terminus in the order described.
(1) N term-[signal sequence]-[GPC3-binding domain]-[hinge region]-[transmembrane domain]-[dimerization domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10;
   wherein the dimerization domain comprises, or consists of, an amino acid sequence having at least 80% sequence identity to the amino acid sequence of SEQ ID NO:20; and
   wherein the signaling domain comprises:
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(2) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11; and
   wherein the signaling domain comprises:
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(3) N term-[signal sequence]-[GPC3-binding domain]-[hinge region]-[transmembrane domain]-[dimerization domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11;
   wherein the dimerization domain comprises, or consists of, an amino acid sequence having at least 80% sequence identity to the amino acid sequence of SEQ ID NO:20; and
   wherein the signaling domain comprises:
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(4) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11; and
   wherein the signaling domain comprises:
      a costimulatory sequence which comprises, or consists of, an amino acid sequence having at least 80% sequence identity to the amino acid sequence of SEQ ID NO:16; and
      an ITAM-containing amino acid sequence which comprises, or consists of, an amino acid sequence having at least 80% sequence identity to the amino acid sequence of SEQ ID NO:14.
(5) N term-[signal sequence]-[GPC3-binding domain]-[hinge region]-[transmembrane domain]-[dimerization domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11;
   wherein the dimerization domain comprises, or consists of, an amino acid sequence having at least 80% sequence identity to the amino acid sequence of SEQ ID NO:20; and
   wherein the signaling domain comprises:
      a costimulatory sequence which comprises, or consists of, an amino acid sequence having at least 80% sequence identity to the amino acid sequence of SEQ ID NO:16; and
      an ITAM-containing amino acid sequence which comprises, or consists of, an amino acid sequence having at least 80% sequence identity to the amino acid sequence of SEQ ID NO:14.
(6) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11; and
   wherein the signaling domain comprises:
      a costimulatory sequence which comprises, or consists of, an amino acid sequence having at least 80% sequence identity to the amino acid sequence of SEQ ID NO:17; and
      an ITAM-containing amino acid sequence which comprises, or consists of, an amino acid sequence having at least 80% sequence identity to the amino acid sequence of SEQ ID NO:14.
(7) N term-[signal sequence]-[GPC3-binding domain]-[hinge region]-[transmembrane domain]-[dimerization domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11;
   wherein the dimerization domain comprises, or consists of, an amino acid sequence having at least 80% sequence identity to the amino acid sequence of SEQ ID NO:20; and
   wherein the signaling domain comprises:
      a costimulatory sequence which comprises, or consists of, an amino acid sequence having at least 80% sequence identity to the amino acid sequence of SEQ ID NO:17; and
      an ITAM-containing amino acid sequence which comprises, or consists of, an amino acid sequence having at least 80% sequence identity to the amino acid sequence of SEQ ID NO:14.
(8) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11; and
   wherein the signaling domain comprises:
      a costimulatory sequence which comprises, or consists of, an amino acid sequence having at least 80% sequence identity to the amino acid sequence of SEQ ID NO:16;
      a costimulatory sequence which comprises, or consists of, an amino acid sequence having at least 80% sequence identity to the amino acid sequence of SEQ ID NO:17; and
      an ITAM-containing amino acid sequence which comprises, or consists of, an amino acid sequence having at least 80% sequence identity to the amino acid sequence of SEQ ID NO:14.
(9) N term-[signal sequence]-[GPC3-binding domain]-[hinge region]-[transmembrane domain]-[dimerization domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11;
   wherein the dimerization domain comprises, or consists of, an amino acid sequence having at least 80% sequence identity to the amino acid sequence of SEQ ID NO:20; and
   wherein the signaling domain comprises:
      a costimulatory sequence which comprises, or consists of, an amino acid sequence having at least 80% sequence identity to the amino acid sequence of SEQ ID NO:16;
      a costimulatory sequence which comprises, or consists of, an amino acid sequence having at least 80% sequence identity to the amino acid sequence of SEQ ID NO:17; and
      an ITAM-containing amino acid sequence which comprises, or consists of, an amino acid sequence having at least 80% sequence identity to the amino acid sequence of SEQ ID NO:14.
(10) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11; and
   wherein the signaling domain comprises:
      a costimulatory sequence which comprises, or consists of, an amino acid sequence having at least 80% sequence identity to the amino acid sequence of SEQ ID NO:16;
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(11) N term-[signal sequence]-[GPC3-binding domain]-[hinge region]-[transmembrane domain]-[dimerization domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11;
   wherein the dimerization domain comprises, or consists of, an amino acid sequence having at least 80% sequence identity to the amino acid sequence of SEQ ID NO:20; and
   wherein the signaling domain comprises:
      a costimulatory sequence which comprises, or consists of, an amino acid sequence having at least 80% sequence identity to the amino acid sequence of SEQ ID NO:16;
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(12) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11; and
   wherein the signaling domain comprises:
      a costimulatory sequence which comprises, or consists of, an amino acid sequence having at least 80% sequence identity to the amino acid sequence of SEQ ID NO:16;
      a costimulatory sequence which comprises, or consists of, an amino acid sequence having at least 80% sequence identity to the amino acid sequence of SEQ ID NO:17;
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(13) N term-[signal sequence]-[GPC3-binding domain]-[hinge region]-[transmembrane domain]-[dimerization domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:11;
   wherein the dimerization domain comprises, or consists of, an amino acid sequence having at least 80% sequence identity to the amino acid sequence of SEQ ID NO:20; and
   wherein the signaling domain comprises:
      a costimulatory sequence which comprises, or consists of, an amino acid sequence having at least 80% sequence identity to the amino acid sequence of SEQ ID NO:16;
      a costimulatory sequence which comprises, or consists of, an amino acid sequence having at least 80% sequence identity to the amino acid sequence of SEQ ID NO:17;
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.

In some embodiments, the chimeric antigen receptor according to the present invention comprises, or consists of, an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:22, 23, 24, 25, 26, 27, 28, 29, 38, 39, 40, 41 or 42:

scFV GC33/hIgG1 hinge/CD8α TMD/CD226/CD3ζ:
(SEQ ID NO: 22)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNIVIFLNRRRRRE
RRDLFTESWDTQKAPNNYRSPISTSQPTNQSMDDTREDIYVNYPTFSRRP
KTRVRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTA
TKDTYDALHMQALPPR
scFV GC33/hIgG1 hinge/CD8α TMD/F36V-FKBP/CD226/
CD3ζ:
(SEQ ID NO: 23)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNGVQVETISPGDG
RTFPKRGQTCVVHYTGMLEDGKKVDSSRDRNKPFKFMLGKQEVIRGWEEG
VAQMSVGQRAKLTISPDYAYGATGHPGIIPPHATLVFDVELLKLEIVIFL
NRRRRRERRDLFTESWDTQKAPNNYRSPISTSQPTNQSMDDTREDIYVNY
PTFSRRPKTRVRVKFSRSADAPAYQQGQNQLYNELNLGRREEYDVLDKRR
GRDPEMGGKPRRKNPQEGLYNELQKDKMAEAYSEIGMKGERRRGKGHDGL
YQGLSTATKDTYDALHMQALPPR
scFV GC33/hIgG1 hinge/CD8α TMD/CD226/CD28/CD3ζ:
(SEQ ID NO: 24)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNIVIFLNRRRRRE
RRDLFTESWDTQKAPNNYRSPISTSQPTNQSMDDTREDIYVNYPTFSRRP
KTRVFWVRSKRSRLLHSDYMNMTPRRPGPTRKHYQPYAPPRDFAAYRSRV
KFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR
scFV GC33/hIgG1 hinge/CD8α TMD/F36V-FKBP/CD226/
CD28/CD3ζ:
(SEQ ID NO: 25)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNGVQVETISPGDG
RTFPKRGQTCVVHYTGMLEDGKKVDSSRDRNKPFKFMLGKQEVIRGWEEG
VAQMSVGQRAKLTISPDYAYGATGHPGIIPPHATLVFDVELLKLEIVIFL
NRRRRRERRDLFTESWDTQKAPNNYRSPISTSQPTNQSMDDTREDIYVNY
PTFSRRPKTRVFWVRSKRSRLLHSDYMNMTPRRPGPTRKHYQPYAPPRDF
AAYRSRVKFSRSADAPAYQQGQNQLYNELNLGRREEYDVLDKRRGRDPEM
GGKPRRKNPQEGLYNELQKDKMAEAYSEIGMKGERRRGKGHDGLYQGLST
ATKDTYDALHMQALPPR
scFV GC33/hIgG1 hinge/CD8α TMD/CD226/4-1BB/CD3ζ:
(SEQ ID NO: 26)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNIVIFLNRRRRRE
RRDLFTESWDTQKAPNNYRSPISTSQPTNQSMDDTREDIYVNYPTFSRRP
KTRVKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPR
scFV GC33/hIgG1 hinge/CD8α TMD/F36V-FKBP/CD226/
41BB/CD3ζ:
(SEQ ID NO: 27)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNGVQVETISPGDG
RTFPKRGQTCVVHYTGMLEDGKKVDSSRDRNKPFKFMLGKQEVIRGWEEG
VAQMSVGQRAKLTISPDYAYGATGHPGIIPPHATLVFDVELLKLEIVIFL
NRRRRRERRDLFTESWDTQKAPNNYRSPISTSQPTNQSMDDTREDIYVNY
PTFSRRPKTRVKRGRKKLLYIFKQPFMRPVQTTQEEDGCSCRFPEEEEGG
CELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPR
scFV GC33/hIgG1 hinge/CD8α TMD/CD226/CD28/4-1BB/
CD3ζ:
(SEQ ID NO: 28)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNIVIFLNRRRRRE
RRDLFTESWDTQKAPNNYRSPISTSQPTNQSMDDTREDIYVNYPTFSRRP
KTRVFWVRSKRSRLLHSDYMNMTPRRPGPTRKHYQPYAPPRDFAAYRSKR
GRKKLLYIFKQPFMRPVQTTQEEDGCSCRFPEEEEGGCELRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PRscFV GC33/hIgG1 hinge/CD8α TMD/F36V-FKBP/
CD226/CD28/41BB/CD3ζ:
(SEQ ID NO: 29)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNGVQVETISPGDG
RTFPKRGQTCVVHYTGMLEDGKKVDSSRDRNKPFKFMLGKQEVIRGWEEG
VAQMSVGQRAKLTISPDYAYGATGHPGIIPPHATLVFDVELLKLEIVIFL
NRRRRRERRDLFTESWDTQKAPNNYRSPISTSQPTNQSMDDTREDIYVNY
PTFSRRPKTRVFWVRSKRSRLLHSDYMNMTPRRPGPTRKHYQPYAPPRDF
AAYRSKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPR
scFV GC33/hIgG1 hinge/CD28 TMD/F36V-FKBP/41BB/
CD3ζ:
(SEQ ID NO: 38)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKFWVLWVGGVLACYSLLVTVAFIIGVQVETISPGDGRTFPK
RGQTCVVHYTGMLEDGKKVDSSRDRNKPFKFMLGKQEVIRGWEEGVAQMS
VGQRAKLTISPDYAYGATGHPGIIPPHATLVFDVELLKLEKRGRKKLLYI
FKQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR
scFV GC33/hIgG1 hinge/CD8α TMD/41BB/CD3ζ:
(SEQ ID NO: 39)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNKRGRKKLLYIFK
QPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR
scFV GC33/hIgG1 hinge/CD8α TMD/F36V-FKBP/41BB/
CD3ζ:
(SEQ ID NO: 40)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNGVQVETISPGDG
RTFPKRGQTCVVHYTGMLEDGKKVDSSRDRNKPFKFMLGKQEVIRGWEEG
VAQMSVGQRAKLTISPDYAYGATGHPGIIPPHATLVFDVELLKLEKRGRK
KLLYIFKQPFMRPVQTTQEEDGCSCRFPEEEEGGCELRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
scFV GC33/hIgG1 hinge/CD8α TMD/CD28/CD3ζ:
(SEQ ID NO: 41)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNFWVRSKRSRLLH
SDYMNMTPRRPGPTRKHYQPYAPPRDFAAYRSRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR
scFV GC33/hIgG1 hinge/CD8α TMD/F36V-FKBP/CD28/
CD3ζ:
(SEQ ID NO: 42)
QVQLQQSGAELVRPGASVKLSCKASGYTFTDYEMHWVKQTPVHGLKWIGA
LDPKTGDTAYSQKFKGKATLTADKSSSTAYMELRSLTSEDSAVYYCTRFY
SYTYWGQGTLVTVSAGGGGSGGGGSGGGGSDVVMTQTPLSLPVSLGDQAS
ISCRSSQSLVHSNGNTYLHWYLQKPGQSPKLLIYKVSNRFSGVPDRFSGS
GSGTDFTLKISRVEAEDLGVYFCSQNTHVPPTFGSGTKLEIKEPKSCDKT
HTCPPCPDPKIYIWAPLAGTCGVLLLSLVITLYCNHRNGVQVETISPGDG
RTFPKRGQTCVVHYTGMLEDGKKVDSSRDRNKPFKFMLGKQEVIRGWEEG
VAQMSVGQRAKLTISPDYAYGATGHPGIIPPHATLVFDVELLKLEFWVRS
KRSRLLHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R

In some embodiments, the chimeric antigen receptor according to the present invention comprises, or consists of, an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:30, 31, 32, 33, 34, 35, 36, 37, 43, 44, 45, 46 or 47:

hIgG heavy chain signal sequence/scFV GC33/hIgG1
hinge/CD8α TMD/CD226/CD3ζ:
(SEQ ID NO: 30)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNIVIFLNRRRRRERRDLFTESWDTQKAPNN
YRSPISTSQPTNQSMDDTREDIYVNYPTFSRRPKTRVRVKFSRSADAPAYQQGQN
QLYNELNLGRREEYDVLDKRRGRDPEMGGKPRRKNPQEGLYNELQKDKMAEAYS
EIGMKGERRRGKGHDGLYQGLSTATKDTYDALHMQALPPR
hIgG heavy chain signal sequence/scFV GC33/hIgG1
hinge/CD8α TMD/F36V-FKBP/CD226/CD3ζ:
(SEQ ID NO: 31)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNGVQVETISPGDGRTFPKRGQTCVVHYTG
MLEDGKKVDSSRDRNKPFKFMLGKQEVIRGWEEGVAQMSVGQRAKLTISPDYAYG
ATGHPGIIPPHATLVFDVELLKLEIVIFLNRRRRRERRDLFTESWDTQKAPNNYRSPI
STSQPTNQSMDDTREDIYVNYPTFSRRPKTRVRVKFSRSADAPAYQQGQNQLYNE
LNLGRREEYDVLDKRRGRDPEMGGKPRRKNPQEGLYNELQKDKMAEAYSEIGMK
GERRRGKGHDGLYQGLSTATKDTYDALHMQALPPR
hIgG heavy chain signal sequence/scFV GC33/hIgG1
hinge/CD8α TMD/CD226/CD28/CD3ζ:
(SEQ ID NO: 32)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNIVIFLNRRRRRERRDLFTESWDTQKAPNN
YRSPISTSQPTNQSMDDTREDIYVNYPTFSRRPKTRVFWVRSKRSRLLHSDYMNM
TPRRPGPTRKHYQPYAPPRDFAAYRSRVKFSRSADAPAYQQGQNQLYNELNLGR
REEYDVLDKRRGRDPEMGGKPRRKNPQEGLYNELQKDKMAEAYSEIGMKGERRR
GKGHDGLYQGLSTATKDTYDALHMQALPPR
hIgG heavy chain signal sequence/scFV GC33/hIgG1
hinge/CD8α TMD/F36V-FKBP/CD226/CD28/CD3ζ:
(SEQ ID NO: 33)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNGVQVETISPGDGRTFPKRGQTCVVHYTG
MLEDGKKVDSSRDRNKPFKFMLGKQEVIRGWEEGVAQMSVGQRAKLTISPDYAYG
ATGHPGIIPPHATLVFDVELLKLEIVIFLNRRRRRERRDLFTESWDTQKAPNNYRSPI
STSQPTNQSMDDTREDIYVNYPTFSRRPKTRVFWVRSKRSRLLHSDYMNMTPRRP
GPTRKHYQPYAPPRDFAAYRSRVKFSRSADAPAYQQGQNQLYNELNLGRREEYD
VLDKRRGRDPEMGGKPRRKNPQEGLYNELQKDKMAEAYSEIGMKGERRRGKGHD
GLYQGLSTATKDTYDALHMQALPPR
hIgG heavy chain signal sequence/scFV GC33/hIgG1
hinge/CD8α TMD/CD226/4-1BB/CD3ζ:
(SEQ ID NO: 34)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNIVIFLNRRRRRERRDLFTESWDTQKAPNN
YRSPISTSQPTNQSMDDTREDIYVNYPTFSRRPKTRVKRGRKKLLYIFKQPFMRPV
QTTQEEDGCSCRFPEEEEGGCELRVKFSRSADAPAYQQGQNQLYNELNLGRREE
YDVLDKRRGRDPEMGGKPRRKNPQEGLYNELQKDKMAEAYSEIGMKGERRRGKG
HDGLYQGLSTATKDTYDALHMQALPPR
hIgG heavy chain signal sequence/scFV GC33/hIgG1
hinge/CD8α TMD/F36V-FKBP/CD226/41BB/CD3ζ:
(SEQ ID NO: 35)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNGVQVETISPGDGRTFPKRGQTCVVHYTG
MLEDGKKVDSSRDRNKPFKFMLGKQEVIRGWEEGVAQMSVGQRAKLTISPDYAYG
ATGHPGIIPPHATLVFDVELLKLEIVIFLNRRRRRERRDLFTESWDTQKAPNNYRSPI
STSQPTNQSMDDTREDIYVNYPTFSRRPKTRVKRGRKKLLYIFKQPFMRPVQTTQE
EDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGL
YQGLSTATKDTYDALHMQALPPR
hIgG heavy chain signal sequence/scFV GC33/hIgG1
hinge/CD8α TMD/CD226/CD28/4-1BB/CD3ζ:
(SEQ ID NO: 36)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNIVIFLNRRRRRERRDLFTESWDTQKAPNN
YRSPISTSQPTNQSMDDTREDIYVNYPTFSRRPKTRVFWVRSKRSRLLHSDYMNM
TPRRPGPTRKHYQPYAPPRDFAAYRSKRGRKKLLYIFKQPFMRPVQTTQEEDGCS
CRFPEEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQGLST
ATKDTYDALHMQALPPR
hIgG heavy chain signal sequence/scFV GC33/hIgG1
hinge/CD8α TMD/F36V-FKBP/CD226/CD28/41BB/CD3ζ:
(SEQ ID NO: 37)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNGVQVETISPGDGRTFPKRGQTCVVHYTG
MLEDGKKVDSSRDRNKPFKFMLGKQEVIRGWEEGVAQMSVGQRAKLTISPDYAYG
ATGHPGIIPPHATLVFDVELLKLEIVIFLNRRRRRERRDLFTESWDTQKAPNNYRSPI
STSQPTNQSMDDTREDIYVNYPTFSRRPKTRVFWVRSKRSRLLHSDYMNMTPRRP
GPTRKHYQPYAPPRDFAAYRSKRGRKKLLYIFKQPFMRPVQTTQEEDGCSCRFPE
EEEGGCELRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDT
YDALHMQALPPR
hIgG heavy chain signal sequence/scFV GC33/hIgG1
hinge/CD28 TMD/F36V-FKBP/41BB/CD3ζ:
(SEQ ID NO: 43)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KFWVLVWGGVLACYSLLVTVAFIIGVQVETISPGDGRTFPKRGQTCVVHYTGMLED
GKKVDSSRDRNKPFKFMLGKQEVIRGWEEGVAQMSVGQRAKLTISPDYAYGATGH
PGIIPPHATLVFDVELLKLEKRGRKKLLYIFKQPFMRPVQTTQEEDGCSCRFPEEEE
GGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDAL
HMQALPPR
hIgG heavy chain signal sequence/scFV GC33/hIgG1
hinge/CD8α TMD/41BB/CD3ζ:
(SEQ ID NO: 44)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNKRGRKKLLYIFKQPFMRPVQTTQEEDGCS
CRFPEEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQGLST
ATKDTYDALHMQALPPR
hIgG heavy chain signal sequence/scFV GC33/hIgG1
hinge/CD8α TMD/F36V-FKBP/41BB/CD3ζ:
(SEQ ID NO: 45)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNGVQVETISPGDGRTFPKRGQTCVVHYTG
MLEDGKKVDSSRDRNKPFKFMLGKQEVIRGWEEGVAQMSVGQRAKLTISPDYAYG
ATGHPGIIPPHATLVFDVELLKLEKRGRKKLLYIFKQPFMRPVQTTQEEDGCSCRFP
EEEEGGCELRVKFSRSADAPAYQQGQNQLYNELNLGRREEYDVLDKRRGRDPEM
GGKPRRKNPQEGLYNELQKDKMAEAYSEIGMKGERRRGKGHDGLYQGLSTATKD
TYDALHMQALPPR
hIgG heavy chain signal sequence/scFV GC33/hIgG1
hinge/CD8α TMD/CD28/CD3ζ:
(SEQ ID NO: 46)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNFWVRSKRSRLLHSDYMNMTPRRPGPTRK
HYQPYAPPRDFAAYRSRVKFSRSADAPAYQQGQNQLYNELNLGRREEYDVLDKR
RGRDPEMGGKPRRKNPQEGLYNELQKDKMAEAYSEIGMKGERRRGKGHDGLYQ
GLSTATKDTYDALHMQALPPR
hIgG heavy chain signal sequence/scFV GC33/hIgG1
hinge/CD8α TMD/F36V-FKBP/CD28/CD3ζ:
(SEQ ID NO: 47)
MDWIWRILFLVGAATGAHSQVQLQQSGAELVRPGASVKLSCKASGYTFTDYEMHW
VKQTPVHGLKWIGALDPKTGDTAYSQKFKGKATLTADKSSSTAYMELRSLTSEDSA
VYYCTRFYSYTYWGQGTLVTVSAGGGGSGGGGSGGGGSDVVMTQTPLSLPVSLG
DQASISCRSSQSLVHSNGNTYLHWYLQKPGQSPKLLIYKVSNRFSGVPDRFSGSGS
GTDFTLKISRVEAEDLGVYFCSQNTHVPPTFGSGTKLEIKEPKSCDKTHTCPPCPDP
KIYIWAPLAGTCGVLLLSLVITLYCNHRNGVQVETISPGDGRTFPKRGQTCVVHYTG
MLEDGKKVDSSRDRNKPFKFMLGKQEVIRGWEEGVAQMSVGQRAKLTISPDYAYG
ATGHPGIIPPHATLVFDVELLKLEFWVRSKRSRLLHSDYMNMTPRRPGPTRKHYQP
YAPPRDFAAYRSRVKFSRSADAPAYQQGQNQLYNELNLGRREEYDVLDKRRGRD
PEMGGKPRRKNPQEGLYNELQKDKMAEAYSEIGMKGERRRGKGHDGLYQGLSTA
TKDTYDALHMQALPPR

In some embodiments, the CAR of the present invention does not comprise the combination of domains/sequences according to N, O, P, Q, R, S, T, or U shown in Table 2:

TABLE 2

| | | | Trans- | |
|---|---|---|---|---|
| | Antigen-binding | | membrane | |
| | domain | Hinge region | domain | Signaling domain |
| N | GPC3-binding scFV | CD8α | CD8α | CD3ζ |
| O | GPC3-binding scFV | CD8α | CD8α | 4-1BB, CD3ζ |
| P | GPC3-binding scFV | CD8α | CD28 | CD28, CD3ζ |
| Q | GPC3-binding scFV | CD8α | CD28 | CD28, 4-1BB, CD3ζ |
| R | GPC3-binding scFV | Human IgG1 | CD28 | CD3ζ |
| S | GPC3-binding scFV | Human IgG1 | CD28 | CD28, CD3ζ |
| T | GPC3-binding scFV | Human IgG1 | CD28 | 4-1BB, CD3ζ |
| U | GPC3-binding scFV | Human IgG1 | CD28 | CD28, 4-1BB, CD3ζ |

In some embodiments, the CAR may comprise the combination of domains/sequences according to any one of V to MM as shown in Table 3:

TABLE 3

| | | Trans- | |
|---|---|---|---|
| | Antigen-binding | membrane | |
| | domain | domain | Signaling domain |
| V | GPC3-binding scFV | CD28 | CD226 ICDv2 |
| W | GPC3-binding scFV | CD28 | CD226 ICDv1, 4-1BB, CD3ζ |
| X | GPC3-binding scFV | CD28 | CD226 ICDv2, 4-1BB, CD3ζ |
| Y | GPC3-binding scFV | CD28 | CD226 ICDv1, CD3ζ |
| Z | GPC3-binding scFV | CD28 | CD226 ICDv2, CD3ζ |
| AA | GPC3-binding scFV | CD28 | CD226 ICDv1, CD28, CD3ζ |
| BB | GPC3-binding scFV | CD28 | CD226 ICDv2, CD28, CD3ζ |
| CC | GPC3-binding scFV | CD28 | CD28, CD226 ICDv1, CD3ζ |
| DD | GPC3-binding scFV | CD28 | CD226 ICDv1, CD28, 4-1BB, |
| | | | CD3ζ |
| EE | GPC3-binding scFV | CD28 | CD226 ICDv2, CD28, 4-1BB, |
| | | | CD3ζ |
| FF | GPC3-binding scFV | CD28 | CD28, CD226 ICDv1, 4-1BB, |
| | | | CD3ζ |
| GG | GPC3-binding scFV | CD226 | CD226 ICDv1, 4-1BB, CD3ζ |
| HH | EpCAM-binding scFV | CD226 | CD226 ICDv1 |
| II | EpCAM-binding scFV | CD226 | CD3ζ |
| JJ | EpCAM-binding scFV | CD226 | CD226 ICDv1, CD3ζ |
| KK | GPC3-binding scFV | CD28 | 4-1BB, CD3ζ, CD226 ICDv1 |
| LL | GPC3-binding scFV | CD28 | 4-1BB, CD226 ICDv1, CD3ζ |
| MM | GPC3-binding scFV | CD226 | CD226 ICDv1 |

It will be appreciated that Table 3 provides short-hand representations for the combinations of domains/sequences of the CARs V to MM. CARs V to MM comprise the following combinations of domains/sequences:

(V) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD28; and
a signalling domain which comprises:
   a costimulatory sequence which comprises or consists of an amino acid sequence which is, or which is derived from, the intracellular domain of CD226.
      (W, X) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD28;
a signalling domain which comprises:
   a costimulatory sequence which comprises or consists of an amino acid sequence which is, or which is derived from, the intracellular domain of CD226,
   a costimulatory sequence which comprises or consists of an amino acid sequence which is, or which is derived from, the intracellular domain of 4-1BB, and
   an ITAM-containing sequence which comprises or consists of an amino acid sequence which is, or which is derived from, the intracellular domain of CD3ζ.
      (Y, Z) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD28;
a signalling domain which comprises:
   a costimulatory sequence which comprises or consists of an amino acid sequence which is, or which is derived from, the intracellular domain of CD226, and
   an ITAM-containing sequence which comprises or consists of an amino acid sequence which is, or which is derived from, the intracellular domain of CD3ζ.
      (AA, BB) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD28;
a signalling domain which comprises:
   a costimulatory sequence which comprises or consists of an amino acid sequence which is, or which is derived from, the intracellular domain of CD226,
   a costimulatory sequence which comprises or consists of an amino acid sequence which is, or which is derived from, the intracellular domain of CD28, and
   an ITAM-containing sequence which comprises or consists of an amino acid sequence which is, or which is derived from, the intracellular domain of CD3ζ.
      (CC) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD28;
a signalling domain which comprises:
   a costimulatory sequence which comprises or consists of an amino acid sequence which is, or which is derived from, the intracellular domain of CD28,
   a costimulatory sequence which comprises or consists of an amino acid sequence which is, or which is derived from, the intracellular domain of CD226, and
   an ITAM-containing sequence which comprises or consists of an amino acid sequence which is, or which is derived from, the intracellular domain of CD3ζ.
      (DD, EE) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD28;
a signalling domain which comprises:
   a costimulatory sequence which comprises or consists of an amino acid sequence which is, or which is derived from, the intracellular domain of CD226,
   a costimulatory sequence which comprises or consists of an amino acid sequence which is, or which is derived from, the intracellular domain of CD28,
   a costimulatory sequence which comprises or consists of an amino acid sequence which is, or which is derived from, the intracellular domain of 4-1BB, and
   an ITAM-containing sequence which comprises or consists of an amino acid sequence which is, or which is derived from, the intracellular domain of CD3ζ.
      (FF) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD28;
a signalling domain which comprises:
   a costimulatory sequence which comprises or consists of an amino acid sequence which is, or which is derived from, the intracellular domain of CD28,
   a costimulatory sequence which comprises or consists of an amino acid sequence which is, or which is derived from, the intracellular domain of CD226,
   a costimulatory sequence which comprises or consists of an amino acid sequence which is, or which is derived from, the intracellular domain of 4-1BB, and
   an ITAM-containing sequence which comprises or consists of an amino acid sequence which is, or which is derived from, the intracellular domain of CD3ζ.
      (GG) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD226;
a signalling domain which comprises:
   a costimulatory sequence which comprises or consists of an amino acid sequence which is, or which is derived from, the intracellular domain of CD226,
   a costimulatory sequence which comprises or consists of an amino acid sequence which is, or which is derived from, the intracellular domain of 4-1BB, and
   an ITAM-containing sequence which comprises or consists of an amino acid sequence which is, or which is derived from, the intracellular domain of CD3ζ.
      (HH) An EpCAM-binding domain which comprises or consists of an amino acid sequence which is, or which is derived from, an EpCAM-binding scFV;
a transmembrane domain which comprises or consists of an amino acid sequence which is, or which is derived from, the transmembrane domain of CD226; and
a signalling domain which comprises:
   a costimulatory sequence which comprises or consists of an amino acid sequence which is, or which is derived from, the intracellular domain of CD226.
      (II) An EpCAM-binding domain which comprises or consists of an amino acid sequence which is, or which is derived from, an EpCAM-binding scFV;
a transmembrane domain which comprises or consists of an amino acid sequence which is, or which is derived from, the transmembrane domain of CD226; and
a signalling domain which comprises:
   an ITAM-containing sequence which comprises or consists of an amino acid sequence which is, or which is derived from, the intracellular domain of CD3ζ.
      (JJ) An EpCAM-binding domain which comprises or consists of an amino acid sequence which is, or which is derived from, an EpCAM-binding scFV;
a transmembrane domain which comprises or consists of an amino acid sequence which is, or which is derived from, the transmembrane domain of CD226; and
a signalling domain which comprises:
   a costimulatory sequence which comprises or consists of an amino acid sequence which is, or which is derived from, the intracellular domain of CD226, and
   an ITAM-containing sequence which comprises or consists of an amino acid sequence which is, or which is derived from, the intracellular domain of CD3ζ.
      (KK) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD28; and
a signalling domain which comprises:
   a costimulatory sequence which comprises or consists of an amino acid sequence which is, or which is derived from, the intracellular domain of 4-1BB,
   an ITAM-containing sequence which comprises or consists of an amino acid sequence which is, or which is derived from, the intracellular domain of CD3ζ, and
   a costimulatory sequence which comprises or consists of an amino acid sequence which is, or which is derived from, the intracellular domain of CD226.
      (LL) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD28; and
a signalling domain which comprises:
   a costimulatory sequence which comprises or consists of an amino acid sequence which is, or which is derived from, the intracellular domain of 4-1BB,
   a costimulatory sequence which comprises or consists of an amino acid sequence which is, or which is derived from, the intracellular domain of CD226, and
   an ITAM-containing sequence which comprises or consists of an amino acid sequence which is, or which is derived from, the intracellular domain of CD3ζ.
      (MM) A GPC3-binding domain which comprises or consists of an amino acid sequence which is, or which is derived from, a GPC3-binding scFV;
a transmembrane domain which comprises or consists of an amino acid sequence which is, or which is derived from, the transmembrane domain of CD226; and
a signalling domain which comprises:
   a costimulatory sequence which comprises or consists of an amino acid sequence which is, or which is derived from, the intracellular domain of CD226.

In some embodiments, the CAR may comprise the combination of domains/sequences according to BB. In some embodiments, the CAR may comprise the combination of domains/sequences according to W. In some embodiments, the CAR may comprise the combination of domains/sequences according to X.

In some embodiments, the CAR according to any one of V, W, X, Y, Z, AA, BB, CC, DD, EE, FF, GG, HH, II, JJ, KK, LL or MM additionally comprises a hinge region between the antigen-binding domain and the transmembrane domain as described herein. In some embodiments the CARs comprise a hinge region which comprises or consists of an amino acid sequence which is, or which is derived from, the human IgG1 hinge region.

In some embodiments, the CAR according to any one of V, W, X, Y, Z, AA, BB, CC, DD, EE, FF, GG, HH, II, JJ, KK, LL or MM additionally comprises a signal sequence as described herein. In some embodiments the CARs comprise a signal sequence which comprises or consists of an amino acid sequence which is, or which is derived from, the human Ig heavy chain signal sequence.

In some embodiments, the CAR may comprise the combination of domains/sequences arranged as set out in one of (14) to (31) below. Optionally, in some embodiments the CAR may exclude the signal sequence. In some preferred embodiments the domains and sequences are present in the CAR from the N terminus to C terminus in the order described.
(14) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:59.
(15) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:58;
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(16) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:59;
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(17) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:58; and
      an ITAM-containing amino acid sequence which comprises, or consists of, an amino acid sequence having at least 80% sequence identity to the amino acid sequence of SEQ ID NO:14.
(18) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:59; and
      an ITAM-containing amino acid sequence which comprises, or consists of, an amino acid sequence having at least 80% sequence identity to the amino acid sequence of SEQ ID NO:14.
(19) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:58;
      a costimulatory sequence which comprises, or consists of, an amino acid sequence having at least 80% sequence identity to the amino acid sequence of SEQ ID NO:17; and
      an ITAM-containing amino acid sequence which comprises, or consists of, an amino acid sequence having at least 80% sequence identity to the amino acid sequence of SEQ ID NO:14.
(20) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:59;
      a costimulatory sequence which comprises, or consists of, an amino acid sequence having at least 80% sequence identity to the amino acid sequence of SEQ ID NO:17; and
      an ITAM-containing amino acid sequence which comprises, or consists of, an amino acid sequence having at least 80% sequence identity to the amino acid sequence of SEQ ID NO:14.
(21) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:17;
      a costimulatory sequence which comprises, or consists of, an amino acid sequence having at least 80% sequence identity to the amino acid sequence of SEQ ID NO:58; and
      an ITAM-containing amino acid sequence which comprises, or consists of, an amino acid sequence having at least 80% sequence identity to the amino acid sequence of SEQ ID NO:14.
(22) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:58;
      a costimulatory sequence which comprises, or consists of, an amino acid sequence having at least 80% sequence identity to the amino acid sequence of SEQ ID NO:17;
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(23) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:59;
      a costimulatory sequence which comprises, or consists of, an amino acid sequence having at least 80% sequence identity to the amino acid sequence of SEQ ID NO:17;
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(24) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:17;
      a costimulatory sequence which comprises, or consists of, an amino acid sequence having at least 80% sequence identity to the amino acid sequence of SEQ ID NO:58;
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(25) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:60; and
   wherein the signaling domain comprises:
      a costimulatory sequence which comprises, or consists of, an amino acid sequence having at least 80% sequence identity to the amino acid sequence of SEQ ID NO:58;
      a costimulatory sequence which comprises, or consists of, an amino acid sequence having at least 80% sequence identity to the amino acid sequence of SEQ ID NO:18; and
      an ITAM-containing amino acid sequence which comprises, or consists of, an amino acid sequence having at least 80% sequence identity to the amino acid sequence of SEQ ID NO:14.
(26) N term-[signal sequence]-[EpCAM-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the EpCAM-binding domain comprises:
      a heavy chain variable region sequence having at least 85% sequence identity to the amino acid sequence of SEQ ID NO:48, and
      a light chain variable region sequence having at least 85% sequence identity to the amino acid sequence of SEQ ID NO:52;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:60; and
   wherein the signaling domain comprises:
      a costimulatory sequence which comprises, or consists of, an amino acid sequence having at least 80% sequence identity to the amino acid sequence of SEQ ID NO:58.
(27) N term-[signal sequence]-[EpCAM-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the EpCAM-binding domain comprises:
      a heavy chain variable region sequence having at least 85% sequence identity to the amino acid sequence of SEQ ID NO:48, and
      a light chain variable region sequence having at least 85% sequence identity to the amino acid sequence of SEQ ID NO:52;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:60; and
   wherein the signaling domain comprises:
      an ITAM-containing amino acid sequence which comprises, or consists of, an amino acid sequence having at least 80% sequence identity to the amino acid sequence of SEQ ID NO:14.
(28) N term-[signal sequence]-[EpCAM-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the EpCAM-binding domain comprises:
      a heavy chain variable region sequence having at least 85% sequence identity to the amino acid sequence of SEQ ID NO:48, and
      a light chain variable region sequence having at least 85% sequence identity to the amino acid sequence of SEQ ID NO:52;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:60; and
   wherein the signaling domain comprises:
      a costimulatory sequence which comprises, or consists of, an amino acid sequence having at least 80% sequence identity to the amino acid sequence of SEQ ID NO:58; and
      an ITAM-containing amino acid sequence which comprises, or consists of, an amino acid sequence having at least 80% sequence identity to the amino acid sequence of SEQ ID NO:14.
(29) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:18;
      an ITAM-containing amino acid sequence which comprises, or consists of, an amino acid sequence having at least 80% sequence identity to the amino acid sequence of SEQ ID NO:14; and
      a costimulatory sequence which comprises, or consists of, an amino acid sequence having at least 80% sequence identity to the amino acid sequence of SEQ ID NO:58.
(30) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:10; and
   wherein the signaling domain comprises:
      a costimulatory sequence which comprises, or consists of, an amino acid sequence having at least 80% sequence identity to the amino acid sequence of SEQ ID NO:18;
      a costimulatory sequence which comprises, or consists of, an amino acid sequence having at least 80% sequence identity to the amino acid sequence of SEQ ID NO:58; and
      an ITAM-containing amino acid sequence which comprises, or consists of, an amino acid sequence having at least 80% sequence identity to the amino acid sequence of SEQ ID NO:14.
(31) N term-[signal sequence]-[GPC3-binding domain]-[hinge region]-[transmembrane domain]-[signalling domain]-C term;
   wherein the signal sequence comprises or consists of an amino acid sequence having at least 80% sequence identity to the amino acid sequence of SEQ ID NO:21;
   wherein the GPC3-binding domain comprises:
      a heavy chain variable region sequence having at least 85% sequence identity to the amino acid sequence of SEQ ID NO:1, and
      a light chain variable region sequence having at least 85% sequence identity to the amino acid sequence of SEQ ID NO:5;
   wherein the hinge region comprises, or consists of, an amino acid sequence having at least 80% sequence identity to the amino acid sequence of SEQ ID NO:19;
   wherein the transmembrane domain comprises, or consists of, an amino acid sequence having at least 80% sequence identity to the amino acid sequence of SEQ ID NO:60; and
   wherein the signaling domain comprises:
      a costimulatory sequence which comprises, or consists of, an amino acid sequence having at least 80% sequence identity to the amino acid sequence of SEQ ID NO:58.

In some embodiments, the CAR may comprise the combination of domains/sequences arranged as set out in (15) above. In some embodiments, the CAR may comprise the combination of domains/sequences arranged as set out in (20) above.

In some embodiments, the chimeric antigen receptor according to the present invention comprises, or consists of, an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:61, 62, 63, 64, 65, 66, 67, 68, 69, 70, 71, 72, 73, 74, 75, 76, 77, 78, 79, 118, 119 or 120.

In some embodiments, the chimeric antigen receptor according to the present invention comprises, or consists of, an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:70. In some embodiments, the chimeric antigen receptor according to the present invention comprises, or consists of, an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:64.

In some embodiments, the chimeric antigen receptor according to the present invention comprises, or consists of, an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:80, 81, 82, 83, 84, 85, 86, 87, 88, 89, 90, 91, 92, 93, 94, 95, 96, 97, 98, 121, 122 or 123.

In some embodiments, the chimeric antigen receptor according to the present invention comprises, or consists of, an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:89. In some embodiments, the chimeric antigen receptor according to the present invention comprises, or consists of, an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:83. In some embodiments, the chimeric antigen receptor according to the present invention comprises, or consists of, an amino acid sequence having at least 60%, 65%, 70%, 75%, 80%, 85%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:84.

### Nucleic Acids Encoding the CARs

The present invention provides a nucleic acid encoding a chimeric antigen receptor according to the present invention. In some embodiments, the nucleic acid is purified or isolated, e.g. from other nucleic acid, or naturally-occurring biological material.

The present invention also provides a vector comprising nucleic acid encoding a chimeric antigen receptor according to the present invention.

A “vector” as used herein is a nucleic acid (DNA or RNA) used as a vehicle to transfer exogenous nucleic acid into a cell. The vector may be an expression vector for expression of the nucleic acid in the cell. Such vectors may include a promoter sequence operably linked to the nucleic acid encoding the sequence to be expressed. A vector may also include a termination codon and expression enhancers. Any suitable vectors, promoters, enhancers and termination codons known in the art may be used to express a CAR according to the invention from a vector according to the invention.

Suitable vectors include plasmids, binary vectors, DNA vectors, mRNA vectors, viral vectors (e.g. gammaretroviral vectors, lentiviral vectors, adenovirus vectors), transposon-based vectors, and artificial chromosomes (e.g. yeast artificial chromosomes), e.g. as described in Maus et al., Annu Rev Immunol (2014) 32:189-225, which is hereby incorporated by reference in its entirety. In some embodiments, the viral vector may be a lentiviral, retroviral, adenoviral, or Herpes Simplex Virus vector. In some embodiments, the lentiviral vector may be pELNS, or may be derived from pELNS. In some embodiments, the vector may be a vector encoding CRISPR/Cas9.

In this specification the term “operably linked” may include the situation where a selected nucleic acid sequence and regulatory nucleic acid sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. Where appropriate, the resulting transcript may then be translated into a desired polypeptide.

In some embodiments, the nucleic acid according to the present invention comprises, or consists of, a nucleic acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO:99, 100, 101, 102, 103, 104, 105, 106, 107, 108, 109, 110, 111, 112, 113, 114, 115, 116, 117, 124, 125 or 126, or a nucleic acid sequence encoding the same amino acid sequence as one of SEQ ID NO:99, 100, 101, 102, 103, 104, 105, 106, 107, 108, 109, 110, 111, 112, 113, 114, 115, 116, 117, 124, 125 or 126 as a result of codon degeneracy.

In some embodiments, the nucleic acid according to the present invention comprises, or consists of, a nucleic acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO:108, or a nucleic acid sequence encoding the same amino acid sequence as one of SEQ ID NO:108 as a result of codon degeneracy. In some embodiments, the nucleic acid according to the present invention comprises, or consists of, a nucleic acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO:102, or a nucleic acid sequence encoding the same amino acid sequence as one of SEQ ID NO:102 as a result of codon degeneracy. In some embodiments, the nucleic acid according to the present invention comprises, or consists of, a nucleic acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO:103, or a nucleic acid sequence encoding the same amino acid sequence as one of SEQ ID NO:103 as a result of codon degeneracy.

### Cells Expressing the CARs

The present invention also provides a cell expressing a CAR according to the present invention. Also provided is a cell comprising a nucleic acid or vector according to the invention.

The cell may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell may be from, or may have been obtained from, a human subject.

The cell may be a cell of hematopoietic origin, e.g. a neutrophil, eosinophil, basophil, lymphocyte, or monocyte. The lymphocyte may be e.g. a T cell, B cell or NK cell or precursor. The cell may express e.g. CD3 polypeptides (e.g. CD3γ CD3ε CD3ζ or CD3δ), TCR polypeptides (TCRα or TCRβ), CD27, CD28, CD4 or CD8.

In some embodiments, the cell is a T cell. In some embodiments, the T cell is a CD3+ T cell. In some embodiments, the T cell is a CD3+, CD8+ T cell. In some embodiments, the T cell is a cytotoxic T cell (e.g. a cytotoxic T lymphocyte (CTL)).

In some embodiments, the cell is a target protein-reactive CAR-T cell. In embodiments herein, a “target protein-reactive” CAR-T cell is a cell which displays certain functional properties of a T cell in response to the target protein for which the antigen-binding domain of the CAR is specific, e.g. expressed at the surface of a cell. In some embodiments, the properties are functional properties associated with effector T cells, e.g. cytotoxic T cells.

In some embodiments, a target protein-reactive CAR-T cell may display one or more of the following properties: cytotoxicity to a cell comprising or expressing the target protein;

proliferation, increased IFNγ expression, increased CD107a expression, increased IL-2 expression, increased TNFα expression, increased perforin expression, increased granzyme expression, increased granulysin expression, and/or increased FAS ligand (FASL) expression in response to the target protein, or a cell comprising or expressing the target protein.

Herein, “expression” of IFNγ, CD107a, IL-2, TNFα, perforin, granzyme and/or FASL may refer to gene expression or protein expression. Gene expression can be measured by a various means known to those skilled in the art, for example by measuring levels of mRNA by quantitative real-time PCR (qRT-PCR), or by reporter-based methods. Similarly, protein expression can be measured by various methods well known in the art, e.g. by antibody-based methods, for example by western blot, immunohistochemistry, immunocytochemistry, flow cytometry, ELISA, ELISPOT, or reporter-based methods. “Increased expression” refers to a level of expression which is greater than the level of expression of the gene/protein by a T cell which has not been contacted with the target protein or a cell comprising or expressing the target protein, or the level of expression by a T cell in response to a cell not comprising or expressing the target protein.

The present invention also provides a method for producing a cell comprising a nucleic acid or vector according to the present invention, comprising introducing a nucleic acid or vector according to the present invention into a cell. The present invention also provides a method for producing a cell expressing a CAR according to the present invention, comprising introducing a nucleic acid or vector according to the present invention in a cell. In some embodiments, the methods additionally comprise culturing the cell under conditions suitable for expression of the nucleic acid or vector by the cell. In some embodiments, the methods are performed in vitro.

In some embodiments, introducing an isolated nucleic acid or vector according to the invention into a cell comprises transduction, e.g. retroviral transduction. Accordingly, in some embodiments the isolated nucleic acid or vector is comprised in a viral vector, or the vector is a viral vector. In some embodiments, the method comprises introducing a nucleic acid or vector according to the invention by electroporation, e.g. as described in Koh et al., Molecular Therapy—Nucleic Acids (2013) 2, e114, which is hereby incorporated by reference in its entirety.

The present invention also provides cells obtained or obtainable by the methods for producing a cell according to the present invention.

### Compositions

The present invention also provides compositions comprising a chimeric antigen receptor, nucleic acid, vector or cell according to the invention.

CARs, nucleic acids, vectors and cells according to the present invention may be formulated as pharmaceutical compositions for clinical use and may comprise a pharmaceutically acceptable carrier, diluent, excipient or adjuvant.

In accordance with the present invention methods are also provided for the production of pharmaceutically useful compositions, such methods of production may comprise one or more steps selected from: isolating a CAR, cell, nucleic acid or vector as described herein; and/or mixing a CAR, cell, nucleic acid or vector as described herein with a pharmaceutically acceptable carrier, adjuvant, excipient or diluent.

For example, a further aspect of the present invention relates to a method of formulating or producing a medicament or pharmaceutical composition for use in the treatment of a cancer, the method comprising formulating a pharmaceutical composition or medicament by mixing a CAR, cell, nucleic acid or vector as described herein with a pharmaceutically acceptable carrier, adjuvant, excipient or diluent.

### Properties of the CARs/Cells Expressing the CARs

The CAR of the present invention may also be defined by reference to properties of the CAR. A cell expressing the CAR may also be defined by reference properties of the cell expressing the CAR.

A CAR according to the present invention may display an increased level surface expression when expressed in a cell, as compared to the level of surface expression for another CAR, e.g. a CAR lacking a costimulatory sequence which is, or which is derived from, the intracellular domain of CD226. The increased level of cell surface expression of a CAR according to the present invention may be associated with one or more domains of the CAR of the present invention, or the particular combination of domains.

Cell surface expression for a CAR expressed in a cell can be analyzed by methods well known to the skilled person including, e.g. flow cytometry or immunofluorescence analysis, e.g. using labelled ligand for the antigen-binding domain.

A CAR according to the present invention comprising a dimerization domain may display increased expression at the cell surface of a cell expressing the CAR as compared to the level of expression at the cell surface for a comparable CAR lacking the dimerization domain. In some embodiments, the cell may exhibit increased expression at the cell surface following treatment with an agent. For example, in embodiments wherein the dimerization domain is an inducible dimerization domain, the cell may display increased surface expression as compared to a comparable CAR lacking the dimerization domain following treatment with the appropriate agent for inducing dimerization, oligomerization, or multimerization of the CAR.

A cell expressing a CAR according to the present invention may possess a certain property, or may display an increased level of a certain activity, as compared to the level of activity for a cell expressing another CAR, e.g. a CAR lacking a costimulatory sequence which is, or which is derived from, the intracellular domain of CD226.

For example, a cell expressing a CAR according to the present invention may display one or more of the following properties as compared to a cell expressing another CAR, e.g. a CAR lacking a costimulatory sequence which is, or which is derived from, the intracellular domain of CD226:
(a) increased rate of proliferation
(b) increased expression of one or more growth factors (e.g. IL-2)
(c) increased survival
(d) increased expression of one or more cytotoxic/effector factors (e.g. IFNγ, granzyme, perforin, granulysin, CD107a, TNFα, FASL)
(e) increased cytotoxicity (e.g. against target protein-expressing cells)
(f) reduced sensitivity to one or more immunosuppressive factors, e.g. tumor-derived immunosuppressive factors (e.g. IL-10, TGF-β, IDO, VEGF, IL-6)
(g) increased persistence (thereby providing a longer-lasting/sustained T cell response) and durability (ability to withstand stress)
(h) increased tumor penetration
(i) increased sensitivity to trafficking information (e.g. increased sensitivity to cytokine/chemokine gradients/milieu)
(j) reduced level of production of one or more proinflammatory and/or effector factors in response to target protein-expressing cells
(k) increased cytotoxicity against target protein-expressing cells, and reduced level of production of one or more proinflammatory and/or effector factors in response to target protein-expressing cells
(l) increased proliferation in response to target protein-expressing cells
(m) reduced proliferation in the absence of target protein-expressing cells
(n) increased proliferation in the absence of target protein-expressing cells

These properties can be analyzed by methods well known to the skilled person. The rate of proliferation can be measured e.g. by measuring the number of cells at different time points, or by analysis of incorporation of ³H-thymidine or CFSE dilution assay, e.g. as described in Fulcher and Wong, Immunol Cell Biol (1999) 77(6): 559-564. Gene or protein expression of growth factors and cytotoxic/effector factors can be measured e.g. by qPCR analysis of mRNA levels, and/or by immunoassay based methods for detecting the relevant protein, such as ELISA, flow cytometry, immunoblot, etc. Survival of cells may be determined by labelling cells, and monitoring cell number over time. Cytotoxicity can be investigated, for example, using any of the methods reviewed in Zaritskaya et al., Expert Rev Vaccines (2011), 9(6):601-616, hereby incorporated by reference in its entirety, e.g. by ⁵¹Cr release assay. Sensitivity to immunosuppressive factors can be determined by analyzing the rate of proliferation/expression of growth factors/survival/expression of cytotoxic or effector factors/cytotoxicity for cells expressing the CAR in the presence of an immunosuppressive factor.

Cell proliferation can be determined by analysing cell division over a period of time. Cell division for a given cell or population of cells can be analysed, for example, by in vitro analysis of incorporation of ³H-thymidine or by CFSE dilution assay, e.g. as described in Fulcher and Wong, Immunol Cell Biol (1999) 77(6): 559-564, hereby incorporated by reference in entirety. Proliferating cells may also be identified by analysis of incorporation of 5-ethynyl-2′-deoxyuridine (EdU) by an appropriate assay, as described e.g. in Buck et al., Biotechniques. 2008 June; 44(7):927-9, and Sali and Mitchison, PNAS USA 2008 Feb. 19; 105(7): 2415-2420, both hereby incorporated by reference in their entirety.

In some embodiments, the cell may exhibit one or more of the properties of (a)-(n) following activation of the CAR. In some embodiments, the cell may exhibit one of more of the properties of (a)-(n) following exposure to the target molecule for which the antigen-binding domain of the CAR is specific, e.g. in the form of a cell expressing/overexpressing the target protein.

Increased gene or protein expression, survival, cytotoxicity or proliferation by a cell expressing a CAR according to the present invention may be one of more than 1 times, more than 1.1 times, more than 1.2 times, more than 1.3 times, more than 1.4 times, more than 1.5 times, more than 1.6 times, more than 1.7 times, more than 1.8 times, more than 1.9 times, more than 2 times, more than 2.1 times, more than 2.2 times, more than 2.3 times, more than 2.4 times, more than 2.5 times, more than 2.6 times, more than 2.7 times, more than 2.8 times, more than 2.9 times, more than 3 times, more than 3.1 times, more than 3.2 times, more than 3.3 times, more than 3.4 times, more than 3.5 times, more than 3.6 times, more than 3.7 times, more than 3.8 times, more than 3.9 times, more than 4 times, more than 4.1 times, more than 4.2 times, more than 4.3 times, more than 4.4 times, more than 4.5 times, more than 4.6 times, more than 4.7 times, more than 4.8 times, more than 4.9 times, or more than 5 times the level of expression, survival, cytotoxicity or proliferation displayed by a cell expressing another CAR, e.g. a CAR lacking a costimulatory sequence which is, or which is derived from, the intracellular domain of CD226, in a comparable assay.

Reduced proliferation by a cell expressing a CAR according to the present invention may be one of less than 1 times, less than 0.95 times, less than 0.9 times, less than 0.85 times, less than 0.8 times, less than 0.75 times, less than 0.7 times, less than 0.65 times, less than 0.6 times, less than 0.55 times, less than 0.5 times, less than 0.45 times, less than 0.4 times, less than 0.35 times, less than 0.3 times, less than 0.25 times, less than 0.2 less than 0.15 times, or less than 0.1 times the level of proliferation by a cell expressing another CAR, e.g. a CAR lacking a costimulatory sequence which is, or which is derived from, the intracellular domain of CD226, in a comparable assay.

Reduced sensitivity of a cell expressing a CAR according to the present invention to one or more immunosuppressive factors may be determined by observation of a level of inhibition of proliferation/expression of growth factors/survival/expression of cytotoxic or effector factors/cytotoxicity in response to the immunosuppressive factor(s) which is less than the level of inhibition of the relevant property observed for a cell expressing another CAR, e.g. a CAR according to Table 1, in a comparable assay. In some embodiments, the level of inhibition is one of less than 1 times, less than 0.95 times, less than 0.9 times, less than 0.85 times, less than 0.8 times, less than 0.75 times, less than 0.7 times, less than 0.65 times, less than 0.6 times, less than 0.55 times, less than 0.5 times, less than 0.45 times, less than 0.4 times, less than 0.35 times, less than 0.3 times, less than 0.25 times, less than 0.2 less than 0.15 times, or less than 0.1 times the level of inhibition of the relevant property observed for a cell expressing another CAR, e.g. a CAR lacking a costimulatory sequence which is, or which is derived from, the intracellular domain of CD226, in a comparable assay.

In some embodiments, a cell expressing a CAR according to the present invention may display reduced sensitivity to TGFβ as compared to a cell expressing another CAR, e.g. a CAR lacking a costimulatory sequence which is, or which is derived from, the intracellular domain of CD226 (e.g. a CAR according to Table 1), in a comparable assay. An example of a suitable assay for analyzing sensitivity of T cells to TGFβ-mediated suppression of effector function is described at Example 16.

Reduced level of production of a proinflammatory/effector factor by a cell expressing a CAR according to the present invention may be determined by detection of a reduced level of the factor e.g. the cell culture supernatant following co-culture of the cell expressing the CAR with a cell expressing the target protein, as compared to the level of the factor detected following co-culture of a cell expressing another CAR, e.g. a CAR lacking a costimulatory sequence which is, or which is derived from, the intracellular domain of CD226 (e.g. a CAR according to Table 1), with a cell expressing the target protein, in a comparable assay. In some embodiments, a reduced level of production is one of less than 1 times, less than 0.99 times, less than 0.98 times, less than 0.97 times, less than 0.96 times, less than 0.95 times, less than 0.9 times, less than 0.85 times, less than 0.8 times, less than 0.75 times, less than 0.7 times, less than 0.65 times, less than 0.6 times, less than 0.55 times, less than 0.5 times, less than 0.45 times, less than 0.4 times, less than 0.35 times, less than 0.3 times, less than 0.25 times, less than 0.2 less than 0.15 times, or less than 0.1 times the level of production of the factor detected following co-culture of a cell expressing another CAR, e.g. a CAR lacking a costimulatory sequence which is, or which is derived from, the intracellular domain of CD226 (e.g. a CAR according to Table 1), with a cell expressing the target protein, in a comparable assay.

Particular activities or functional properties for a cell expressing the CAR of the invention may be associated with one or more domains of the CAR of the present invention, or the particular combination of domains.

For example, a cell expressing a CAR comprising a signaling domain comprising a costimulatory sequence which is, or which is derived from, the intracellular domain of CD226 may display increased expression of one or more cytotoxic factors, increased cytotoxicity and/or reduced sensitivity to immunosuppressive factors as compared to a CAR not comprising a costimulatory sequence which is, or which is derived from, the intracellular domain of CD226. In some embodiments a cell expressing a CAR comprising a signaling domain comprising a costimulatory sequence which is, or which is derived from, the intracellular domain of CD226 may display reduced expression of one of more a proinflammatory or effector factors. In some embodiments a proinflammatory factor or an effector factor may be selected from one or more of: IL-2, IFNγ, TNFα, GM-CSF, MIP-1α, MIP-1β, RANTES and TNFβ.

A cell expressing a CAR comprising a dimerization domain may display an increased rate of proliferation, increased expression of one or more growth factors and/or increased survival as compared to a cell expressing a CAR lacking the dimerization domain. In some embodiments, the cell may exhibit one or more of these properties following treatment with an agent. For example, in embodiments wherein the dimerization domain is an inducible dimerization domain, the cell may display one or more of these properties following treatment with the appropriate agent for inducing dimerization, oligomerization, or multimerization of the CAR.

A CAR comprising a dimerization domain may more readily form dimers, or may form more stable dimers, than a CAR lacking the dimerization domain.

Dimer formation may promote CAR-mediated signaling, and so a CAR comprising a dimerization domain according to the invention may exhibit an increased level of CAR-mediated signaling as compared to a CAR lacking the dimerization domain. Similarly, cells expressing a CAR comprising a dimerization domain may exhibit a phenotype associated with increased level of CAR-mediated signaling as compared to cells expressing a comparable CAR lacking the dimerization domain.

### Uses of and Methods of Using the CARs, Nucleic Acids, Cells and Compositions

The CARs, nucleic acids, vectors cells and pharmaceutical compositions according to the present invention find use in therapeutic and prophylactic methods.

The present invention provides a chimeric antigen receptor, nucleic acid, vector, cell or pharmaceutical composition according to the present invention for use in a method of medical treatment or prophylaxis.

The present invention also provides the use of a chimeric antigen receptor, nucleic acid, vector, cell or pharmaceutical composition according to the present invention in the manufacture of a medicament for treating or preventing a disease or disorder.

The present invention also provides a method of treating or preventing a disease or disorder, comprising administering to a subject a therapeutically or prophylactically effective amount of a chimeric antigen receptor, nucleic acid, vector, cell or pharmaceutical composition according to the present invention.

In particular, the CAR, nucleic acid, vector, cell or pharmaceutical composition according to the present invention finds use to prevent or treat a disease or disorder which is associated with expression/upregulated expression of the target protein.

### Administration

Administration of a CAR, nucleic acid, vector, cell or composition according to the invention is preferably in a “therapeutically effective” or “prophylactically effective” amount, this being sufficient to show benefit to the subject. The actual amount administered, and rate and time-course of administration, will depend on the nature and severity of the disease or disorder. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

The CARs, nucleic acids, vectors, cells, compositions and other therapeutic agents, medicaments and pharmaceutical compositions according to aspects of the present invention may be formulated for administration by a number of routes, including but not limited to, parenteral, intravenous, intra-arterial, intramuscular, subcutaneous, intradermal, intratumoral and oral. The CARs, nucleic acids, vectors, cells, composition and other therapeutic agents and therapeutic agents may be formulated in fluid or solid form. Fluid formulations may be formulated for administration by injection to a selected region of the human or animal body, or by infusion to the blood. Administration may be by injection or infusion to the blood, e.g. intravenous or intra-arterial administration.

Administration may be alone or in combination with other treatments, either simultaneously or sequentially dependent upon the condition to be treated. The CAR, nucleic acid, vector, cell or composition according to the present invention and a therapeutic agent may be administered simultaneously or sequentially.

In some embodiments, treatment with CAR, nucleic acid, vector, cell or composition of the present invention may be accompanied by other therapeutic or prophylactic intervation, e.g. chemotherapy, immunotherapy, radiotherapy, surgery, vaccination and/or hormone therapy.

Simultaneous administration refers to administration of the CAR, nucleic acid, vector, cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one of the CAR, nucleic acid, vector, cell or composition or therapeutic agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or y-rays).

The drug may be a chemical entity, e.g. small molecule pharmaceutical, antibiotic, DNA intercalator, protein inhibitor (e.g. kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs and biologics may be selected from: alkylating agents such as cisplatin, carboplatin, mechlorethamine, cyclophosphamide, chlorambucil, ifosfamide; purine or pyrimidine anti-metabolites such as azathiopurine or mercaptopurine; alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel; topoisomerase inhibitors such as the type I topoisomerase inhibitors camptothecins irinotecan and topotecan, or the type II topoisomerase inhibitors amsacrine, etoposide, etoposide phosphate, teniposide; antitumor antibiotics (e.g. anthracyline antibiotics) such as dactinomycin, doxorubicin (Adriamycin™), epirubicin, bleomycin, rapamycin; antibody based agents, such as anti-PD-1 antibodies, anti-PD-L1 antibodies, anti-TIM-3 antibodies, anti-CTLA-4, anti-4-1BB, anti-GITR, anti-CD27, anti-BLTA, anti-OX43, anti-VEGF, anti-TNFα, anti-IL-2, antiGpIIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab; EGFR inihibitors such as erlotinib, cetuximab and gefitinib; anti-angiogenic agents such as bevacizumab (Avastin®); cancer vaccines such as Sipuleucel-T (Provenge®).

Further chemotherapeutic drugs may be selected from: 13-cis-Retinoic Acid, 2-Chlorodeoxyadenosine, 5-Azacitidine 5-Fluorouracil, 6-Mercaptopurine, 6-Thioguanine, Abraxane, Accutane®, Actinomycin-D Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Aranesp®, Aredia®, Arimidex®, Aromasin®, Arranon®, Arsenic Trioxide, Asparaginase, ATRA Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, Calcium Leucovorin, Campath®, Camptosar®, Camptothecin-11, Capecitabine, Carac™, Carboplatin, Carmustine, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine Cytosar-U®, Cytoxan®, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Faslodex®, Femara®, Filgrastim, Floxuridine, Fludara®, Fludarabine, Fluoroplex®, Fluorouracil, Fluoxymesterone, Flutamide, Folinic Acid, FUDR®, Fulvestrant, Gefitinib, Gemcitabine, Gemtuzumab ozogamicin, Gleevec™, Gliadel® Wafer, Goserelin, Granulocyte—Colony Stimulating Factor, Granulocyte Macrophage Colony Stimulating Factor, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant Purinethol®, Raloxifene, Revlimid®, Rheumatrex®, Rituxan®, Rituximab, Roferon-A® (Interferon Alfa-2a), Rubex®, Rubidomycin hydrochloride, Sandostatin® Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®.

Multiple doses of the CAR, nucleic acid, vector, cell or composition may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

### Cancer

In some embodiments, the disease or disorder to be treated or prevented in accordance with the present invention is a cancer. GPC3 expression is upregulated in a various cancers. Accordingly, the disease or disorder to be treated or prevented may be a cancer in which GPC3 expression is upregulated.

EpCAM expression is upregulated in a various cancers. Accordingly, the disease or disorder to be treated or prevented may be a cancer in which EpCAM expression is upregulated.

The cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma.

In some embodiments the cancer to be treated/prevented in accordance with the invention may be a hepatic cancer/liver cancer (e.g. hepatocellular carcinoma, hepatoblastoma). The hepatic cancer may express or overexpress GPC3. The hepatic cancer may express or overexpress EpCAM.

In some embodiments the cancer to be treated/prevented in accordance with the invention may be a lung cancer (e.g. non-small cell lung cancer (NSCLC)). The lung cancer may express or overexpress GPC3. The lung cancer may express or overexpress EpCAM.

In some embodiments the cancer is a cancer expressing the target protein for which the antigen-binding domain of the CAR is specific (e.g. a GPC3-expressing cancer). A cancer may be determined to express a target protein by any suitable means, which are well known to the skilled person. A cancer expressing the target protein may be identified by detection of expression of target protein. In some embodiments, the cancer over-expresses the target protein. Overexpression of a target protein can be determined by detection of a level of expression of the target protein which is greater than the level of expression of target protein by equivalent non-cancerous cells/non-tumor tissue.

Expression may be gene expression or protein expression. Gene expression can be determined e.g. by detection of mRNA encoding the relevant target protein, for example by quantitative real-time PCR (qRT-PCR). Protein expression can be determined e.g. by detection of the target protein, for example by antibody-based methods, for example by western blot, immunohistochemistry, immunocytochemistry, flow cytometry, or ELISA.

In some embodiments, a patient may be selected for treatment according to the present invention based on the detection of a cancer expressing the target protein, or overexpressing the target protein, e.g. in a sample obtained from the subject.

In some embodiments, the target protein is GPC3 and the cancer may express or overexpress GPC3. Cancers that may express GPC3 include melanoma, ovarian clear-cell carcinoma, yolk sac tumors, neuroblastoma, hepatoblastoma, and Wilms' tumor cells (Ho et al. 2011 Eur J Cancer 47(3):333-338).

In some embodiments, the target protein is EpCAM and the cancer may express or overexpress EpCAM. Cancers that may express EpCAM include epithelial cell cancers, breast cancer, ovarian cancer, pancreatic carcinoma, urothelial carcinoma, gastric cancer, esophageal carcinoma, colorectal carcinoma, hepatocellular carcinoma and gallbladder carcinoma.

### Adoptive Transfer

In embodiments of the present invention, a method of treatment or prophylaxis may comprise adoptive transfer of immune cells, e.g. T cells. Adoptive T cell transfer generally refers to a process by which T cells are obtained from a subject, typically by drawing a blood sample from which T cells are isolated. The T cells are then typically treated or altered in some way, optionally expanded, and then administered either to the same subject or to a different subject. The treatment is typically aimed at providing a T cell population with certain desired characteristics to a subject, or increasing the frequency of T cells with such characteristics in that subject. Adoptive transfer of CAR-T cells is described, for example, in Kalos and June 2013, Immunity 39(1): 49-60, which is hereby incorporated by reference in its entirety.

In the present invention, adoptive transfer is performed with the aim of introducing, or increasing the frequency of, target protein-reactive T cells in a subject, in particular target protein-reactive CD8+ T cells and/or CD4+ T cells.

Accordingly, the present invention provides a method of treating or preventing a disease or disorder in a subject, comprising:
(a) isolating at least one T cell from a subject;
(b) modifying the at least one T cell to express or comprise a chimeric antigen receptor or nucleic acid according to the present invention,
(c) optionally expanding the modified at least one T cell, and;
(d) administering the modified at least one T cell to a subject.

In some embodiments, the subject from which the T cell is isolated is the subject administered with the modified T cell (i.e., adoptive transfer is of autologous T cells). In some embodiments, the subject from which the T cell is isolated is a different subject to the subject to which the modified T cell is administered (i.e., adoptive transfer is of allogenic T cells).

The at least one T cell modified according to the present invention can be modified according to methods well known to the skilled person. The modification may comprise nucleic acid transfer for permanent or transient expression of the transferred nucleic acid.

Any suitable genetic engineering platform may be used to modify a T cell according to the present invention. Suitable methods for modifying a T cell include the use of genetic engineering platforms such as gammaretroviral vectors, lentiviral vectors, adenovirus vectors, DNA transfection, transposon-based gene delivery and RNA transfection, for example as described in Maus et al., Annu Rev Immunol (2014) 32:189-225, incorporated by reference hereinabove.

In some embodiments the method may comprise one or more of the following steps: taking a blood sample from a subject; isolating and/or expanding at least one T cell from the blood sample; culturing the at least one T cell in in vitro or ex vivo cell culture; introducing into the at least one T cell a CAR, nucleic acid, or vector according to the present invention, thereby modifying the at least one T cell; expanding the at least one modified T cell, collecting the at least one modified T cell; mixing the modified T cell with an adjuvant, diluent, or carrier; administering the modified T cell to a subject.

In some embodiments, e.g. wherein the CAR comprises a chemically-inducible dimerization domain, the methods may additionally comprise treating the modified T cell with the appropriate dimerization-inducing agent. In some embodiments, treatment may be in vitro or ex vivo, by administration of the agent to the modified T cell in culture. In some embodiments, treatment may be in in vivo by administration of the agent to a subject having been administered with a modified T cell according to the invention. In this way, modified T cells comprising the CAR according to the present invention can be stimulated to proliferate, and thereby expanded, in vitro/ex vivo and/or in vivo.

The skilled person is able to determine appropriate reagents and procedures for adoptive transfer of target protein-reactive CAR-T cells according to the present invention for example by reference to Dai et al., 2016 J Nat Cancer Inst 108(7): djv439, which is incorporated by reference in its entirety.

In a related aspect, the present invention provides a method of preparing a modified T cell, the method comprising introducing into a T cell a CAR, nucleic acid or vector according to the present invention, thereby modifying the at least one T cell. The method is preferably performed in vitro or ex vivo.

In one aspect, the present invention provides a method of treating or preventing a disease or disorder in a subject, comprising:
(a) isolating at least one T cell from a subject;
(b) introducing into the at least one T cell the isolated nucleic acid or vector according to the present invention, thereby modifying the at least one T cell; and
(c) administering the modified at least one T cell to the subject.

In embodiments according to the present invention the subject is preferably a human subject. In some embodiments, the subject to be treated according to a therapeutic or prophylactic method of the invention herein is a subject having, or at risk of developing, a disease or disorder characterised by expression or upregulated expression of the target protein. In some embodiments, the subject to be treated is a subject having, or at risk of developing, a cancer, e.g. a cancer expressing the target protein, or a cancer in which expression of the target protein is upregulated.

In embodiments according to the present invention, a subject may be selected for treatment according to the methods based on characterisation for certain markers of such disease/disorder, e.g. target protein expression. A subject may have been diagnosed with the disease or disorder requiring treatment, or be suspected of having such a disease or disorder.

In some embodiments, the method additionally comprise therapeutic or prophylactic intervention for the treatment or prevention of a disease or disorder, e.g. chemotherapy, immunotherapy, radiotherapy, surgery, vaccination and/or hormone therapy. In some embodiments, the method additionally comprises therapeutic or prophylactic intervention, for the treatment or prevention of a cancer, such as a hepatic cancer, e.g. hepatocellular carcinoma.

### Subjects

The subject to be treated according to the invention may be any animal or human. The subject is preferably mammalian, more preferably human. The subject may be a non-human mammal, but is more preferably human. The subject may be male or female. The subject may be a patient. A subject may have been diagnosed with a disease or condition requiring treatment, may be suspected of having such a disease or condition, or may be at risk from developing such a disease or condition.

### Numbered Statements of Invention

Following numbered paragraphs (paras) describe particular aspects and embodiments of the present invention:

1. A chimeric antigen receptor (CAR) which is capable of binding to GPC3, comprising: a GPC3-binding domain, a hinge region, a transmembrane domain, and a signalling domain;
wherein the hinge region comprises or consists of an amino acid sequence which is, or which is derived from, the human IgG1 hinge region, and;
wherein the transmembrane domain comprises or consists of an amino acid sequence which is, or which is derived from, the transmembrane domain of CD8α.
   2. The CAR according to para 1, wherein the hinge region comprises or consists of an amino acid sequence having at least 80% sequence identity to the amino acid sequence of SEQ ID NO:19, and wherein the transmembrane domain comprises or consists of an amino acid sequence having at least 80% sequence identity to the amino acid sequence of SEQ ID NO:11.
   3. A chimeric antigen receptor (CAR) which is capable of binding to GPC3, comprising: a GPC3-binding domain, a transmembrane domain, a signalling domain, and an inducible dimerization domain.
   4. The CAR according to para 3, wherein the dimerization domain comprises or consists of an amino acid sequence which is, or which is derived from, the amino acid sequence of F36V-FKBP.
   5. The CAR according to any one of paras 1 to 4, wherein the CAR comprises a dimerization domain which comprises or consists of an amino acid sequence having at least 80% sequence identity to the amino acid sequence of SEQ ID NO:20.
   6. A chimeric antigen receptor (CAR) which is capable of binding to GPC3, comprising: a GPC3-binding domain, a transmembrane domain, and a signalling domain; wherein the signalling domain comprises a costimulatory sequence which comprises or consists of an amino acid sequence which is, or which is derived from, the intracellular domain of CD226.
   7. The CAR according to any one of paras 1 to 6, wherein the signalling domain comprises a costimulatory sequence which comprises or consists of an amino acid sequence having at least 80% sequence identity to the amino acid sequence of SEQ ID NO:16.
   8. The CAR according to any one of paras 1 to 7, wherein the signalling domain comprises a costimulatory sequence which comprises or consists of an amino acid sequence which is, or which is derived from, the intracellular domain of CD28.
   9. The CAR according to any one of paras 1 to 8, wherein the signalling domain comprises a costimulatory sequence which comprises or consists of an amino acid sequence which is, or which is derived from, the intracellular domain of 4-1BB.
   10. The CAR according to any one of paras 1 to 9, wherein the signalling domain comprises a costimulatory sequence which comprises or consists of an amino acid sequence having at least 80% sequence identity to the amino acid sequence of SEQ ID NO:17.
   11. The CAR according to any one of paras 1 to 10, wherein the signalling domain comprises a costimulatory sequence which comprises or consists of an amino acid sequence having at least 80% sequence identity to the amino acid sequence of SEQ ID NO:18.
   12. A chimeric antigen receptor (CAR) which is capable of binding to GPC3 according to any one of A, B, C, D, E, F, G, H, I, J, K, L or M as shown in Table 1.
   13. A chimeric antigen receptor (CAR) which is capable of binding to GPC3, comprising or consisting of, an amino acid sequence having at least 60% sequence identity to the amino acid sequence of SEQ ID NO:38, 39, 40, 22, 23, 41, 42, 24, 25, 26, 27, 28 or 29.
   14. A chimeric antigen receptor (CAR) which is capable of binding to GPC3, comprising or consisting of, an amino acid sequence having at least 60% sequence identity to the amino acid sequence of SEQ ID NO:43, 44, 45, 30, 31, 46, 47, 32, 33, 34, 35, 36 or 37.
   15. A nucleic acid encoding the chimeric antigen receptor (CAR) according to any one of paras 1 to 14.
   16. A vector comprising the nucleic acid of para 15.
   17. A cell comprising the chimeric antigen receptor (CAR) according to any one of paras 1 to 14, the nucleic acid according to para 15, or the vector according to para 16.
   18. A method for producing a cell expressing a chimeric antigen receptor (CAR) which is capable of binding to GPC3, comprising introducing into a cell a nucleic acid according to para 15, or a vector according to para 16, and culturing the cell under conditions suitable for expression of the nucleic acid or vector by the cell.
   19. A cell which is obtained or obtainable by the method according to para 18.
   20. A pharmaceutical composition comprising a chimeric antigen receptor (CAR) according to any one of paras 1 to 14, a nucleic acid according to para 15, a vector according to para 16, or a cell according to para 17 or para 19, and a pharmaceutically acceptable carrier, adjuvant, excipient, or diluent.
   21. A chimeric antigen receptor (CAR) according to any one of paras 1 to 14, a nucleic acid according to para 15, a vector according to para 16, a cell according to para 17 or para 19, or a pharmaceutical composition according to para 20, for use in a method of treating or preventing a disease or disorder.
   22. Use of a chimeric antigen receptor (CAR) according to any one of paras 1 to 14, a nucleic acid according to para 15, a vector according to para 16, a cell according to para 17 or para 19, or a pharmaceutical composition according to para 20, in the manufacture of a medicament for treating or preventing a disease or disorder.
   23. A method of treating or preventing a disease or disorder, comprising administering to a subject a therapeutically or prophylactically effective amount of a chimeric antigen receptor (CAR) according to any one of paras 1 to 14, a nucleic acid according to para 15, a vector according to para 16, a cell according to para 17 or para 19, or a pharmaceutical composition according to para 20.
   24. A method of treating or preventing a disease or disorder in a subject, comprising:
(a) isolating at least one T cell from a subject;
(b) modifying the at least one T cell to express or comprise a chimeric antigen receptor (CAR) according to any one of paras 1 to 14, a nucleic acid according to para 15, or a vector according to para 16, and;
(c) administering the modified at least one T cell to a subject.
   25. A method of treating or preventing a disease or disorder in a subject, comprising:
(a) isolating at least one T cell from a subject;
(b) introducing into the at least one T cell a nucleic acid according to para 15, or a vector according to para 16, thereby modifying the at least one T cell and;
(c) administering the modified at least one T cell to a subject.
   26. The CAR, nucleic acid, vector, cell, or pharmaceutical composition for use according to para 21, the use according to para 22, or the method according to any one of paras 23 to 25, wherein the disease or disorder is a GPC3-expressing cancer.
   27. The CAR, nucleic acid, vector, cell, or pharmaceutical composition for use, the use, or the method according to according to para 26, wherein the GPC3-expressing cancer is a hepatocellular carcinoma.
   28. A kit of parts comprising a predetermined quantity of a chimeric antigen receptor (CAR) according to any one of paras 1 to 14, a nucleic acid according to para 15, a vector according to para 16, a cell according to para 17 or para 19, or a pharmaceutical composition according to para 20.

Sequences

| SEQ | | |
|---|---|---|
| ID NO: | DESCRIPTION | SEQUENCE |
| 1 | GC33 heavy chain | QVQLQQSGAELVRPGASVKLSCKASGYTFTDYEMHWVKQTPVHGLKWIG |
| | variable region | ALDPKTGDTAYSQKFKGKATLTADKSSSTAYMELRSLTSEDSAVYYCTRFY |
| | sequence | SYTYWGQGTLVTVSA |
| 2 | GC33 HC-CDR1 | DYEMH |
| 3 | GC33 HC-CDR2 | ALDPKTGDTAYSQKFKG |
| 4 | GC33 HC-CDR3 | FYSYTY |
| 5 | GC33 light chain | DVVMTQTPLSLPVSLGDQASISCRSSQSLVHSNGNTYLHWYLQKPGQSPK |
| | variable region | LLIYKVSNRFSGVPDRFSGSGSGTDFTLKISRVEAEDLGVYFCSQNTHVPPT |
| | sequence | FGSGTKLEIK |
| 6 | GC33 LC-CDR1 | RSSQSLVHSNGNTYLH |
| 7 | GC33 LC-CDR2 | KVSNRFS |
| 8 | GC33 LC-CDR3 | SQNTHVPPT |
| 9 | GC33 scFv | QVQLQQSGAELVRPGASVKLSCKASGYTFTDYEMHWVKQTPVHGLKWIG |
| | | ALDPKTGDTAYSQKFKGKATLTADKSSSTAYMELRSLTSEDSAVYYCTRFY |
| | | SYTYWGQGTLVTVSAGGGGSGGGGSGGGGSDVVMTQTPLSLPVSLGDQ |
| | | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIK |
| 10 | CD28 | FWVLVVVGGVLACYSLLVTVAFII |
| | transmembrane | |
| | domain | |
| 11 | CD8α | IYIWAPLAGTCGVLLLSLVITLYCNHRN |
| | transmembrane | |
| | domain | |
| 12 | ITAM motif | YXXL/I |
| | | X = any amino acid |
| 13 | ITAM motif | YXXL/I(X)₆₋₈YXXL/I |
| | | X = any amino acid |
| 14 | CD3-ζ intracellular | RVKFSRSADAPAYQQGQNQLYNELNLGRREEYDVLDKRRGRDPEMGGKP |
| | domain | RRKNPQEGLYNELQKDKMAEAYSEIGMKGERRRGKGHDGLYQGLSTATK |
| | | DTYDALHMQALPPR |
| 15 | Human CD226; | MDYPTLLLALLHVYRALCEEVLWHTSVPFAENMSLECVYPSMGILTQVEWF |
| | UniProt Q15762 | KIGTQQDSIAIFSPTHGMVIRKPYAERVYFLNSTMASNNMTLFFRNASEDDV |
| | (CD226_HUMAN) | GYYSCSLYTYPQGTWQKVIQVVQSDSFEAAVPSNSHIVSEPGKNVTLTCQP |
| | | QMTWPVQAVRWEKIQPRQIDLLTYCNLVHGRNFTSKFPRQIVSNCSHGRW |
| | | SVIVIPDVTVSDSGLYRCYLQASAGENETFVMRLTVAEGKTDNQYTLFVAG |
| | | GTVLLLLFVISITTIIVIFLNRRRRRERRDLFTESWDTQKAPNNYRSPISTSQP |
| | | TNQSMDDTREDIYVNYPTFSRRPKTRV |
| 16 | CD226 intracellular | IVIFLNRRRRRERRDLFTESWDTQKAPNNYRSPISTSQPTNQSMDDTREDIY |
| | domain (amino acid | VNYPTFSRRPKTRV |
| | positions 271 to | |
| | 336 of UniProt | |
| | Q15762) | |
| 17 | CD28 intracellular | FWVRSKRSRLLHSDYMNMTPRRPGPTRKHYQPYAPPRDFAAYRS |
| | domain | |
| 18 | 4-1BB intracellular | KRGRKKLLYIFKQPFMRPVQTTQEEDGCSCRFPEEEEGGCEL |
| | domain | |
| 19 | Human IgG1 hinge | EPKSCDKTHTCPPCP |
| | region | |
| 20 | F36V-FKBP | GVQVETISPGDGRTFPKRGQTCVVHYTGMLEDGKKVDSSRDRNKPFKFML |
| | dimerization | GKQEVIRGWEEGVAQMSVGQRAKLTISPDYAYGATGHPGIIPPHATLVFDV |
| | sequence | ELLKLE |
| 21 | Human Ig heavy | MDWIWRILFLVGAATGAHS |
| | chain signal | |
| | sequence | |
| 22 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/CD226/CD3ζ | SYTYWGQGTLVTVSAGGGGSGGGGSGGGGSDVVMTQTPLSLPVSLGDQ |
| | | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNIVIFLNRRRRRERRDL |
| | | FTESWDTQKAPNNYRSPISTSQPTNQSMDDTREDIYVNYPTFSRRPKTRVR |
| | | VKFSRSADAPAYQQGQNQLYNELNLGRREEYDVLDKRRGRDPEMGGKPR |
| | | RKNPQEGLYNELQKDKMAEAYSEIGMKGERRRGKGHDGLYQGLSTATKDT |
| | | YDALHMQALPPR |
| 23 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/F36V- | SYTYWGQGTLVTVSAGGGGSGGGGSGGGGSDVVMTQTPLSLPVSLGDQ |
| | FKBP/CD226/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNGVQVETISPGDGRTFP |
| | | KRGQTCVVHYTGMLEDGKKVDSSRDRNKPFKFMLGKQEVIRGWEEGVAQ |
| | | MSVGQRAKLTISPDYAYGATGHPGIIPPHATLVFDVELLKLEIVIFLNRRRRR |
| | | ERRDLFTESWDTQKAPNNYRSPISTSQPTNQSMDDTREDIYVNYPTFSRRP |
| | | KTRVRVKFSRSADAPAYQQGQNQLYNELNLGRREEYDVLDKRRGRDPEM |
| | | GGKPRRKNPQEGLYNELQKDKMAEAYSEIGMKGERRRGKGHDGLYQGLS |
| | | TATKDTYDALHMQALPPR |
| 24 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/CD226/CD28/ | SYTYWGQGTLVTVSAGGGGSGGGGSGGGGSDVVMTQTPLSLPVSLGDQ |
| | CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNIVIFLNRRRRRERRDL |
| | | FTESWDTQKAPNNYRSPISTSQPTNQSMDDTREDIYVNYPTFSRRPKTRVF |
| | | WVRSKRSRLLHSDYMNMTPRRPGPTRKHYQPYAPPRDFAAYRSRVKFSR |
| | | SADAPAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTYDALH |
| | | MQALPPR |
| 25 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/F36V- | SYTYWGQGTLVTVSAGGGGSGGGGSGGGGSDVVMTQTPLSLPVSLGDQ |
| | FKBP/CD226/CD28/ | ASISCRSSQSLVHSNGNTYLHWYLQKPGQSPKLLIYKVSNRFSGVPDRFSG |
| | CD3ζ | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNGVQVETISPGDGRTFP |
| | | KRGQTCVVHYTGMLEDGKKVDSSRDRNKPFKFMLGKQEVIRGWEEGVAQ |
| | | MSVGQRAKLTISPDYAYGATGHPGIIPPHATLVFDVELLKLEIVIFLNRRRRR |
| | | ERRDLFTESWDTQKAPNNYRSPISTSQPTNQSMDDTREDIYVNYPTFSRRP |
| | | KTRVFWVRSKRSRLLHSDYMNMTPRRPGPTRKHYQPYAPPRDFAAYRSR |
| | | VKFSRSADAPAYQQGQNQLYNELNLGRREEYDVLDKRRGRDPEMGGKPR |
| | | RKNPQEGLYNELQKDKMAEAYSEIGMKGERRRGKGHDGLYQGLSTATKDT |
| | | YDALHMQALPPR |
| 26 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/CD226/4- | SYTYWGQGTLVTVSAGGGGSGGGGSGGGGSDVVMTQTPLSLPVSLGDQ |
| | 1BB/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNIVIFLNRRRRRERRDL |
| | | FTESWDTQKAPNNYRSPISTSQPTNQSMDDTREDIYVNYPTFSRRPKTRVK |
| | | RGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQEGL |
| | | YNELQKDKMAEAYSEIGMKGERRRGKGHDGLYQGLSTATKDTYDALHMQ |
| | | ALPPR |
| 27 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/F36V- | SYTYWGQGTLVTVSAGGGGSGGGGSGGGGSDVVMTQTPLSLPVSLGDQ |
| | FKBP/CD226/41BB/ | ASISCRSSQSLVHSNGNTYLHWYLQKPGQSPKLLIYKVSNRFSGVPDRFSG |
| | CD3ζ | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNGVQVETISPGDGRTFP |
| | | KRGQTCVVHYTGMLEDGKKVDSSRDRNKPFKFMLGKQEVIRGWEEGVAQ |
| | | MSVGQRAKLTISPDYAYGATGHPGIIPPHATLVFDVELLKLEIVIFLNRRRRR |
| | | ERRDLFTESWDTQKAPNNYRSPISTSQPTNQSMDDTREDIYVNYPTFSRRP |
| | | KTRVKRGRKKLLYIFKQPFMRPVQTTQEEDGCSCRFPEEEEGGCELRVKF |
| | | SRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDA |
| | | LHMQALPPR |
| 28 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/CD226/CD28/ | SYTYWGQGTLVTVSAGGGGSGGGGSGGGGSDVVMTQTPLSLPVSLGDQ |
| | 4-1BB/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNIVIFLNRRRRRERRDL |
| | | FTESWDTQKAPNNYRSPISTSQPTNQSMDDTREDIYVNYPTFSRRPKTRVF |
| | | WVRSKRSRLLHSDYMNMTPRRPGPTRKHYQPYAPPRDFAAYRSKRGRKK |
| | | LLYIFKQPFMRPVQTTQEEDGCSCRFPEEEEGGCELRVKFSRSADAPAYQ |
| | | QGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| 29 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/F36V- | SYTYWGQGTLVTVSAGGGGSGGGGSGGGGSDVVMTQTPLSLPVSLGDQ |
| | FKBP/CD226/CD28/ | ASISCRSSQSLVHSNGNTYLHWYLQKPGQSPKLLIYKVSNRFSGVPDRFSG |
| | 41BB/CD3ζ | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNGVQVETISPGDGRTFP |
| | | KRGQTCVVHYTGMLEDGKKVDSSRDRNKPFKFMLGKQEVIRGWEEGVAQ |
| | | MSVGQRAKLTISPDYAYGATGHPGIIPPHATLVFDVELLKLEIVIFLNRRRRR |
| | | ERRDLFTESWDTQKAPNNYRSPISTSQPTNQSMDDTREDIYVNYPTFSRRP |
| | | KTRVFWVRSKRSRLLHSDYMNMTPRRPGPTRKHYQPYAPPRDFAAYRSK |
| | | RGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQEGL |
| | | YNELQKDKMAEAYSEIGMKGERRRGKGHDGLYQGLSTATKDTYDALHMQ |
| | | ALPPR |
| 30 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/CD226/CD3ζ | FGSGTKLEIKEPKSCDKTHTCPPCPDPKIYIWAPLAGTCGVLLLSLVITLYCN |
| | | HRNIVIFLNRRRRRERRDLFTESWDTQKAPNNYRSPISTSQPTNQSMDDTR |
| | | EDIYVNYPTFSRRPKTRVRVKFSRSADAPAYQQGQNQLYNELNLGRREEY |
| | | DVLDKRRGRDPEMGGKPRRKNPQEGLYNELQKDKMAEAYSEIGMKGERR |
| | | RGKGHDGLYQGLSTATKDTYDALHMQALPPR |
| 31 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/F36V- | FGSGTKLEIKEPKSCDKTHTCPPCPDPKIYIWAPLAGTCGVLLLSLVITLYCN |
| | FKBP/CD226/CD3ζ | HRNGVQVETISPGDGRTFPKRGQTCVVHYTGMLEDGKKVDSSRDRNKPFK |
| | | FMLGKQEVIRGWEEGVAQMSVGQRAKLTISPDYAYGATGHPGIIPPHATLV |
| | | FDVELLKLEIVIFLNRRRRRERRDLFTESWDTQKAPNNYRSPISTSQPTNQS |
| | | MDDTREDIYVNYPTFSRRPKTRVRVKFSRSADAPAYQQGQNQLYNELNLG |
| | | RREEYDVLDKRRGRDPEMGGKPRRKNPQEGLYNELQKDKMAEAYSEIGM |
| | | KGERRRGKGHDGLYQGLSTATKDTYDALHMQALPPR |
| 32 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/CD226/CD28/ | FGSGTKLEIKEPKSCDKTHTCPPCPDPKIYIWAPLAGTCGVLLLSLVITLYCN |
| | CD3ζ | HRNIVIFLNRRRRRERRDLFTESWDTQKAPNNYRSPISTSQPTNQSMDDTR |
| | | EDIYVNYPTFSRRPKTRVFWVRSKRSRLLHSDYMNMTPRRPGPTRKHYQP |
| | | YAPPRDFAAYRSRVKFSRSADAPAYQQGQNQLYNELNLGRREEYDVLDKR |
| | | RGRDPEMGGKPRRKNPQEGLYNELQKDKMAEAYSEIGMKGERRRGKGH |
| | | DGLYQGLSTATKDTYDALHMQALPPR |
| 33 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/F36V- | FGSGTKLEIKEPKSCDKTHTCPPCPDPKIYIWAPLAGTCGVLLLSLVITLYCN |
| | FKBP/CD226/CD28/ | HRNGVQVETISPGDGRTFPKRGQTCVVHYTGMLEDGKKVDSSRDRNKPFK |
| | CD3ζ | FMLGKQEVIRGWEEGVAQMSVGQRAKLTISPDYAYGATGHPGIIPPHATLV |
| | | FDVELLKLEIVIFLNRRRRRERRDLFTESWDTQKAPNNYRSPISTSQPTNQS |
| | | MDDTREDIYVNYPTFSRRPKTRVFWVRSKRSRLLHSDYMNMTPRRPGPTR |
| | | KHYQPYAPPRDFAAYRSRVKFSRSADAPAYQQGQNQLYNELNLGRREEY |
| | | DVLDKRRGRDPEMGGKPRRKNPQEGLYNELQKDKMAEAYSEIGMKGERR |
| | | RGKGHDGLYQGLSTATKDTYDALHMQALPPR |
| 34 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/CD226/4- | FGSGTKLEIKEPKSCDKTHTCPPCPDPKIYIWAPLAGTCGVLLLSLVITLYCN |
| | 1BB/CD3ζ | HRNIVIFLNRRRRRERRDLFTESWDTQKAPNNYRSPISTSQPTNQSMDDTR |
| | | EDIYVNYPTFSRRPKTRVKRGRKKLLYIFKQPFMRPVQTTQEEDGCSCRFP |
| | | EEEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHDGL |
| | | YQGLSTATKDTYDALHMQALPPR |
| 35 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/F36V- | FGSGTKLEIKEPKSCDKTHTCPPCPDPKIYIWAPLAGTCGVLLLSLVITLYCN |
| | FKBP/CD226/41BB/ | HRNGVQVETISPGDGRTFPKRGQTCVVHYTGMLEDGKKVDSSRDRNKPFK |
| | CD3ζ | FMLGKQEVIRGWEEGVAQMSVGQRAKLTISPDYAYGATGHPGIIPPHATLV |
| | | FDVELLKLEIVIFLNRRRRRERRDLFTESWDTQKAPNNYRSPISTSQPTNQS |
| | | MDDTREDIYVNYPTFSRRPKTRVKRGRKKLLYIFKQPFMRPVQTTQEEDGC |
| | | SCRFPEEEEGGCELRVKFSRSADAPAYQQGQNQLYNELNLGRREEYDVLD |
| | | KRRGRDPEMGGKPRRKNPQEGLYNELQKDKMAEAYSEIGMKGERRRGKG |
| | | HDGLYQGLSTATKDTYDALHMQALPPR |
| 36 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/CD226/CD28/ | FGSGTKLEIKEPKSCDKTHTCPPCPDPKIYIWAPLAGTCGVLLLSLVITLYCN |
| | 4-1BB/CD3ζ | HRNIVIFLNRRRRRERRDLFTESWDTQKAPNNYRSPISTSQPTNQSMDDTR |
| | | EDIYVNYPTFSRRPKTRVFWVRSKRSRLLHSDYMNMTPRRPGPTRKHYQP |
| | | YAPPRDFAAYRSKRGRKKLLYIFKQPFMRPVQTTQEEDGCSCRFPEEEEG |
| | | GCELRVKFSRSADAPAYQQGQNQLYNELNLGRREEYDVLDKRRGRDPEM |
| | | GGKPRRKNPQEGLYNELQKDKMAEAYSEIGMKGERRRGKGHDGLYQGLS |
| | | TATKDTYDALHMQALPPR |
| 37 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/F36V- | FGSGTKLEIKEPKSCDKTHTCPPCPDPKIYIWAPLAGTCGVLLLSLVITLYCN |
| | FKBP/CD226/CD28/ | HRNGVQVETISPGDGRTFPKRGQTCVVHYTGMLEDGKKVDSSRDRNKPFK |
| | 41BB/CD3ζ | FMLGKQEVIRGWEEGVAQMSVGQRAKLTISPDYAYGATGHPGIIPPHATLV |
| | | FDVELLKLEIVIFLNRRRRRERRDLFTESWDTQKAPNNYRSPISTSQPTNQS |
| | | MDDTREDIYVNYPTFSRRPKTRVFWVRSKRSRLLHSDYMNMTPRRPGPTR |
| | | KHYQPYAPPRDFAAYRSKRGRKKLLYIFKQPFMRPVQTTQEEDGCSCRFP |
| | | EEEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHDGL |
| | | YQGLSTATKDTYDALHMQALPPR |
| 38 | scFV GC33/hIgG1 | QVQLQQSGAELVRPGASVKLSCKASGYTFTDYEMHWVKQTPVHGLKWIG |
| | hinge/CD28 | ALDPKTGDTAYSQKFKGKATLTADKSSSTAYMELRSLTSEDSAVYYCTRFY |
| | TMD/F36V- | SYTYWGQGTLVTVSAGGGGSGGGGSGGGGSDVVMTQTPLSLPVSLGDQ |
| | FKBP/41BB/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIIGVQVETISPGDGRTFPKRG |
| | | QTCVVHYTGMLEDGKKVDSSRDRNKPFKFMLGKQEVIRGWEEGVAQMSV |
| | | GQRAKLTISPDYAYGATGHPGIIPPHATLVFDVELLKLEKRGRKKLLYIFKQP |
| | | FMRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 39 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/41BB/CD3ζ | SYTYWGQGTLVTVSAGGGGSGGGGSGGGGSDVVMTQTPLSLPVSLGDQ |
| | | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNKRGRKKLLYIFKQPFM |
| | | RPVQTTQEEDGCSCRFPEEEEGGCELRVKFSRSADAPAYQQGQNQLYNE |
| | | LNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 40 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/F36V- | SYTYWGQGTLVTVSAGGGGSGGGGSGGGGSDVVMTQTPLSLPVSLGDQ |
| | FKBP/41BB/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNGVQVETISPGDGRTFP |
| | | KRGQTCVVHYTGMLEDGKKVDSSRDRNKPFKFMLGKQEVIRGWEEGVAQ |
| | | MSVGQRAKLTISPDYAYGATGHPGIIPPHATLVFDVELLKLEKRGRKKLLYIF |
| | | KQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 41 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/CD28/CD3ζ | SYTYWGQGTLVTVSAGGGGSGGGGSGGGGSDVVMTQTPLSLPVSLGDQ |
| | | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNFWVRSKRSRLLHSDY |
| | | MNMTPRRPGPTRKHYQPYAPPRDFAAYRSRVKFSRSADAPAYQQGQNQL |
| | | YNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 42 | scFV GC33/hIgG1 | QVQLQQSGAELVRPGASVKLSCKASGYTFTDYEMHWVKQTPVHGLKWIG |
| | hinge/CD8α | ALDPKTGDTAYSQKFKGKATLTADKSSSTAYMELRSLTSEDSAVYYCTRFY |
| | TMD/F36V- | SYTYWGQGTLVTVSAGGGGSGGGGSGGGGSDVVMTQTPLSLPVSLGDQ |
| | FKBP/CD28/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKIYIWAPLAGTCGVLLLSLVITLYCNHRNGVQVETISPGDGRTFP |
| | | KRGQTCVVHYTGMLEDGKKVDSSRDRNKPFKFMLGKQEVIRGWEEGVAQ |
| | | MSVGQRAKLTISPDYAYGATGHPGIIPPHATLVFDVELLKLEFWVRSKRSRL |
| | | LHSDYMNMTPRRPGPTRKHYQPYAPPRDFAAYRSRVKFSRSADAPAYQQ |
| | | GQNQLYNELNLGRREEYDVLDKRRGRDPEMGGKPRRKNPQEGLYNELQK |
| | | DKMAEAYSEIGMKGERRRGKGHDGLYQGLSTATKDTYDALHMQALPPR |
| 43 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 | LLIYKVSNRFSGVPDRFSGSGSGTDFTLKISRVEAEDLGVYFCSQNTHVPPT |
| | TMD/F36V- | FGSGTKLEIKEPKSCDKTHTCPPCPDPKFWVLVVVGGVLACYSLLVTVAFII |
| | FKBP/41BB/CD3ζ | GVQVETISPGDGRTFPKRGQTCVVHYTGMLEDGKKVDSSRDRNKPFKFML |
| | | GKQEVIRGWEEGVAQMSVGQRAKLTISPDYAYGATGHPGIIPPHATLVFDV |
| | | ELLKLEKRGRKKLLYIFKQPFMRPVQTTQEEDGCSCRFPEEEEGGCELRVK |
| | | FSRSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDTYD |
| | | ALHMQALPPR |
| 44 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/41BB/CD3ζ | FGSGTKLEIKEPKSCDKTHTCPPCPDPKIYIWAPLAGTCGVLLLSLVITLYCN |
| | | HRNKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRKNP |
| | | QEGLYNELQKDKMAEAYSEIGMKGERRRGKGHDGLYQGLSTATKDTYDAL |
| | | HMQALPPR |
| 45 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/F36V- | FGSGTKLEIKEPKSCDKTHTCPPCPDPKIYIWAPLAGTCGVLLLSLVITLYCN |
| | FKBP/41BB/CD3ζ | HRNGVQVETISPGDGRTFPKRGQTCVVHYTGMLEDGKKVDSSRDRNKPFK |
| | | FMLGKQEVIRGWEEGVAQMSVGQRAKLTISPDYAYGATGHPGIIPPHATLV |
| | | FDVELLKLEKRGRKKLLYIFKQPFMRPVQTTQEEDGCSCRFPEEEEGGCEL |
| | | RVKFSRSADAPAYQQGQNQLYNELNLGRREEYDVLDKRRGRDPEMGGKP |
| | | RRKNPQEGLYNELQKDKMAEAYSEIGMKGERRRGKGHDGLYQGLSTATK |
| | | DTYDALHMQALPPR |
| 46 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/CD28/CD3ζ | FGSGTKLEIKEPKSCDKTHTCPPCPDPKIYIWAPLAGTCGVLLLSLVITLYCN |
| | | HRNFWVRSKRSRLLHSDYMNMTPRRPGPTRKHYQPYAPPRDFAAYRSRV |
| | | KFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTY |
| | | DALHMQALPPR |
| 47 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD8α | LLIYKVSNRFSGVPDRFSGSGSGTDFTLKISRVEAEDLGVYFCSQNTHVPPT |
| | TMD/F36V- | FGSGTKLEIKEPKSCDKTHTCPPCPDPKIYIWAPLAGTCGVLLLSLVITLYCN |
| | FKBP/CD28/CD3ζ | HRNGVQVETISPGDGRTFPKRGQTCVVHYTGMLEDGKKVDSSRDRNKPFK |
| | | FMLGKQEVIRGWEEGVAQMSVGQRAKLTISPDYAYGATGHPGIIPPHATLV |
| | | FDVELLKLEFWVRSKRSRLLHSDYMNMTPRRPGPTRKHYQPYAPPRDFAA |
| | | YRSRVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQGLST |
| | | ATKDTYDALHMQALPPR |
| 48 | 3-17I heavy chain | QVQLVQSGAEVKKPGSSVKVSCKASGGTFSSYAISWVRQAPGQGLEWMG |
| | variable region | GIIPIFGTANYAQKFQGRVTITADESTSTAYMELSSLRSEDTAVYYCARGLL |
| | sequence | WNYWGQGTLVTV |
| 49 | 3-17I HC-CDR1 | SYAIS |
| 50 | 3-17I HC-CDR2 | GIIPIFGTANYAQKFQG |
| 51 | 3-17I HC-CDR3 | GLLWNY |
| 52 | 3-17I light chain | EIVMTQSPATLSVSPGERATLSCRASQSVSSNLAWYQQKPGQAPRLIIYGA |
| | variable region | STTASGIPARFSASGSGTDFTLTISSLQSEDFAVYYCQQYNNWPPAYTFGQ |
| | sequence | GTKLEIK |
| 53 | 3-17I LC-CDR1 | RASQSVSSNLA |
| 54 | 3-17I LC-CDR2 | GASTTAS |
| 55 | 3-17I LC-CDR3 | QQYNNWPPAYT |
| 56 | 3-17I scFv | QVQLVQSGAEVKKPGSSVKVSCKASGGTFSSYAISWVRQAPGQGLEWMG |
| | | GIIPIFGTANYAQKFQGRVTITADESTSTAYMELSSLRSEDTAVYYCARGLL |
| | | WNYWGQGTLVTVSSKLSGSASAPKLEEGEFSEARVEIVMTQSPATLSVSP |
| | | GERATLSCRASQSVSSNLAWYQQKPGQAPRLIIYGASTTASGIPARFSASG |
| | | SGTDFTLTISSLQSEDFAVYYCQQYNNWPPAYTFGQGTKLEIK |
| 57 | CD226 TMD | GGTVLLLLFVISITTIIVIFL |
| 58 | CD226 ICD v1 | NRRRRRERRDLFTESWDTQKAPNNYRSPISTSQPTNQSMDDTREDIYVNY |
| | | PTFSRRPKTRV |
| 59 | CD226 ICD v2 | FLNRRRRRERRDLFTESWDTQKAPNNYRSPISTSQPTNQSMDDTREDIYV |
| | | NYPTFSRRPKTRV |
| 60 | CD226 TMD | GGTVLLLLFVISITTIIVIFL |
| 61 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 | LLIYKVSNRFSGVPDRFSGSGSGTDFTLKISRVEAEDLGVYFCSQNTHVPPT |
| | TMD/CD3ζ | FGSGTKLEIKEPKSCDKTHTCPPCPDPKFWVLVVVGGVLACYSLLVTVAFII |
| | | RVKFSRSADAPAYQQGQNQLYNELNLGRREEYDVLDKRRGRDPEMGGKP |
| | | RRKNPQEGLYNELQKDKMAEAYSEIGMKGERRRGKGHDGLYQGLSTATK |
| | | DTYDALHMQALPPR |
| 62 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 | LLIYKVSNRFSGVPDRFSGSGSGTDFTLKISRVEAEDLGVYFCSQNTHVPPT |
| | TMD/CD226 ICD | FGSGTKLEIKEPKSCDKTHTCPPCPDPKFWVLVVVGGVLACYSLLVTVAFIIF |
| | v2 | LNRRRRRERRDLFTESWDTQKAPNNYRSPISTSQPTNQSMDDTREDIYVN |
| | | YPTFSRRPKTRV |
| 63 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 | LLIYKVSNRFSGVPDRFSGSGSGTDFTLKISRVEAEDLGVYFCSQNTHVPPT |
| | TMD/41BB/CD3ζ | FGSGTKLEIKEPKSCDKTHTCPPCPDPKFWVLVVVGGVLACYSLLVTVAFIIK |
| | | RGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQEGL |
| | | YNELQKDKMAEAYSEIGMKGERRRGKGHDGLYQGLSTATKDTYDALHMQ |
| | | ALPPR |
| 64 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD226 ICD | FGSGTKLEIKEPKSCDKTHTCPPCPDPKFWVLVVVGGVLACYSLLVTVAFII |
| | v1/41BB/CD3ζ | NRRRRRERRDLFTESWDTQKAPNNYRSPISTSQPTNQSMDDTREDIYVNY |
| | | PTFSRRPKTRVKRGRKKLLYIFKQPFMRPVQTTQEEDGCSCRFPEEEEGG |
| | | CELRVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQGLST |
| | | ATKDTYDALHMQALPPR |
| 65 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD226 ICD | FGSGTKLEIKEPKSCDKTHTCPPCPDPKFWVLVVVGGVLACYSLLVTVAFIIF |
| | v2/41BB/CD3ζ | LNRRRRRERRDLFTESWDTQKAPNNYRSPISTSQPTNQSMDDTREDIYVN |
| | | YPTFSRRPKTRVKRGRKKLLYIFKQPFMRPVQTTQEEDGCSCRFPEEEEG |
| | | GCELRVKFSRSADAPAYQQGQNQLYNELNLGRREEYDVLDKRRGRDPEM |
| | | GGKPRRKNPQEGLYNELQKDKMAEAYSEIGMKGERRRGKGHDGLYQGLS |
| | | TATKDTYDALHMQALPPR |
| 66 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD226 ICD | FGSGTKLEIKEPKSCDKTHTCPPCPDPKFWVLVVVGGVLACYSLLVTVAFII |
| | v1/CD3ζ | NRRRRRERRDLFTESWDTQKAPNNYRSPISTSQPTNQSMDDTREDIYVNY |
| | | PTFSRRPKTRVRVKFSRSADAPAYQQGQNQLYNELNLGRREEYDVLDKRR |
| | | GRDPEMGGKPRRKNPQEGLYNELQKDKMAEAYSEIGMKGERRRGKGHD |
| | | GLYQGLSTATKDTYDALHMQALPPR |
| 67 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD226 ICD | FGSGTKLEIKEPKSCDKTHTCPPCPDPKFWVLVVVGGVLACYSLLVTVAFIIF |
| | v2/CD3ζ | LNRRRRRERRDLFTESWDTQKAPNNYRSPISTSQPTNQSMDDTREDIYVN |
| | | YPTFSRRPKTRVRVKFSRSADAPAYQQGQNQLYNELNLGRREEYDVLDKR |
| | | RGRDPEMGGKPRRKNPQEGLYNELQKDKMAEAYSEIGMKGERRRGKGH |
| | | DGLYQGLSTATKDTYDALHMQALPPR |
| 68 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD28 ICD/CD3ζ | FGSGTKLEIKEPKSCDKTHTCPPCPDPKFWVLVVVGGVLACYSLLVTVAFIIF |
| | | WVRSKRSRLLHSDYMNMTPRRPGPTRKHYQPYAPPRDFAAYRSRVKFSR |
| | | SADAPAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTYDALH |
| | | MQALPPR |
| 69 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD226 ICD v1/ | FGSGTKLEIKEPKSCDKTHTCPPCPDPKFWVLVVVGGVLACYSLLVTVAFII |
| | CD28 ICD/CD3ζ | NRRRRRERRDLFTESWDTQKAPNNYRSPISTSQPTNQSMDDTREDIYVNY |
| | | PTFSRRPKTRVFWVRSKRSRLLHSDYMNMTPRRPGPTRKHYQPYAPPRD |
| | | FAAYRSRVKFSRSADAPAYQQGQNQLYNELNLGRREEYDVLDKRRGRDP |
| | | EMGGKPRRKNPQEGLYNELQKDKMAEAYSEIGMKGERRRGKGHDGLYQG |
| | | LSTATKDTYDALHMQALPPR |
| 70 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD226 ICD v2/ | FGSGTKLEIKEPKSCDKTHTCPPCPDPKFWVLVVVGGVLACYSLLVTVAFIIF |
| | CD28 ICD/CD3ζ | LNRRRRRERRDLFTESWDTQKAPNNYRSPISTSQPTNQSMDDTREDIYVN |
| | | YPTFSRRPKTRVFWVRSKRSRLLHSDYMNMTPRRPGPTRKHYQPYAPPR |
| | | DFAAYRSRVKFSRSADAPAYQQGQNQLYNELNLGRREEYDVLDKRRGRD |
| | | PEMGGKPRRKNPQEGLYNELQKDKMAEAYSEIGMKGERRRGKGHDGLYQ |
| | | GLSTATKDTYDALHMQALPPR |
| 71 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD28 ICD/CD226 | FGSGTKLEIKEPKSCDKTHTCPPCPDPKFWVLVVVGGVLACYSLLVTVAFIIF |
| | ICD v1/CD3ζ | WVRSKRSRLLHSDYMNMTPRRPGPTRKHYQPYAPPRDFAAYRSNRRRRR |
| | | ERRDLFTESWDTQKAPNNYRSPISTSQPTNQSMDDTREDIYVNYPTFSRRP |
| | | KTRVRVKFSRSADAPAYQQGQNQLYNELNLGRREEYDVLDKRRGRDPEM |
| | | GGKPRRKNPQEGLYNELQKDKMAEAYSEIGMKGERRRGKGHDGLYQGLS |
| | | TATKDTYDALHMQALPPR |
| 72 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD28 | FGSGTKLEIKEPKSCDKTHTCPPCPDPKFWVLVVVGGVLACYSLLVTVAFIIF |
| | ICD/41BB/CD3ζ | WVRSKRSRLLHSDYMNMTPRRPGPTRKHYQPYAPPRDFAAYRSKRGRKK |
| | | LLYIFKQPFMRPVQTTQEEDGCSCRFPEEEEGGCELRVKFSRSADAPAYQ |
| | | QGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| 73 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD226 ICD v1/ | FGSGTKLEIKEPKSCDKTHTCPPCPDPKFWVLVVVGGVLACYSLLVTVAFII |
| | CD28 | NRRRRRERRDLFTESWDTQKAPNNYRSPISTSQPTNQSMDDTREDIYVNY |
| | ICD/41BB/CD3ζ | PTFSRRPKTRVFWVRSKRSRLLHSDYMNMTPRRPGPTRKHYQPYAPPRD |
| | | FAAYRSKRGRKKLLYIFKQPFMRPVQTTQEEDGCSCRFPEEEEGGCELRV |
| | | KFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTY |
| | | DALHMQALPPR |
| 74 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD226 ICD v2/ | FGSGTKLEIKEPKSCDKTHTCPPCPDPKFWVLVVVGGVLACYSLLVTVAFIIF |
| | CD28 | LNRRRRRERRDLFTESWDTQKAPNNYRSPISTSQPTNQSMDDTREDIYVN |
| | ICD/41BB/CD3ζ | YPTFSRRPKTRVFWVRSKRSRLLHSDYMNMTPRRPGPTRKHYQPYAPPR |
| | | DFAAYRSKRGRKKLLYIFKQPFMRPVQTTQEEDGCSCRFPEEEEGGCELR |
| | | VKFSRSADAPAYQQGQNQLYNELNLGRREEYDVLDKRRGRDPEMGGKPR |
| | | RKNPQEGLYNELQKDKMAEAYSEIGMKGERRRGKGHDGLYQGLSTATKDT |
| | | YDALHMQALPPR |
| 75 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD28 TMD/ | LLIYKVSNRFSGVPDRFSGSGSGTDFTLKISRVEAEDLGVYFCSQNTHVPPT |
| | CD28 ICD/CD226 | FGSGTKLEIKEPKSCDKTHTCPPCPDPKFWVLVVVGGVLACYSLLVTVAFIIF |
| | ICD v1/41BB/CD3ζ | WVRSKRSRLLHSDYMNMTPRRPGPTRKHYQPYAPPRDFAAYRSNRRRRR |
| | | ERRDLFTESWDTQKAPNNYRSPISTSQPTNQSMDDTREDIYVNYPTFSRRP |
| | | KTRVKRGRKKLLYIFKQPFMRPVQTTQEEDGCSCRFPEEEEGGCELRVKF |
| | | SRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDA |
| | | LHMQALPPR |
| 76 | hIgG heavy chain | MDWIWRILFLVGAATGAHSQVQLQQSGAELVRPGASVKLSCKASGYTFTD |
| | signal | YEMHWVKQTPVHGLKWIGALDPKTGDTAYSQKFKGKATLTADKSSSTAYM |
| | sequence/scFV | ELRSLTSEDSAVYYCTRFYSYTYWGQGTLVTVSAGGGGSGGGGSGGGGS |
| | GC33/hIgG1 | DVVMTQTPLSLPVSLGDQASISCRSSQSLVHSNGNTYLHWYLQKPGQSPK |
| | hinge/CD226 | LLIYKVSNRFSGVPDRFSGSGSGTDFTLKISRVEAEDLGVYFCSQNTHVPPT |
| | TMD/CD226 ICD | FGSGTKLEIKEPKSCDKTHTCPPCPDPKGGTVLLLLFVISITTIIVIFLNRRRRR |
| | v1/41BB/CD3ζ | ERRDLFTESWDTQKAPNNYRSPISTSQPTNQSMDDTREDIYVNYPTFSRRP |
| | | KTRVKRGRKKLLYIFKQPFMRPVQTTQEEDGCSCRFPEEEEGGCELRVKF |
| | | SRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDA |
| | | LHMQALPPR |
| 77 | hIgG heavy chain | MDWIWRILFLVGAATGAHSQVQLVQSGAEVKKPGSSVKVSCKASGGTFSS |
| | signal | YAISWVRQAPGQGLEWMGGIIPIFGTANYAQKFQGRVTITADESTSTAYMEL |
| | sequence/scFV 3- | SSLRSEDTAVYYCARGLLWNYWGQGTLVTVSSKLSGSASAPKLEEGEFSE |
| | 17I/hIgG1 | ARVEIVMTQSPATLSVSPGERATLSCRASQSVSSNLAWYQQKPGQAPRLII |
| | hinge/CD226 | YGASTTASGIPARFSASGSGTDFTLTISSLQSEDFAVYYCQQYNNWPPAYT |
| | TMD/CD226 ICD | FGQGTKLEIKEPKSCDKTHTCPPCPDPKGGTVLLLLFVISITTIIVIFLNRRRR |
| | v1 | RERRDLFTESWDTQKAPNNYRSPISTSQPTNQSMDDTREDIYVNYPTFSRR |
| | | PKTRV |
| 78 | hIgG heavy chain | MDWIWRILFLVGAATGAHSQVQLVQSGAEVKKPGSSVKVSCKASGGTFSS |
| | signal | YAISWVRQAPGQGLEWMGGIIPIFGTANYAQKFQGRVTITADESTSTAYMEL |
| | sequence/scFV 3- | SSLRSEDTAVYYCARGLLWNYWGQGTLVTVSSKLSGSASAPKLEEGEFSE |
| | 17I/hIgG1 | ARVEIVMTQSPATLSVSPGERATLSCRASQSVSSNLAWYQQKPGQAPRLII |
| | hinge/CD226 TMD/ | YGASTTASGIPARFSASGSGTDFTLTISSLQSEDFAVYYCQQYNNWPPAYT |
| | CD3ζ | FGQGTKLEIKEPKSCDKTHTCPPCPDPKGGTVLLLLFVISITTIIVIFLRVKFSR |
| | | SADAPAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTYDALH |
| | | MQALPPR |
| 79 | hIgG heavy chain | MDWIWRILFLVGAATGAHSQVQLVQSGAEVKKPGSSVKVSCKASGGTFSS |
| | signal | YAISWVRQAPGQGLEWMGGIIPIFGTANYAQKFQGRVTITADESTSTAYMEL |
| | sequence/scFV 3- | SSLRSEDTAVYYCARGLLWNYWGQGTLVTVSSKLSGSASAPKLEEGEFSE |
| | 17I/hIgG1 | ARVEIVMTQSPATLSVSPGERATLSCRASQSVSSNLAWYQQKPGQAPRLII |
| | hinge/CD226 | YGASTTASGIPARFSASGSGTDFTLTISSLQSEDFAVYYCQQYNNWPPAYT |
| | TMD/CD226 ICD | FGQGTKLEIKEPKSCDKTHTCPPCPDPKGGTVLLLLFVISITTIIVIFLNRRRR |
| | v1/CD3ζ | RERRDLFTESWDTQKAPNNYRSPISTSQPTNQSMDDTREDIYVNYPTFSRR |
| | | PKTRVRVKFSRSADAPAYQQGQNQLYNELNLGRREEYDVLDKRRGRDPE |
| | | MGGKPRRKNPQEGLYNELQKDKMAEAYSEIGMKGERRRGKGHDGLYQGL |
| | | STATKDTYDALHMQALPPR |
| 80 | scFV GC33/hIgG1 | QVQLQQSGAELVRPGASVKLSCKASGYTFTDYEMHWVKQTPVHGLKWIG |
| | hinge/CD28 | ALDPKTGDTAYSQKFKGKATLTADKSSSTAYMELRSLTSEDSAVYYCTRFY |
| | TMD/CD3ζ | SYTYWGQGTLVTVSAGGGGSGGGGSGGGGSDVVMTQTPLSLPVSLGDQ |
| | | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIIRVKFSRSADAPAYQQGQN |
| | | QLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 81 | scFV GC33/hIgG1 | QVQLQQSGAELVRPGASVKLSCKASGYTFTDYEMHWVKQTPVHGLKWIG |
| | hinge/CD28 | ALDPKTGDTAYSQKFKGKATLTADKSSSTAYMELRSLTSEDSAVYYCTRFY |
| | TMD/CD226 ICD | SYTYWGQGTLVTVSAGGGGSGGGGSGGGGSDVVMTQTPLSLPVSLGDQ |
| | v2 | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIIFLNRRRRRERRDLFTESW |
| | | DTQKAPNNYRSPISTSQPTNQSMDDTREDIYVNYPTFSRRPKTRV |
| 82 | scFV GC33/hIgG1 | QVQLQQSGAELVRPGASVKLSCKASGYTFTDYEMHWVKQTPVHGLKWIG |
| | hinge/CD28 | ALDPKTGDTAYSQKFKGKATLTADKSSSTAYMELRSLTSEDSAVYYCTRFY |
| | TMD/41BB/CD3ζ | SYTYWGQGTLVTVSAGGGGSGGGGSGGGGSDVVMTQTPLSLPVSLGDQ |
| | | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIIKRGRKKLLYIFKQPFMRPV |
| | | QTTQEEDGCSCRFPEEEEGGCELRVKFSRSADAPAYQQGQNQLYNELNL |
| | | GRREEYDVLDKRRGRDPEMGGKPRRKNPQEGLYNELQKDKMAEAYSEIG |
| | | MKGERRRGKGHDGLYQGLSTATKDTYDALHMQALPPR |
| 83 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD226 ICD | SYTYWGQGTLVTVSAGGGGSGGGGSGGGGSDVVMTQTPLSLPVSLGDQ |
| | v1/41BB/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIINRRRRRERRDLFTESWDT |
| | | QKAPNNYRSPISTSQPTNQSMDDTREDIYVNYPTFSRRPKTRVKRGRKKLL |
| | | YIFKQPFMRPVQTTQEEDGCSCRFPEEEEGGCELRVKFSRSADAPAYQQG |
| | | QNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| 84 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD226 ICD | SYTYWGQGTLVTVSAGGGGSGGGGSGGGGSDVVMTQTPLSLPVSLGDQ |
| | v2/41BB/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIIFLNRRRRRERRDLFTESW |
| | | DTQKAPNNYRSPISTSQPTNQSMDDTREDIYVNYPTFSRRPKTRVKRGRKK |
| | | LLYIFKQPFMRPVQTTQEEDGCSCRFPEEEEGGCELRVKFSRSADAPAYQ |
| | | QGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| 85 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD226 ICD | SYTYWGQGTLVTVSAGGGGSGGGGSGGGGSDVVMTQTPLSLPVSLGDQ |
| | v1/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIINRRRRRERRDLFTESWDT |
| | | QKAPNNYRSPISTSQPTNQSMDDTREDIYVNYPTFSRRPKTRVRVKFSRSA |
| | | DAPAYQQGQNQLYNELNLGRREEYDVLDKRRGRDPEMGGKPRRKNPQE |
| | | GLYNELQKDKMAEAYSEIGMKGERRRGKGHDGLYQGLSTATKDTYDALHM |
| | | QALPPR |
| 86 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD226 ICD | SYTYWGQGTLVTVSAGGGGSGGGGSGGGGSDVVMTQTPLSLPVSLGDQ |
| | v2/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIIFLNRRRRRERRDLFTESW |
| | | DTQKAPNNYRSPISTSQPTNQSMDDTREDIYVNYPTFSRRPKTRVRVKFSR |
| | | SADAPAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTYDALH |
| | | MQALPPR |
| 87 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD28 ICD/CD3ζ | SYTYWGQGTLVTVSAGGGGSGGGGSGGGGSDVVMTQTPLSLPVSLGDQ |
| | | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIIFWVRSKRSRLLHSDYMNM |
| | | TPRRPGPTRKHYQPYAPPRDFAAYRSRVKFSRSADAPAYQQGQNQLYNEL |
| | | NLGRREEYDVLDKRRGRDPEMGGKPRRKNPQEGLYNELQKDKMAEAYSE |
| | | IGMKGERRRGKGHDGLYQGLSTATKDTYDALHMQALPPR |
| 88 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD226 ICD v1/ | SYTYWGQGTLVTVSAGGGGSGGGGSGGGGSDVVMTQTPLSLPVSLGDQ |
| | CD28 ICD/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIINRRRRRERRDLFTESWDT |
| | | QKAPNNYRSPISTSQPTNQSMDDTREDIYVNYPTFSRRPKTRVFWVRSKR |
| | | SRLLHSDYMNMTPRRPGPTRKHYQPYAPPRDFAAYRSRVKFSRSADAPAY |
| | | QQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPPR |
| 89 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD226 ICD v2/ | SYTYWGQGTLVTVSAGGGGSGGGGSGGGGSDVVMTQTPLSLPVSLGDQ |
| | CD28 ICD/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIIFLNRRRRRERRDLFTESW |
| | | DTQKAPNNYRSPISTSQPTNQSMDDTREDIYVNYPTFSRRPKTRVFWVRSK |
| | | RSRLLHSDYMNMTPRRPGPTRKHYQPYAPPRDFAAYRSRVKFSRSADAPA |
| | | YQQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALPPR |
| 90 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD28 ICD/CD226 | SYTYWGQGTLVTVSAGGGGSGGGGSGGGGSDVVMTQTPLSLPVSLGDQ |
| | ICD v1/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIIFWVRSKRSRLLHSDYMNM |
| | | TPRRPGPTRKHYQPYAPPRDFAAYRSNRRRRRERRDLFTESWDTQKAPN |
| | | NYRSPISTSQPTNQSMDDTREDIYVNYPTFSRRPKTRVRVKFSRSADAPAY |
| | | QQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPPR |
| 91 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD28 | SYTYWGQGTLVTVSAGGGGSGGGGSGGGGSDVVMTQTPLSLPVSLGDQ |
| | ICD/41BB/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIIFWVRSKRSRLLHSDYMNM |
| | | TPRRPGPTRKHYQPYAPPRDFAAYRSKRGRKKLLYIFKQPFMRPVQTTQE |
| | | EDGCSCRFPEEEEGGCELRVKFSRSADAPAYQQGQNQLYNELNLGRREE |
| | | YDVLDKRRGRDPEMGGKPRRKNPQEGLYNELQKDKMAEAYSEIGMKGER |
| | | RRGKGHDGLYQGLSTATKDTYDALHMQALPPR |
| 92 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD226 ICD v1/ | SYTYWGQGTLVTVSAGGGGSGGGGSGGGGSDVVMTQTPLSLPVSLGDQ |
| | CD28 | ASISCRSSQSLVHSNGNTYLHWYLQKPGQSPKLLIYKVSNRFSGVPDRFSG |
| | ICD/41BB/CD3ζ | SGSGTDFTLKISRVEAEDLGVYFCSQNTHVPPTFGSGTKLEIKEPKSCDKTH |
| | | TCPPCPDPKFWVLVVVGGVLACYSLLVTVAFIINRRRRRERRDLFTESWDT |
| | | QKAPNNYRSPISTSQPTNQSMDDTREDIYVNYPTFSRRPKTRVFWVRSKR |
| | | SRLLHSDYMNMTPRRPGPTRKHYQPYAPPRDFAAYRSKRGRKKLLYIFKQ |
| | | PFMRPVQTTQEEDGCSCRFPEEEEGGCELRVKFSRSADAPAYQQGQNQL |
| | | YNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 93 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD226 ICD v2/ | SYTYWGQGTLVTVSAGGGGSGGGGSGGGGSDVVMTQTPLSLPVSLGDQ |
| | CD28 | ASISCRSSQSLVHSNGNTYLHWYLQKPGQSPKLLIYKVSNRFSGVPDRFSG |
| | ICD/41BB/CD3ζ | SGSGTDFTLKISRVEAEDLGVYFCSQNTHVPPTFGSGTKLEIKEPKSCDKTH |
| | | TCPPCPDPKFWVLVVVGGVLACYSLLVTVAFIIFLNRRRRRERRDLFTESW |
| | | DTQKAPNNYRSPISTSQPTNQSMDDTREDIYVNYPTFSRRPKTRVFWVRSK |
| | | RSRLLHSDYMNMTPRRPGPTRKHYQPYAPPRDFAAYRSKRGRKKLLYIFK |
| | | QPFMRPVQTTQEEDGCSCRFPEEEEGGCELRVKFSRSADAPAYQQGQNQ |
| | | LYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 94 | scFV GC33/hIgG1 | QVQLQQSGAELVRPGASVKLSCKASGYTFTDYEMHWVKQTPVHGLKWIG |
| | hinge/CD28 TMD/ | ALDPKTGDTAYSQKFKGKATLTADKSSSTAYMELRSLTSEDSAVYYCTRFY |
| | CD28 ICD/CD226 | SYTYWGQGTLVTVSAGGGGSGGGGSGGGGSDVVMTQTPLSLPVSLGDQ |
| | ICD v1/41BB/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKFWVLVVVGGVLACYSLLVTVAFIIFWVRSKRSRLLHSDYMNM |
| | | TPRRPGPTRKHYQPYAPPRDFAAYRSNRRRRRERRDLFTESWDTQKAPN |
| | | NYRSPISTSQPTNQSMDDTREDIYVNYPTFSRRPKTRVKRGRKKLLYIFKQP |
| | | FMRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 95 | scFV GC33/hIgG1 | QVQLQQSGAELVRPGASVKLSCKASGYTFTDYEMHWVKQTPVHGLKWIG |
| | hinge/CD226 | ALDPKTGDTAYSQKFKGKATLTADKSSSTAYMELRSLTSEDSAVYYCTRFY |
| | TMD/CD226 ICD | SYTYWGQGTLVTVSAGGGGSGGGGSGGGGSDVVMTQTPLSLPVSLGDQ |
| | v1/41BB/CD3ζ | ASISCRSSQSLVHSNGNTYLHWYLQKPGQSPKLLIYKVSNRFSGVPDRFSG |
| | | SGSGTDFTLKISRVEAEDLGVYFCSQNTHVPPTFGSGTKLEIKEPKSCDKTH |
| | | TCPPCPDPKGGTVLLLLFVISITTIIVIFLNRRRRRERRDLFTESWDTQKAPNN |
| | | YRSPISTSQPTNQSMDDTREDIYVNYPTFSRRPKTRVKRGRKKLLYIFKQPF |
| | | MRPVQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMAEAY |
| | | SEIGMKGERRRGKGHDGLYQGLSTATKDTYDALHMQALPPR |
| 96 | scFV 3-17I/hIgG1 | QVQLVQSGAEVKKPGSSVKVSCKASGGTFSSYAISWVRQAPGQGLEWMG |
| | hinge/CD226 | GIIPIFGTANYAQKFQGRVTITADESTSTAYMELSSLRSEDTAVYYCARGLL |
| | TMD/CD226 ICD | WNYWGQGTLVTVSSKLSGSASAPKLEEGEFSEARVEIVMTQSPATLSVSP |
| | v1 | GERATLSCRASQSVSSNLAWYQQKPGQAPRLIIYGASTTASGIPARFSASG |
| | | SGTDFTLTISSLQSEDFAVYYCQQYNNWPPAYTFGQGTKLEIKEPKSCDKT |
| | | HTCPPCPDPKGGTVLLLLFVISITTIIVIFLNRRRRRERRDLFTESWDTQKAPN |
| | | NYRSPISTSQPTNQSMDDTREDIYVNYPTFSRRPKTRV |
| 97 | scFV 3-17I/hIgG1 | QVQLVQSGAEVKKPGSSVKVSCKASGGTFSSYAISWVRQAPGQGLEWMG |
| | hinge/CD226 TMD/ | GIIPIFGTANYAQKFQGRVTITADESTSTAYMELSSLRSEDTAVYYCARGLL |
| | CD3ζ | WNYWGQGTLVTVSSKLSGSASAPKLEEGEFSEARVEIVMTQSPATLSVSP |
| | | GERATLSCRASQSVSSNLAWYQQKPGQAPRLIIYGASTTASGIPARFSASG |
| | | SGTDFTLTISSLQSEDFAVYYCQQYNNWPPAYTFGQGTKLEIKEPKSCDKT |
| | | HTCPPCPDPKGGTVLLLLFVISITTIIVIFLRVKFSRSADAPAYQQGQNQLYNE |
| | | LNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 98 | scFV 3-17I/hIgG1 | QVQLVQSGAEVKKPGSSVKVSCKASGGTFSSYAISWVRQAPGQGLEWMG |
| | hinge/CD226 | GIIPIFGTANYAQKFQGRVTITADESTSTAYMELSSLRSEDTAVYYCARGLL |
| | TMD/CD226 ICD | WNYWGQGTLVTVSSKLSGSASAPKLEEGEFSEARVEIVMTQSPATLSVSP |
| | v1/CD3ζ | GERATLSCRASQSVSSNLAWYQQKPGQAPRLIIYGASTTASGIPARFSASG |
| | | SGTDFTLTISSLQSEDFAVYYCQQYNNWPPAYTFGQGTKLEIKEPKSCDKT |
| | | HTCPPCPDPKGGTVLLLLFVISITTIIVIFLNRRRRRERRDLFTESWDTQKAPN |
| | | NYRSPISTSQPTNQSMDDTREDIYVNYPTFSRRPKTRVRVKFSRSADAPAY |
| | | QQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPPR |
| 99 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 | CCCCCGTGCACGGTCTGAAGTGGATTGGGGCCCTGGATCCCAA |
| | TMD/CD3ζ | GACCGGCGATACTGCGTACTCACAGAAGTTCAAGGGAAAGGCCA |
| | | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGCCCGGATCC |
| | | GAAGTTCTGGGTGCTGGTGGTGGTCGGGGGAGTGCTCGCCTGC |
| | | TACTCTCTGCTGGTGACCGTGGCCTTCATCATCCGCGTGAAGTTC |
| | | AGCCGGAGCGCTGATGCCCCTGCATACCAGCAGGGGCAGAACC |
| | | AGCTCTACAACGAACTGAACCTTGGACGGCGGGAGGAATACGAT |
| | | GTGCTGGATAAGCGAAGAGGCCGCGACCCAGAAATGGGCGGGA |
| | | AGCCCAGACGCAAGAATCCTCAGGAGGGACTGTACAACGAGCTC |
| | | CAGAAAGACAAGATGGCCGAAGCGTACAGCGAGATCGGCATGAA |
| | | GGGGGAACGGAGAAGGGGAAAGGGCCATGACGGATTGTACCAG |
| | | GGCCTGTCGACCGCTACCAAAGACACCTACGACGCCCTCCATAT |
| | | GCAAGCACTGCCGCCACGCTGAACGCGT |
| 100 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 | CCCCCGTGCACGGTCTGAAGTGGATTGGGGCCCTGGATCCCAA |
| | TMD/CD226 ICD | GACCGGCGATACTGCGTACTCACAGAAGTTCAAGGGAAAGGCCA |
| | v2 | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGTCCGGATCC |
| | | GAAGTTCTGGGTGCTGGTGGTGGTCGGGGGAGTGCTCGCCTGC |
| | | TACTCTCTGCTGGTGACCGTGGCATTCATTATCTTCCTGAACAGA |
| | | AGGAGGCGCCGGGAGCGGCGCGACCTGTTCACTGAATCCTGGG |
| | | ACACCCAGAAGGCCCCCAACAACTACAGGTCCCCTATCTCAACC |
| | | TCCCAACCCACCAACCAGAGCATGGACGATACTCGCGAGGACAT |
| | | CTACGTGAACTACCCCACTTTCTCCCGGCGGCCTAAGACACGGG |
| | | TGTGAACGCGT |
| 101 | (nucleotide) hIgG | ATGGACTGGATCTGGAGGATTTTGTTCCTTGTGGGAGCTGCCAC |
| | heavy chain signal | CGGTGCACATTCGCAGGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 | CCCCCGTGCACGGTCTGAAGTGGATTGGGGCCCTGGATCCCAA |
| | TMD/41BB/CD3ζ | GACCGGCGATACTGCGTACTCACAGAAGTTCAAGGGAAAGGCCA |
| | | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGCCCGGATCC |
| | | AAAGTTCTGGGTGCTGGTGGTGGTCGGGGGAGTGCTCGCCTGC |
| | | TACTCTCTGCTGGTGACCGTGGCCTTTATAATCAAGCGCGGTCG |
| | | GAAGAAGCTGCTGTACATCTTCAAGCAGCCCTTCATGCGGCCTG |
| | | TGCAGACCACACAGGAAGAGGATGGCTGCTCCTGCCGCTTCCCG |
| | | GAGGAAGAGGAGGGCGGATGCGAACTGCGCGTGAAGTTCAGCC |
| | | GGAGCGCTGATGCCCCTGCATACCAGCAGGGGCAGAACCAGCT |
| | | CTACAACGAACTGAACCTTGGACGGCGGGAGGAATACGATGTGC |
| | | TGGATAAGCGAAGAGGCCGCGACCCAGAAATGGGCGGGAAGCC |
| | | CAGACGCAAGAATCCTCAGGAGGGACTGTACAACGAGCTCCAGA |
| | | AAGACAAGATGGCCGAAGCGTACAGCGAGATCGGCATGAAGGG |
| | | GGAACGGAGAAGGGGAAAGGGCCATGACGGATTGTACCAGGGC |
| | | CTGTCGACCGCTACCAAAGACACCTACGACGCCCTCCATATGCA |
| | | AGCACTGCCGCCACGCTGAACGCGT |
| 102 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD226 ICD | GACCGGCGATACTGCGTACTCACAGAAGTTCAAGGGAAAGGCCA |
| | v1/41BB/CD3ζ | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGTCCGGATCC |
| | | GAAGTTCTGGGTGCTGGTGGTGGTCGGGGGAGTGCTCGCCTGC |
| | | TACTCTCTGCTGGTGACCGTGGCATTCATTATCAACAGAAGGAGG |
| | | CGCCGGGAGCGGCGCGACCTGTTCACTGAATCCTGGGACACCC |
| | | AGAAGGCCCCCAACAACTACAGGTCCCCTATCTCAACCTCCCAA |
| | | CCCACCAACCAGAGCATGGACGATACTCGCGAGGACATCTACGT |
| | | GAACTACCCCACTTTCTCCCGGCGGCCTAAGACCCGCGTGAAGC |
| | | GCGGTCGGAAGAAGCTGCTGTACATCTTCAAGCAGCCCTTCATG |
| | | CGGCCTGTGCAGACCACACAGGAAGAGGATGGCTGCTCCTGCC |
| | | GCTTCCCGGAGGAAGAGGAGGGCGGATGCGAACTGCGCGTGAA |
| | | GTTCAGCCGGAGCGCTGATGCCCCTGCATACCAGCAGGGGCAG |
| | | AACCAGCTCTACAACGAACTGAACCTTGGACGGCGGGAGGAATA |
| | | CGATGTGCTGGATAAGCGAAGAGGCCGCGACCCAGAAATGGGC |
| | | GGGAAGCCCAGACGCAAGAATCCTCAGGAGGGACTGTACAACG |
| | | AGCTCCAGAAAGACAAGATGGCCGAAGCGTACAGCGAGATCGG |
| | | CATGAAGGGGGAACGGAGAAGGGGAAAGGGCCATGACGGATTG |
| | | TACCAGGGCCTGTCGACCGCTACCAAAGACACCTACGACGCCCT |
| | | CCATATGCAAGCACTGCCGCCTAGATGAACGCGT |
| 103 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD226 ICD | GACCGGCGATACTGCGTACTCACAGAAGTTCAAGGGAAAGGCCA |
| | v2/41BB/CD3ζ | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGTCCGGATCC |
| | | GAAGTTCTGGGTGCTGGTGGTGGTCGGGGGAGTGCTCGCCTGC |
| | | TACTCTCTGCTGGTGACCGTGGCATTCATTATCTTCCTGAACAGA |
| | | AGGAGGCGCCGGGAGCGGCGCGACCTGTTCACTGAATCCTGGG |
| | | ACACCCAGAAGGCCCCCAACAACTACAGGTCCCCTATCTCAACC |
| | | TCCCAACCCACCAACCAGAGCATGGACGATACTCGCGAGGACAT |
| | | CTACGTGAACTACCCCACTTTCTCCCGGCGGCCTAAGACCCGCG |
| | | TGAAGCGCGGTCGGAAGAAGCTGCTGTACATCTTCAAGCAGCCC |
| | | TTCATGCGGCCTGTGCAGACCACACAGGAAGAGGATGGCTGCTC |
| | | CTGCCGCTTCCCGGAGGAAGAGGAGGGCGGATGCGAACTGCGC |
| | | GTGAAGTTCAGCCGGAGCGCTGATGCCCCTGCATACCAGCAGG |
| | | GGCAGAACCAGCTCTACAACGAACTGAACCTTGGACGGCGGGA |
| | | GGAATACGATGTGCTGGATAAGCGAAGAGGCCGCGACCCAGAAA |
| | | TGGGCGGGAAGCCCAGACGCAAGAATCCTCAGGAGGGACTGTA |
| | | CAACGAGCTCCAGAAAGACAAGATGGCCGAAGCGTACAGCGAGA |
| | | TCGGCATGAAGGGGGAACGGAGAAGGGGAAAGGGCCATGACGG |
| | | ATTGTACCAGGGCCTGTCGACCGCTACCAAAGACACCTACGACG |
| | | CCCTCCATATGCAAGCACTGCCGCCTAGATGAACGCGT |
| 104 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD226 ICD | GACCGGCGATACTGCGTACTCACAGAAGTTCAAGGGAAAGGCCA |
| | v1/CD3ζ | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGCCCGGATCC |
| | | GAAGTTTTGGGTCTTGGTGGTGGTGGGAGGCGTCCTCGCCTGCT |
| | | ACAGCTTGCTCGTGACCGTTGCCTTCATCATCAACCGCAGGCGG |
| | | AGAAGGGAACGGCGCGACCTGTTCACTGAGTCATGGGACACCCA |
| | | GAAGGCCCCGAACAACTACCGCTCCCCGATCTCCACCTCCCAAC |
| | | CGACTAATCAAAGCATGGACGACACCAGGGAGGACATCTACGTG |
| | | AACTACCCTACTTTCTCCCGCCGGCCTAAGACTCGGGTGCGCGT |
| | | GAAGTTCAGCCGGAGCGCTGATGCCCCTGCATACCAGCAGGGG |
| | | CAGAACCAGCTCTACAACGAACTGAACCTTGGACGGCGGGAGGA |
| | | ATACGATGTGCTGGATAAGCGAAGAGGCCGCGACCCAGAAATGG |
| | | GCGGGAAGCCCAGACGCAAGAATCCTCAGGAGGGACTGTACAA |
| | | CGAGCTCCAGAAAGACAAGATGGCCGAAGCGTACAGCGAGATC |
| | | GGCATGAAGGGGGAACGGAGAAGGGGAAAGGGCCATGACGGAT |
| | | TGTACCAGGGCCTGTCGACCGCTACCAAAGACACCTACGACGCC |
| | | CTCCATATGCAAGCACTGCCGCCACGCTGATAG |
| 105 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD226 ICD | GACCGGCGATACTGCGTACTCACAGAAGTTCAAGGGAAAGGCCA |
| | v2/CD3ζ | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGCCCGGATCC |
| | | GAAGTTCTGGGTGTTGGTCGTGGTCGGAGGCGTCCTCGCCTGCT |
| | | ACAGCCTGCTCGTGACCGTGGCCTTCATCATCTTCCTGAACCGC |
| | | AGGCGGAGAAGGGAACGGCGCGACCTGTTCACTGAGTCATGGG |
| | | ACACCCAGAAGGCCCCGAACAACTACCGCTCCCCGATCTCCACC |
| | | TCCCAACCGACTAATCAAAGCATGGACGACACCAGGGAGGACAT |
| | | CTACGTGAACTACCCTACTTTCTCCCGCCGGCCTAAGACTCGGG |
| | | TGCGCGTGAAGTTCAGCCGGAGCGCTGATGCCCCTGCATACCAG |
| | | CAGGGGCAGAACCAGCTCTACAACGAACTGAACCTTGGACGGCG |
| | | GGAGGAATACGATGTGCTGGATAAGCGAAGAGGCCGCGACCCA |
| | | GAAATGGGCGGGAAGCCCAGACGCAAGAATCCTCAGGAGGGAC |
| | | TGTACAACGAGCTCCAGAAAGACAAGATGGCCGAAGCGTACAGC |
| | | GAGATCGGCATGAAGGGGGAACGGAGAAGGGGAAAGGGCCATG |
| | | ACGGATTGTACCAGGGCCTGTCGACCGCTACCAAAGACACCTAC |
| | | GACGCCCTCCATATGCAAGCACTGCCGCCACGCTGATAG |
| 106 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD28 ICD/CD3ζ | GACCGGCGATACTGCGTACTCACAGAAGTTCAAGGGAAAGGCCA |
| | | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGCCCGGATCC |
| | | GAAGTTCTGGGTCTTGGTGGTCGTGGGAGGCGTCCTCGCCTGCT |
| | | ACAGCCTCCTCGTGACCGTCGCCTTCATCATCTTCTGGGTGCGC |
| | | TCCAAGCGCTCCAGACTGCTGCACAGCGACTACATGAACATGAC |
| | | CCCAAGACGCCCAGGACCTACTAGGAAGCATTATCAACCTTATG |
| | | CCCCGCCGAGAGACTTCGCGGCGTACCGGTCCCGCGTGAAGTT |
| | | CAGCCGGAGCGCTGATGCCCCTGCATACCAGCAGGGGCAGAAC |
| | | CAGCTCTACAACGAACTGAACCTTGGACGGCGGGAGGAATACGA |
| | | TGTGCTGGATAAGCGAAGAGGCCGCGACCCAGAAATGGGCGGG |
| | | AAGCCCAGACGCAAGAATCCTCAGGAGGGACTGTACAACGAGCT |
| | | CCAGAAAGACAAGATGGCCGAAGCGTACAGCGAGATCGGCATGA |
| | | AGGGGGAACGGAGAAGGGGAAAGGGCCATGACGGATTGTACCA |
| | | GGGCCTGTCGACCGCTACCAAAGACACCTACGACGCCCTCCATA |
| | | TGCAAGCACTGCCGCCACGCTGATAG |
| 107 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD226 ICD v1/ | GACCGGCGATACTGCGTACTCACAGAAGTTCAAGGGAAAGGCCA |
| | CD28 ICD/CD3ζ | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGCCCGGATCC |
| | | GAAGTTTTGGGTGTTGGTGGTCGTGGGAGGCGTCCTCGCGTGCT |
| | | ACAGCCTCCTCGTGACCGTCGCCTTCATCATCAACCGCAGGCGG |
| | | AGAAGGGAACGGCGCGACCTGTTCACTGAGTCATGGGACACCCA |
| | | GAAGGCCCCGAACAACTACCGCTCCCCGATCTCCACCTCCCAAC |
| | | CGACTAATCAAAGCATGGACGACACCAGGGAGGACATCTACGTG |
| | | AACTACCCTACTTTCTCCCGCCGGCCTAAGACCCGCGTGTTCTG |
| | | GGTGCGCTCCAAGCGCTCCAGACTGCTGCACAGCGACTACATGA |
| | | ACATGACCCCAAGACGCCCAGGACCTACTAGGAAGCATTATCAA |
| | | CCTTATGCCCCGCCGAGAGACTTCGCGGCGTACCGGTCCCGCG |
| | | TGAAGTTCAGCCGGAGCGCTGATGCCCCTGCATACCAGCAGGG |
| | | GCAGAACCAGCTCTACAACGAACTGAACCTTGGACGGCGGGAG |
| | | GAATACGATGTGCTGGATAAGCGAAGAGGCCGCGACCCAGAAAT |
| | | GGGCGGGAAGCCCAGACGCAAGAATCCTCAGGAGGGACTGTAC |
| | | AACGAGCTCCAGAAAGACAAGATGGCCGAAGCGTACAGCGAGAT |
| | | CGGCATGAAGGGGGAACGGAGAAGGGGAAAGGGCCATGACGGA |
| | | TTGTACCAGGGCCTGTCGACCGCTACCAAAGACACCTACGACGC |
| | | CCTCCATATGCAAGCACTGCCGCCACGCTGATAG |
| 108 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD226 ICD v2/ | GACCGGCGATACTGCGTACTCACAGAAGTTCAAGGGAAAGGCCA |
| | CD28 ICD/CD3ζ | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGCCCGGATCC |
| | | GAAGTTCTGGGTCTTGGTGGTGGTCGGAGGCGTCCTCGCTTGTT |
| | | ACAGCCTGCTCGTGACCGTTGCCTTCATCATCTTCCTGAACCGCA |
| | | GGCGGAGAAGGGAACGGCGCGACCTGTTCACTGAGTCATGGGA |
| | | CACCCAGAAGGCCCCGAACAACTACCGCTCCCCGATCTCCACCT |
| | | CCCAACCGACTAATCAAAGCATGGACGACACCAGGGAGGACATC |
| | | TACGTGAACTACCCTACTTTCTCCCGCCGGCCTAAGACCCGCGT |
| | | GTTCTGGGTGCGCTCCAAGCGCTCCAGACTGCTGCACAGCGACT |
| | | ACATGAACATGACCCCAAGACGCCCAGGACCTACTAGGAAGCAT |
| | | TATCAACCTTATGCCCCGCCGAGAGACTTCGCGGCGTACCGGTC |
| | | CCGCGTGAAGTTCAGCCGGAGCGCTGATGCCCCTGCATACCAG |
| | | CAGGGGCAGAACCAGCTCTACAACGAACTGAACCTTGGACGGCG |
| | | GGAGGAATACGATGTGCTGGATAAGCGAAGAGGCCGCGACCCA |
| | | GAAATGGGCGGGAAGCCCAGACGCAAGAATCCTCAGGAGGGAC |
| | | TGTACAACGAGCTCCAGAAAGACAAGATGGCCGAAGCGTACAGC |
| | | GAGATCGGCATGAAGGGGGAACGGAGAAGGGGAAAGGGCCATG |
| | | ACGGATTGTACCAGGGCCTGTCGACCGCTACCAAAGACACCTAC |
| | | GACGCCCTCCATATGCAAGCACTGCCGCCACGCTGATAG |
| 109 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD28 ICD/CD226 | GACCGGCGATACTGCGTACTCACAGAAGTTCAAGGGAAAGGCCA |
| | ICD v1/CD3ζ | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGCCCGGATCC |
| | | GAAGTTCTGGGTGTTGGTGGTCGTGGGAGGCGTCCTCGCATGTT |
| | | ACTCCCTGCTGGTGACTGTGGCCTTCATCATTTTTTGGGTGAGGT |
| | | CCAAGCGGTCGCGGCTGCTGCACTCCGACTACATGAACATGACC |
| | | CCCAGAAGACCCGGTCCTACACGGAAGCATTACCAACCCTACGC |
| | | CCCCCCGAGGGACTTCGCCGCCTACCGGTCCAACCGCAGGCGG |
| | | AGAAGGGAACGGCGCGACCTGTTCACTGAGTCATGGGACACCCA |
| | | GAAGGCCCCGAACAACTACCGCTCCCCGATCTCCACCTCCCAAC |
| | | CGACTAATCAAAGCATGGACGACACCAGGGAGGACATCTACGTG |
| | | AACTACCCTACTTTCTCCCGCCGGCCTAAGACCCGCGTGCGCGT |
| | | GAAGTTCAGCCGGAGCGCTGATGCCCCTGCATACCAGCAGGGG |
| | | CAGAACCAGCTCTACAACGAACTGAACCTTGGACGGCGGGAGGA |
| | | ATACGATGTGCTGGATAAGCGAAGAGGCCGCGACCCAGAAATGG |
| | | GCGGGAAGCCCAGACGCAAGAATCCTCAGGAGGGACTGTACAA |
| | | CGAGCTCCAGAAAGACAAGATGGCCGAAGCGTACAGCGAGATC |
| | | GGCATGAAGGGGGAACGGAGAAGGGGAAAGGGCCATGACGGAT |
| | | TGTACCAGGGCCTGTCGACCGCTACCAAAGACACCTACGACGCC |
| | | CTCCATATGCAAGCACTGCCGCCACGCTGATAG |
| 110 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD28 | GACCGGCGATACTGCGTACTCACAGAAGTTCAAGGGAAAGGCCA |
| | ICD/41BB/CD3ζ | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGCCCGGATCC |
| | | GAAGTTCTGGGTGCTGGTGGTGGTCGGGGGAGTGCTCGCCTGC |
| | | TACTCTCTGCTGGTGACCGTGGCCTTCATCATCTTTTGGGTGAGA |
| | | TCCAAGCGGTCTCGCCTGCTGCACAGCGACTACATGAACATGAC |
| | | CCCGCGCAGACCTGGCCCGACTAGGAAGCACTACCAGCCCTAC |
| | | GCCCCCCCCAGGGATTTCGCCGCCTACAGATCCAAGCGCGGTC |
| | | GGAAGAAGCTGCTGTACATCTTCAAGCAGCCCTTCATGCGGCCT |
| | | GTGCAGACCACACAGGAAGAGGATGGCTGCTCCTGCCGCTTCCC |
| | | GGAGGAAGAGGAGGGCGGATGCGAACTGCGCGTGAAGTTCAGC |
| | | CGGAGCGCTGATGCCCCTGCATACCAGCAGGGGCAGAACCAGC |
| | | TCTACAACGAACTGAACCTTGGACGGCGGGAGGAATACGATGTG |
| | | CTGGATAAGCGAAGAGGCCGCGACCCAGAAATGGGCGGGAAGC |
| | | CCAGACGCAAGAATCCTCAGGAGGGACTGTACAACGAGCTCCAG |
| | | AAAGACAAGATGGCCGAAGCGTACAGCGAGATCGGCATGAAGG |
| | | GGGAACGGAGAAGGGGAAAGGGCCATGACGGATTGTACCAGGG |
| | | CCTGTCGACCGCTACCAAAGACACCTACGACGCCCTCCATATGC |
| | | AAGCACTGCCGCCACGCTGATAG |
| 111 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD226 ICD v1/ | GACCGGCGATACTGCGTACTCACAGAAGTTCAAGGGAAAGGCCA |
| | CD28 | CGCTCACTGCGGACAAATCGTCCAGCACCGCGTACATGGAACTC |
| | ICD/41BB/CD3ζ | AGGAGCCTGACTTCCGAGGACAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGCCCGGATCC |
| | | GAAGTTTTGGGTGTTGGTGGTGGTCGGAGGCGTCCTCGCATGCT |
| | | ATAGCCTGCTCGTGACCGTGGCCTTCATCATCAACCGCAGGCGG |
| | | AGAAGGGAACGGCGCGACCTGTTCACTGAGTCATGGGACACCCA |
| | | GAAGGCCCCGAACAACTACCGCTCCCCGATCTCCACCTCCCAAC |
| | | CGACTAATCAAAGCATGGACGACACCAGGGAGGACATCTACGTG |
| | | AACTACCCTACTTTCTCCCGCCGGCCTAAGACCCGCGTGTTCTG |
| | | GGTGCGCTCCAAGCGCTCCAGACTGCTGCACAGCGACTACATGA |
| | | ACATGACCCCAAGACGCCCAGGACCTACTAGGAAGCATTATCAA |
| | | CCTTATGCCCCGCCGAGAGACTTCGCGGCGTATAGGTCCAAGCG |
| | | CGGTCGGAAGAAGCTGCTGTACATCTTCAAGCAGCCCTTCATGC |
| | | GGCCTGTGCAGACCACACAGGAAGAGGATGGCTGCTCCTGCCG |
| | | CTTCCCGGAGGAAGAGGAGGGCGGATGCGAACTGCGCGTGAAG |
| | | TTCAGCCGGAGCGCTGATGCCCCTGCATACCAGCAGGGGCAGA |
| | | ACCAGCTCTACAACGAACTGAACCTTGGACGGCGGGAGGAATAC |
| | | GATGTGCTGGATAAGCGAAGAGGCCGCGACCCAGAAATGGGCG |
| | | GGAAGCCCAGACGCAAGAATCCTCAGGAGGGACTGTACAACGA |
| | | GCTCCAGAAAGACAAGATGGCCGAAGCGTACAGCGAGATCGGC |
| | | ATGAAGGGGGAACGGAGAAGGGGAAAGGGCCATGACGGATTGT |
| | | ACCAGGGCCTGTCGACCGCTACCAAAGACACCTACGACGCCCTC |
| | | CATATGCAAGCACTGCCGCCACGCTGATAG |
| 112 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD226 ICD v2/ | GACCGGCGATACTGCGTACTCACAGAAGTTCAAGGGAAAGGCCA |
| | CD28 | CGCTCACTGCGGACAAATCGTCCAGCACCGCGTACATGGAACTC |
| | ICD/41BB/CD3ζ | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGCCCGGATCC |
| | | GAAGTTCTGGGTCTTGGTGGTGGTCGGAGGCGTCCTCGCTTGCT |
| | | ACAGCCTGCTCGTGACCGTGGCCTTCATCATCTTCCTGAACCGC |
| | | AGGCGGAGAAGGGAACGGCGCGACCTGTTCACTGAGTCATGGG |
| | | ACACCCAGAAGGCCCCGAACAACTACCGCTCCCCGATCTCCACC |
| | | TCCCAACCGACTAATCAAAGCATGGACGACACCAGGGAGGACAT |
| | | CTACGTGAACTACCCTACTTTCTCCCGCCGGCCTAAGACCCGCG |
| | | TGTTCTGGGTGCGCTCCAAGCGCTCCAGACTGCTGCACAGCGAC |
| | | TACATGAACATGACCCCAAGACGCCCAGGACCTACTAGGAAGCA |
| | | TTATCAACCTTATGCCCCGCCGAGAGACTTCGCGGCGTATAGGT |
| | | CCAAGCGCGGTCGGAAGAAGCTGCTGTACATCTTCAAGCAGCCC |
| | | TTCATGCGGCCTGTGCAGACCACACAGGAAGAGGATGGCTGCTC |
| | | CTGCCGCTTCCCGGAGGAAGAGGAGGGCGGATGCGAACTGCGC |
| | | GTGAAGTTCAGCCGGAGCGCTGATGCCCCTGCATACCAGCAGG |
| | | GGCAGAACCAGCTCTACAACGAACTGAACCTTGGACGGCGGGA |
| | | GGAATACGATGTGCTGGATAAGCGAAGAGGCCGCGACCCAGAAA |
| | | TGGGCGGGAAGCCCAGACGCAAGAATCCTCAGGAGGGACTGTA |
| | | CAACGAGCTCCAGAAAGACAAGATGGCCGAAGCGTACAGCGAGA |
| | | TCGGCATGAAGGGGGAACGGAGAAGGGGAAAGGGCCATGACGG |
| | | ATTGTACCAGGGCCTGTCGACCGCTACCAAAGACACCTACGACG |
| | | CCCTCCATATGCAAGCACTGCCGCCACGCTGATAG |
| 113 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD28 TMD/ | CCCCCGTGCACGGTCTGAAGTGGATTGGGGCCCTGGATCCCAA |
| | CD28 ICD/CD226 | GACCGGCGATACTGCGTACTCACAGAAGTTCAAGGGAAAGGCCA |
| | ICD v1/41BB/CD3ζ | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGTCCGGATCC |
| | | GAAGTTCTGGGTGCTGGTGGTGGTCGGGGGAGTGCTCGCCTGC |
| | | TACTCTCTGCTGGTGACCGTGGCATTCATTATCTTCTGGGTCCGC |
| | | TCCAAACGGTCCCGGCTGCTGCACTCCGACTACATGAACATGAC |
| | | CCCGAGACGGCCAGGACCGACTCGCAAGCACTACCAGCCGTAC |
| | | GCCCCACCGAGAGACTTCGCCGCATACCGCTCAAACAGAAGGAG |
| | | GCGCCGGGAGCGGCGCGACCTGTTCACTGAATCCTGGGACACC |
| | | CAGAAGGCCCCCAACAACTACAGGTCCCCTATCTCAACCTCCCA |
| | | ACCCACCAACCAGAGCATGGACGATACTCGCGAGGACATCTACG |
| | | TGAACTACCCCACTTTCTCCCGGCGGCCTAAGACCCGCGTGAAG |
| | | CGCGGTCGGAAGAAGCTGCTGTACATCTTCAAGCAGCCCTTCAT |
| | | GCGGCCTGTGCAGACCACACAGGAAGAGGATGGCTGCTCCTGC |
| | | CGCTTCCCGGAGGAAGAGGAGGGCGGATGCGAACTGCGCGTGA |
| | | AGTTCAGCCGGAGCGCTGATGCCCCTGCATACCAGCAGGGGCA |
| | | GAACCAGCTCTACAACGAACTGAACCTTGGACGGCGGGAGGAAT |
| | | ACGATGTGCTGGATAAGCGAAGAGGCCGCGACCCAGAAATGGG |
| | | CGGGAAGCCCAGACGCAAGAATCCTCAGGAGGGACTGTACAAC |
| | | GAGCTCCAGAAAGACAAGATGGCCGAAGCGTACAGCGAGATCG |
| | | GCATGAAGGGGGAACGGAGAAGGGGAAAGGGCCATGACGGATT |
| | | GTACCAGGGCCTGTCGACCGCTACCAAAGACACCTACGACGCCC |
| | | TCCATATGCAAGCACTGCCGCCTAGATGATAG |
| 114 | (nucleotide) hIgG | ATGGACTGGATCTGGAGGATTTTGTTCCTTGTGGGAGCTGCCAC |
| | heavy chain signal | CGGTGCACATTCGCAAGTGCAGTTGCAGCAGTCGGGAGCCGAA |
| | sequence/scFV | CTGGTCCGGCCTGGAGCTTCCGTGAAGCTTAGCTGCAAGGCCTC |
| | GC33/hIgG1 | CGGCTACACCTTTACCGACTACGAGATGCACTGGGTCAAGCAGA |
| | hinge/CD226 | CCCCCGTGCACGGTCTGAAGTGGATTGGGGCCCTGGATCCCAA |
| | TMD/CD226 ICD | GACCGGCGATACTGCGTACTCACAGAAGTTCAAGGGAAAGGCCA |
| | v1/41BB/CD3ζ | CGCTCACTGCGGACAAATCGTCCAGCACCGCGTACATGGAACTC |
| | | AGGAGCCTGACTTCCGAGGATAGCGCAGTGTACTACTGCACCCG |
| | | CTTTTACTCCTACACTTACTGGGGACAGGGCACCTTGGTGACTGT |
| | | GTCAGCCGGTGGAGGCGGATCAGGGGGTGGAGGATCCGGGGG |
| | | AGGAGGATCCGATGTGGTCATGACCCAGACCCCACTGTCCCTTC |
| | | CCGTGTCCCTGGGTGACCAAGCCTCGATCAGCTGCAGATCCTCC |
| | | CAGTCACTGGTCCACAGCAACGGCAACACCTATCTGCATTGGTA |
| | | CCTCCAGAAGCCGGGACAATCCCCCAAGCTCCTGATCTACAAGG |
| | | TGTCCAACCGGTTCAGCGGAGTGCCGGATCGATTCTCAGGGTCG |
| | | GGTTCGGGAACCGACTTCACCCTTAAGATTTCCCGGGTGGAAGC |
| | | CGAGGATCTCGGAGTGTACTTCTGCTCCCAAAATACCCACGTGC |
| | | CGCCTACATTCGGATCGGGAACTAAGCTGGAGATCAAGGAGCCC |
| | | AAAAGCTGCGACAAGACCCACACTTGCCCACCTTGTCCGGATCC |
| | | GAAGGGGGGAACTGTGCTCCTCCTGCTGTTCGTGATTTCGATCA |
| | | CGACCATCATTGTGATCTTCCTGAACCGCCGGCGAAGACGCGAG |
| | | CGGCGCGACCTGTTCACTGAATCCTGGGACACCCAGAAGGCCC |
| | | CCAACAACTACAGGTCCCCTATCTCAACCTCCCAACCCACCAACC |
| | | AGAGCATGGACGATACTCGCGAGGACATCTACGTGAACTACCCC |
| | | ACTTTCTCCCGGCGGCCTAAGACCCGCGTGAAGCGCGGTCGGA |
| | | AGAAGCTGCTGTACATCTTCAAGCAGCCCTTCATGCGGCCTGTG |
| | | CAGACCACACAGGAAGAGGATGGCTGCTCCTGCCGCTTCCCGG |
| | | AGGAAGAGGAGGGCGGATGCGAACTGCGCGTGAAGTTCAGCCG |
| | | GAGCGCTGATGCCCCTGCATACCAGCAGGGGCAGAACCAGCTC |
| | | TACAACGAACTGAACCTTGGACGGCGGGAGGAATACGATGTGCT |
| | | GGATAAGCGAAGAGGCCGCGACCCAGAAATGGGCGGGAAGCCC |
| | | AGACGCAAGAATCCTCAGGAGGGACTGTACAACGAGCTCCAGAA |
| | | AGACAAGATGGCCGAAGCGTACAGCGAGATCGGCATGAAGGGG |
| | | GAACGGAGAAGGGGAAAGGGCCATGACGGATTGTACCAGGGCC |
| | | TGTCGACCGCTACCAAAGACACCTACGACGCCCTCCATATGCAA |
| | | GCACTGCCGCCTAGATGAACGCGT |
| 115 | (nucleotide) hIgG | ATGGACTGGATCTGGAGAATTTTGTTCCTTGTCGGTGCTGCCACT |
| | heavy chain signal | GGAGCCCACTCGCAAGTGCAGCTCGTGCAGTCTGGAGCAGAAG |
| | sequence/scFV 3- | TCAAGAAGCCTGGGTCCTCGGTCAAAGTGTCCTGCAAAGCCTCC |
| | 17I/hIgG1 | GGGGGCACTTTCAGCTCGTACGCAATCTCCTGGGTCCGCCAAGC |
| | hinge/CD226 | GCCCGGACAGGGTCTGGAGTGGATGGGCGGGATTATCCCCATTT |
| | TMD/CD226 ICD | TCGGCACCGCCAACTATGCTCAAAAGTTCCAGGGCCGCGTGACT |
| | v1 | ATTACCGCGGACGAGTCAACCTCCACTGCGTACATGGAACTGAG |
| | | CTCACTTCGGTCCGAGGACACCGCCGTCTATTACTGTGCGCGGG |
| | | GACTCCTGTGGAACTACTGGGGACAAGGCACCCTCGTGACCGTG |
| | | TCCTCGAAGCTGTCCGGGTCGGCCAGCGCCCCCAAGTTGGAAG |
| | | AGGGCGAATTCTCCGAGGCCCGGGTGGAAATCGTGATGACCCA |
| | | GTCCCCGGCCACTCTCTCCGTGTCCCCGGGAGAAAGGGCGACC |
| | | CTGTCATGCCGGGCCTCGCAATCCGTGTCATCCAACCTGGCCTG |
| | | GTACCAGCAGAAGCCAGGACAGGCGCCCCGCCTGATTATCTACG |
| | | GCGCCAGCACCACCGCGAGCGGAATTCCGGCCCGGTTTTCCGC |
| | | CTCCGGTTCGGGAACTGACTTCACGCTGACTATCAGCAGCCTGC |
| | | AATCGGAGGACTTCGCCGTCTACTACTGCCAGCAGTATAACAACT |
| | | GGCCCCCGGCTTACACGTTTGGCCAGGGAACTAAGCTGGAGATC |
| | | AAGGAGCCCAAAAGCTGCGACAAGACCCACACTTGCCCACCTTG |
| | | TCCGGACCCGAAGGGGGGAACCGTGCTCCTGCTGCTGTTCGTG |
| | | ATCTCCATCACCACAATCATCGTGATCTTCCTGAACCGCCGGCGA |
| | | AGACGCGAAAGACGCGATCTGTTCACCGAGTCATGGGACACCCA |
| | | GAAGGCCCCTAACAACTACAGAAGCCCGATCAGCACCAGCCAGC |
| | | CTACTAATCAGTCGATGGATGATACCCGCGAGGACATCTACGTG |
| | | AATTACCCAACCTTCTCCCGGCGGCCGAAAACCCGCGTGTGAAC |
| | | GCGT |
| 116 | (nucleotide) hIgG | ATGGACTGGATCTGGAGAATTTTGTTCCTTGTCGGTGCTGCCACT |
| | heavy chain signal | GGAGCCCACTCGCAAGTGCAGCTCGTGCAGTCTGGAGCAGAAG |
| | sequence/scFV 3- | TCAAGAAGCCTGGGTCCTCGGTCAAAGTGTCCTGCAAAGCCTCC |
| | 17I/hIgG1 | GGGGGCACTTTCAGCTCGTACGCAATCTCCTGGGTCCGCCAAGC |
| | hinge/CD226 TMD/ | GCCCGGACAGGGTCTGGAGTGGATGGGCGGGATTATCCCCATTT |
| | CD3ζ | TCGGCACCGCCAACTATGCTCAAAAGTTCCAGGGCCGCGTGACT |
| | | ATTACCGCGGACGAGTCAACCTCCACTGCGTACATGGAACTGAG |
| | | CTCACTTCGGTCCGAGGACACCGCCGTCTATTACTGTGCGCGGG |
| | | GACTCCTGTGGAACTACTGGGGACAAGGCACCCTCGTGACCGTG |
| | | TCCTCGAAGCTGTCCGGGTCGGCCAGCGCCCCCAAGTTGGAAG |
| | | AGGGCGAATTCTCCGAGGCCCGGGTGGAAATCGTGATGACCCA |
| | | GTCCCCGGCCACTCTCTCCGTGTCCCCGGGAGAAAGGGCGACC |
| | | CTGTCATGCCGGGCCTCGCAATCCGTGTCATCCAACCTGGCCTG |
| | | GTACCAGCAGAAGCCAGGACAGGCGCCCCGCCTGATTATCTACG |
| | | GCGCCAGCACCACCGCGAGCGGAATTCCGGCCCGGTTTTCCGC |
| | | CTCCGGTTCGGGAACTGACTTCACGCTGACTATCAGCAGCCTGC |
| | | AATCGGAGGACTTCGCCGTCTACTACTGCCAGCAGTATAACAACT |
| | | GGCCCCCGGCTTACACGTTTGGCCAGGGAACTAAGCTGGAGATC |
| | | AAGGAGCCCAAAAGCTGCGACAAGACCCACACTTGCCCACCTTG |
| | | TCCGGACCCGAAGGGGGGAACCGTGCTCCTGCTGCTGTTCGTG |
| | | ATCTCCATCACCACAATCATCGTGATCTTCCTGCGCGTGAAGTTC |
| | | AGCCGGAGCGCTGATGCCCCTGCATACCAGCAGGGGCAGAACC |
| | | AGCTCTACAACGAACTGAACCTTGGACGGCGGGAGGAATACGAT |
| | | GTGCTGGATAAGCGAAGAGGCCGCGACCCAGAAATGGGCGGGA |
| | | AGCCCAGACGCAAGAATCCTCAGGAGGGACTGTACAACGAGCTC |
| | | CAGAAAGACAAGATGGCCGAAGCGTACAGCGAGATCGGCATGAA |
| | | GGGGGAACGGAGAAGGGGAAAGGGCCATGACGGATTGTACCAG |
| | | GGCCTGTCGACCGCTACCAAAGACACCTACGACGCCCTCCATAT |
| | | GCAAGCACTGCCGCCACGCTGAACGCGT |
| 117 | (nucleotide) hIgG | ATGGACTGGATCTGGAGAATTTTGTTCCTTGTCGGTGCTGCCACT |
| | heavy chain signal | GGAGCCCACTCGCAAGTGCAGCTCGTGCAGTCTGGAGCAGAAG |
| | sequence/scFV 3- | TCAAGAAGCCTGGGTCCTCGGTCAAAGTGTCCTGCAAAGCCTCC |
| | 17I/hIgG1 | GGGGGCACTTTCAGCTCGTACGCAATCTCCTGGGTCCGCCAAGC |
| | hinge/CD226 | GCCCGGACAGGGTCTGGAGTGGATGGGCGGGATTATCCCCATTT |
| | TMD/CD226 ICD | TCGGCACCGCCAACTATGCTCAAAAGTTCCAGGGCCGCGTGACT |
| | v1/CD3ζ | ATTACCGCGGACGAGTCAACCTCCACTGCGTACATGGAACTGAG |
| | | CTCACTTCGGTCCGAGGACACCGCCGTCTATTACTGTGCGCGGG |
| | | GACTCCTGTGGAACTACTGGGGACAAGGCACCCTCGTGACCGTG |
| | | TCCTCGAAGCTGTCCGGGTCGGCCAGCGCCCCCAAGTTGGAAG |
| | | AGGGCGAATTCTCCGAGGCCCGGGTGGAAATCGTGATGACCCA |
| | | GTCCCCGGCCACTCTCTCCGTGTCCCCGGGAGAAAGGGCGACC |
| | | CTGTCATGCCGGGCCTCGCAATCCGTGTCATCCAACCTGGCCTG |
| | | GTACCAGCAGAAGCCAGGACAGGCGCCCCGCCTGATTATCTACG |
| | | GCGCCAGCACCACCGCGAGCGGAATTCCGGCCCGGTTTTCCGC |
| | | CTCCGGTTCGGGAACTGACTTCACGCTGACTATCAGCAGCCTGC |
| | | AATCGGAGGACTTCGCCGTCTACTACTGCCAGCAGTATAACAACT |
| | | GGCCCCCGGCTTACACGTTTGGCCAGGGAACTAAGCTGGAGATC |
| | | AAGGAGCCCAAAAGCTGCGACAAGACCCACACTTGCCCACCTTG |
| | | TCCGGACCCGAAGGGGGGAACCGTGCTCCTGCTGCTGTTCGTG |
| | | ATCTCCATCACCACAATCATCGTGATCTTCCTGAACCGCCGGCGA |
| | | AGACGCGAAAGACGCGATCTGTTCACCGAGTCATGGGACACCCA |
| | | GAAGGCCCCTAACAACTACAGAAGCCCGATCAGCACCAGCCAGC |
| | | CTACTAATCAGTCGATGGATGATACCCGCGAGGACATCTACGTG |
| | | AATTACCCAACCTTCTCCCGGCGGCCGAAAACCCGCGTGCGCGT |
| | | GAAGTTCAGCCGGAGCGCTGATGCCCCTGCATACCAGCAGGGG |
| | | CAGAACCAGCTCTACAACGAACTGAACCTTGGACGGCGGGAGGA |
| | | ATACGATGTGCTGGATAAGCGAAGAGGCCGCGACCCAGAAATGG |
| | | GCGGGAAGCCCAGACGCAAGAATCCTCAGGAGGGACTGTACAA |
| | | CGAGCTCCAGAAAGACAAGATGGCCGAAGCGTACAGCGAGATC |
| | | GGCATGAAGGGGGAACGGAGAAGGGGAAAGGGCCATGACGGAT |
| | | TGTACCAGGGCCTGTCGACCGCTACCAAAGACACCTACGACGCC |
| | | CTCCATATGCAAGCACTGCCGCCACGCTGAACGCGT |
| 118 | hIgG heavy chain | MDWIWRILFLVGAATGAHSQVQLQQSGAELVRPGASVKLSCKASGY |
| | signal | TFTDYEMHWVKQTPVHGLKWIGALDPKTGDTAYSQKFKGKATLTAD |
| | sequence/scFV | KSSSTAYMELRSLTSEDSAVYYCTRFYSYTYWGQGTLVTVSAGGG |
| | GC33/hIgG1 | GSGGGGSGGGGSDVVMTQTPLSLPVSLGDQASISCRSSQSLVHSN |
| | hinge/CD28 | GNTYLHWYLQKPGQSPKLLIYKVSNRFSGVPDRFSGSGSGTDFTLKI |
| | TMD/41BB/CD3ζ/ | SRVEAEDLGVYFCSQNTHVPPTFGSGTKLEIKEPKSCDKTHTCPPC |
| | CD226 ICD v1 | PDPKFWVLVVVGGVLACYSLLVTVAFIIKRGRKKLLYIFKQPFMRPVQ |
| | | TTQEEDGCSCRFPEEEEGGCELRVKFSRSADAPAYQQGQNQLYNE |
| | | LNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| | | NRRRRRERRDLFTESWDTQKAPNNYRSPISTSQPTNQSMDDTREDI |
| | | YVNYPTFSRRPKTRV |
| 119 | hIgG heavy chain | MDWIWRILFLVGAATGAHSQVQLQQSGAELVRPGASVKLSCKASGY |
| | signal | TFTDYEMHWVKQTPVHGLKWIGALDPKTGDTAYSQKFKGKATLTAD |
| | sequence/scFV | KSSSTAYMELRSLTSEDSAVYYCTRFYSYTYWGQGTLVTVSAGGG |
| | GC33/hIgG1 | GSGGGGSGGGGSDVVMTQTPLSLPVSLGDQASISCRSSQSLVHSN |
| | hinge/CD28 | GNTYLHWYLQKPGQSPKLLIYKVSNRFSGVPDRFSGSGSGTDFTLKI |
| | TMD/41BB/CD226 | SRVEAEDLGVYFCSQNTHVPPTFGSGTKLEIKEPKSCDKTHTCPPC |
| | ICD v1/CD3ζ | PDPKFWVLVVVGGVLACYSLLVTVAFIIKRGRKKLLYIFKQPFMRPVQ |
| | | TTQEEDGCSCRFPEEEEGGCELNRRRRRERRDLFTESWDTQKAPN |
| | | NYRSPISTSQPTNQSMDDTREDIYVNYPTFSRRPKTRVRVKFSRSA |
| | | DAPAYQQGQNQLYNELNLGRREEYDVLDKRRGRDPEMGGKPRRK |
| | | NPQEGLYNELQKDKMAEAYSEIGMKGERRRGKGHDGLYQGLSTAT |
| | | KDTYDALHMQALPPR |
| 120 | hIgG heavy chain | MDWIWRILFLVGAATGAHSQVQLQQSGAELVRPGASVKLSCKASGY |
| | signal | TFTDYEMHWVKQTPVHGLKWIGALDPKTGDTAYSQKFKGKATLTAD |
| | sequence/scFV | KSSSTAYMELRSLTSEDSAVYYCTRFYSYTYWGQGTLVTVSAGGG |
| | GC33/hIgG1 | GSGGGGSGGGGSDVVMTQTPLSLPVSLGDQASISCRSSQSLVHSN |
| | hinge/CD226 | GNTYLHWYLQKPGQSPKLLIYKVSNRFSGVPDRFSGSGSGTDFTLKI |
| | TMD/CD226 ICD | SRVEAEDLGVYFCSQNTHVPPTFGSGTKLEIKEPKSCDKTHTCPPC |
| | v1 | PDPKGGTVLLLLFVISITTIIVIFLNRRRRRERRDLFTESWDTQKAPNN |
| | | YRSPISTSQPTNQSMDDTREDIYVNYPTFSRRPKTRV |
| 121 | scFV GC33/hIgG1 | QVQLQQSGAELVRPGASVKLSCKASGYTFTDYEMHWVKQTPVHGL |
| | hinge/CD28 | KWIGALDPKTGDTAYSQKFKGKATLTADKSSSTAYMELRSLTSEDS |
| | TMD/41BB/CD3ζ/ | AVYYCTRFYSYTYWGQGTLVTVSAGGGGSGGGGSGGGGSDVVMT |
| | CD226 ICD v1 | QTPLSLPVSLGDQASISCRSSQSLVHSNGNTYLHWYLQKPGQSPKL |
| | | LIYKVSNRFSGVPDRFSGSGSGTDFTLKISRVEAEDLGVYFCSQNTH |
| | | VPPTFGSGTKLEIKEPKSCDKTHTCPPCPDPKFWVLVVVGGVLACY |
| | | SLLVTVAFIIKRGRKKLLYIFKQPFMRPVQTTQEEDGCSCRFPEEEEG |
| | | GCELRVKFSRSADAPAYQQGQNQLYNELNLGRREEYDVLDKRRGR |
| | | DPEMGGKPRRKNPQEGLYNELQKDKMAEAYSEIGMKGERRRGKG |
| | | HDGLYQGLSTATKDTYDALHMQALPPRNRRRRRERRDLFTESWDT |
| | | QKAPNNYRSPISTSQPTNQSMDDTREDIYVNYPTFSRRPKTRV |
| 122 | scFV GC33/hIgG1 | QVQLQQSGAELVRPGASVKLSCKASGYTFTDYEMHWVKQTPVHGL |
| | hinge/CD28 | KWIGALDPKTGDTAYSQKFKGKATLTADKSSSTAYMELRSLTSEDS |
| | TMD/41BB/CD226 | AVYYCTRFYSYTYWGQGTLVTVSAGGGGSGGGGSGGGGSDVVMT |
| | ICD v1/CD3ζ | QTPLSLPVSLGDQASISCRSSQSLVHSNGNTYLHWYLQKPGQSPKL |
| | | LIYKVSNRFSGVPDRFSGSGSGTDFTLKISRVEAEDLGVYFCSQNTH |
| | | VPPTFGSGTKLEIKEPKSCDKTHTCPPCPDPKFWVLVVVGGVLACY |
| | | SLLVTVAFIIKRGRKKLLYIFKQPFMRPVQTTQEEDGCSCRFPEEEEG |
| | | GCELNRRRRRERRDLFTESWDTQKAPNNYRSPISTSQPTNQSMDD |
| | | TREDIYVNYPTFSRRPKTRVRVKFSRSADAPAYQQGQNQLYNELNL |
| | | GRREEYDVLDKRRGRDPEMGGKPRRKNPQEGLYNELQKDKMAEA |
| | | YSEIGMKGERRRGKGHDGLYQGLSTATKDTYDALHMQALPPR |
| 123 | scFV GC33/hIgG1 | QVQLQQSGAELVRPGASVKLSCKASGYTFTDYEMHWVKQTPVHGL |
| | hinge/CD226 | KWIGALDPKTGDTAYSQKFKGKATLTADKSSSTAYMELRSLTSEDS |
| | TMD/CD226 ICD | AVYYCTRFYSYTYWGQGTLVTVSAGGGGSGGGGSGGGGSDVVMT |
| | v1 | QTPLSLPVSLGDQASISCRSSQSLVHSNGNTYLHWYLQKPGQSPKL |
| | | LIYKVSNRFSGVPDRFSGSGSGTDFTLKISRVEAEDLGVYFCSQNTH |
| | | VPPTFGSGTKLEIKEPKSCDKTHTCPPCPDPKGGTVLLLLFVISITTIIV |
| | | IFLNRRRRRERRDLFTESWDTQKAPNNYRSPISTSQPTNQSMDDTR |
| | | EDIYVNYPTFSRRPKTRV |
| 124 | (nucleotide) hIgG | ATGGATTGGATTTGGCGCATTCTGTTTCTGGTGGGCGCGGCGAC |
| | heavy chain signal | CGGCGCGCATAGCCAGGTGCAGCTGCAGCAGAGCGGCGCGGAA |
| | sequence/scFV | CTGGTGCGCCCGGGCGCGAGCGTGAAACTGAGCTGCAAAGCGA |
| | GC33/hIgG1 | GCGGCTATACCTTTACCGATTATGAAATGCATTGGGTGAAACAGA |
| | hinge/CD28 | CCCCGGTGCATGGCCTGAAATGGATTGGCGCGCTGGATCCGAAA |
| | TMD/41BB/CD3ζ/ | ACCGGCGATACCGCGTATAGCCAGAAATTTAAAGGCAAAGCGAC |
| | CD226 ICD v1 | CCTGACCGCGGATAAAAGCAGCAGCACCGCGTATATGGAACTGC |
| | | GCAGCCTGACCAGCGAAGATAGCGCGGTGTATTATTGCACCCGC |
| | | TTTTATAGCTATACCTATTGGGGCCAGGGCACCCTGGTGACCGT |
| | | GAGCGCGGGCGGCGGCGGCAGCGGCGGCGGCGGCAGCGGCG |
| | | GCGGCGGCAGCGATGTGGTGATGACCCAGACCCCGCTGAGCCT |
| | | GCCGGTGAGCCTGGGCGATCAGGCGAGCATTAGCTGCCGCAGC |
| | | AGCCAGAGCCTGGTGCATAGCAACGGCAACACCTATCTGCATTG |
| | | GTATCTGCAGAAACCGGGCCAGAGCCCGAAACTGCTGATTTATA |
| | | AAGTGAGCAACCGCTTTAGCGGCGTGCCGGATCGCTTTAGCGGC |
| | | AGCGGCAGCGGCACCGATTTTACCCTGAAAATTAGCCGCGTGGA |
| | | AGCGGAAGATCTGGGCGTGTATTTTTGCAGCCAGAACACCCATG |
| | | TGCCGCCGACCTTTGGCAGCGGCACCAAACTGGAAATTAAAGAA |
| | | CCGAAAAGCTGCGATAAAACCCATACCTGCCCGCCGTGCCCGGA |
| | | TCCGAAATTTTGGGTGCTGGTGGTGGTGGGCGGCGTGCTGGCG |
| | | TGCTATAGCCTGCTGGTGACCGTGGCGTTTATTATTAAACGCGGC |
| | | CGCAAAAAACTGCTGTATATTTTTAAACAGCCGTTTATGCGCCCG |
| | | GTGCAGACCACCCAGGAAGAAGATGGCTGCAGCTGCCGCTTTCC |
| | | GGAAGAAGAAGAAGGCGGCTGCGAACTGCGCGTGAAATTTAGCC |
| | | GCAGCGCGGATGCGCCGGCGTATCAGCAGGGCCAGAACCAGCT |
| | | GTATAACGAACTGAACCTGGGCCGCCGCGAAGAATATGATGTGC |
| | | TGGATAAACGCCGCGGCCGCGATCCGGAAATGGGCGGCAAACC |
| | | GCGCCGCAAAAACCCGCAGGAAGGCCTGTATAACGAACTGCAGA |
| | | AAGATAAAATGGCGGAAGCGTATAGCGAAATTGGCATGAAAGGC |
| | | GAACGCCGCCGCGGCAAAGGCCATGATGGCCTGTATCAGGGCC |
| | | TGAGCACCGCGACCAAAGATACCTATGATGCGCTGCATATGCAG |
| | | GCGCTGCCGCCGCGCAACCGCCGCCGCCGCCGCGAACGCCGC |
| | | GATCTGTTTACCGAAAGCTGGGATACCCAGAAAGCGCCGAACAA |
| | | CTATCGCAGCCCGATTAGCACCAGCCAGCCGACCAACCAGAGCA |
| | | TGGATGATACCCGCGAAGATATTTATGTGAACTATCCGACCTTTA |
| | | GCCGCCGCCCGAAAACCCGCGTG |
| 125 | (nucleotide) hIgG | ATGGATTGGATTTGGCGCATTCTGTTTCTGGTGGGCGCGGCGAC |
| | heavy chain signal | CGGCGCGCATAGCCAGGTGCAGCTGCAGCAGAGCGGCGCGGAA |
| | sequence/scFV | CTGGTGCGCCCGGGCGCGAGCGTGAAACTGAGCTGCAAAGCGA |
| | GC33/hIgG1 | GCGGCTATACCTTTACCGATTATGAAATGCATTGGGTGAAACAGA |
| | hinge/CD28 | CCCCGGTGCATGGCCTGAAATGGATTGGCGCGCTGGATCCGAAA |
| | TMD/41BB/CD226 | ACCGGCGATACCGCGTATAGCCAGAAATTTAAAGGCAAAGCGAC |
| | ICD v1/CD3ζ | CCTGACCGCGGATAAAAGCAGCAGCACCGCGTATATGGAACTGC |
| | | GCAGCCTGACCAGCGAAGATAGCGCGGTGTATTATTGCACCCGC |
| | | TTTTATAGCTATACCTATTGGGGCCAGGGCACCCTGGTGACCGT |
| | | GAGCGCGGGCGGCGGCGGCAGCGGCGGCGGCGGCAGCGGCG |
| | | GCGGCGGCAGCGATGTGGTGATGACCCAGACCCCGCTGAGCCT |
| | | GCCGGTGAGCCTGGGCGATCAGGCGAGCATTAGCTGCCGCAGC |
| | | AGCCAGAGCCTGGTGCATAGCAACGGCAACACCTATCTGCATTG |
| | | GTATCTGCAGAAACCGGGCCAGAGCCCGAAACTGCTGATTTATA |
| | | AAGTGAGCAACCGCTTTAGCGGCGTGCCGGATCGCTTTAGCGGC |
| | | AGCGGCAGCGGCACCGATTTTACCCTGAAAATTAGCCGCGTGGA |
| | | AGCGGAAGATCTGGGCGTGTATTTTTGCAGCCAGAACACCCATG |
| | | TGCCGCCGACCTTTGGCAGCGGCACCAAACTGGAAATTAAAGAA |
| | | CCGAAAAGCTGCGATAAAACCCATACCTGCCCGCCGTGCCCGGA |
| | | TCCGAAATTTTGGGTGCTGGTGGTGGTGGGCGGCGTGCTGGCG |
| | | TGCTATAGCCTGCTGGTGACCGTGGCGTTTATTATTAAACGCGGC |
| | | CGCAAAAAACTGCTGTATATTTTTAAACAGCCGTTTATGCGCCCG |
| | | GTGCAGACCACCCAGGAAGAAGATGGCTGCAGCTGCCGCTTTCC |
| | | GGAAGAAGAAGAAGGCGGCTGCGAACTGAACCGCCGCCGCCGC |
| | | CGCGAACGCCGCGATCTGTTTACCGAAAGCTGGGATACCCAGAA |
| | | AGCGCCGAACAACTATCGCAGCCCGATTAGCACCAGCCAGCCGA |
| | | CCAACCAGAGCATGGATGATACCCGCGAAGATATTTATGTGAACT |
| | | ATCCGACCTTTAGCCGCCGCCCGAAAACCCGCGTGCGCGTGAAA |
| | | TTTAGCCGCAGCGCGGATGCGCCGGCGTATCAGCAGGGCCAGA |
| | | ACCAGCTGTATAACGAACTGAACCTGGGCCGCCGCGAAGAATAT |
| | | GATGTGCTGGATAAACGCCGCGGCCGCGATCCGGAAATGGGCG |
| | | GCAAACCGCGCCGCAAAAACCCGCAGGAAGGCCTGTATAACGAA |
| | | CTGCAGAAAGATAAAATGGCGGAAGCGTATAGCGAAATTGGCAT |
| | | GAAAGGCGAACGCCGCCGCGGCAAAGGCCATGATGGCCTGTAT |
| | | CAGGGCCTGAGCACCGCGACCAAAGATACCTATGATGCGCTGCA |
| | | TATGCAGGCGCTGCCGCCGCGC |
| 126 | (nucleotide) hIgG | ATGGATTGGATTTGGCGCATTCTGTTTCTGGTGGGCGCGGCGAC |
| | heavy chain signal | CGGCGCGCATAGCCAGGTGCAGCTGCAGCAGAGCGGCGCGGAA |
| | sequence/scFV | CTGGTGCGCCCGGGCGCGAGCGTGAAACTGAGCTGCAAAGCGA |
| | GC33/hIgG1 | GCGGCTATACCTTTACCGATTATGAAATGCATTGGGTGAAACAGA |
| | hinge/CD226 | CCCCGGTGCATGGCCTGAAATGGATTGGCGCGCTGGATCCGAAA |
| | TMD/CD226 ICD | ACCGGCGATACCGCGTATAGCCAGAAATTTAAAGGCAAAGCGAC |
| | v1 | CCTGACCGCGGATAAAAGCAGCAGCACCGCGTATATGGAACTGC |
| | | GCAGCCTGACCAGCGAAGATAGCGCGGTGTATTATTGCACCCGC |
| | | TTTTATAGCTATACCTATTGGGGCCAGGGCACCCTGGTGACCGT |
| | | GAGCGCGGGCGGCGGCGGCAGCGGCGGCGGCGGCAGCGGCG |
| | | GCGGCGGCAGCGATGTGGTGATGACCCAGACCCCGCTGAGCCT |
| | | GCCGGTGAGCCTGGGCGATCAGGCGAGCATTAGCTGCCGCAGC |
| | | AGCCAGAGCCTGGTGCATAGCAACGGCAACACCTATCTGCATTG |
| | | GTATCTGCAGAAACCGGGCCAGAGCCCGAAACTGCTGATTTATA |
| | | AAGTGAGCAACCGCTTTAGCGGCGTGCCGGATCGCTTTAGCGGC |
| | | AGCGGCAGCGGCACCGATTTTACCCTGAAAATTAGCCGCGTGGA |
| | | AGCGGAAGATCTGGGCGTGTATTTTTGCAGCCAGAACACCCATG |
| | | TGCCGCCGACCTTTGGCAGCGGCACCAAACTGGAAATTAAAGAA |
| | | CCGAAAAGCTGCGATAAAACCCATACCTGCCCGCCGTGCCCGGA |
| | | TCCGAAAGGCGGCACCGTGCTGCTGCTGCTGTTTGTGATTAGCA |
| | | TTACCACCATTATTGTGATTTTTCTGAACCGCCGCCGCCGCCGCG |
| | | AACGCCGCGATCTGTTTACCGAAAGCTGGGATACCCAGAAAGCG |
| | | CCGAACAACTATCGCAGCCCGATTAGCACCAGCCAGCCGACCAA |
| | | CCAGAGCATGGATGATACCCGCGAAGATATTTATGTGAACTATCC |
| | | GACCTTTAGCCGCCGCCCGAAAACCCGCGTG |

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### BRIEF DESCRIPTION OF THE FIGURES

Embodiments and experiments illustrating the principles of the invention will now be discussed with reference to the accompanying figures in which:

FIGS. 1A and 1B. Schematic representations of the GPC3 targeted CAR constructs of the present invention in the pELNS lentiviral vector.

FIGS. 2A and 2B. Scatterplots showing expression of anti-GPC3 at the cell surface of T cells transduced with anti-GPC3 CAR constructs, as determined by flow cytometry. FIG. 2A show the results of analysis of non-transduced T cells (negative control), T cells transduced with a construct encoding GFP (transduction control), or T cells transduced with (FIG. 2A) T, KK, LL, W or X (FIG. 2B) S, CC, FF, U, Z, BB, or EE GPC3-CAR constructs.

FIGS. 3A to 3C. Bar charts showing cell killing of GPC3-expressing cells by T cells transduced with anti-GPC3 CAR constructs having different domains, as determined by Delfia cytotoxicity assay. FIGS. 3A and 3B show specific cytolysis of HepG2 cells by non-transduced T cells (negative control), or T cells transduced with T, KK, LL, W, or X GPC3-CAR constructs, at target cell:CAR-T cell ratios of (FIG. 3A) 10:1 and (FIG. 3B) 20:1.

FIG. 3C shows specific cytolysis of HepG2 cells by T cells transduced with construct encoding GFP (negative control), or transduced with Z, S, BB, CC, U, EE, FF GPC3-CAR constructs, at target cell:CAR-T cell ratios of 10:1 and 20:1.

FIG. 4. Bar chart showing cell killing of GPC3-expressing cells by T cells transduced with anti-GPC3 CAR constructs having different domains. Percent cytolysis of HepG2 cells in the absence of T cells, in the presence of Triton X-100 (positive control), by T cells transduced with construct encoding GFP (negative control), or transduced with T or X GPC3-CAR constructs is shown, as determined by xCELLigence assay.

FIGS. 5A and 5B. Graph and Bar chart showing cell killing of GPC3-expressing cells by T cells transduced with anti-GPC3 CAR constructs having different domains. FIG. 5A shows cytolysis over time. FIG. 5B shows percent cytolysis of HepG2 cells in the absence of T cells, by non-transduced T cells (negative control), or transduced with T, KK, LL, W or X GPC3-CAR constructs, as determined by xCELLigence assay.

FIGS. 6A and 6B. Graph and Bar chart showing cell killing of GPC3-expressing cells by T cells transduced with anti-GPC3 CAR constructs having different domains. FIG. 6A shows cytolysis over time. FIG. 6B shows percent cytolysis of HepG2 cells in the absence of T cells, by T cells transduced with construct encoding GFP (negative control), or transduced with T, KK, LL, W, X, GG or MM GPC3-CAR constructs, as determined by xCELLigence assay.

FIGS. 7A and 7B. Graph and Bar chart showing cell killing of GPC3-expressing cells by T cells transduced with anti-GPC3 CAR constructs having different domains. FIG. 7A shows cytolysis over time. FIG. 7B shows percent cytolysis of HepG2 cells by T cells transduced with construct encoding GFP (negative control), or transduced with T, W or X GPC3-CAR constructs, as determined by xCELLigence assay.

FIGS. 8A to 8D. Graph and Bar charts showing cell killing of GPC3-expressing cells by T cells transduced with anti-GPC3 CAR constructs having different domains. FIG. 8A shows cytolysis over time. FIGS. 8B to 8D show specific cytolysis of HepG2 cells by T cells transduced with construct encoding GFP (negative control), or transduced with Z, S, BB, CC, T, EE or FF GPC3-CAR constructs, at (FIG. 8B) 4 hours, (FIG. 8C) 12 hours, and (FIG. 8D) 36 hours, as determined by xCELLigence assay.

FIGS. 9A to 9D. Graph and Bar charts showing cell killing of GPC3-expressing cells by T cells transduced with anti-GPC3 CAR constructs having different domains. FIG. 9A shows cytolysis over time. FIGS. 9B to 9D show specific cytolysis of HepG2 cells by T cells transduced with construct encoding GFP (negative control), or transduced with Z, S, BB, CC, T, EE or FF GPC3-CAR constructs, at (FIG. 9B) 4 hours, (FIG. 9C) 12 hours, and (FIG. 9D) 24 hours, as determined by xCELLigence assay.

FIGS. 10A to 10D. Graph and Bar charts showing cell killing of GPC3-expressing cells by T cells transduced with anti-GPC3 CAR constructs having different domains. FIG. 10A shows cytolysis over time. FIGS. 10B to 10D show specific cytolysis of HepG2 cells by T cells transduced with construct encoding GFP (negative control), or transduced with Z, S, BB, CC, T, EE or FF GPC3-CAR constructs, at (FIG. 10B) 4 hours, (FIG. 100) 8 hours, and (FIG. 10D) 16 hours, as determined by xCELLigence assay.

FIGS. 11A and 7B. Graph and Bar chart showing cell killing of GPC3-expressing cells by T cells transduced with anti-GPC3 CAR constructs having different domains. FIG. 11A shows cytolysis over time. FIG. 11B shows percent cytolysis of HepG2 cells by T cells transduced with construct encoding GFP (negative control), or transduced with S or BB GPC3-CAR constructs, as determined by xCELLigence assay.

FIGS. 12A and 12B. Graph and Bar chart showing cell killing of GPC3-expressing cells by T cells transduced with anti-GPC3 CAR constructs having different domains. FIG. 12A shows cytolysis over time. FIG. 12B shows percent cytolysis of HepG2 cells by T cells transduced with construct encoding GFP (negative control), or transduced with S or BB GPC3-CAR constructs, as determined by xCELLigence assay.

FIGS. 13A to 13H. Bar charts showing levels of cytokines in co-cultures of GPC3-expressing cells and T cells transduced with anti-GPC3 CAR constructs. Bar charts shown the level of (FIG. 13A) IL-2, (FIG. 13B) IFNg, (FIG. 13C) TNFa, (FIG. 13D) GM-CSF, (FIG. 13E) MIP-1a, (FIG. 13F) MIP-1b, (FIG. 13G) RANTES, and (FIG. 13H) TNFb in cell culture supernatants of co-cultures of HepG2 cells with T cells transduced construct encoding GFP (negative control), or transduced with T, W or X GPC3-CAR constructs after 16 hours of co-culture.

FIGS. 14A to 14H. Graphs showing levels of cytokines in co-cultures of GPC3-expressing cells and T cells transduced with anti-GPC3 CAR constructs. Bar charts shown the level of (FIG. 14A) IL-2, (FIG. 14B) IFNg, (FIG. 14C) TNFa, (FIG. 14D) GM-CSF, (FIG. 14E) MIP-1a, (FIG. 14F) MIP-1b, (FIG. 14G) RANTES, and (FIG. 14H) TNFb in cell culture supernatants of co-cultures of HepG2 cells with T cells transduced construct encoding GFP (negative control), or transduced with T, W or X GPC3-CAR constructs after 16 hours of co-culture.

FIGS. 15A and 15B. Bar charts showing proliferation by T cells transduced with anti-GPC3 CAR constructs following co-culture with GPC3-expressing cells. Bar charts show proliferation of (FIG. 15A) CD4+ and (FIG. 15B) CD8+ T cells transduced with construct encoding GFP (negative control), or transduced with T, W or X GPC3-CAR constructs following 5 days of co-culture with HepG2 cells.

FIGS. 16A and 16B. Bar charts showing proliferation by T cells transduced with anti-GPC3 CAR constructs following co-culture with GPC3-expressing cells. Bar charts show proliferation of (FIG. 16A) CD4+ and (FIG. 16B) CD8+ T cells transduced with construct encoding GFP (negative control), or transduced with S, AA or BB GPC3-CAR constructs following 5 days of co-culture with HepG2 cells.

FIGS. 17A and 17B. Graph and Bar chart showing cell killing of GPC3-expressing cells by T cells transduced with anti-GPC3 CAR constructs having different domains, in the presence or absence of TGFβ. FIG. 17A shows cytolysis over time. FIG. 17B shows percent cytolysis of HepG2 cells by T cells transduced with construct encoding GFP (negative control), or transduced with S or BB GPC3-CAR constructs, as determined by xCELLigence assay.

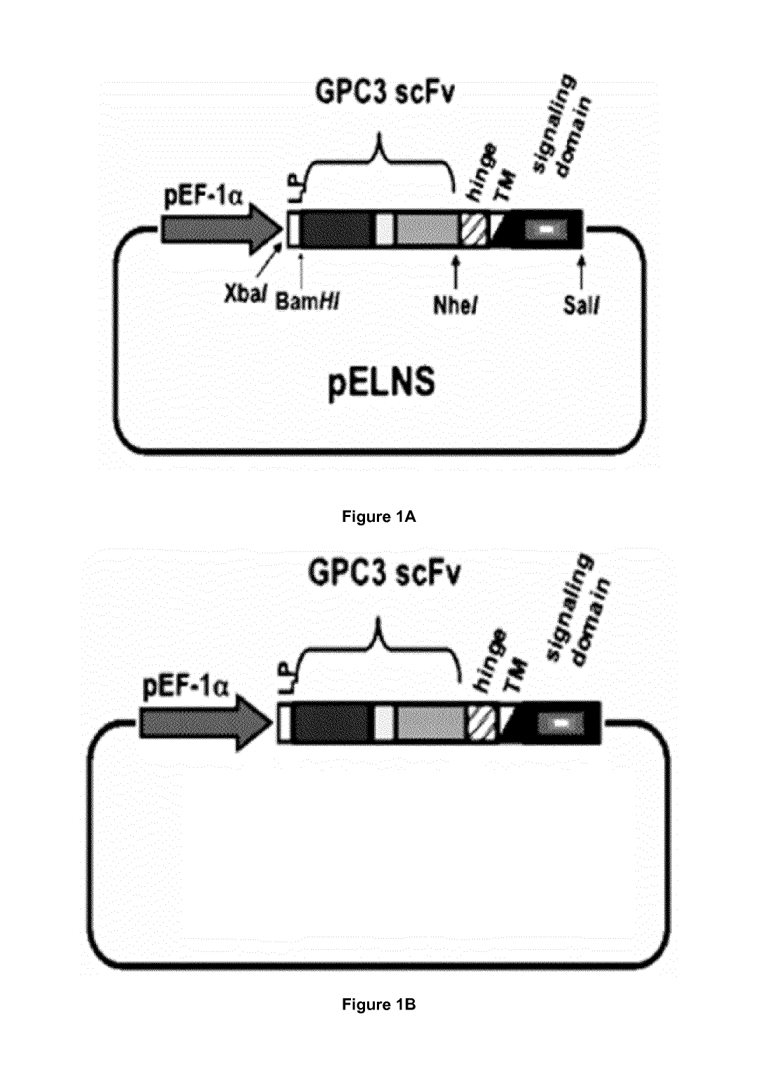

### EXAMPLES

The inventors describe in the following Examples construction of GPC3-targeted CARs, transduction into human T lymphocytes to generate GPC3-targeted CAR-T cells, antigen-specific killing of GPC3-expressing cells by the GPC3-targeted CAR-T cells, and anti-cancer activity of GPC3-targeted CAR-T cells in vivo, and reduced sensitivity to immunosuppressive factors, improved selectivity for tumour targets, improved priming of CTL to eradicate tumour cells, improved trafficking, tumour migration and penetration, and increased expression of growth factors for CAR-T cells expressing CARs comprising a CD226 costimulatory region as compared to CAR-T cells expressing CARs lacking a CD226 intracellular domain.

### Example 1: Generation of CARs Comprising CD226 Intracellular Domain and Lentivirally-Transduced Human T Lymphocytes

The cDNA of GC33 scFv and CD226 intracellular domain is amplified by PCR and inserted into the lentiviral vector pELNs using BamHI and NheI restriction sites to generate lentiviral vector pELNs/GC33 CARs having a CD226 intracellular domain.

For lentiviral transduction, 5×10⁶ HEK 293T cells are plated on 10 cm² dish pre-coated with 0.002% Poly-L-lysine (Sigma, St. Louis Mo.). The lentiviral vector pELNS-CARs are then co-transfected with the plasmid pMD.G, pMDLg/pRRE, and pRSV-Rev. The virus-containing supernatant is collected and passed through a 0.45 μm filter. The supernatant is then concentrated by ultracentrifugation at 25,000 rpm, titered, and then stored at −80° C. until use.

Primary human T lymphocytes isolated from healthy donors are acquired. T cells are cultured in complete medium (RPMI 1640 supplemented with 10% inactivated FBS, penicillin and streptomycin sulfate), and activated by stimulation with anti-CD3 and anti-CD28mAb-coated beads (Invitrogen). 12 hours after activation, the T cells are transduced with lentiviral vectors in presence of polybrene. Human T lymphocytes are expanded and maintained by addition of IL-2 every other day.

### Example 2: GPC3-Specific CAR Construction and T Lymphocyte Transduction

GC33 scFv is selected to construct GPC3-specific CARs with high antigen-binding affinity. A lentiviral CAR vector is used to make CAR constructs including different domains by sub-cloning of cDNA sequences encoding the domains into the vector. The following constructs are generated:

TABLE 1

| | | Trans- | | |
|---|---|---|---|---|
| | Antigen-binding | membrane | Dimerization | |
| | domain | domain | domain | Signaling domain |
| A | GPC3-binding scFV | CD8α | — | CD226, CD3ζ |
| B | GPC3-binding scFV | CD8α | F36V-FKBP | CD226, CD3ζ |
| C | GPC3-binding scFV | CD8α | — | CD226, CD28, CD3ζ |
| D | GPC3-binding scFV | CD8α | F36V-FKBP | CD226, CD28, CD3ζ |
| E | GPC3-binding scFV | CD8α | — | CD226, 4-1BB, |
| | | | | CD3ζ |
| F | GPC3-binding scFV | CD8α | F36V-FKBP | CD226, 4-1BB, |
| | | | | CD3ζ |
| G | GPC3-binding scFV | CD8α | — | CD226, CD28, |
| | | | | 4-1BB, CD3ζ |
| H | GPC3-binding scFV | CD8α | F36V-FKBP | CD226, CD28, |
| | | | | 4-1BB, CD3ζ |
| I | GPC3-binding scFV | CD28 | F36V-FKBP | 4-1BB, CD3ζ |
| J | GPC3-binding scFV | CD8α | — | 4-1BB, CD3ζ |
| K | GPC3-binding scFV | CD8α | F36V-FKBP | 4-1BB, CD3ζ |
| L | GPC3-binding scFV | CD8α | — | CD28, CD3ζ |
| M | GPC3-binding scFV | CD8α | F36V-FKBP | CD28, CD3ζ |

A signalling deficient construct containing a truncated CD3ζ intracellular domain is prepared as a negative control for evaluating initiation of signal transduction by the constructs.

The vectors are transformed into 293T cells, and lysates are analysed by western blot to confirm successful expression of the vectors.

For effective transduction, human T lymphocytes isolated from peripheral blood samples are activated by stimulation with CD3/CD28 beads. To evaluate transduction efficiency, T cells are transduced with GFP-expressing lentiviral vector, and stable consistent GFP expression is observed 10 days after transduction.

To analyse CAR expression at the T cell membrane, a FLAG-tag is artificially inserted at the N-terminus of the CAR, and expression is detected by flow cytometry following staining of the cells with an anti-FLAG mAb. The results suggest that around 50% T cells are transduced and express CAR receptor at the cell surface.

### Example 3: Comparison of T Cells Expressing a GPC3 CAR Including a CD226 Costimulatory Region to T Cells Expressing a GPC3 CAR Lacking a CD226 Costimulatory Region

GPC3 CART T cells with CD226 costimulatory regions display reduced sensitivity to immunosuppressive factors as compared to a CAR not comprising a costimulatory sequence of CD226. Expression of GC33/CD226 CARs in T cells is sufficient to protect CAR T cells from the potent inhibitory effect of treatment with TGF-β.

Selectivity of T cells expressing GC33/CD226 CAR is compared to T cells expressing GC33 CAR lacking a CD226 intracellular domain in vitro using targets that recapitulate normal vs. tumor tissue. CAR T cells expressing GC33/CD226 CAR selectively eliminate only tumor targets and not “normal” surrogate targets. The selectively of these CAR-T cells is confirmed in vivo.

### Example 4: Efficacy of T Cells Expressing GPC3/CD226 CAR in CTL Priming to Eradicate Tumor Cells

T cells expressing a GPC3 CAR having a CD266 intracellular domain are tested in in vitro priming systems and compared to T cells expressing GC33 CAR lacking a CD226 intracellular domain. Human CAR-expressing T cells are co-cultured with irradiated tumor cells, in the presence of a pool of non-engineered T cells and optionally DCs.

T cells expressing a GPC3/CD226 CAR display improved priming of CTL to eradicate tumour cells as compared to CARs lacking a CD226 intracellular domain.

### Example 5: Migration Assays Determining Cellular Localization after Infusion

A transwell migration assay indicates that GPC3/CD226 CAR-T cells are able to migrate towards tumor cell line supernatant more efficiently than GPC3 CAR-T cells lacking a CD226 intracellular domain.

GPC3 CAR-T cells are labeled with GFP and placed in the upper chamber of the 24-well transwell chamber. Media alone or LCL tumor supernatant is placed in the bottom chamber. Plates are then incubated for 3 h at 37° C. Cells in the bottom chamber are then harvested and analysed to determine migration of T cells from the upper chamber to the lower chamber. Specific migration is calculated using the following equation:

Specific Migration (%)=(Experimental[LCL supernatant]−Spontaneous[media alone])/(Maximum[1.5×10⁵ cells]−Spontaneous[media alone])×100.

CAR-T cells expressing the CAR construct including a CD226 intracellular domain exhibit trafficking to the lower chamber, and display better tumor migration and penetration as compared to GPC3 CAR-T cells lacking a CD226 intracellular domain.

### Example 6: Cytokine Assays Using Multiplex Cytokine Analysis

Levels of interleukin-2 (IL-2), IL-4, IL-5, IL-6, IL-10, IL-12, IL-13, IL-17, interferon-gamma (IFN-gamma), granulocyte/macrophage colony-stimulating factor, and tumor necrosis factor-alpha (TNF-alpha) in the cell culture supernatant of the CAR T cells co-expressing CD226 is analysed by multiplex technique. Intracellular cytokine staining (ICS) is employed to detect IFN-gamma, IL-2, IL-4 and IL-13 in CD3⁺ cells.

The multiplex analysis detects representative cytokine profiles for the majority of the cytokines on day 7 by identifying peak levels or good correlation with peak levels.

CAR-T cells expressing CAR including a CD226 intracellular domain have increased expression of growth factors as compared to GPC3 CAR-T cells lacking a CD226 intracellular domain.

### Example 7: Generation of GP3C-Specific CAR and Lentivirally-Transduced Human T Lymphocytes

The cDNA of GC33 scFv is amplified by PCR and inserted into the lentiviral vector pELNs using BamHI and NheI restriction sites, to generate lentiviral vector pELNs/GC33 CARs.

For lentiviral transduction, 5×10⁶ HEK 293T cells are plated on 10 cm² dish pre-coated with 0.002% poly-L-lysine (Sigma, St. Louis Mo.). The lentiviral vector pELNS-CARs are then co-transfected with the plasmid pMD.G, pMDLg/pRRE, and pRSV-Rev. The virus-containing supernatant is collected and passed through a 0.45 μm filter. The supernatant is then concentrated by ultracentrifugation at 25,000 rpm, titered, and then stored at −80° C. until use.

Primary human T lymphocytes isolated from healthy donors are acquired. T cells are cultured in complete medium (RPMI 1640 supplemented with 10% inactivated FBS, penicillin and streptomycin sulfate), and activated by stimulation with anti-CD3 and anti-CD28mAb-coated beads (Invitrogen). 12 hours after activation, the T cells are transduced with lentiviral vectors in presence of polybrene. Human T lymphocytes are expanded and maintained by addition of IL-2 every other day.

### Example 8: Validation of the Ability of the CAR to Direct T-Cells Against G3PC Expressing Target Cells

The ability of the transduced T lymphocytes to lyse GPC3-positive tumor cells is confirmed by in vitro analysis by fluorescence-based killing assay, cytokine release assay, and high dimension flow cytometry.

### Example 9: CAR+ T Cells Showed GPC3-Specific Cytotoxicity In Vitro

Engineered T cells are co-cultured with GPC3-positive or GPC3-negative tumor cells to determine whether the CAR-expressing T cells display antigen-specific cytotoxicity.

T cells are transduced by lentiviral vector and their transduction efficiency is assessed by FACS, and further equilibrated. T cells transduced with GFP lentiviral vector are included as a control. For target cells, several established tumor cell lines are selected and GPC3 protein expression levels are determined by FACS. Two tumor cell lines, hs578T (a GPC3-negative cell line) and HepG2.sh57 (a cell line which displays lower level of GPC3 expression), are also selected.

The results indicate that the GPC3-CAR transduced T cells display antigen-specific cytotoxicity to target cells expressing GPC3.

### Example 10: Improved In Vivo Proliferation and Persistence and Enhanced Antitumor Efficacy of GPC3-CAR T Cells after Adoptive Transfer

GPC3-CAR T cells are injected subcutaneously into immune-compromised mice with GPC3-positive xenograft tumours.

The mice in the untreated control group start dying after 50 days. By contrast, mice treated with GPC3-CAR T cells continue to survive. After 130 days of treatment, most of the mice from the control group have died, but ˜80% of mice from CAR T group remain alive. These data indicate that GPC3-CAR T cells have improved in vivo persistence and proliferation, and enhanced long-term antitumor effects in vivo.

### Example 11: Generation of Further CAR Constructs

Further CAR constructs were generated by PCR amplification and sub-cloning of cDNA encoding the different CAR domains into a lentiviral CAR vector. The following constructs were generated:

| | | | Trans- | |
|---|---|---|---|---|
| | Antigen-binding | Hinge | membrane | Signaling |
| | domain | region | domain | domain |
| R | GPC3-binding scFV | Human IgG1 | CD28 | CD3ζ |
| S | GPC3-binding scFV | Human IgG1 | CD28 | CD28, CD3ζ |
| T | GPC3-binding scFV | Human IgG1 | CD28 | 4-1BB, CD3ζ |
| U | GPC3-binding scFV | Human IgG1 | CD28 | CD28, 4-1BB, |
| | | | | CD3ζ |
| V | GPC3-binding scFV | Human IgG1 | CD28 | CD226 ICDv2 |
| W | GPC3-binding scFV | Human IgG1 | CD28 | CD226 ICDv1, |
| | | | | 4-1BB, CD3ζ |
| X | GPC3-binding scFV | Human IgG1 | CD28 | CD226 ICDv2, |
| | | | | 4-1BB, CD3ζ |
| Y | GPC3-binding scFV | Human IgG1 | CD28 | CD226 ICDv1, |
| | | | | CD3ζ |
| Z | GPC3-binding scFV | Human IgG1 | CD28 | CD226 ICDv2, |
| | | | | CD3ζ |
| AA | GPC3-binding scFV | Human IgG1 | CD28 | CD226 ICDv1, |
| | | | | CD28, CD3ζ |
| BB | GPC3-binding scFV | Human IgG1 | CD28 | CD226 ICDv2, |
| | | | | CD28, CD3ζ |
| CC | GPC3-binding scFV | Human IgG1 | CD28 | CD28, CD226 |
| | | | | ICDv1, CD3ζ |
| DD | GPC3-binding scFV | Human IgG1 | CD28 | CD226 ICDv1, |
| | | | | CD28, 4-1BB, |
| | | | | CD3ζ |
| EE | GPC3-binding scFV | Human IgG1 | CD28 | CD226 ICDv2, |
| | | | | CD28, 4-1BB, |
| | | | | CD3ζ |
| FF | GPC3-binding scFV | Human IgG1 | CD28 | CD28, CD226 |
| | | | | ICDv1, 4-1BB, |
| | | | | CD3ζ |
| GG | GPC3-binding scFV | Human IgG1 | CD226 | CD226 ICDv1, |
| | | | | 4-1BB, CD3ζ |
| HH | EpCAM-binding scFV | Human IgG1 | CD226 | CD226 ICDv1 |
| II | EpCAM-binding scFV | Human IgG1 | CD226 | CD3ζ |
| JJ | EpCAM-binding scFV | Human IgG1 | CD226 | CD226 ICDv1, |
| | | | | CD3ζ |
| KK | GPC3-binding scFV | Human IgG1 | CD28 | 4-1BB, CD3ζ, |
| | | | | CD226 ICDv1 |
| LL | GPC3-binding scFV | Human IgG1 | CD28 | 4-1BB, CD226 |
| | | | | ICDv1, CD3ζ |
| MM | GPC3-binding scFV | Human IgG1 | CD226 | CD226 ICDv1 |

### Example 12: Expression of Anti-GPC3 CARs on Transduced T Cells

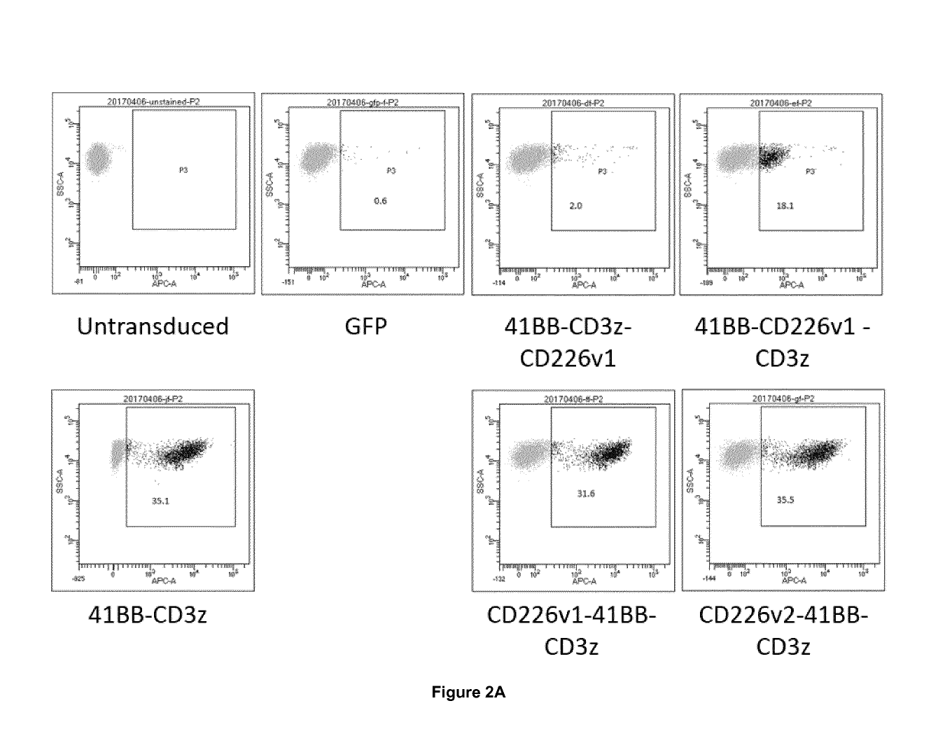

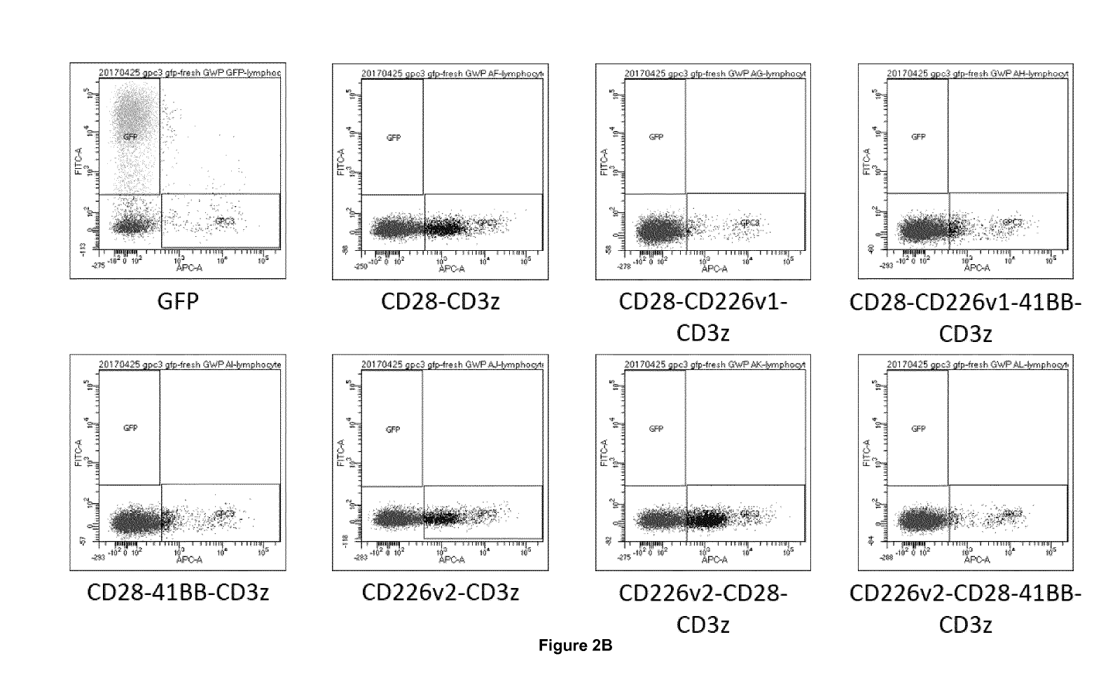

CD3+ cells were obtained from peripheral blood samples, activated by stimulation with anti-CD3/anti-CD28 beads and then transduced with the following GPC3-CAR constructs described in Example 11: T, KK, LL, W or X (FIG. 2A), S, CC, FF, U, Z, BB, or EE (FIG. 2B) or lentivirus encoding GFP, as a transduction control.

Expression of the GPC3-CARs at the cell surface of the transduced cells was analysed by flow cytometry using biotinylated, anti-mouse-fab′ antibody and fluorescently-conjugated strepatavidin.

The results are shown in FIGS. 2A and 2B. GPC3-CAR expression was detected at the cell surface of the transduced cells.

### Example 13: Analysis of Cell Killing by GPC3-Targeted CAR-T Cells

### 13.1 Analysis by Delfia Cytotoxicity Assay

Transduced T cells expressing GPC3-specific CAR constructs were analysed for their ability to lyse GPC3-expressing cells.

GPC3-expressing HepG2 hepatocarcinoma cells were loaded with Delfia fluorescence enhancer reagent. Lysis of target cells by the GPC3-targeted CAR-T cells releases the enhancer reagent into the culture media.

Culture media were collected after 2 hours co-incubation of HepG2 cells and GPC3-CAR-T cells; experiments were performed at target cell:CAR-T cell ratios of 10:1 and 20:1.

Fluorescence was measured with a fluorescence plate reader and compared to fluorescence released spontaneously, and fluorescence released by chemical lysis of Delfia-loaded HepG2 cells, to calculate percent specific cytolysis.

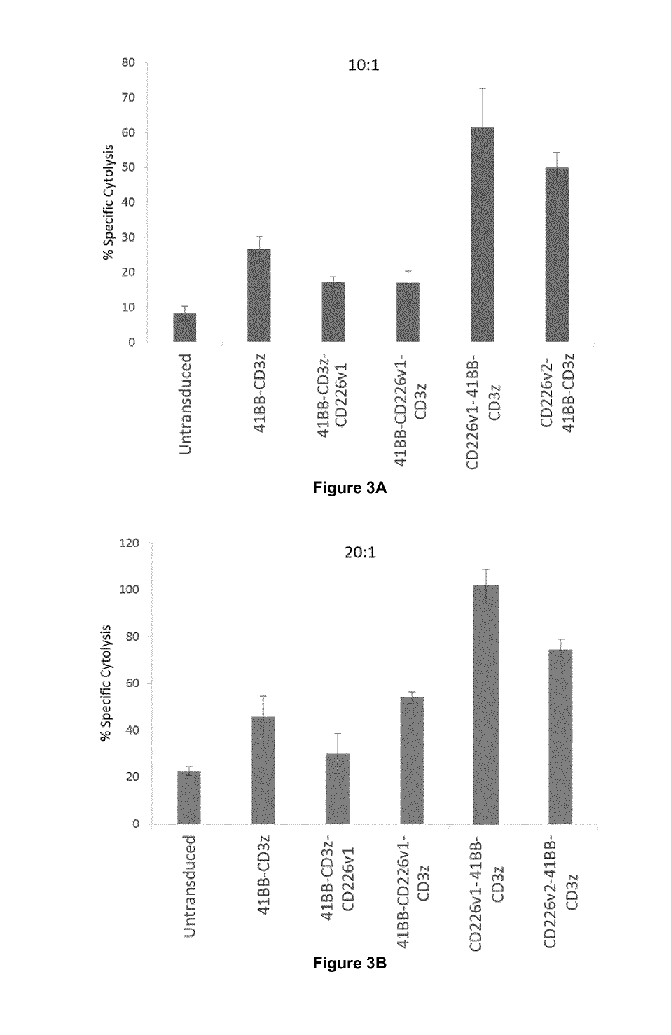

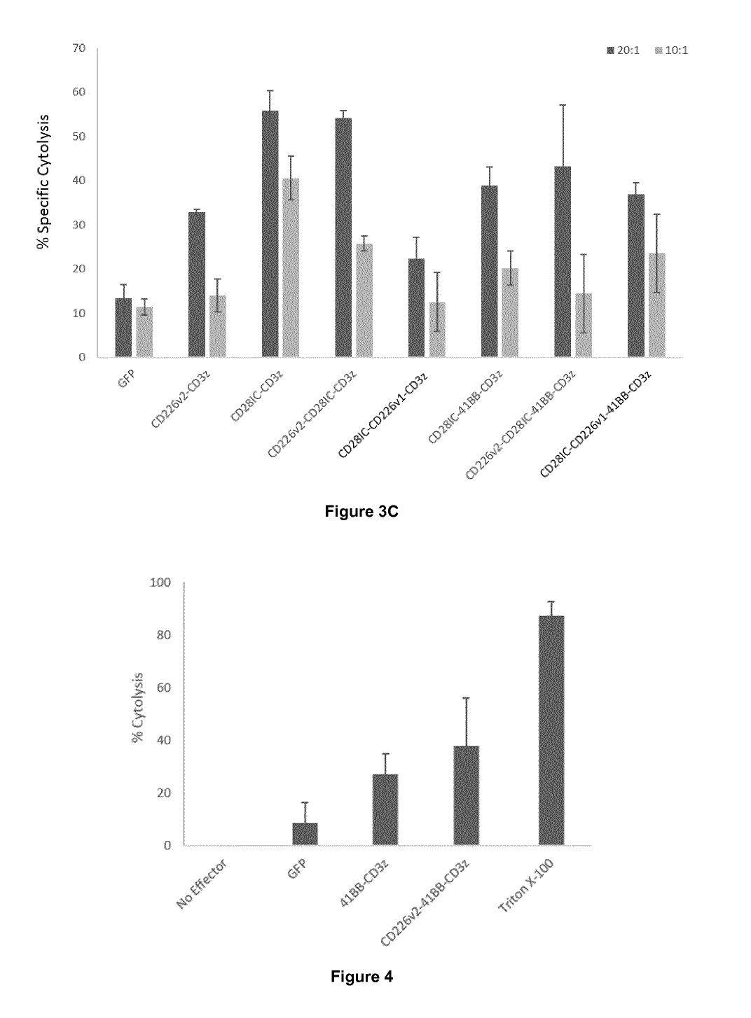

The results of experiments performed using T cells transduced with constructs T, KK, LL, W, or X constructs (see Example 11) are shown in FIGS. 3A and 3B.

The results of experiments performed using T cells transduced with constructs Z, S, BB, CC, U, EE or FF constructs (see Example 11) are shown in FIG. 3C.

The GPC3-CAR-T cells were shown to be capable of killing GPC3-expressing cells.

13.2 Analysis by xCELLigence Assay

Further analysis of lysis of HepG2 cells by GPC3-targeted CAR-T cells was performed using xCELLigence (ACEA Biosciences Inc) system, which measures changes in electrical resistance associated with changes in adherence of cells to gold microelectrodes. Interaction between the cells with the gold microelectrodes changes the flow of electric current between electrodes, and this impedance value is calculated as a “Cell Index”.

Briefly, HepG2 cells were seeded in xCELLigence plates and growth was monitored. When near-confluent or confluent, CAR-T cells were added to cultures at an effector:target cell ratio of 0.5:1. Lysis of HepG2 cells by CAR-T cells was monitored by xCELLigence machine and percent cytolysis was calculated using XIMT software.

The results performed using T cells transduced with GPC3-targeted constructs T or X are shown in FIG. 4. FIG. 4 shows percent cytolysis of the HepG2 cells at the end of the experiment. The T cells transduced with the X construct were found to display increased cytolytic activity against the GPC3-expressing cells as compared to T cells transduced with the T construct.

Further experiments were performed, using T cells isolated from blood samples obtained from different donors.

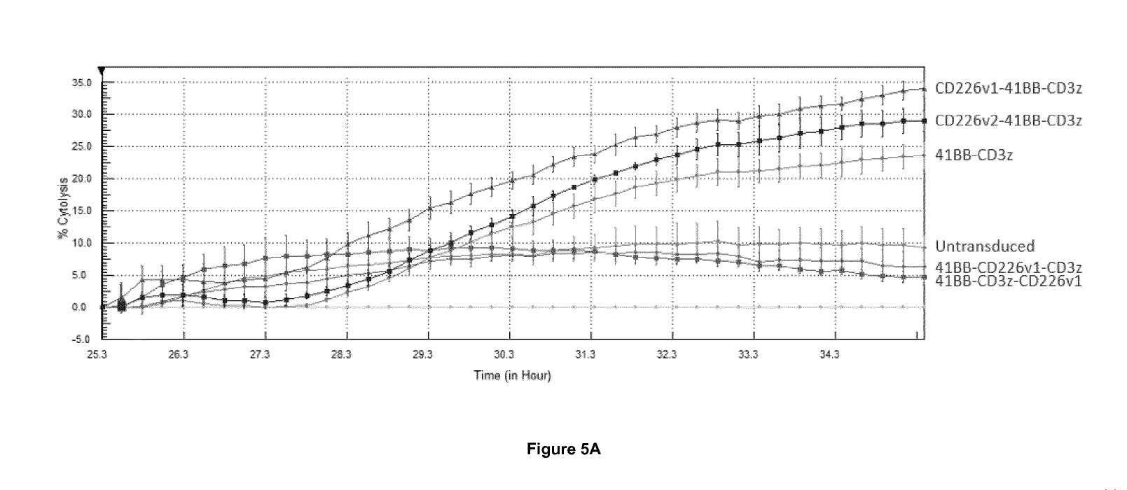

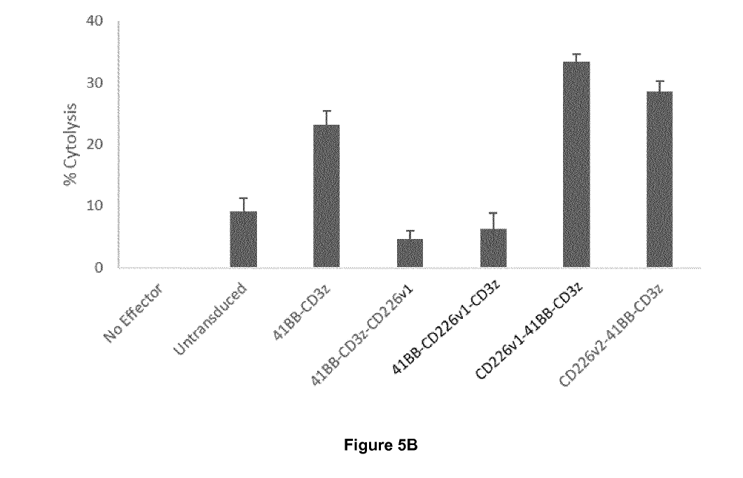

FIGS. 5A and 5B show the results obtained using T cells from donor ID1, 28 days after transduction with construct T, KK, LL, W or X. T cells transduced with the W and X constructs were found to display increased cytolytic activity against the GPC3-expressing cells as compared to T cells transduced with the T construct.

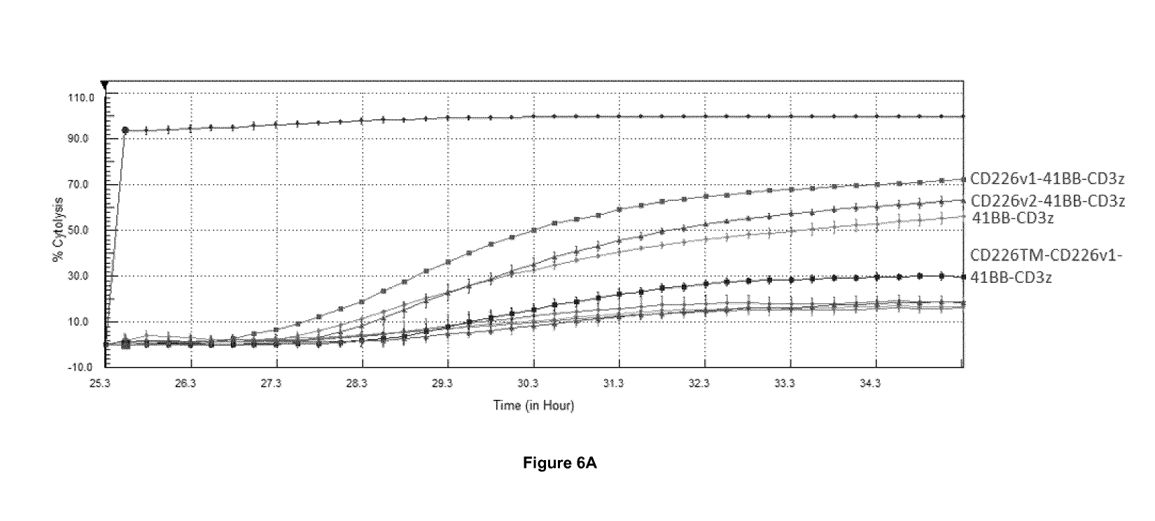

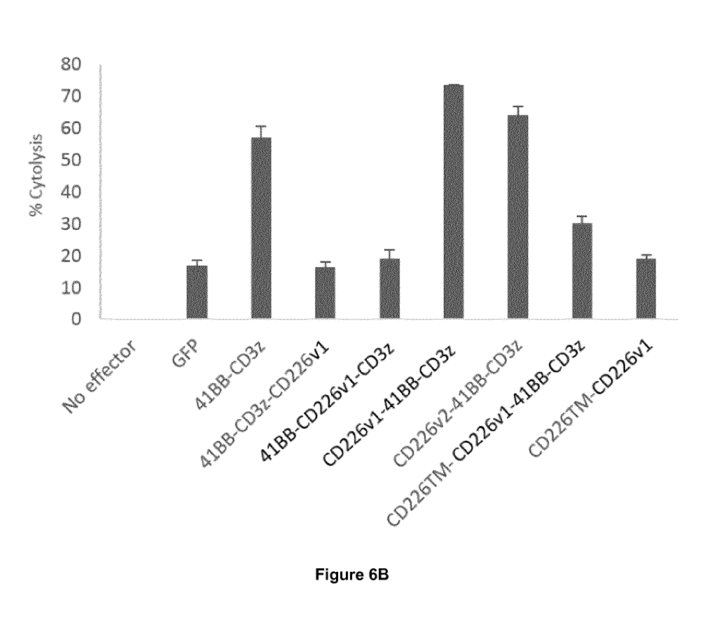

FIGS. 6A and 6B show the results obtained using T cells from donor ID2, 14 days after transduction with construct T, KK, LL, W, X, GG or MM. Once again, T cells transduced with the W and X constructs were found to display increased cytolytic activity against the GPC3-expressing cells as compared to T cells transduced with the T construct.

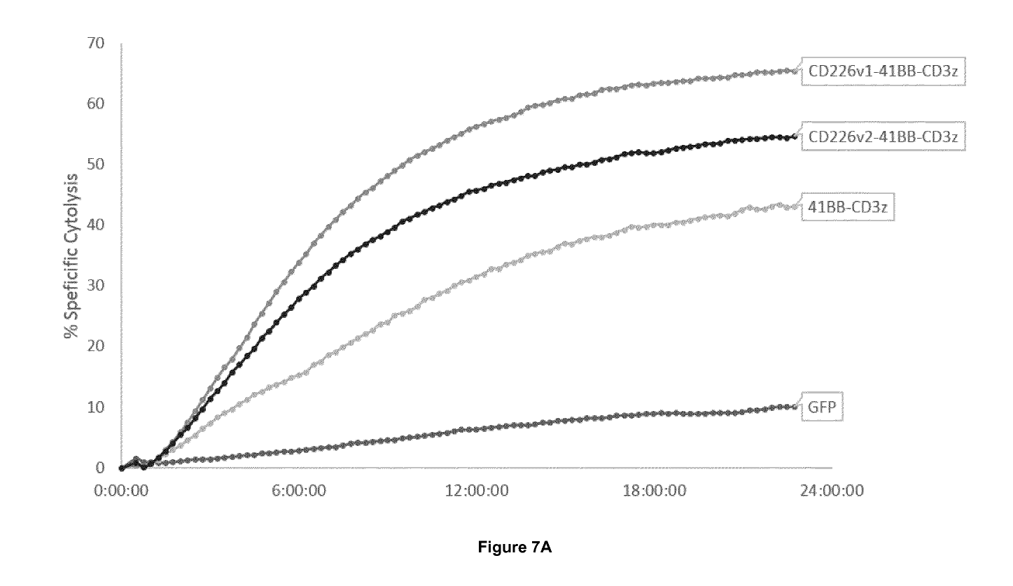

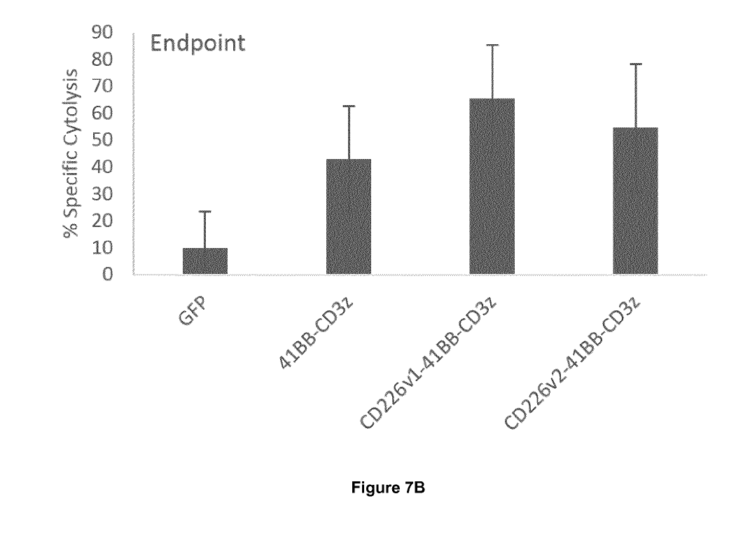

FIGS. 7A and 7B show the results obtained using T cells from donor ID4, 19 days after transduction with construct T, W or X. Once again, T cells transduced with the W and X constructs were found to display increased cytolytic activity against the GPC3-expressing cells as compared to T cells transduced with the T construct.

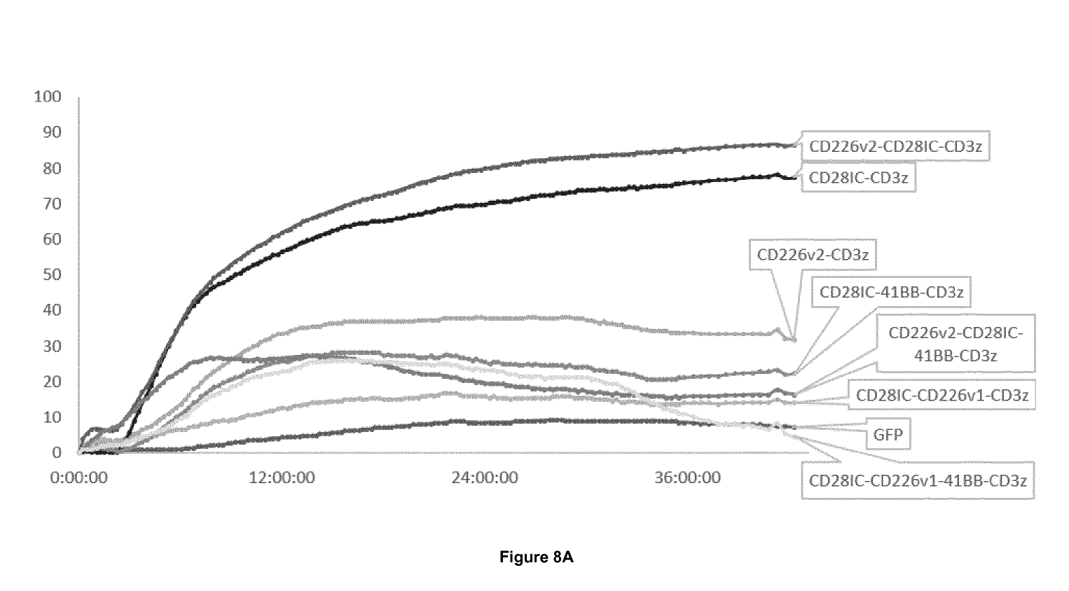

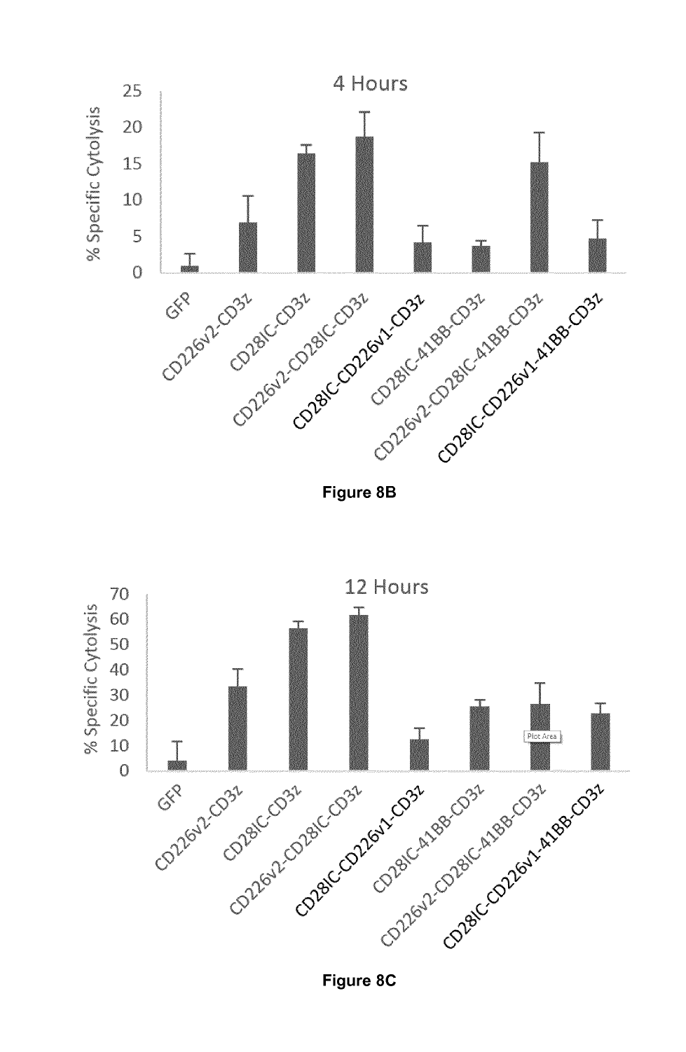

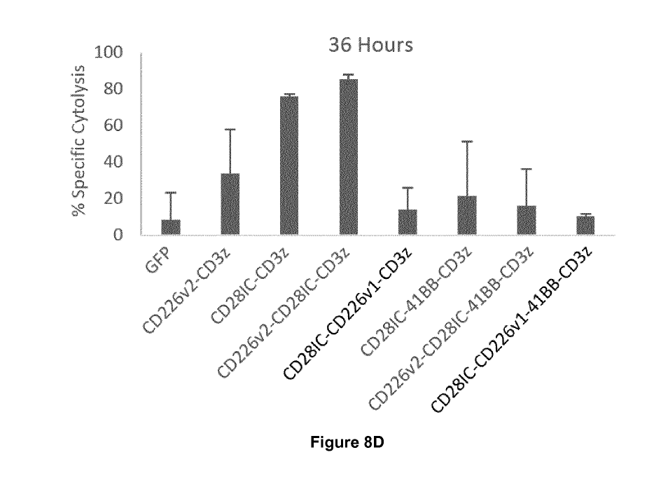

FIGS. 8A to 8D show the results obtained using T cells from donor ID3, at different time points in the experiment, from 10 days after transduction with construct Z, S, BB, CC, T, EE, FF.

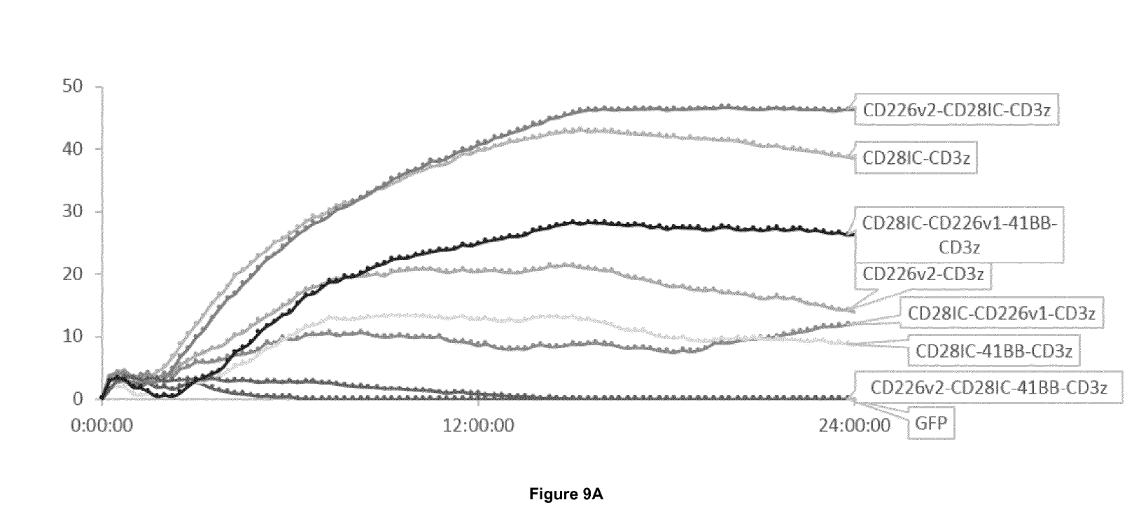

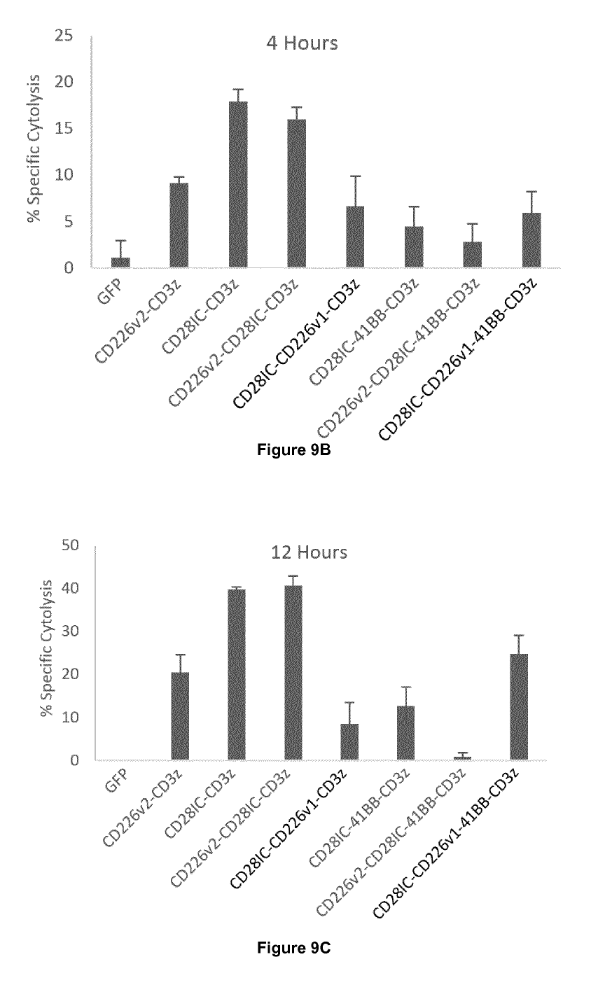

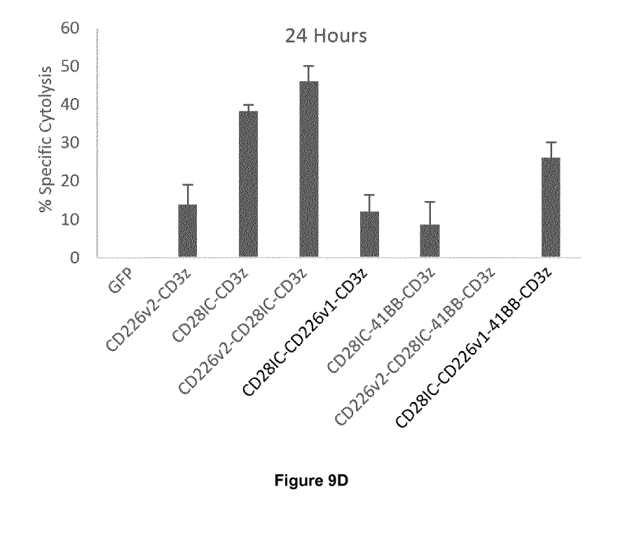

FIGS. 9A to 9D show the results obtained using T cells from donor ID3, at different time points in the experiment, from 12 days after transduction with construct Z, S, BB, CC, T, EE or FF.

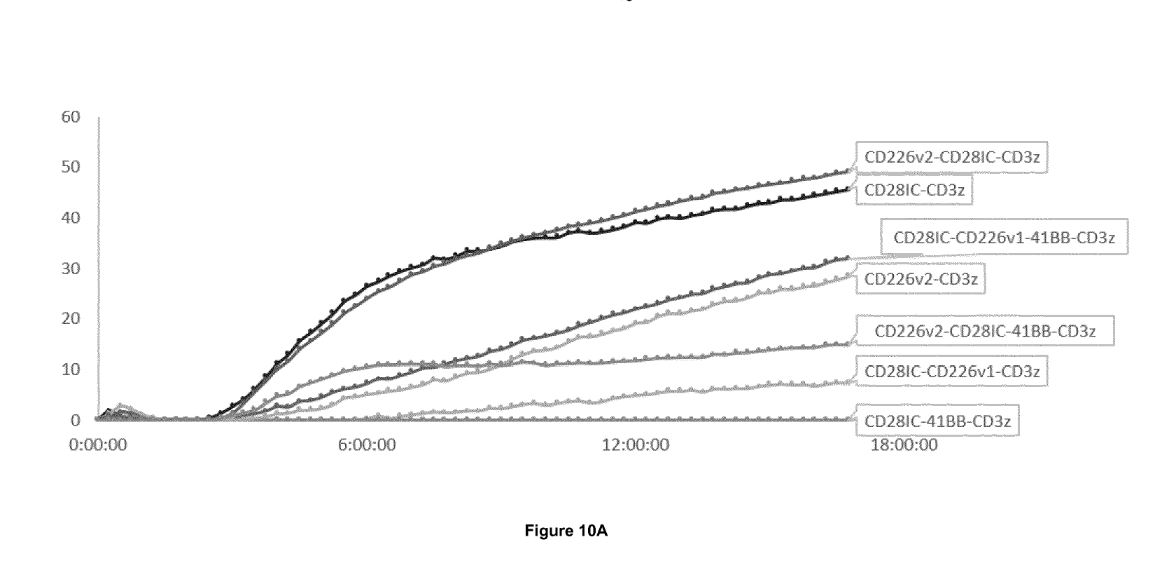

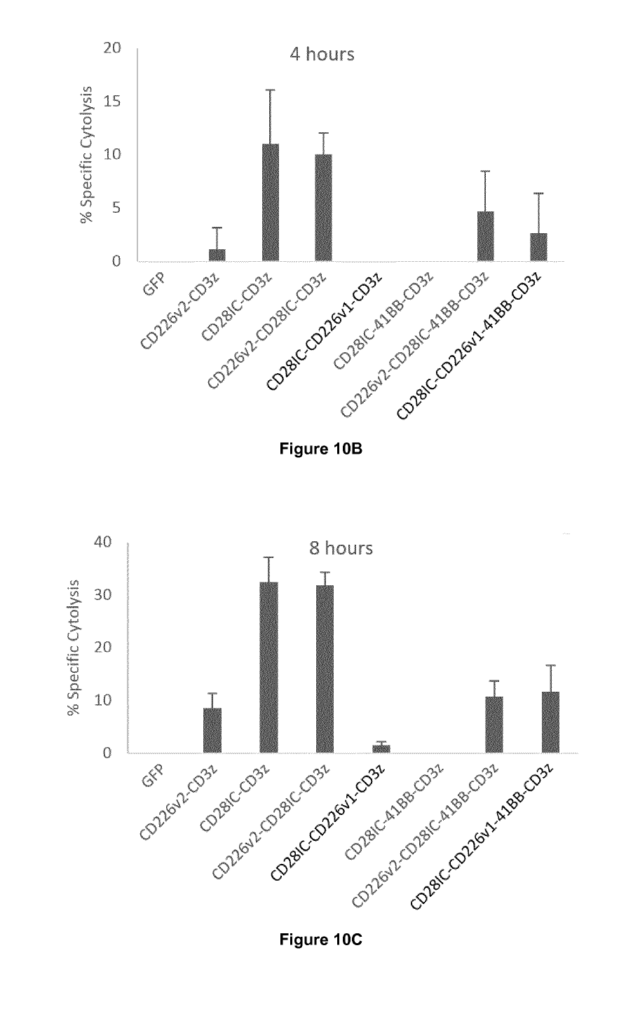

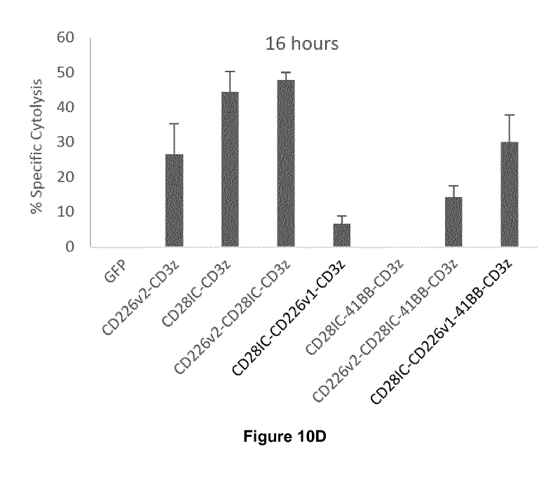

FIGS. 10A to 10D show the results obtained using T cells from donor ID3, at different time points in the experiment, from 20 days after transduction with construct Z, S, BB, CC, T, EE or FF.

FIGS. 11A and 11B show the results obtained using T cells from donor ID4, 19 days after transduction with construct S or BB. T cells transduced with the BB construct were found to display increased cytolytic activity against the GPC3-expressing cells as compared to T cells transduced with the S construct.

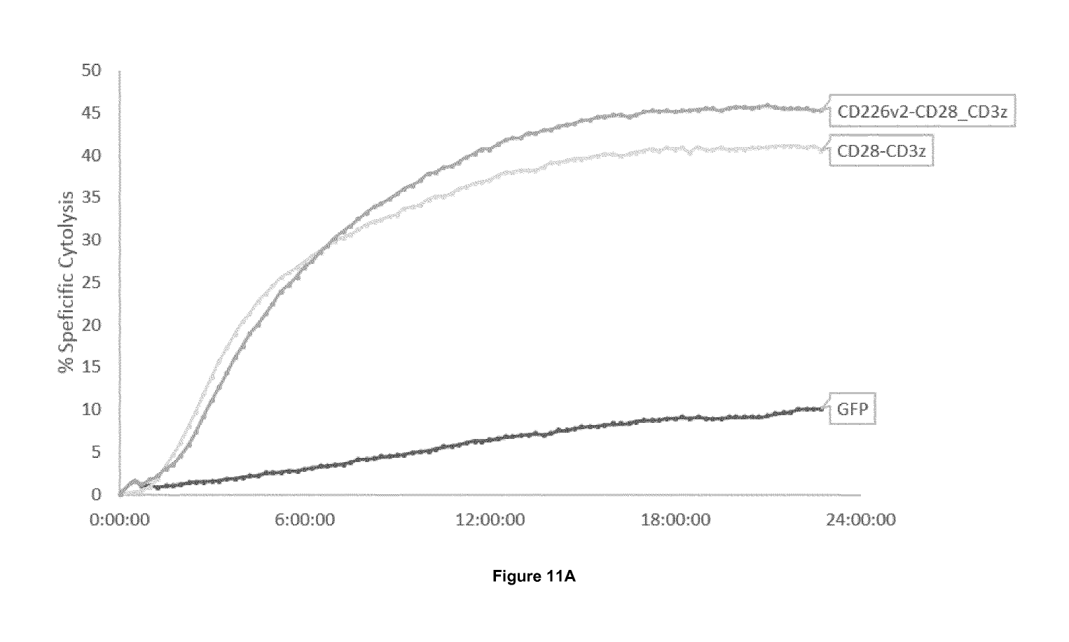

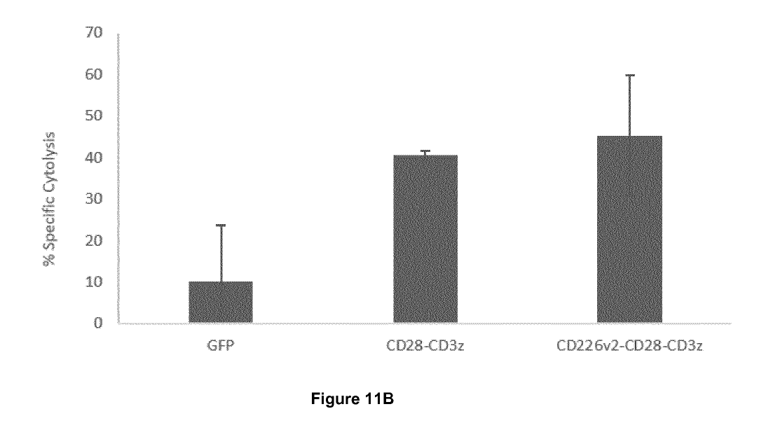

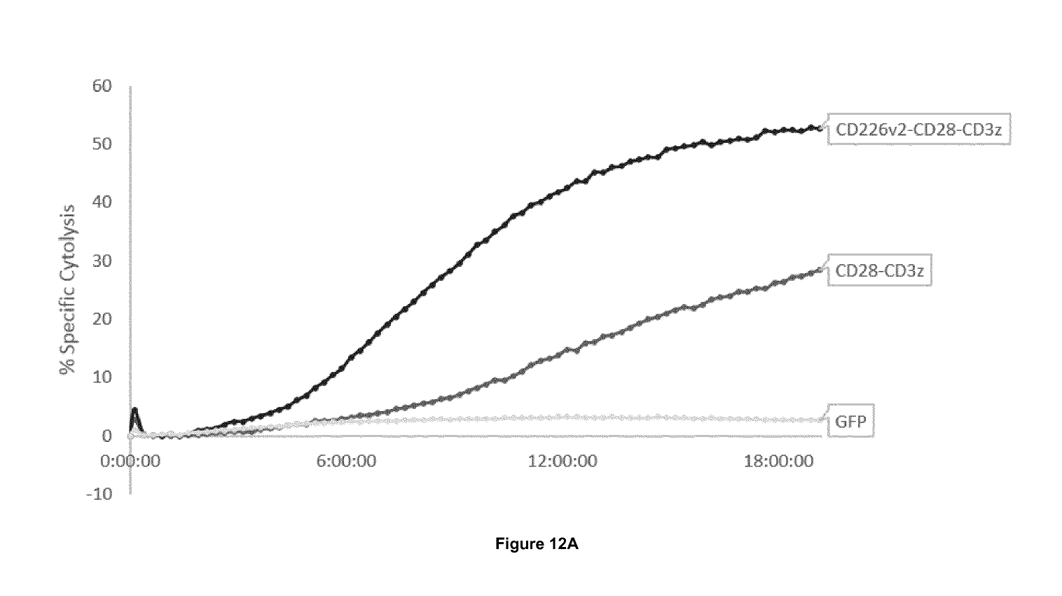

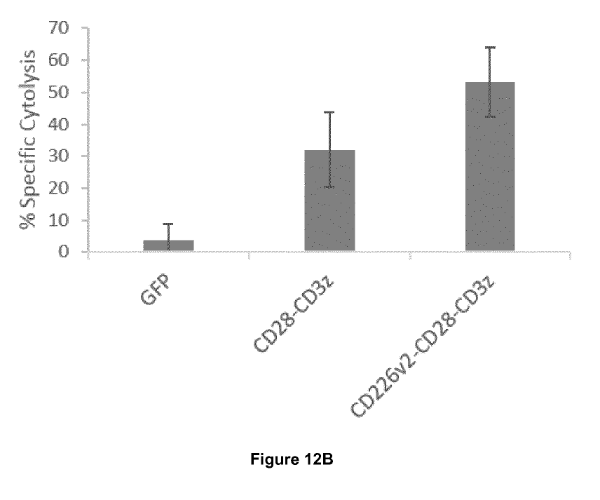

FIGS. 12A and 12B show the results obtained using T cells from donor ID5, 16 days after transduction with construct S or BB. T cells transduced with the BB construct were again found to display increased cytolytic activity against the GPC3-expressing cells as compared to T cells transduced with the S construct.

### Example 14: Cytokine Production by GPC3-CAR Expressing CAR-T Cells

Cytokine production was analysed in 16 hour co-cultures of CAR-T cells transduced with GPC3-CAR lentivirus constructs and HepG2 cells. Cell-free supernatants were collected and analysed or frozen at −80 to be analysed later.

Multiplex analysis of the level of cytokines MIP-1a, MIP-1b, RANTES and TNFb produced by the cells in culture was performed using the Merck Immuno-monitoring reagent set, and the Luminex plate reader system.

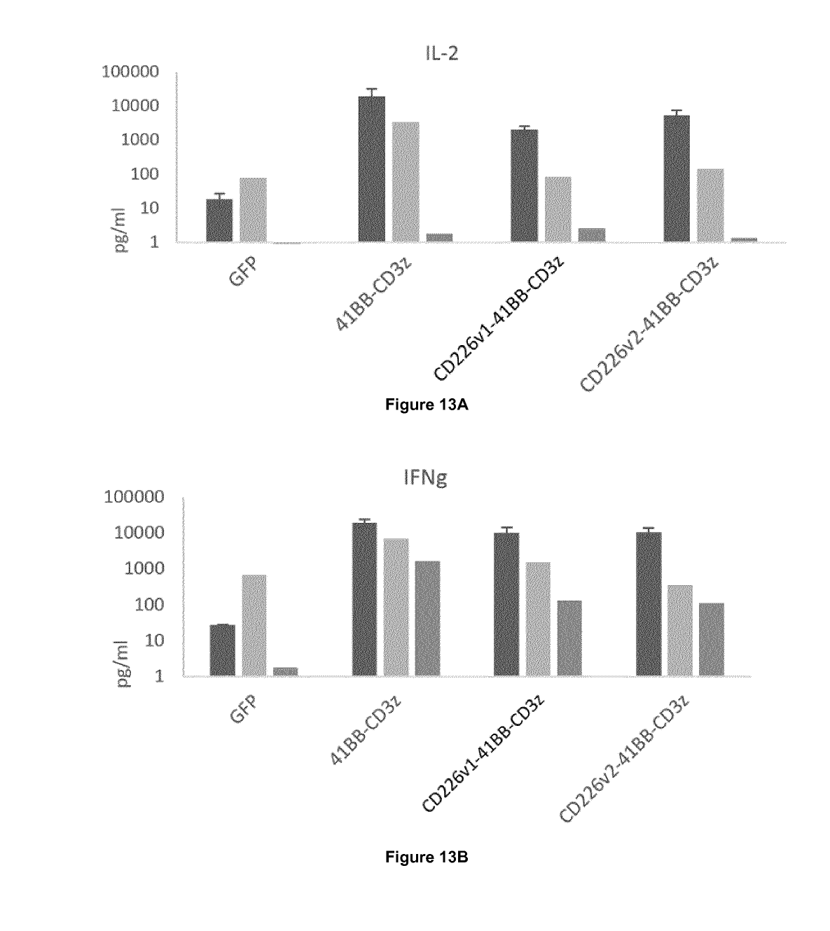

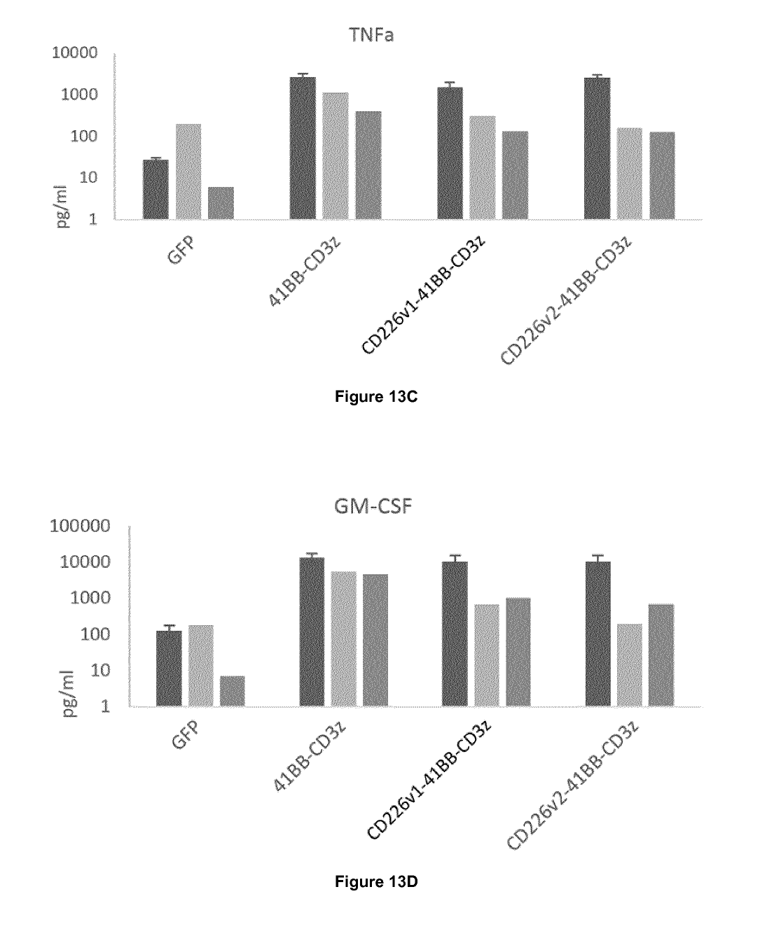

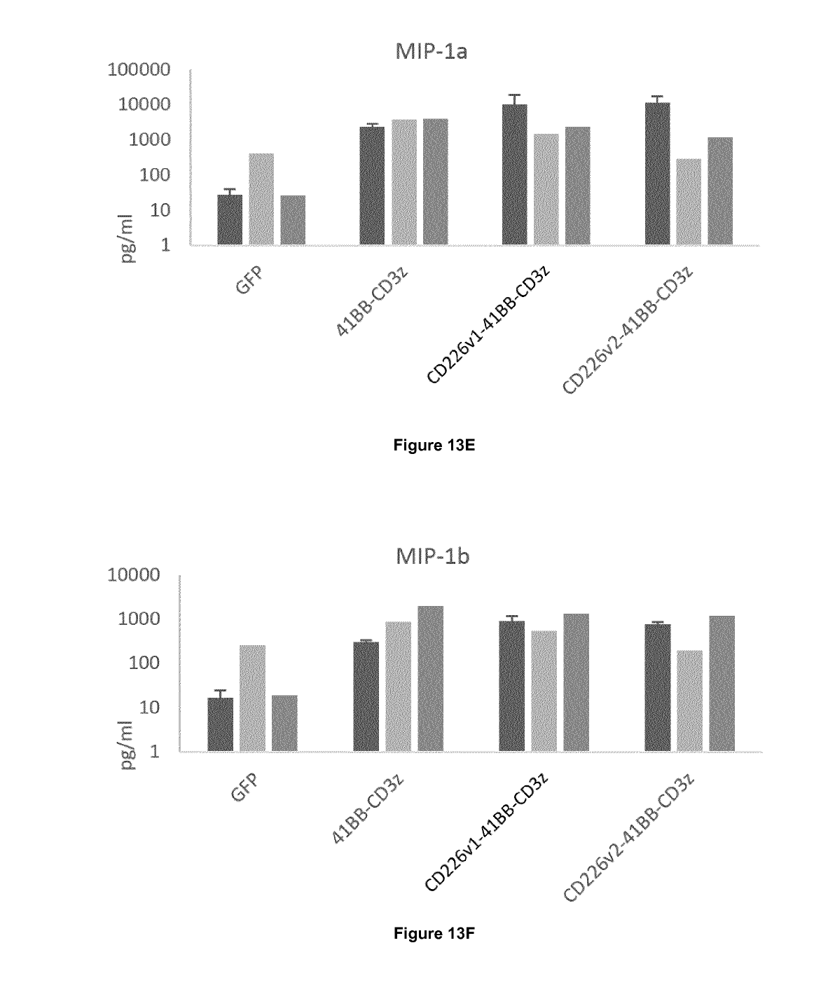

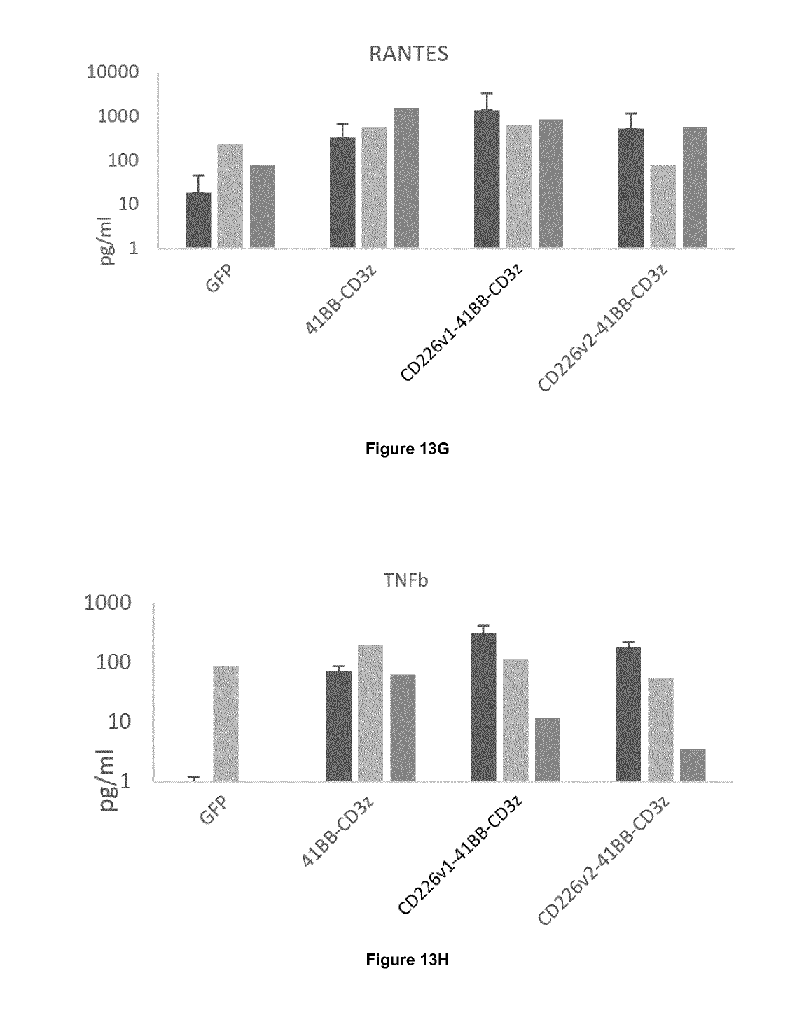

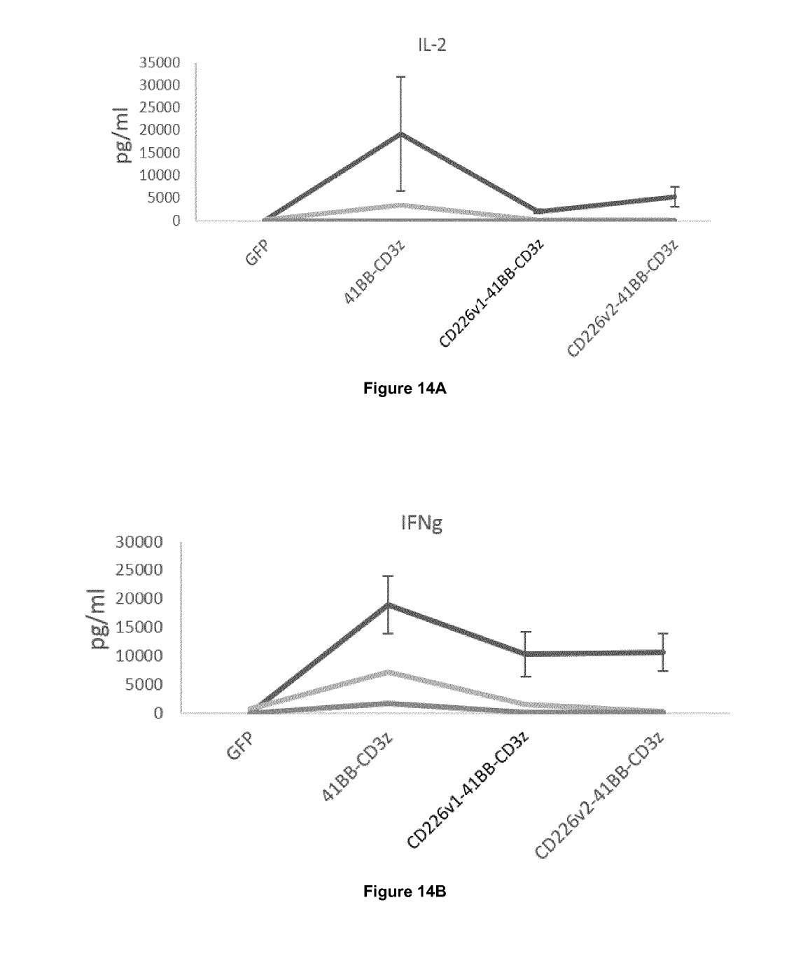

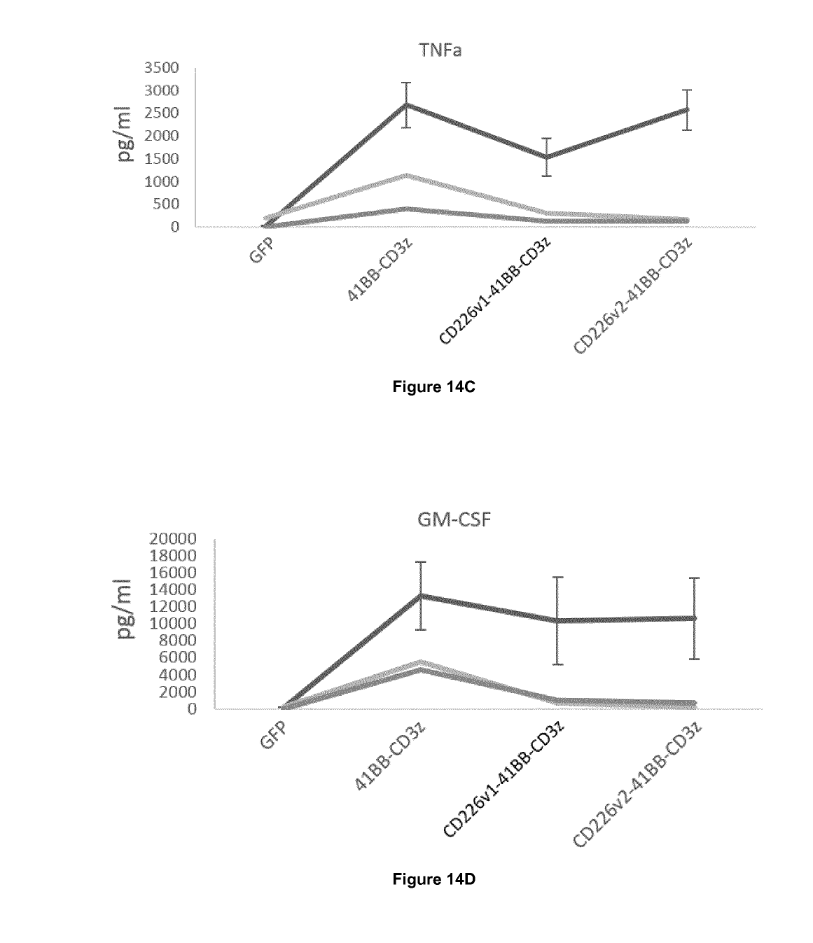

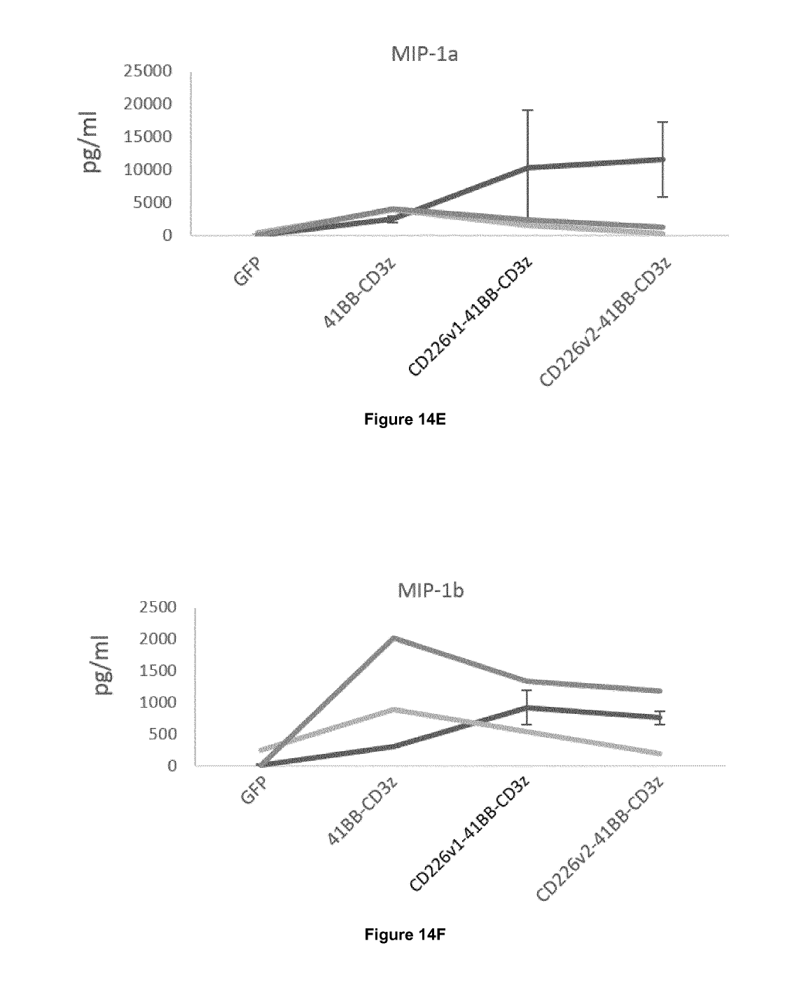

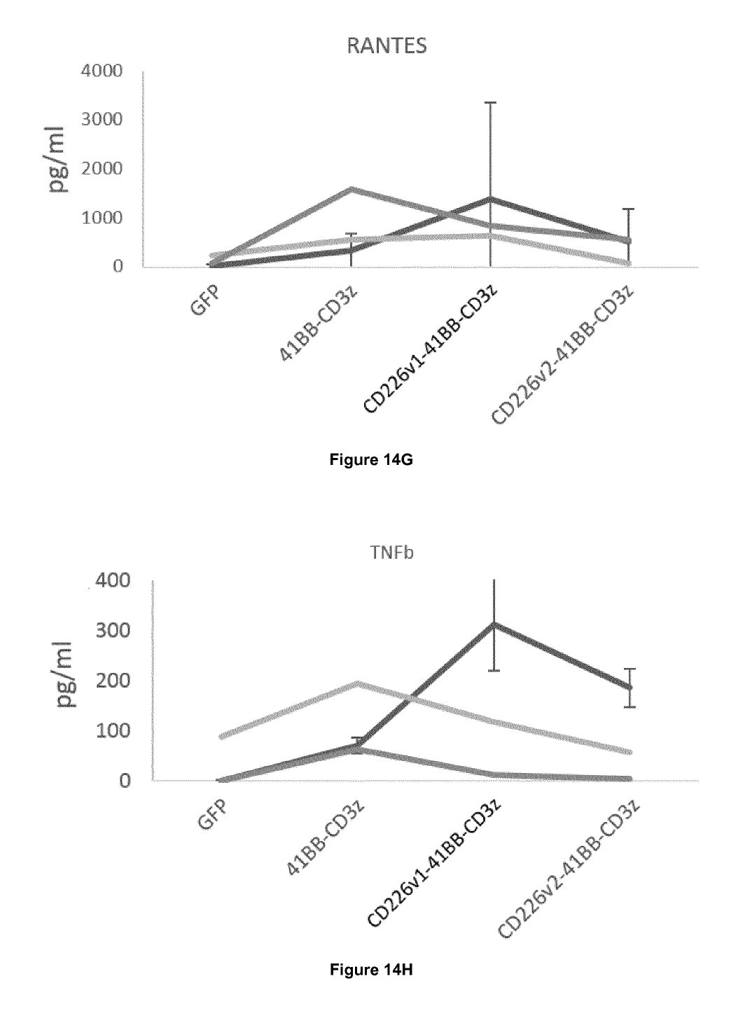

The results obtained using T cells from three different donors are shown in FIGS. 13A to 13H and 14A to 14H. Overall, higher levels of the indicated cytokines were found in co-cultures comprising T cells transduced with the T construct, as compared to co-cultures comprising T cells transduced with the W and X constructs.

### Example 15: Proliferation of GPC3-CAR Expressing CAR-T Cells

Proliferation of T cells transduced with different GPC3-CAR constructs was analysed following coculture with HepG2 cells for 5 days, or following culture for the same period in the absence of HepG2 cells.

Briefly, T cells were labelled with CFSE, a fluorescent label whose intensity is halved each time a labelled cell divide in 2. After labelling, T cells were analysed to ensure uniform labelling. HepG2 cells were irradiated to prevent further proliferation and co-incubated with labelled T cells. After 5 days, T cells were analysed by flow cytometry. Cells with fluorescence approximately equal to the original fluorescence were determined to be non-proliferating cells, and those cells with half or less than half of the original fluorescence intensity were determined to be proliferating cells.

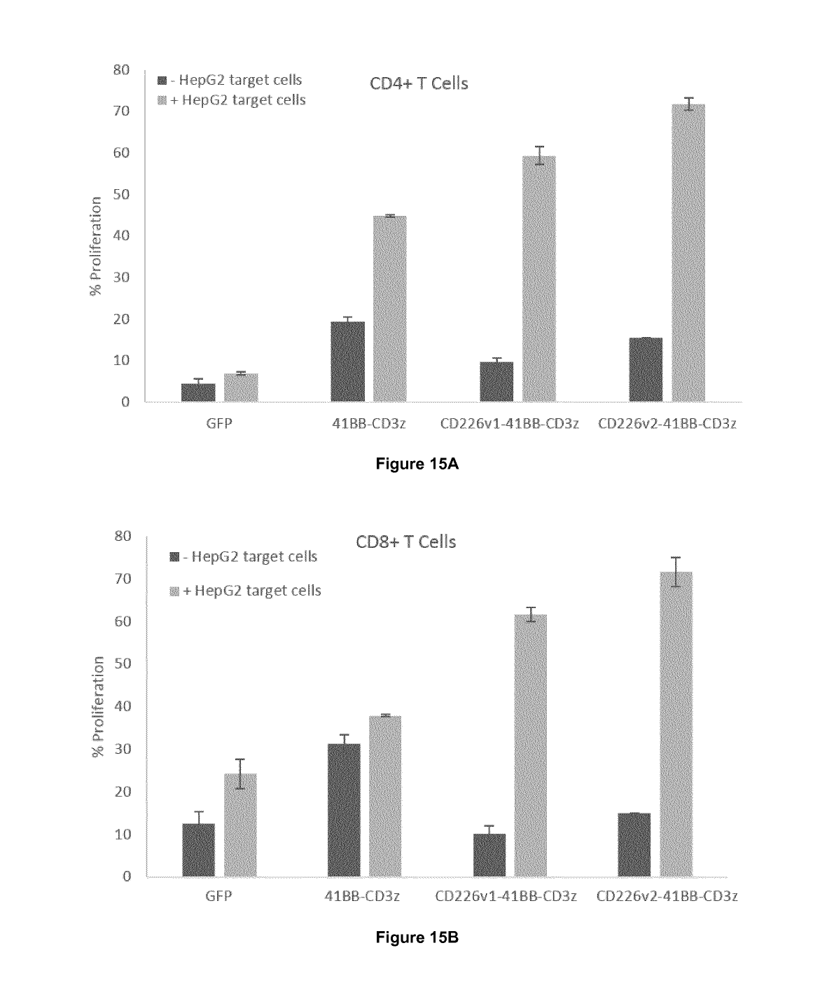

FIGS. 15A and 15B shows the results of proliferation assays performed with T cells from donor ID4, performed 8 days after transduction with construct T, W or X constructs. T cells transduced with the W and X constructs were found to proliferate more following coculture with HepG2 cells as compared to T cells transduced with the T construct. T cells transduced with the W and X constructs were also found to proliferate more less than T cells transduced with the T construct in the absence of HepG2 cells.

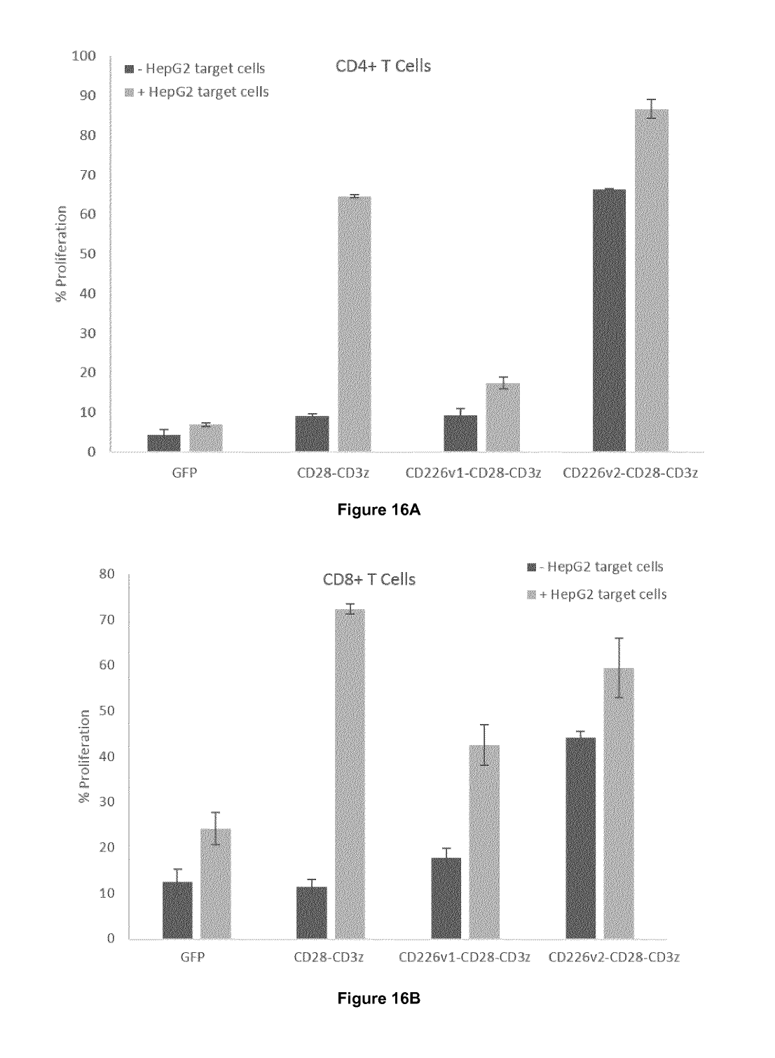

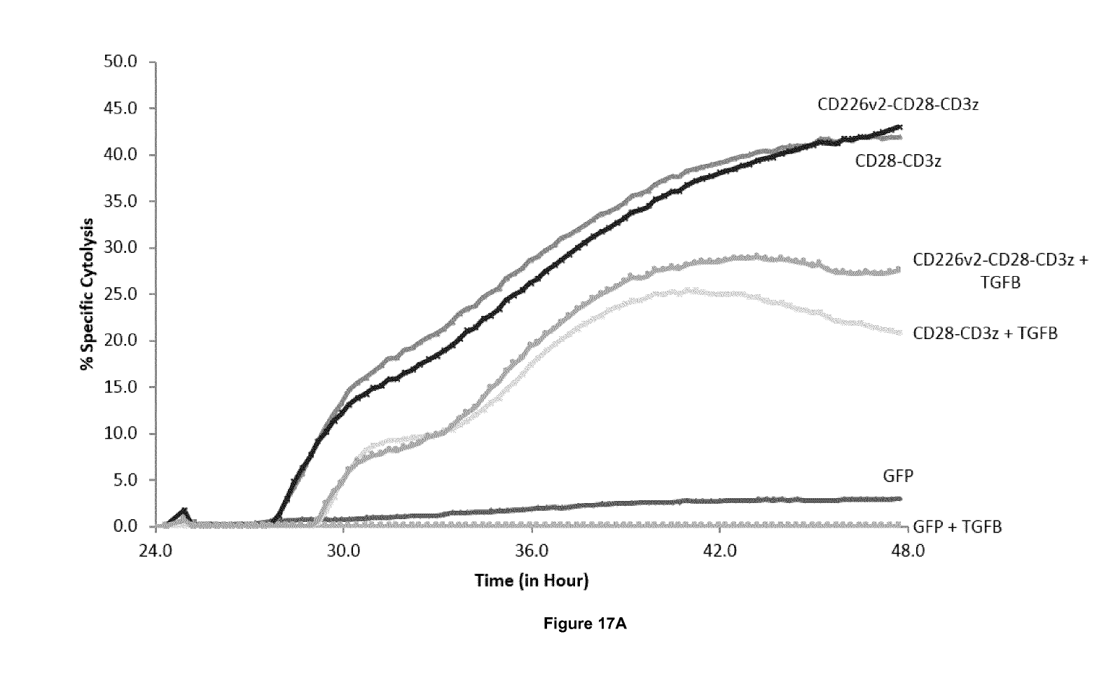

FIGS. 16A and 16B shows the results of proliferation assays performed with T cells from donor ID4, performed 8 days after transduction with construct S, AA or BB. CD4+ T cells transduced with the BB construct were found to proliferate more following coculture with HepG2 cells as compared to CD4+ T cells transduced with the S construct. T cells transduced with the BB construct were also found to display substantial proliferation in the absence of HepG2 cells.

### Example 16: Sensitivity to TGFβ

T cells transduced with different GPC3-CAR constructs were analysed for their sensitivity to immunosuppression by TGFβ.

Briefly, HepG2 cells were seeded on xCELLigence plates, and after 24 hours, T cells transduced with the GFP construct (negative control), or transduced with constructs S or BB, were added to wells in the presence or absence of 125 ng/ml TGFβ, and cytolysis was measured using the xCELLigence (ACEA Biosciences Inc) system.

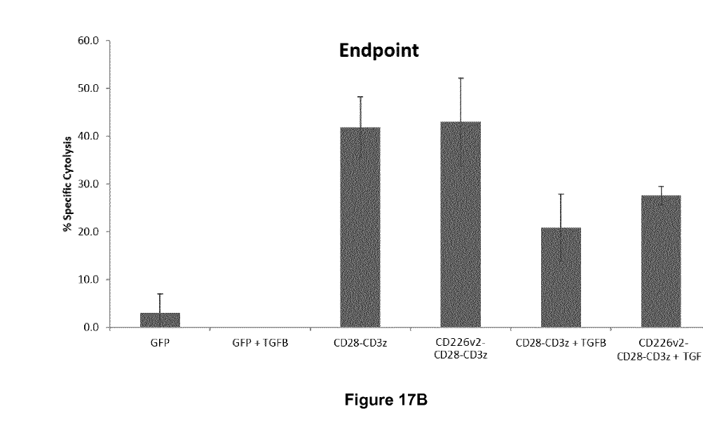

The results are shown in FIGS. 17A and 17B. T cells transduced with the BB construct were found to be less sensitive to TGFβ-mediated suppression of cytolytic activity as compared to d T cells transduced with the S construct (compare FIG. 17B bars 3 and 5 with columns 4 and 6).

### Example 17: Conclusions

Unexpectedly, T cells expressing CARs comprising CD226 intracellular domains were found to display enhanced cytotoxicity against target antigen-expressing cells as compared to T cells expressing equivalent CAR lacking a CD226 intracellular domain, whilst at the same time producing reduced levels of proinflammatory/effector cytokines in co-cultures with target antigen-expressing cells. Furthermore, T cells expressing CARs comprising CD226 intracellular domains were found to proliferate more following coculture with target-antigen expressing cells as compared to T cells expressing equivalent CAR lacking a CD226 intracellular domain.

For example, T cells transduced with constructs W and X displayed enhanced cytotoxicity against target antigen-expressing cells, and increased proliferation following coculture with target antigen expressing cells, as compared to T cells expressing construct T.

T cells transduced with construct BB displayed enhanced cytotoxicity against target antigen-expressing cells as compared to T cells expressing construct S, and were less susceptible to TGFβ-mediated suppression of effector function.

1. A chimeric antigen receptor (CAR), comprising a costimulatory sequence which is, or which is derived from, the intracellular domain of CD226, or a fragment thereof.

2. The CAR according to claim 1, wherein the costimulatory sequence which is, or which is derived from, the intracellular domain of CD226, or a fragment thereof comprises or consists of an amino acid sequence having at least 80% sequence identity to the amino acid sequence of SEQ ID NO:16, 58 or 59.

3. The CAR according to claim 1 or claim 2, wherein the CAR additionally comprises a costimulatory sequence which comprises or consists of an amino acid sequence which is, or which is derived from, the intracellular domain of CD28.

4. The CAR according to any one of claims 1 to 3, wherein the CAR additionally comprises a costimulatory sequence which comprises or consists of an amino acid sequence which is, or which is derived from, the intracellular domain of 4-1BB.

5. The CAR according to any one of claims claim 1 to 4, wherein the CAR comprises a costimulatory sequence which comprises or consists of an amino acid sequence having at least 80% sequence identity to the amino acid sequence of SEQ ID NO:17.

6. The CAR according to any one of claims 1 to 5, wherein the CAR comprises a costimulatory sequence which comprises or consists of an amino acid sequence having at least 80% sequence identity to the amino acid sequence of SEQ ID NO:18.

7. The CAR according to any one of claims 1 to 6, wherein the CAR additionally comprises a dimerization domain.

8. The CAR according to claim 7, wherein the dimerization domain is an inducible dimerization domain.

9. The CAR according to claim 7 or claim 8, wherein the dimerization domain comprises or consists of an amino acid sequence having at least 80% sequence identity to the amino acid sequence of SEQ ID NO:20.

10. The CAR according to any one of claims 1 to 9, wherein the CAR comprises a transmembrane domain which comprises or consists of an amino acid sequence which is, or which is derived from, the transmembrane domain of CD28, CD8a or CD226.

11. The CAR according to any one of claims 1 to 10, wherein the CAR comprises a transmembrane domain which comprises or consists of an amino acid sequence having at least 80% sequence identity to the amino acid sequence of SEQ ID NO:11, 10 or 57.

12. The CAR according to any one of claims 1 to 11, wherein the CAR additionally comprises a hinge region which is, or which is derived from, the human IgG1 hinge region.

13. The CAR according to claim 12, wherein the hinge region comprises or consists of an amino acid sequence having at least 80% sequence identity to the amino acid sequence of SEQ ID NO:19.

14. The CAR according to any one of claims 1 to 13, wherein the CAR comprises an antigen-binding domain which comprises:
a heavy chain variable region sequence having at least 85% sequence identity to the amino acid sequence of SEQ ID NO:1, and
a light chain variable region sequence having at least 85% sequence identity to the amino acid sequence of SEQ ID NO:5.

15. The CAR according to any one of claims 1 to 13, wherein the CAR comprises an antigen-binding domain which comprises:
a heavy chain variable region sequence having at least 85% sequence identity to the amino acid sequence of SEQ ID NO:48, and
a light chain variable region sequence having at least 85% sequence identity to the amino acid sequence of SEQ ID NO:52.

16. A chimeric antigen receptor (CAR) according to any one of A, B, C, D, E, F, G or H, I, J, K, L or M as shown in Table 1, or V, W, X, Z, AA, BB, CC, DD, EE, FF, GG, HH, II, JJ, KK, LL or MM as shown in Table 3.

17. A chimeric antigen receptor (CAR) comprising, or consisting of, an amino acid sequence having at least 60% sequence identity to the amino acid sequence of SEQ ID NO:22, 23, 24, 25, 26, 27, 28, 29, 38, 39, 40, 41, 42, 81, 83, 84, 85, 86, 88, 89, 90, 92, 93, 94, 95, 96, 97 or 98.

18. A chimeric antigen receptor (CAR) comprising, or consisting of, an amino acid sequence having at least 60% sequence identity to the amino acid sequence of SEQ ID NO:30, 31, 32, 33, 34, 35, 36, 37, 43, 44, 45, 46, 47, 62, 64, 65, 66, 67, 69, 70, 71, 73, 74, 75, 76, 77, 78 or 79.

19. A nucleic acid encoding the chimeric antigen receptor (CAR) according to any one of claims 1 to 18.

20. A vector comprising the nucleic acid of claim 19.

21. A cell comprising the chimeric antigen receptor (CAR) according to any one of claims 1 to 18, the nucleic acid according to claim 19, or the vector according to claim 20.

22. A method for producing a cell expressing a chimeric antigen receptor (CAR), comprising introducing into a cell a nucleic acid according to claim 19, or a vector according to claim 20, and culturing the cell under conditions suitable for expression of the nucleic acid or vector by the cell.

23. A cell which is obtained or obtainable by the method according to claim 22.

24. A pharmaceutical composition comprising a chimeric antigen receptor (CAR) according to any one of claims 1 to 18, a nucleic acid according to claim 19, a vector according to claim 20, or a cell according to claim 21 or claim 23, and a pharmaceutically acceptable carrier, adjuvant, excipient, or diluent.

25. A chimeric antigen receptor (CAR) according to any one of claims 1 to 18, a nucleic acid according to claim 19, a vector according to claim 20, a cell according to claim 21 or claim 23, or a pharmaceutical composition according to claim 24, for use in a method of treating or preventing a disease or disorder.

26. Use of a chimeric antigen receptor (CAR) according to any one of claims 1 to 18, a nucleic acid according to claim 19, a vector according to claim 20, a cell according to claim 21 or claim 23, or a pharmaceutical composition according to claim 24, in the manufacture of a medicament for treating or preventing a disease or disorder.

27. A method of treating or preventing a disease or disorder, comprising administering to a subject a therapeutically or prophylactically effective amount of a chimeric antigen receptor (CAR) according to any one of claims 1 to 18, a nucleic acid according to claim 19, a vector according to claim 20, a cell according to claim 21 or claim 23, or a pharmaceutical composition according to claim 24.

28. A method of treating or preventing a disease or disorder in a subject, comprising:
(a) isolating at least one T cell from a subject;
(b) modifying the at least one T cell to express or comprise a chimeric antigen receptor (CAR) according to any one of claims 1 to 18, a nucleic acid according to claim 19, or a vector according to claim 20, and;
(c) administering the modified at least one T cell to a subject.

29. A method of treating or preventing a disease or disorder in a subject, comprising:
(a) isolating at least one T cell from a subject;
(b) introducing into the at least one T cell a nucleic acid according to claim 19, or a vector according to claim 20, thereby modifying the at least one T cell and;
(c) administering the modified at least one T cell to a subject.

30. The CAR, nucleic acid, vector, cell, or pharmaceutical composition for use according to claim 25, the use according to claim 26, or the method according to any one of claims 27 to 29, wherein the disease or disorder is a cancer.

31. The CAR, nucleic acid, vector, cell, or pharmaceutical composition for use, the use, or the method according to according to claim 30, wherein the cancer is a GPC3-expressing cancer or an EpCAM-expressing cancer.

32. A kit of parts comprising a predetermined quantity of a chimeric antigen receptor (CAR) according to any one of claims 1 to 18, a nucleic acid according to claim 19, a vector according to claim 20, a cell according to claim 21 or claim 23, or a pharmaceutical composition according to claim 24.


SEQUENCE LISTING (126 sequences)

SEQ ID NO: 1 - protein, 115 aa, Artificial Sequence
  GC33 heavy chain variable region sequence
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSA

SEQ ID NO: 2 - protein, 5 aa, Artificial Sequence
  GC33 HC-CDR1
DYEMH

SEQ ID NO: 3 - protein, 17 aa, Artificial Sequence
  GC33 HC-CDR2
ALDPKTGDTAYSQKFKG

SEQ ID NO: 4 - protein, 6 aa, Artificial Sequence
  GC33 HC-CDR3
FYSYTY

SEQ ID NO: 5 - protein, 112 aa, Artificial Sequence
  GC33 light chain variable region sequence
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 6 - protein, 16 aa, Artificial Sequence
  GC33 LC-CDR1
RSSQSLVHSNGNTYLH

SEQ ID NO: 7 - protein, 7 aa, Artificial Sequence
  GC33 LC-CDR2
KVSNRFS

SEQ ID NO: 8 - protein, 9 aa, Artificial Sequence
  GC33 LC-CDR3
SQNTHVPPT

SEQ ID NO: 9 - protein, 242 aa, Artificial Sequence
  GC33 scFv
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 10 - protein, 24 aa, Artificial Sequence
  CD28 transmembrane domain
FWVLVVVGGVLACYSLLVTVAFII

SEQ ID NO: 11 - protein, 28 aa, Artificial Sequence
  CD8alpha transmembrane domain
IYIWAPLAGTCGVLLLSLVITLYCNHRN

SEQ ID NO: 12 - protein, 4 aa, Artificial Sequence
  ITAM motif
  misc_feature (2)..(3): Xaa can be any naturally occurring amino acid
  MISC_FEATURE (4)..(4): Xaa = Leu or Ile
YXXX

SEQ ID NO: 13 - protein, 11 aa, Artificial Sequence
  ITAM motif
  misc_feature (2)..(3): Xaa can be any naturally occurring amino acid
  MISC_FEATURE (4)..(4): Xaa = Leu or Ile
  misc_feature (5)..(7): Xaa can be any naturally occurring amino acid
  misc_feature (9)..(10): Xaa can be any naturally occurring amino acid
  MISC_FEATURE (11)..(11): Xaa = Leu or Ile
YXXXXXXYXXX

SEQ ID NO: 14 - protein, 112 aa, Artificial Sequence
  CD3-zeta intracellular domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 336 aa, Artificial Sequence
  Human CD226; UniProt Q15762 (CD226_HUMAN)
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 16 - protein, 66 aa, Artificial Sequence
  CD226 intracellular domain (amino acid positions 271 to 336 of UniProt Q15762)
IVIFLNRRRRRERRDLFTESWDTQKAPNNYRSPISTSQPTNQSMDDTREDIYVNYPTFSRRPKTRV

SEQ ID NO: 17 - protein, 44 aa, Artificial Sequence
  CD28 intracellular domain
FWVRSKRSRLLHSDYMNMTPRRPGPTRKHYQPYAPPRDFAAYRS

SEQ ID NO: 18 - protein, 42 aa, Artificial Sequence
  4-1BB intracellular domain
KRGRKKLLYIFKQPFMRPVQTTQEEDGCSCRFPEEEEGGCEL

SEQ ID NO: 19 - protein, 15 aa, Artificial Sequence
  Human IgG1 hinge region
EPKSCDKTHTCPPCP

SEQ ID NO: 20 - protein, 107 aa, Artificial Sequence
  F36V-FKBP dimerization sequence
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 21 - protein, 19 aa, Artificial Sequence
  Human Ig heavy chain signal sequence
MDWIWRILFLVGAATGAHS

SEQ ID NO: 22 - protein, 466 aa, Artificial Sequence
  scFV GC33/hIgG1 hinge/CD8alpha TMD/CD226/ CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNIVIFLNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 573 aa, Artificial Sequence
  scFV GC33/hIgG1 hinge/CD8alpha TMD/F36V-FKBP/ CD226/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEIVIFLNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 510 aa, Artificial Sequence
  scFV GC33/hIgG1 hinge/CD8alpha TMD/CD226/CD28/ CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNIVIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 617 aa, Artificial Sequence
  scFV GC33/hIgG1 hinge/CD8alpha TMD/F36V-FKBP/ CD226/CD28/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEIVIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 508 aa, Artificial Sequence
  scFV GC33/hIgG1 hinge/CD8alpha TMD/CD226/4-1BB/ CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNIVIFLNRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 615 aa, Artificial Sequence
  scFV GC33/hIgG1 hinge/CD8alpha TMD/F36V-FKBP/ CD226/41BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEIVIFLNRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 552 aa, Artificial Sequence
  scFV GC33/hIgG1 hinge/CD8alpha TMD/CD226/CD28/ 4-1BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNIVIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 659 aa, Artificial Sequence
  scFV GC33/hIgG1 hinge/CD8alpha TMD/F36V-FKBP/CD226/CD28/41BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEIVIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 485 aa, Artificial Sequence
  hlgG heavy chain signal sequence/scFV GC33/ hIgG1 hinge/CD8alpha TMD/CD226/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNIVIFLNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 592 aa, Artificial Sequence
  hIgG heavy chain signal sequence/scFV GC33/ hIgG1 hinge/CD8alpha TMD/F36V-FKBP/CD226/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEIVIFLNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 529 aa, Artificial Sequence
  hIgG heavy chain signal sequence/scFV GC33/ hIgG1 hinge/CD8alpha TMD/CD226/CD28/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNIVIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 636 aa, Artificial Sequence
  hIgG heavy chain signal sequence/scFV GC33/ hIgG1 hinge/CD8alpha TMD/F36V-FKBP/CD226/CD28/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEIVIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 527 aa, Artificial Sequence
  hIgG heavy chain signal sequence/scFV GC33/ hIgG1 hinge/CD8alpha TMD/CD226/4-1BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNIVIFLNRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 634 aa, Artificial Sequence
  hIgG heavy chain signal sequence/scFV GC33/ hIgG1 hinge/CD8alpha TMD/F36V-FKBP/CD226/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEIVIFLNRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 571 aa, Artificial Sequence
  hIgG heavy chain signal sequence/scFV GC33/ hIgG1 hinge/CD8alpha TMD/CD226/CD28/4-1BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNIVIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 678 aa, Artificial Sequence
  hIgG heavy chain signal sequence/scFV GC33/ hIgG1 hinge/CD8alpha TMD/F36V-FKBP/CD226/CD28/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEIVIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 545 aa, Artificial Sequence
  scFV GC33/hIgG1 hinge/CD28 TMD/F36V-FKBP/41BB/ CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 442 aa, Artificial Sequence
  scFV GC33/hIgG1 hinge/CD8alpha TMD/41BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 549 aa, Artificial Sequence
  scFV GC33/hIgG1 hinge/CD8alpha TMD/F36V-FKBP/ 41BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 444 aa, Artificial Sequence
  scFV GC33/hIgG1 hinge/CD8alpha TMD/CD28/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 551 aa, Artificial Sequence
  scFV GC33/hIgG1 hinge/CD8alpha TMD/F36V-FKBP/ CD28/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 564 aa, Artificial Sequence
  hIgG heavy chain signal sequence/scFV GC33/ hIgG1 hinge/CD28 TMD/F36V-FKBP/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 461 aa, Artificial Sequence
  hIgG heavy chain signal sequence/scFV GC33/ hIgG1 hinge/CD8alpha TMD/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 568 aa, Artificial Sequence
  hIgG heavy chain signal sequence/scFV GC33/ hIgG1 hinge/CD8alpha TMD/F36V-FKBP/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 463 aa, Artificial Sequence
  hIgG heavy chain signal sequence/scFV GC33/ hIgG1 hinge/CD8alpha TMD/CD28/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 570 aa, Artificial Sequence
  hIgG heavy chain signal sequence/scFV GC33/ hIgG1 hinge/CD8alpha TMD/F36V-FKBP/CD28/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKIYIWAPLAGTCGVLLLSLVITLYCNHRNGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 113 aa, Artificial Sequence
  3-17I heavy chain variable region sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTV

SEQ ID NO: 49 - protein, 5 aa, Artificial Sequence
  3-17I HC-CDR1
SYAIS

SEQ ID NO: 50 - protein, 17 aa, Artificial Sequence
  3-17I HC-CDR2
GIIPIFGTANYAQKFQG

SEQ ID NO: 51 - protein, 6 aa, Artificial Sequence
  3-17I HC-CDR3
GLLWNY

SEQ ID NO: 52 - protein, 109 aa, Artificial Sequence
  3-17I light chain variable region sequence
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIK

SEQ ID NO: 53 - protein, 11 aa, Artificial Sequence
  3-17I LC-CDR1
RASQSVSSNLA

SEQ ID NO: 54 - protein, 7 aa, Artificial Sequence
  3-17I LC-CDR2
GASTTAS

SEQ ID NO: 55 - protein, 11 aa, Artificial Sequence
  3-17I LC-CDR3
QQYNNWPPAYT

SEQ ID NO: 56 - protein, 245 aa, Artificial Sequence
  3-17I scFv
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSKLSGSASAPKLEEGEFSEARVEIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIK

SEQ ID NO: 57 - protein, 21 aa, Artificial Sequence
  CD226 TMD
GGTVLLLLFVISITTIIVIFL

SEQ ID NO: 58 - protein, 61 aa, Artificial Sequence
  CD226 ICD v1
NRRRRRERRDLFTESWDTQKAPNNYRSPISTSQPTNQSMDDTREDIYVNYPTFSRRPKTRV

SEQ ID NO: 59 - protein, 63 aa, Artificial Sequence
  CD226 ICD v2
FLNRRRRRERRDLFTESWDTQKAPNNYRSPISTSQPTNQSMDDTREDIYVNYPTFSRRPKTRV

SEQ ID NO: 60 - protein, 21 aa, Artificial Sequence
  CD226 TMD
GGTVLLLLFVISITTIIVIFL

SEQ ID NO: 61 - protein, 415 aa, Artificial Sequence
  hIgG heavy chain signal sequence/scFV GC33/ hIgG1 hinge/CD28 TMD/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 366 aa, Artificial Sequence
  hIgG heavy chain signal sequence/scFV GC33/ hIgG1 hinge/CD28 TMD/CD226 ICD v2
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFLNRRRRRERRDLFTESWDTQKAPNNYRSPISTSQPTNQSMDDTREDIYVNYPTFSRRPKTRV

SEQ ID NO: 63 - protein, 457 aa, Artificial Sequence
  hIgG heavy chain signal sequence/scFV GC33/ hIgG1 hinge/CD28 TMD/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 518 aa, Artificial Sequence
  hIgG heavy chain signal sequence/scFV GC33/ hIgG1 hinge/CD28 TMD/CD226 ICD v1/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIINRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 520 aa, Artificial Sequence
  hIgG heavy chain signal sequence/scFV GC33/ hIgG1 hinge/CD28 TMD/CD226 ICD v2/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFLNRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 476 aa, Artificial Sequence
  hIgG heavy chain signal sequence/scFV GC33/ hIgG1 hinge/CD28 TMD/CD226 ICD v1/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIINRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 478 aa, Artificial Sequence
  hIgG heavy chain signal sequence/scFV GC33/ hIgG1 hinge/CD28 TMD/CD226 ICD v2/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFLNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 459 aa, Artificial Sequence
  hIgG heavy chain signal sequence/scFV GC33/ hIgG1 hinge/CD28 TMD/CD28 ICD/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 520 aa, Artificial Sequence
  hIgG heavy chain signal sequence/scFV GC33/ hIgG1 hinge/CD28 TMD/CD226 ICD v1/ CD28 ICD/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIINRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 522 aa, Artificial Sequence
  hIgG heavy chain signal sequence/scFV GC33/ hIgG1 hinge/CD28 TMD/CD226 ICD v2/ CD28 ICD/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 520 aa, Artificial Sequence
  hIgG heavy chain signal sequence/scFV GC33/ hIgG1 hinge/CD28 TMD/CD28 ICD/CD226 ICD v1/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFWVRSKRSRLLHSDYMNMTPRRPGPTRKHYQPYAPPRDFAAYRSNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 501 aa, Artificial Sequence
  hIgG heavy chain signal sequence/scFV GC33/ hIgG1 hinge/CD28 TMD/CD28 ICD/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 562 aa, Artificial Sequence
  hIgG heavy chain signal sequence/scFV GC33/ hIgG1 hinge/CD28 TMD/CD226 ICD v1/ CD28 ICD/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIINRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 564 aa, Artificial Sequence
  hIgG heavy chain signal sequence/scFV GC33/ hIgG1 hinge/CD28 TMD/CD226 ICD v2/ CD28 ICD/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 562 aa, Artificial Sequence
  hIgG heavy chain signal sequence/scFV GC33/ hIgG1 hinge/CD28 TMD/CD28 ICD/CD226 ICD v1/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFWVRSKRSRLLHSDYMNMTPRRPGPTRKHYQPYAPPRDFAAYRSNRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 515 aa, Artificial Sequence
  hIgG heavy chain signal sequence/scFV GC33/ hIgG1 hinge/CD226 TMD/CD226 ICD v1/41BB/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKGGTVLLLLFVISITTIIVIFLNRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 364 aa, Artificial Sequence
  hIgG heavy chain signal sequence/scFV 3-17I/ hIgG1 hinge/CD226 TMD/CD226 ICD v1
MDWIWRILFLVGAATGAHSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSKLSGSASAPKLEEGEFSEARVEIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKEPKSCDKTHTCPPCPDPKGGTVLLLLFVISITTIIVIFLNRRRRRERRDLFTESWDTQKAPNNYRSPISTSQPTNQSMDDTREDIYVNYPTFSRRPKTRV

SEQ ID NO: 78 - protein, 415 aa, Artificial Sequence
  hIgG heavy chain signal sequence/scFV 3-17I/ hIgG1 hinge/CD226 TMD/CD3zeta
MDWIWRILFLVGAATGAHSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSKLSGSASAPKLEEGEFSEARVEIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKEPKSCDKTHTCPPCPDPKGGTVLLLLFVISITTIIVIFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 476 aa, Artificial Sequence
  hIgG heavy chain signal sequence/scFV 3-17I/ hIgG1 hinge/CD226 TMD/CD226 ICD v1/CD3zeta
MDWIWRILFLVGAATGAHSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSKLSGSASAPKLEEGEFSEARVEIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKEPKSCDKTHTCPPCPDPKGGTVLLLLFVISITTIIVIFLNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 396 aa, Artificial Sequence
  scFV GC33/hIgG1 hinge/CD28 TMD/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 347 aa, Artificial Sequence
  scFV GC33/hIgG1 hinge/CD28 TMD/CD226 ICD v2
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFLNRRRRRERRDLFTESWDTQKAPNNYRSPISTSQPTNQSMDDTREDIYVNYPTFSRRPKTRV

SEQ ID NO: 82 - protein, 438 aa, Artificial Sequence
  scFV GC33/hIgG1 hinge/CD28 TMD/41BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 499 aa, Artificial Sequence
  scFV GC33/hIgG1 hinge/CD28 TMD/ CD226 ICD v1/ 41BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIINRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 501 aa, Artificial Sequence
  scFV GC33/hIgG1 hinge/CD28 TMD/ CD226 ICD v2/ 41BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFLNRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 457 aa, Artificial Sequence
  scFV GC33/hIgG1 hinge/CD28 TMD/ CD226 ICD v1/ CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIINRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 459 aa, Artificial Sequence
  scFV GC33/hIgG1 hinge/CD28 TMD/ CD226 ICD v2/ CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFLNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 440 aa, Artificial Sequence
  scFV GC33/hIgG1 hinge/CD28 TMD/ CD28 ICD/ CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 501 aa, Artificial Sequence
  scFV GC33/hIgG1 hinge/CD28 TMD/ CD226 ICD v1/ CD28 ICD/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIINRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 503 aa, Artificial Sequence
  scFV GC33/hIgG1 hinge/CD28 TMD/ CD226 ICD v2/ CD28 ICD/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 501 aa, Artificial Sequence
  scFV GC33/hIgG1 hinge/CD28 TMD/ CD28 ICD/CD226 ICD v1/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFWVRSKRSRLLHSDYMNMTPRRPGPTRKHYQPYAPPRDFAAYRSNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 482 aa, Artificial Sequence
  scFV GC33/hIgG1 hinge/CD28 TMD/ CD28 ICD/41BB/ CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 543 aa, Artificial Sequence
  scFV GC33/hIgG1 hinge/CD28 TMD/ CD226 ICD v1/ CD28 ICD/41BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIINRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 545 aa, Artificial Sequence
  scFV GC33/hIgG1 hinge/CD28 TMD/ CD226 ICD v2/ CD28 ICD/41BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFLNRRRRRERRDLFTESWDTQKAPNNYRSPISTSQPTNQSMDDTREDIYVNYPTFSRRPKTRVFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 543 aa, Artificial Sequence
  scFV GC33/hIgG1 hinge/CD28 TMD/ CD28 ICD/CD226 ICD v1/41BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIFWVRSKRSRLLHSDYMNMTPRRPGPTRKHYQPYAPPRDFAAYRSNRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 496 aa, Artificial Sequence
  scFV GC33/hIgG1 hinge/CD226 TMD/CD226 ICD v1/ 41BB/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKGGTVLLLLFVISITTIIVIFLNRRRRRERRDLFTESWDTQKAPNNYRSPISTSQPTNQSMDDTREDIYVNYPTFSRRPKTRVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 345 aa, Artificial Sequence
  scFV 3-17I/hIgG1 hinge/CD226 TMD/CD226 ICD v1
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSKLSGSASAPKLEEGEFSEARVEIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKEPKSCDKTHTCPPCPDPKGGTVLLLLFVISITTIIVIFLNRRRRRERRDLFTESWDTQKAPNNYRSPISTSQPTNQSMDDTREDIYVNYPTFSRRPKTRV

SEQ ID NO: 97 - protein, 396 aa, Artificial Sequence
  scFV 3-17I/hIgG1 hinge/CD226 TMD/ CD3zeta
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSKLSGSASAPKLEEGEFSEARVEIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKEPKSCDKTHTCPPCPDPKGGTVLLLLFVISITTIIVIFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 457 aa, Artificial Sequence
  scFV 3-17I/hIgG1 hinge/CD226 TMD/CD226 ICD v1/ CD3zeta
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSKLSGSASAPKLEEGEFSEARVEIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKEPKSCDKTHTCPPCPDPKGGTVLLLLFVISITTIIVIFLNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - DNA, 1254 bp, Artificial Sequence
  (nucleotide) hIgG heavy chain signal sequence/ scFV GC33/hIgG1 hinge/CD28 TMD/CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgcccggatccgaagttctgggtgctggtggtggtcgggggagtgctcgcctgctactctctgctggtgaccgtggccttcatcatccgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgaacgcgt

SEQ ID NO: 100 - DNA, 1107 bp, Artificial Sequence
  (nucleotide) hIgG heavy chain signal sequence/ scFV GC33/hIgG1 hinge/CD28 TMD/CD226 ICD v2
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgtccggatccgaagttctgggtgctggtggtggtcgggggagtgctcgcctgctactctctgctggtgaccgtggcattcattatcttcctgaacagaaggaggcgccgggagcggcgcgacctgttcactgaatcctgggacacccagaaggcccccaacaactacaggtcccctatctcaacctcccaacccaccaaccagagcatggacgatactcgcgaggacatctacgtgaactaccccactttctcccggcggcctaagacacgggtgtgaacgcgt

SEQ ID NO: 101 - DNA, 1380 bp, Artificial Sequence
  (nucleotide) hIgG heavy chain signal sequence/ scFV GC33/hIgG1 hinge/CD28 TMD/41BB/CD3zeta
atggactggatctggaggattttgttccttgtgggagctgccaccggtgcacattcgcaggtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgcccggatccaaagttctgggtgctggtggtggtcgggggagtgctcgcctgctactctctgctggtgaccgtggcctttataatcaagcgcggtcggaagaagctgctgtacatcttcaagcagcccttcatgcggcctgtgcagaccacacaggaagaggatggctgctcctgccgcttcccggaggaagaggagggcggatgcgaactgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgaacgcgt

SEQ ID NO: 102 - DNA, 1563 bp, Artificial Sequence
  (nucleotide) hIgG heavy chain signal sequence/ scFV GC33/hIgG1 hinge/CD28 TMD/ CD226 ICD v1/41BB/CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgtccggatccgaagttctgggtgctggtggtggtcgggggagtgctcgcctgctactctctgctggtgaccgtggcattcattatcaacagaaggaggcgccgggagcggcgcgacctgttcactgaatcctgggacacccagaaggcccccaacaactacaggtcccctatctcaacctcccaacccaccaaccagagcatggacgatactcgcgaggacatctacgtgaactaccccactttctcccggcggcctaagacccgcgtgaagcgcggtcggaagaagctgctgtacatcttcaagcagcccttcatgcggcctgtgcagaccacacaggaagaggatggctgctcctgccgcttcccggaggaagaggagggcggatgcgaactgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgcctagatgaacgcgt

SEQ ID NO: 103 - DNA, 1569 bp, Artificial Sequence
  (nucleotide) hIgG heavy chain signal sequence/ scFV GC33/hIgG1 hinge/CD28 TMD/ CD226 ICD v2/41BB/CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgtccggatccgaagttctgggtgctggtggtggtcgggggagtgctcgcctgctactctctgctggtgaccgtggcattcattatcttcctgaacagaaggaggcgccgggagcggcgcgacctgttcactgaatcctgggacacccagaaggcccccaacaactacaggtcccctatctcaacctcccaacccaccaaccagagcatggacgatactcgcgaggacatctacgtgaactaccccactttctcccggcggcctaagacccgcgtgaagcgcggtcggaagaagctgctgtacatcttcaagcagcccttcatgcggcctgtgcagaccacacaggaagaggatggctgctcctgccgcttcccggaggaagaggagggcggatgcgaactgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgcctagatgaacgcgt

SEQ ID NO: 104 - DNA, 1434 bp, Artificial Sequence
  (nucleotide) hIgG heavy chain signal sequence/ scFV GC33/hIgG1 hinge/CD28 TMD/ CD226 ICD v1/CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgcccggatccgaagttttgggtcttggtggtggtgggaggcgtcctcgcctgctacagcttgctcgtgaccgttgccttcatcatcaaccgcaggcggagaagggaacggcgcgacctgttcactgagtcatgggacacccagaaggccccgaacaactaccgctccccgatctccacctcccaaccgactaatcaaagcatggacgacaccagggaggacatctacgtgaactaccctactttctcccgccggcctaagactcgggtgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgatag

SEQ ID NO: 105 - DNA, 1440 bp, Artificial Sequence
  (nucleotide) hIgG heavy chain signal sequence/ scFV GC33/hIgG1 hinge/CD28 TMD/ CD226 ICD v2/CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgcccggatccgaagttctgggtgttggtcgtggtcggaggcgtcctcgcctgctacagcctgctcgtgaccgtggccttcatcatcttcctgaaccgcaggcggagaagggaacggcgcgacctgttcactgagtcatgggacacccagaaggccccgaacaactaccgctccccgatctccacctcccaaccgactaatcaaagcatggacgacaccagggaggacatctacgtgaactaccctactttctcccgccggcctaagactcgggtgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgatag

SEQ ID NO: 106 - DNA, 1383 bp, Artificial Sequence
  (nucleotide) hIgG heavy chain signal sequence/ scFV GC33/hIgG1 hinge/CD28 TMD/ CD28 ICD/CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgcccggatccgaagttctgggtcttggtggtcgtgggaggcgtcctcgcctgctacagcctcctcgtgaccgtcgccttcatcatcttctgggtgcgctccaagcgctccagactgctgcacagcgactacatgaacatgaccccaagacgcccaggacctactaggaagcattatcaaccttatgccccgccgagagacttcgcggcgtaccggtcccgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgatag

SEQ ID NO: 107 - DNA, 1566 bp, Artificial Sequence
  (nucleotide) hIgG heavy chain signal sequence/ scFV GC33/hIgG1 hinge/CD28 TMD/ CD226 ICD v1/ CD28 ICD/CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgcccggatccgaagttttgggtgttggtggtcgtgggaggcgtcctcgcgtgctacagcctcctcgtgaccgtcgccttcatcatcaaccgcaggcggagaagggaacggcgcgacctgttcactgagtcatgggacacccagaaggccccgaacaactaccgctccccgatctccacctcccaaccgactaatcaaagcatggacgacaccagggaggacatctacgtgaactaccctactttctcccgccggcctaagacccgcgtgttctgggtgcgctccaagcgctccagactgctgcacagcgactacatgaacatgaccccaagacgcccaggacctactaggaagcattatcaaccttatgccccgccgagagacttcgcggcgtaccggtcccgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgatag

SEQ ID NO: 108 - DNA, 1572 bp, Artificial Sequence
  (nucleotide) hIgG heavy chain signal sequence/ scFV GC33/hIgG1 hinge/CD28 TMD/ CD226 ICD v2/ CD28 ICD/CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgcccggatccgaagttctgggtcttggtggtggtcggaggcgtcctcgcttgttacagcctgctcgtgaccgttgccttcatcatcttcctgaaccgcaggcggagaagggaacggcgcgacctgttcactgagtcatgggacacccagaaggccccgaacaactaccgctccccgatctccacctcccaaccgactaatcaaagcatggacgacaccagggaggacatctacgtgaactaccctactttctcccgccggcctaagacccgcgtgttctgggtgcgctccaagcgctccagactgctgcacagcgactacatgaacatgaccccaagacgcccaggacctactaggaagcattatcaaccttatgccccgccgagagacttcgcggcgtaccggtcccgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgatag

SEQ ID NO: 109 - DNA, 1566 bp, Artificial Sequence
  (nucleotide) hIgG heavy chain signal sequence/ scFV GC33/hIgG1 hinge/CD28 TMD/ CD28 ICD/CD226 ICD v1/CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgcccggatccgaagttctgggtgttggtggtcgtgggaggcgtcctcgcatgttactccctgctggtgactgtggccttcatcattttttgggtgaggtccaagcggtcgcggctgctgcactccgactacatgaacatgacccccagaagacccggtcctacacggaagcattaccaaccctacgcccccccgagggacttcgccgcctaccggtccaaccgcaggcggagaagggaacggcgcgacctgttcactgagtcatgggacacccagaaggccccgaacaactaccgctccccgatctccacctcccaaccgactaatcaaagcatggacgacaccagggaggacatctacgtgaactaccctactttctcccgccggcctaagacccgcgtgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgatag

SEQ ID NO: 110 - DNA, 1509 bp, Artificial Sequence
  (nucleotide) hIgG heavy chain signal sequence/ scFV GC33/hIgG1hinge/CD28 TMD/ CD28 ICD/41BB/CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgcccggatccgaagttctgggtgctggtggtggtcgggggagtgctcgcctgctactctctgctggtgaccgtggccttcatcatcttttgggtgagatccaagcggtctcgcctgctgcacagcgactacatgaacatgaccccgcgcagacctggcccgactaggaagcactaccagccctacgccccccccagggatttcgccgcctacagatccaagcgcggtcggaagaagctgctgtacatcttcaagcagcccttcatgcggcctgtgcagaccacacaggaagaggatggctgctcctgccgcttcccggaggaagaggagggcggatgcgaactgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgatag

SEQ ID NO: 111 - DNA, 1692 bp, Artificial Sequence
  (nucleotide) hIgG heavy chain signal sequence/ scFV GC33/hIgG1 hinge/CD28 TMD/ CD226 ICD v1/ CD28 ICD/41BB/ CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggacagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgcccggatccgaagttttgggtgttggtggtggtcggaggcgtcctcgcatgctatagcctgctcgtgaccgtggccttcatcatcaaccgcaggcggagaagggaacggcgcgacctgttcactgagtcatgggacacccagaaggccccgaacaactaccgctccccgatctccacctcccaaccgactaatcaaagcatggacgacaccagggaggacatctacgtgaactaccctactttctcccgccggcctaagacccgcgtgttctgggtgcgctccaagcgctccagactgctgcacagcgactacatgaacatgaccccaagacgcccaggacctactaggaagcattatcaaccttatgccccgccgagagacttcgcggcgtataggtccaagcgcggtcggaagaagctgctgtacatcttcaagcagcccttcatgcggcctgtgcagaccacacaggaagaggatggctgctcctgccgcttcccggaggaagaggagggcggatgcgaactgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgatag

SEQ ID NO: 112 - DNA, 1698 bp, Artificial Sequence
  (nucleotide) hIgG heavy chain signal sequence/ scFV GC33/hIgG1 hinge/CD28 TMD/ CD226 ICD v2/ CD28 ICD/41BB/ CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgcccggatccgaagttctgggtcttggtggtggtcggaggcgtcctcgcttgctacagcctgctcgtgaccgtggccttcatcatcttcctgaaccgcaggcggagaagggaacggcgcgacctgttcactgagtcatgggacacccagaaggccccgaacaactaccgctccccgatctccacctcccaaccgactaatcaaagcatggacgacaccagggaggacatctacgtgaactaccctactttctcccgccggcctaagacccgcgtgttctgggtgcgctccaagcgctccagactgctgcacagcgactacatgaacatgaccccaagacgcccaggacctactaggaagcattatcaaccttatgccccgccgagagacttcgcggcgtataggtccaagcgcggtcggaagaagctgctgtacatcttcaagcagcccttcatgcggcctgtgcagaccacacaggaagaggatggctgctcctgccgcttcccggaggaagaggagggcggatgcgaactgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgatag

SEQ ID NO: 113 - DNA, 1692 bp, Artificial Sequence
  (nucleotide) hIgG heavy chain signal sequence/ scFV GC33/hIgG1 hinge/CD28 TMD/ CD28 ICD/CD226 ICD v1/41BB/ CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgtccggatccgaagttctgggtgctggtggtggtcgggggagtgctcgcctgctactctctgctggtgaccgtggcattcattatcttctgggtccgctccaaacggtcccggctgctgcactccgactacatgaacatgaccccgagacggccaggaccgactcgcaagcactaccagccgtacgccccaccgagagacttcgccgcataccgctcaaacagaaggaggcgccgggagcggcgcgacctgttcactgaatcctgggacacccagaaggcccccaacaactacaggtcccctatctcaacctcccaacccaccaaccagagcatggacgatactcgcgaggacatctacgtgaactaccccactttctcccggcggcctaagacccgcgtgaagcgcggtcggaagaagctgctgtacatcttcaagcagcccttcatgcggcctgtgcagaccacacaggaagaggatggctgctcctgccgcttcccggaggaagaggagggcggatgcgaactgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgcctagatgatag

SEQ ID NO: 114 - DNA, 1554 bp, Artificial Sequence
  (nucleotide) hIgG heavy chain signal sequence/ scFV GC33/hIgG1 hinge/CD226 TMD/CD226 ICD v1/41BB/CD3zeta
atggactggatctggaggattttgttccttgtgggagctgccaccggtgcacattcgcaagtgcagttgcagcagtcgggagccgaactggtccggcctggagcttccgtgaagcttagctgcaaggcctccggctacacctttaccgactacgagatgcactgggtcaagcagacccccgtgcacggtctgaagtggattggggccctggatcccaagaccggcgatactgcgtactcacagaagttcaagggaaaggccacgctcactgcggacaaatcgtccagcaccgcgtacatggaactcaggagcctgacttccgaggatagcgcagtgtactactgcacccgcttttactcctacacttactggggacagggcaccttggtgactgtgtcagccggtggaggcggatcagggggtggaggatccgggggaggaggatccgatgtggtcatgacccagaccccactgtcccttcccgtgtccctgggtgaccaagcctcgatcagctgcagatcctcccagtcactggtccacagcaacggcaacacctatctgcattggtacctccagaagccgggacaatcccccaagctcctgatctacaaggtgtccaaccggttcagcggagtgccggatcgattctcagggtcgggttcgggaaccgacttcacccttaagatttcccgggtggaagccgaggatctcggagtgtacttctgctcccaaaatacccacgtgccgcctacattcggatcgggaactaagctggagatcaaggagcccaaaagctgcgacaagacccacacttgcccaccttgtccggatccgaaggggggaactgtgctcctcctgctgttcgtgatttcgatcacgaccatcattgtgatcttcctgaaccgccggcgaagacgcgagcggcgcgacctgttcactgaatcctgggacacccagaaggcccccaacaactacaggtcccctatctcaacctcccaacccaccaaccagagcatggacgatactcgcgaggacatctacgtgaactaccccactttctcccggcggcctaagacccgcgtgaagcgcggtcggaagaagctgctgtacatcttcaagcagcccttcatgcggcctgtgcagaccacacaggaagaggatggctgctcctgccgcttcccggaggaagaggagggcggatgcgaactgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgcctagatgaacgcgt

SEQ ID NO: 115 - DNA, 1101 bp, Artificial Sequence
  (nucleotide) hIgG heavy chain signal sequence/ scFV 3-17I/hIgG1 hinge/CD226 TMD/CD226 ICD v1
atggactggatctggagaattttgttccttgtcggtgctgccactggagcccactcgcaagtgcagctcgtgcagtctggagcagaagtcaagaagcctgggtcctcggtcaaagtgtcctgcaaagcctccgggggcactttcagctcgtacgcaatctcctgggtccgccaagcgcccggacagggtctggagtggatgggcgggattatccccattttcggcaccgccaactatgctcaaaagttccagggccgcgtgactattaccgcggacgagtcaacctccactgcgtacatggaactgagctcacttcggtccgaggacaccgccgtctattactgtgcgcggggactcctgtggaactactggggacaaggcaccctcgtgaccgtgtcctcgaagctgtccgggtcggccagcgcccccaagttggaagagggcgaattctccgaggcccgggtggaaatcgtgatgacccagtccccggccactctctccgtgtccccgggagaaagggcgaccctgtcatgccgggcctcgcaatccgtgtcatccaacctggcctggtaccagcagaagccaggacaggcgccccgcctgattatctacggcgccagcaccaccgcgagcggaattccggcccggttttccgcctccggttcgggaactgacttcacgctgactatcagcagcctgcaatcggaggacttcgccgtctactactgccagcagtataacaactggcccccggcttacacgtttggccagggaactaagctggagatcaaggagcccaaaagctgcgacaagacccacacttgcccaccttgtccggacccgaaggggggaaccgtgctcctgctgctgttcgtgatctccatcaccacaatcatcgtgatcttcctgaaccgccggcgaagacgcgaaagacgcgatctgttcaccgagtcatgggacacccagaaggcccctaacaactacagaagcccgatcagcaccagccagcctactaatcagtcgatggatgatacccgcgaggacatctacgtgaattacccaaccttctcccggcggccgaaaacccgcgtgtgaacgcgt

SEQ ID NO: 116 - DNA, 1254 bp, Artificial Sequence
  (nucleotide) hIgG heavy chain signal sequence/ scFV 3-17I/hIgG1 hinge/CD226 TMD/ CD3zeta
atggactggatctggagaattttgttccttgtcggtgctgccactggagcccactcgcaagtgcagctcgtgcagtctggagcagaagtcaagaagcctgggtcctcggtcaaagtgtcctgcaaagcctccgggggcactttcagctcgtacgcaatctcctgggtccgccaagcgcccggacagggtctggagtggatgggcgggattatccccattttcggcaccgccaactatgctcaaaagttccagggccgcgtgactattaccgcggacgagtcaacctccactgcgtacatggaactgagctcacttcggtccgaggacaccgccgtctattactgtgcgcggggactcctgtggaactactggggacaaggcaccctcgtgaccgtgtcctcgaagctgtccgggtcggccagcgcccccaagttggaagagggcgaattctccgaggcccgggtggaaatcgtgatgacccagtccccggccactctctccgtgtccccgggagaaagggcgaccctgtcatgccgggcctcgcaatccgtgtcatccaacctggcctggtaccagcagaagccaggacaggcgccccgcctgattatctacggcgccagcaccaccgcgagcggaattccggcccggttttccgcctccggttcgggaactgacttcacgctgactatcagcagcctgcaatcggaggacttcgccgtctactactgccagcagtataacaactggcccccggcttacacgtttggccagggaactaagctggagatcaaggagcccaaaagctgcgacaagacccacacttgcccaccttgtccggacccgaaggggggaaccgtgctcctgctgctgttcgtgatctccatcaccacaatcatcgtgatcttcctgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgaacgcgt

SEQ ID NO: 117 - DNA, 1437 bp, Artificial Sequence
  (nucleotide) hIgG heavy chain signal sequence/ scFV 3-17I/hIgG1 hinge/CD226 TMD/CD226 ICD v1/CD3zeta
atggactggatctggagaattttgttccttgtcggtgctgccactggagcccactcgcaagtgcagctcgtgcagtctggagcagaagtcaagaagcctgggtcctcggtcaaagtgtcctgcaaagcctccgggggcactttcagctcgtacgcaatctcctgggtccgccaagcgcccggacagggtctggagtggatgggcgggattatccccattttcggcaccgccaactatgctcaaaagttccagggccgcgtgactattaccgcggacgagtcaacctccactgcgtacatggaactgagctcacttcggtccgaggacaccgccgtctattactgtgcgcggggactcctgtggaactactggggacaaggcaccctcgtgaccgtgtcctcgaagctgtccgggtcggccagcgcccccaagttggaagagggcgaattctccgaggcccgggtggaaatcgtgatgacccagtccccggccactctctccgtgtccccgggagaaagggcgaccctgtcatgccgggcctcgcaatccgtgtcatccaacctggcctggtaccagcagaagccaggacaggcgccccgcctgattatctacggcgccagcaccaccgcgagcggaattccggcccggttttccgcctccggttcgggaactgacttcacgctgactatcagcagcctgcaatcggaggacttcgccgtctactactgccagcagtataacaactggcccccggcttacacgtttggccagggaactaagctggagatcaaggagcccaaaagctgcgacaagacccacacttgcccaccttgtccggacccgaaggggggaaccgtgctcctgctgctgttcgtgatctccatcaccacaatcatcgtgatcttcctgaaccgccggcgaagacgcgaaagacgcgatctgttcaccgagtcatgggacacccagaaggcccctaacaactacagaagcccgatcagcaccagccagcctactaatcagtcgatggatgatacccgcgaggacatctacgtgaattacccaaccttctcccggcggccgaaaacccgcgtgcgcgtgaagttcagccggagcgctgatgcccctgcataccagcaggggcagaaccagctctacaacgaactgaaccttggacggcgggaggaatacgatgtgctggataagcgaagaggccgcgacccagaaatgggcgggaagcccagacgcaagaatcctcaggagggactgtacaacgagctccagaaagacaagatggccgaagcgtacagcgagatcggcatgaagggggaacggagaaggggaaagggccatgacggattgtaccagggcctgtcgaccgctaccaaagacacctacgacgccctccatatgcaagcactgccgccacgctgaacgcgt

SEQ ID NO: 118 - protein, 518 aa, Artificial Sequence
  hIgG heavy chain signal sequence/scFV GC33/ hIgG1 hinge/CD28 TMD/41BB/CD3zeta /CD226 ICD v1
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNRRRRRERRDLFTESWDTQKAPNNYRSPISTSQPTNQSMDDTREDIYVNYPTFSRRPKTRV

SEQ ID NO: 119 - protein, 518 aa, Artificial Sequence
  hIgG heavy chain signal sequence/scFV GC33/ hIgG1 hinge/CD28 TMD/41BB/CD226 ICD v1/CD3zeta
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIKRGRKKLLYIFKQPFMRPVQTTQEEDGCSCRFPEEEEGGCELNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 361 aa, Artificial Sequence
  hIgG heavy chain signal sequence/scFV GC33/ hIgG1 hinge/CD226 TMD/CD226 ICD v1
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKGGTVLLLLFVISITTIIVIFLNRRRRRERRDLFTESWDTQKAPNNYRSPISTSQPTNQSMDDTREDIYVNYPTFSRRPKTRV

SEQ ID NO: 121 - protein, 499 aa, Artificial Sequence
  scFV GC33/hIgG1 hinge/CD28 TMD/41BB/CD3zeta / CD226 ICD v1
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNRRRRRERRDLFTESWDTQKAPNNYRSPISTSQPTNQSMDDTREDIYVNYPTFSRRPKTRV

SEQ ID NO: 122 - protein, 499 aa, Artificial Sequence
  scFV GC33/hIgG1 hinge/CD28 TMD/41BB/CD226 ICD v1/CD3zeta
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIKRGRKKLLYIFKQPFMRPVQTTQEEDGCSCRFPEEEEGGCELNRRRRRERRDLFTESWDTQKAPNNYRSPISTSQPTNQSMDDTREDIYVNYPTFSRRPKTRVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 342 aa, Artificial Sequence
  scFV GC33/hIgG1 hinge/CD226 TMD/CD226 ICD v1
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKGGTVLLLLFVISITTIIVIFLNRRRRRERRDLFTESWDTQKAPNNYRSPISTSQPTNQSMDDTREDIYVNYPTFSRRPKTRV

SEQ ID NO: 124 - DNA, 1554 bp, Artificial Sequence
  (nucleotide) hIgG heavy chain signal sequence/ scFV GC33/hIgG1 hinge/CD28 TMD/41BB/CD3zeta /CD226 ICD v1
atggattggatttggcgcattctgtttctggtgggcgcggcgaccggcgcgcatagccaggtgcagctgcagcagagcggcgcggaactggtgcgcccgggcgcgagcgtgaaactgagctgcaaagcgagcggctatacctttaccgattatgaaatgcattgggtgaaacagaccccggtgcatggcctgaaatggattggcgcgctggatccgaaaaccggcgataccgcgtatagccagaaatttaaaggcaaagcgaccctgaccgcggataaaagcagcagcaccgcgtatatggaactgcgcagcctgaccagcgaagatagcgcggtgtattattgcacccgcttttatagctatacctattggggccagggcaccctggtgaccgtgagcgcgggcggcggcggcagcggcggcggcggcagcggcggcggcggcagcgatgtggtgatgacccagaccccgctgagcctgccggtgagcctgggcgatcaggcgagcattagctgccgcagcagccagagcctggtgcatagcaacggcaacacctatctgcattggtatctgcagaaaccgggccagagcccgaaactgctgatttataaagtgagcaaccgctttagcggcgtgccggatcgctttagcggcagcggcagcggcaccgattttaccctgaaaattagccgcgtggaagcggaagatctgggcgtgtatttttgcagccagaacacccatgtgccgccgacctttggcagcggcaccaaactggaaattaaagaaccgaaaagctgcgataaaacccatacctgcccgccgtgcccggatccgaaattttgggtgctggtggtggtgggcggcgtgctggcgtgctatagcctgctggtgaccgtggcgtttattattaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgcaaccgccgccgccgccgcgaacgccgcgatctgtttaccgaaagctgggatacccagaaagcgccgaacaactatcgcagcccgattagcaccagccagccgaccaaccagagcatggatgatacccgcgaagatatttatgtgaactatccgacctttagccgccgcccgaaaacccgcgtg

SEQ ID NO: 125 - DNA, 1554 bp, Artificial Sequence
  (nucleotide) hIgG heavy chain signal sequence/ scFV GC33/hIgG1 hinge/CD28 TMD/41BB/CD226 ICD v1/CD3zeta
atggattggatttggcgcattctgtttctggtgggcgcggcgaccggcgcgcatagccaggtgcagctgcagcagagcggcgcggaactggtgcgcccgggcgcgagcgtgaaactgagctgcaaagcgagcggctatacctttaccgattatgaaatgcattgggtgaaacagaccccggtgcatggcctgaaatggattggcgcgctggatccgaaaaccggcgataccgcgtatagccagaaatttaaaggcaaagcgaccctgaccgcggataaaagcagcagcaccgcgtatatggaactgcgcagcctgaccagcgaagatagcgcggtgtattattgcacccgcttttatagctatacctattggggccagggcaccctggtgaccgtgagcgcgggcggcggcggcagcggcggcggcggcagcggcggcggcggcagcgatgtggtgatgacccagaccccgctgagcctgccggtgagcctgggcgatcaggcgagcattagctgccgcagcagccagagcctggtgcatagcaacggcaacacctatctgcattggtatctgcagaaaccgggccagagcccgaaactgctgatttataaagtgagcaaccgctttagcggcgtgccggatcgctttagcggcagcggcagcggcaccgattttaccctgaaaattagccgcgtggaagcggaagatctgggcgtgtatttttgcagccagaacacccatgtgccgccgacctttggcagcggcaccaaactggaaattaaagaaccgaaaagctgcgataaaacccatacctgcccgccgtgcccggatccgaaattttgggtgctggtggtggtgggcggcgtgctggcgtgctatagcctgctggtgaccgtggcgtttattattaaacgcggccgcaaaaaactgctgtatatttttaaacagccgtttatgcgcccggtgcagaccacccaggaagaagatggctgcagctgccgctttccggaagaagaagaaggcggctgcgaactgaaccgccgccgccgccgcgaacgccgcgatctgtttaccgaaagctgggatacccagaaagcgccgaacaactatcgcagcccgattagcaccagccagccgaccaaccagagcatggatgatacccgcgaagatatttatgtgaactatccgacctttagccgccgcccgaaaacccgcgtgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 126 - DNA, 1083 bp, Artificial Sequence
  (nucleotide) hIgG heavy chain signal sequence/ scFV GC33/hIgG1 hinge/CD226 TMD/CD226 ICD v1
atggattggatttggcgcattctgtttctggtgggcgcggcgaccggcgcgcatagccaggtgcagctgcagcagagcggcgcggaactggtgcgcccgggcgcgagcgtgaaactgagctgcaaagcgagcggctatacctttaccgattatgaaatgcattgggtgaaacagaccccggtgcatggcctgaaatggattggcgcgctggatccgaaaaccggcgataccgcgtatagccagaaatttaaaggcaaagcgaccctgaccgcggataaaagcagcagcaccgcgtatatggaactgcgcagcctgaccagcgaagatagcgcggtgtattattgcacccgcttttatagctatacctattggggccagggcaccctggtgaccgtgagcgcgggcggcggcggcagcggcggcggcggcagcggcggcggcggcagcgatgtggtgatgacccagaccccgctgagcctgccggtgagcctgggcgatcaggcgagcattagctgccgcagcagccagagcctggtgcatagcaacggcaacacctatctgcattggtatctgcagaaaccgggccagagcccgaaactgctgatttataaagtgagcaaccgctttagcggcgtgccggatcgctttagcggcagcggcagcggcaccgattttaccctgaaaattagccgcgtggaagcggaagatctgggcgtgtatttttgcagccagaacacccatgtgccgccgacctttggcagcggcaccaaactggaaattaaagaaccgaaaagctgcgataaaacccatacctgcccgccgtgcccggatccgaaaggcggcaccgtgctgctgctgctgtttgtgattagcattaccaccattattgtgatttttctgaaccgccgccgccgccgcgaacgccgcgatctgtttaccgaaagctgggatacccagaaagcgccgaacaactatcgcagcccgattagcaccagccagccgaccaaccagagcatggatgatacccgcgaagatatttatgtgaactatccgacctttagccgccgcccgaaaacccgcgtg